(19) United States
(12) Patent Application Publication
(10) Pub. No.: US 2025/0341526 A1
(43) Pub. Date: Nov. 6, 2025

(54) NOVEL DYES

(71) Applicant: Quantum-Si Incorporated, Branford, CT (US)

(72) Inventors: Matthew T. Richers, Clinton, CT (US); Haidong Huang, Madison, CT (US)

(73) Assignee: Quantum-Si Incorporated, Branford, CT (US)

(21) Appl. No.: 19/009,829

(22) Filed: Jan. 3, 2025

Related U.S. Application Data
(60) Provisional application No. 63/618,050, filed on Jan. 5, 2024.

(51) Int. Cl. G01N 33/68; C09B 57/00; C09K 11/06; G01N 33/58

(52) U.S. Cl. CPC G01N 33/6818; C09B 57/00; C09K 11/06; G01N 33/582; C09K 2211/1007; C09K 2211/1018

(57) ABSTRACT

Provided herein are compounds of Formula (I). Also provided herein are amino acid recognition molecules, and salts thereof, comprising at least one instance of Formula (II), and compositions thereof. Further provided herein are methods of sequencing a polypeptide using an amino acid recognition molecule, or salt thereof, comprising at least one instance of Formulae (II) or (IV).

### CROSS-REFERENCE TO RELATED APPLICATIONS

The present application claims the benefit of priority of U.S. Provisional Application No. 63/618,050, filed Jan. 5, 2024, the entire content of which is incorporated herein by reference.

### REFERENCE TO AN ELECTRONIC SEQUENCE LISTING

The contents of the electronic sequence listing (R070870168US01-SEQ-WLC.xml; Size: 227,666 bytes; and Date of Creation: Jan. 3, 2025) are herein incorporated by reference in its entirety.

### BACKGROUND OF THE INVENTION

Luminescent labels are often used in systems and methods for detecting and/or characterizing biological analytes. Some of these systems and methods involve monitoring a biological reaction in real time using a plurality of types of luminescently labeled reaction components. In order to identify specific types of luminescently labeled reaction components, it is important that each type of reaction component be labeled with a luminescent label having readily differentiable luminescent properties. However, the sensitivity of complex biological processes requires careful consideration when designing luminescent labels for use in these systems and methods.

Boron dipyrromethane (BODIPY) dyes are versatile and widely used chromophores for labeling nucleotides, amino acids, and other substrates. However, some BODIPY dyes are limited by their thermal instability (e.g., degradation in solution over time), photochemical instability (e.g., photobleaching in solution), and/or photophysical instability (e.g., photoblinking). BODIPY dyes with long spacers (e.g., 12 atoms) between the dye and conjugated moiety can lead to unwanted dye-dye interactions (e.g., quenching) when more than one equivalent of the dye is conjugated to a single biomolecule. Additionally, it is challenging to use multiple types of dyes in a single system, where the spectral properties of the dyes must be sufficiently distinguishable. There is a need for alternative BODIPY dyes that overcome the disadvantages associated with current dyes. However, the syntheses of such BODIPY dyes are often limited by low chemical yields associated with known synthesis methods.

### SUMMARY OF THE INVENTION

The present disclosure provides novel, improved boron dipyrromethene (BODIPY) dyes of Formula (I). Compounds of Formula (I) have improved thermal and photochemical stability compared to other dyes and are therefore more suitable for use in biomolecular labeling methods.

Accordingly, in one aspect, provided herein is a compound of Formula (I):

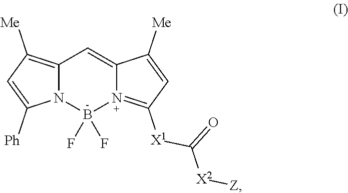

or a salt thereof, wherein X¹, X², and Z are defined herein.

In another aspect, provided herein is an amino acid recognition molecule, or a salt thereof, comprising at least one instance of Formula (II):

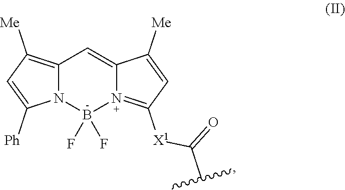

or a salt thereof, wherein X¹ is defined herein.

In another aspect, provided herein is a composition comprising an amino acid recognition molecule, or a salt thereof, comprising at least one instance of Formula (II):

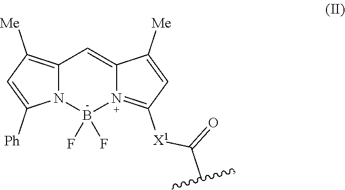

or a salt thereof, wherein X¹ is defined herein. In some embodiments, the composition further comprises a triplet quencher of Formula (V):

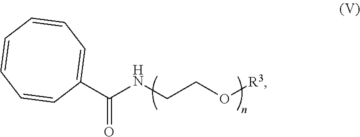

or a salt thereof, wherein R³ and n are defined herein.

In another aspect, provided herein is a method of sequencing a polypeptide, the method comprising:
(i) directing a series of pulses of one or more excitation energies towards a composition comprising a polypeptide, and an amino acid recognition molecule, or a salt thereof, wherein the amino acid recognition molecule, or salt thereof, comprises at least one instance of Formula (II):

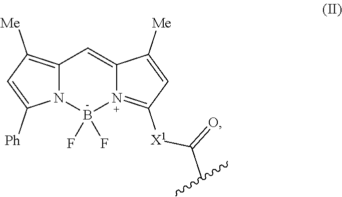

or a salt thereof, wherein X¹ is defined herein;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

In another aspect, provided herein is a method of sequencing a polypeptide, the method comprising:
(i) directing a series of pulses of one or more excitation energies towards a composition comprising a polypeptide, and an amino acid recognition molecule, or a salt thereof, wherein the amino acid recognition molecule, or salt thereof, comprises at least one instance of Formula (IV):

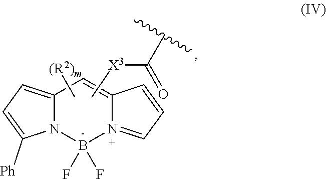

or a salt thereof, wherein R², X³, m, and [symbol] are defined herein;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

In another aspect, provided herein is a system for performing a method of sequencing a polypeptide.

The details of certain embodiments of the disclosure are set forth in the Detailed Description of Certain Embodiments, as described below. Other features, objects, and advantages of the disclosure will be apparent from the Definitions, Examples, and Claims.

### BRIEF DESCRIPTION OF THE DRAWINGS

FIG. 1 shows a recognition run of BDP R6G (PS610-tris-BDP R6G). Thermal instability leads to three intensity levels of pulses and three clusters, each representing a binder with a different amount of intact dye. Bin ratio is good, and pulses are stable. 10 mM COT-PEG3-sulfonate used as triplet state quencher.

FIG. 2 shows a recognition run of BDP2114. BDP2114 exhibited unstable pulsing with intensities going up and down, which may be a sign of photoblinking. This leads to diffuse clustering. BDP2114 may also be undergoing photobleaching. 10 mM COT-PEG3-sulfonate used as triplet state quencher.

FIG. 3 shows a recognition run of BDP528. BDP528 produced pulses that did not appear to suffer from photoblinking. Photobleaching can be seen as stepwise decreases in intensity within a pulse, leading to the formation of subclusters in the plot. 10 mM COT-PEG3-sulfonate used as triplet state quencher.

FIGS. 4A-4C show a series of five novel dyes (FIG. 4A), and the synthesis of BDP2156 (FIG. 4B) and BDP3037 (FIG. 4C). Yields are unoptimized.

FIGS. 5A-5B show thermal stability of BODIPY-mPEG5 conjugates in 1×PBS (5% DMSO) at 37° C. (FIG. 5A) and 65° C. (FIG. 5B).

FIG. 6 shows a recognition run of BDP3037 (PS610-tris-BDP3037). BDP3037 shows better clustering than BDP R6G due to increased thermal stability, and a good bin ratio (0.68-0.69). 10 mM COT-PEG3-sulfonate used as triplet state quencher.

FIG. 7 shows a recognition run of BDP2156 (PS610-tris-BDP2156). BDP2156 shows better clustering than BDP R6G due to increase thermal stability, and a good bin ratio (0.68-0.69). 10 mM COT-PEG3-sulfonate used as triplet state quencher.

FIG. 8 shows multiplex recognition runs of dyes. Multiplexed recognition runs of PS610-tris-BDP2156 and PS610-tris-BDP3037 with current five-dye set show six well-defined clusters. BDP3037 shows higher relative intensity compared to BDP2156. 10 mM COT-PEG3-sulfonate used as triplet state quencher.

FIG. 9 shows dynamic sequencing with BDP3037. PS610-bis-BDP3037 is sufficiently bright for sequencing and is differentiable in intensity from tris-AttoRho6G. Peptide: QP1088 DQFRLAGGK(N3). Binders: 2-BDP3037-PS610, 4-Cy3B-PS1220, 3-ATRho6G-PS961. Cutters: AP30/AP37.

FIG. 10 shows the structures and synthesis of triplet-state quenchers (TSQs) used in the present disclosure.

FIGS. 11A-11B show the persistence of cyclooctatetraene (FIG. 11A) and COT-PEG3-sulfonate (triethylammonium salt) (FIG. 11B) in solution. COT-PEG3-sulfonate (triethylammonium salt) was more stable in solution that the parent COT compound, even in the presence of mineral oil.

FIG. 12 shows a recognition run of BDP3037 (PS610-tris-BDP3037) with Trolox TSQ. Pulses are noisy, and intensity and bin ratio are low. 5 mM Trolox was used as triplet state quencher.

FIG. 13 shows a recognition run of BDP3037 (PS610-tris-BDP3037) with COT-PEG3-sulfonate (triethylammonium salt) TSQ in place of Trolox. Clean pulses and improved intensity and bin ratio are observed. 10 mM COT-PEG3-sulfonate (triethylammonium salt) was used as triplet state quencher.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C_{3- 5}, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁_7 alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

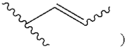

may be in the (E)- or (Z)-configuration.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}—, —C(═NR^{bb})OR^{aa}—, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(Rb)₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R′)₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃+X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, =NR^{bb}, or =NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{a}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{a}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{a}, —OC(═NR^{bb})N(R^{bb})₂, —OS(═O)R^{aa}, —OSO₂R^{aa}, —OSi(R^{a})₃, —OP(R^{aa})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and R^{cc} are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR—, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR—, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R′)₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{a}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″—[N-(imidazolylmethyl)]trityl Ether (IDTr-OR), 4,4′-Dimethoxy-3′″—[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

These and other exemplary substituents are described in more detail in the Detailed Description, Examples, and Claims. The present disclosure is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

As used herein, the term “about X,” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Amino acid residues may be indicated by their corresponding single letter codes, e.g., R (arginine), H (histidine), K (lysine), D (aspartic acid), E (glutamic acid), S (serine), T (threonine), N (asparagine), Q (glutamine), C (cysteine), G (glycine), P (proline), A (alanine), V (valine), I (isoleucine), L (leucine), M (methionine), F (phenylalanine), Y (tyrosine), W (tryptophan).

A “peptidase,” “protease,” or “proteinase” is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. An exopeptidase in accordance with the application may be an “aminopeptidase” or a “carboxypeptidase,” which cleaves a single amino acid from an amino- or a carboxy-terminus, respectively. A peptidase (e.g., an aminopeptidase) may also be referred to as a “cutter” or a “cleaving reagent.”

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, provided herein is a compound of Formula (I):

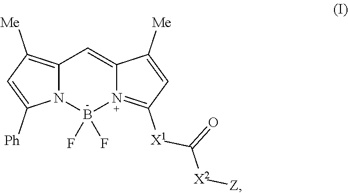

or a salt thereof, wherein:
X¹ is substituted or unsubstituted C₁-C₆ alkylene;
X² is a bond, —O—, or —N(R¹)—;
R¹ is hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, or substituted or unsubstituted heteroaryl; and
Z is hydrogen, halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a polypeptide, or a polynucleotide moiety.

As generally described herein, X¹ is substituted or unsubstituted C₁-C₆ alkylene.

In some embodiments, X¹ is substituted C₁-C₆ alkylene, substituted C₁-C₅ alkylene, substituted C₁-C₄ alkylene, substituted C₁-C₃ alkylene, or substituted C₁-C₂ alkylene. In some embodiments, X¹ is substituted C₁-C₆ alkylene. In some embodiments, X¹ is substituted C₁-C₅ alkylene. In some embodiments, X¹ is substituted C₁-C₄ alkylene. In some embodiments, X¹ is substituted C₁-C₃ alkylene. In some embodiments, X¹ is substituted C₁-C₂ alkylene.

In some embodiments, X¹ is C₁-C₆ alkylene, C₁-C₅ alkylene, C₁-C₄ alkylene, C₁-C₃ alkylene, or C₁-C₂ alkylene substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂; wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring.

In some embodiments, X¹ is unsubstituted C₁-C₆ alkylene, unsubstituted C₁-C₅ alkylene, unsubstituted C₁-C₄ alkylene, unsubstituted C₁-C₃ alkylene, or unsubstituted C₁-C₂ alkylene. In some embodiments, X¹ is unsubstituted C₁-C₆ alkylene or unsubstituted C₁-C₃ alkylene. In some embodiments, X¹ is unsubstituted C₁-C₆ alkylene. In some embodiments, X¹ is unsubstituted C₁-C₅ alkylene. In some embodiments, X¹ is unsubstituted C₁-C₄ alkylene. In some embodiments, X¹ is unsubstituted C₁-C₃ alkylene. In some embodiments, X¹ is unsubstituted C₁-C₂ alkylene.

In some embodiments, X¹ is methylene, ethylene, n-propylene, isopropylene, n-butylene, tert-butylene, sec-butylene, isobutylene, n-pentylene, 3-pentanylene, amylene, neopentylene, 3-methyl-2-butanylene, tert-amylene, or n-hexylene.

In some embodiments, X¹ is methylene (—CH₂—), ethylene (—(CH₂)₂—), n-propylene (—(CH₂)₃—), n-butylene (—(CH₂)₄—), n-pentylene (—(CH₂)₅—), or n-hexylene (—(CH₂)₆—). In some embodiments, X¹ is —CH₂—, —(CH₂)₂—, or —(CH₂)₃—. In some embodiments, X¹ is —(CH₂)₂—.

In some embodiments, the compound of Formula (I) is of Formula (I-a):

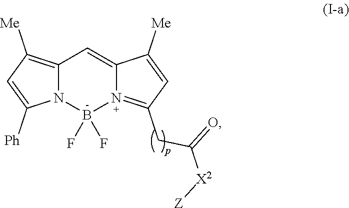

or a salt thereof, wherein p is 1, 2, 3, 4, 5, or 6. In some embodiments of Formula (I-a), p is 1. In some embodiments of Formula (I-a), p is 2. In some embodiments of Formula (I-a), p is 3. In some embodiments of Formula (I-a), p is 4. In some embodiments of Formula (I-a), p is 5. In some embodiments of Formula (I-a), p is 6.

In some embodiments, the compound of Formula (I) is of Formula (I-b):

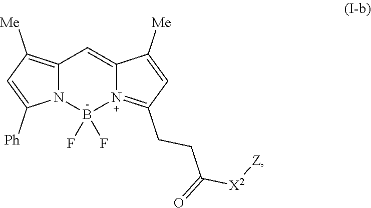

or a salt thereof.

As generally described herein, X² is a bond, —O—, or —N(R¹)—.

In some embodiments, X² is a bond.

In some embodiments, X² is —O—.

In some embodiments, the compound of Formula (I) is of Formula (I-c):

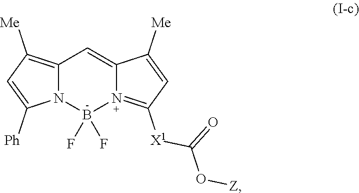

or a salt thereof.

In some embodiments, the compound of Formula (I) is of Formula (I-d):

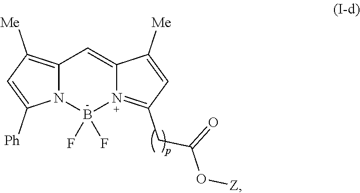

or a salt thereof, wherein p is 1, 2, 3, 4, 5, or 6. In some embodiments of Formula (I-d), p is 1. In some embodiments of Formula (I-d), p is 2. In some embodiments of Formula (I-d), p is 3. In some embodiments of Formula (I-d), p is 4. In some embodiments of Formula (I-d), p is 5. In some embodiments of Formula (I-d), p is 6.

In some embodiments, the compound of Formula (I) is of Formula (I-e):

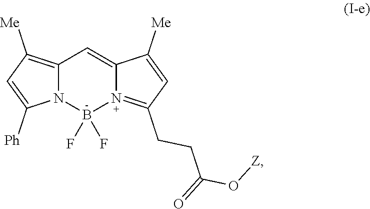

or a salt thereof.

In some embodiments, X² is —N(R¹)—. In some embodiments, X² is —NH—, —N(substituted or unsubstituted aliphatic)-, —N(substituted or unsubstituted heteroaliphatic)-, —N(substituted or unsubstituted carbocyclyl)-, —N(substituted or unsubstituted heterocyclyl)-, —N(substituted or unsubstituted aryl)-, or —N(substituted or unsubstituted heteroaryl)-. In some embodiments, X² is —NH— or —N(substituted or unsubstituted aliphatic)-.

In some embodiments, X² is —NH—. In some embodiments, X² is —N(substituted or unsubstituted aliphatic)-. In some embodiments, X² is —N(substituted or unsubstituted alkyl)-, —N(substituted or unsubstituted alkenyl)-, or —N(substituted or unsubstituted alkynyl)-. In some embodiments, X² is —N(substituted or unsubstituted alkyl)-. In some embodiments, X² is —N(substituted or unsubstituted C₁-C₆ alkyl)-. In some embodiments, X² is —N(substituted or unsubstituted C₁-C₃ alkyl)-.

In some embodiments, X² is —N(substituted aliphatic)-. In some embodiments, X² is —N(substituted alkyl)-, —N(substituted alkenyl)-, or —N(substituted alkynyl)-. In some embodiments, X² is —N(substituted alkyl)-. In some embodiments, X² is —N(substituted C₁-C₆ alkyl)-. In some embodiments, X² is —N(substituted C₁-C₃ alkyl)-.

In some embodiments, X² is —N(unsubstituted aliphatic)-. In some embodiments, X² is —N(unsubstituted alkyl)-, —N(unsubstituted alkenyl)-, or —N(unsubstituted alkynyl)-. In some embodiments, X² is —N(unsubstituted alkyl)-. In some embodiments, X² is —N(unsubstituted C₁-C₆ alkyl)-. In some embodiments, X² is —N(unsubstituted C₁-C₃ alkyl)-. In some embodiments, X² is —N(CH₃)—, —N(CH₂CH₃)—, —N(CH₂CH₂CH₃)—, or —N(CH(CH₃)₂)—.

As generally described herein, R¹ is hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, or substituted or unsubstituted heteroaryl.

In some embodiments, R¹ is hydrogen or substituted or unsubstituted aliphatic.

In some embodiments, R¹ is hydrogen.

In some embodiments, R¹ is substituted or unsubstituted aliphatic. In some embodiments, R¹ is substituted or unsubstituted alkyl, substituted or unsubstituted alkenyl, or substituted or unsubstituted alkynyl.

In some embodiments, R¹ is substituted or unsubstituted alkyl. In some embodiments, R¹ is substituted or unsubstituted C₁-C₁₂ alkyl. In some embodiments, R¹ is substituted or unsubstituted C₁-C₆ alkyl. In some embodiments, R¹ is substituted or unsubstituted C₁-C₃ alkyl. In some embodiments, R¹ is substituted alkyl. In some embodiments, R¹ is substituted C₁-C₁₂ alkyl. In some embodiments, R¹ is substituted C₁-C₆ alkyl. In some embodiments, R¹ is substituted C₁-C₃ alkyl. In some embodiments, R¹ is acyl. In some embodiments, R¹ is unsubstituted alkyl. In some embodiments, R¹ is unsubstituted C₁-C₁₂ alkyl. In some embodiments, R¹ is unsubstituted C₁-C₆ alkyl. In some embodiments, R¹ is unsubstituted C₁-C₃ alkyl.

In some embodiments, R¹ is substituted or unsubstituted alkenyl. In some embodiments, R¹ is substituted or unsubstituted C₁-C₁₂ alkenyl. In some embodiments, R¹ is substituted or unsubstituted C₁-C₆ alkenyl. In some embodiments, R¹ is substituted or unsubstituted C₁-C₃ alkenyl. In some embodiments, R¹ is substituted alkenyl. In some embodiments, R¹ is substituted C₁-C₁₂ alkenyl. In some embodiments, R¹ is substituted C₁-C₆ alkenyl. In some embodiments, R¹ is substituted C₁-C₃ alkenyl. In some embodiments, R¹ is unsubstituted alkenyl. In some embodiments, R¹ is unsubstituted C₁-C₁₂ alkenyl. In some embodiments, R¹ is unsubstituted C₁-C₆ alkenyl. In some embodiments, R¹ is unsubstituted C₁-C₃ alkenyl.

In some embodiments, R¹ is substituted or unsubstituted alkynyl. In some embodiments, R¹ is substituted or unsubstituted C₁-C₁₂ alkynyl. In some embodiments, R¹ is substituted or unsubstituted C₁-C₆ alkynyl. In some embodiments, R¹ is substituted or unsubstituted C₁-C₃ alkynyl. In some embodiments, R¹ is substituted alkynyl. In some embodiments, R¹ is substituted C₁-C₁₂ alkynyl. In some embodiments, R¹ is substituted C₁-C₆ alkynyl. In some embodiments, R¹ is substituted C₁-C₃ alkynyl. In some embodiments, R¹ is unsubstituted alkynyl. In some embodiments, R¹ is unsubstituted C₁-C₁₂ alkynyl. In some embodiments, R¹ is unsubstituted C₁-C₆ alkynyl. In some embodiments, R¹ is unsubstituted C₁-C₃ alkynyl.

In some embodiments, R¹ is substituted or unsubstituted heteroaliphatic. In some embodiments, R¹ is substituted or unsubstituted heteroalkyl. In some embodiments, R¹ is substituted or unsubstituted C₁-C₁₂ heteroalkyl. In some embodiments, R¹ is substituted or unsubstituted C₁-C₆ heteroalkyl. In some embodiments, R¹ is substituted or unsubstituted C₁-C₃ heteroalkyl. In some embodiments, R¹ is substituted heteroalkyl. In some embodiments, R¹ is substituted C₁-C₁₂ heteroalkyl. In some embodiments, R¹ is substituted C₁-C₆ heteroalkyl. In some embodiments, R¹ is substituted C₁-C₃ heteroalkyl. In some embodiments, R¹ is unsubstituted heteroalkyl. In some embodiments, R¹ is unsubstituted C₁-C₁₂ heteroalkyl. In some embodiments, R¹ is unsubstituted C₁-C₆ heteroalkyl. In some embodiments, R¹ is unsubstituted C₁-C₃ heteroalkyl.

In some embodiments, R¹ is substituted or unsubstituted carbocyclyl. In some embodiments, R¹ is substituted or unsubstituted C₃-C₁₀ carbocyclyl. In some embodiments, R¹ is substituted or unsubstituted C₃-C₆ carbocyclyl. In some embodiments, R¹ is substituted carbocyclyl. In some embodiments, R¹ is substituted C₃-C₁₀ carbocyclyl. In some embodiments, R¹ is substituted C₃-C₆ carbocyclyl. In some embodiments, R¹ is unsubstituted carbocyclyl. In some embodiments, R¹ is unsubstituted C₃-C₁₀ carbocyclyl. In some embodiments, R¹ is unsubstituted C₃-C₆ carbocyclyl.

In some embodiments, R¹ is substituted or unsubstituted heterocyclyl. In some embodiments, R¹ is substituted or unsubstituted 3-10 membered heterocyclyl. In some embodiments, R¹ is substituted or unsubstituted 3-6 membered heterocyclyl. In some embodiments, R¹ is substituted heterocyclyl. In some embodiments, R¹ is substituted 3-10 membered heterocyclyl. In some embodiments, R¹ is substituted 3-6 membered heterocyclyl. In some embodiments, R¹ is unsubstituted heterocyclyl. In some embodiments, R¹ is unsubstituted 3-10 membered heterocyclyl. In some embodiments, R¹ is unsubstituted 3-6 membered heterocyclyl.

In some embodiments, R¹ is substituted or unsubstituted aryl. In some embodiments, R¹ is substituted or unsubstituted phenyl. In some embodiments, R¹ is substituted aryl. In some embodiments, R¹ is substituted phenyl. In some embodiments, R¹ is unsubstituted aryl. In some embodiments, R¹ is unsubstituted phenyl.

In some embodiments, R¹ is substituted or unsubstituted heteroaryl. In some embodiments, R¹ is substituted or unsubstituted 5-10 membered heteroaryl. In some embodiments, R¹ is substituted or unsubstituted 5-6 membered monocyclic heteroaryl. In some embodiments, R¹ is substituted heteroaryl. In some embodiments, R¹ is substituted 5-10 membered heteroaryl. In some embodiments, R¹ is substituted 5-6 membered monocyclic heteroaryl. In some embodiments, R¹ is unsubstituted heteroaryl. In some embodiments, R¹ is unsubstituted 5-10 membered heteroaryl. In some embodiments, R¹ is unsubstituted 5-6 membered monocyclic heteroaryl.

As generally described herein, Z is hydrogen, halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a polypeptide, or a polynucleotide moiety.

In some embodiments, Z is hydrogen, substituted or unsubstituted heterocyclyl, a polypeptide, or a polynucleotide.

In some embodiments, Z is hydrogen.

In some embodiments, X² is —O—, and Z is hydrogen. In some embodiments, X²—Z is —OH.

In some embodiments, the compound of Formula (I) is of Formula (I-f):

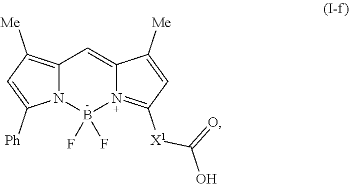

or a salt thereof.

In some embodiments, the compound of Formula (I) is of Formula (I-g):

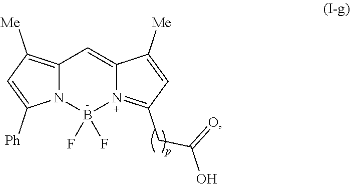

or a salt thereof, wherein p is 1, 2, 3, 4, 5, or 6. In some embodiments of Formula (I-g), p is 1. In some embodiments of Formula (I-g), p is 2. In some embodiments of Formula (I-g), p is 3. In some embodiments of Formula (I-g), p is 4. In some embodiments of Formula (I-g), p is 5. In some embodiments of Formula (I-g), p is 6.

In some embodiments, the compound of Formula (I) is of formula:

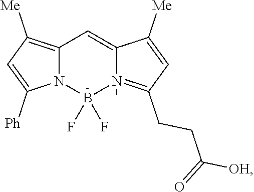

or a salt thereof.

In some embodiments, Z is halogen. In some embodiments, Z is —F, —Cl, or —Br.

In some embodiments, Z is substituted or unsubstituted aliphatic. In some embodiments, Z is substituted or unsubstituted alkyl, substituted or unsubstituted alkenyl, or substituted or unsubstituted alkynyl.

In some embodiments, Z is substituted or unsubstituted alkyl. In some embodiments, Z is substituted or unsubstituted C₁-C₁₂ alkyl. In some embodiments, Z is substituted or unsubstituted C₁-C₆ alkyl. In some embodiments, Z is substituted or unsubstituted C₁-C₃ alkyl. In some embodiments, Z is substituted alkyl. In some embodiments, Z is substituted C₁-C₁₂ alkyl. In some embodiments, Z is substituted C₁-C₆ alkyl. In some embodiments, Z is substituted C₁-C₃ alkyl. In some embodiments, Z is acyl. In some embodiments, Z is unsubstituted alkyl. In some embodiments, Z is unsubstituted C₁-C₁₂ alkyl. In some embodiments, Z is unsubstituted C₁-C₆ alkyl. In some embodiments, Z is unsubstituted C₁-C₃ alkyl.

In some embodiments, Z is substituted or unsubstituted alkenyl. In some embodiments, Z is substituted or unsubstituted C₁-C₁₂ alkenyl. In some embodiments, Z is substituted or unsubstituted C₁-C₆ alkenyl. In some embodiments, Z is substituted or unsubstituted C₁-C₃ alkenyl. In some embodiments, Z is substituted alkenyl. In some embodiments, Z is substituted C₁-C₁₂ alkenyl. In some embodiments, Z is substituted C₁-C₆ alkenyl. In some embodiments, Z is substituted C₁-C₃ alkenyl. In some embodiments, Z is unsubstituted alkenyl. In some embodiments, Z is unsubstituted C₁-C₁₂ alkenyl. In some embodiments, Z is unsubstituted C₁-C₆ alkenyl. In some embodiments, Z is unsubstituted C₁-C₃ alkenyl.

In some embodiments, Z is substituted or unsubstituted alkynyl. In some embodiments, Z is substituted or unsubstituted C₁-C₁₂ alkynyl. In some embodiments, Z is substituted or unsubstituted C₁-C₆ alkynyl. In some embodiments, Z is substituted or unsubstituted C₁-C₃ alkynyl. In some embodiments, Z is substituted alkynyl. In some embodiments, Z is substituted C₁-C₁₂ alkynyl. In some embodiments, Z is substituted C₁-C₆ alkynyl. In some embodiments, Z is substituted C₁-C₃ alkynyl. In some embodiments, Z is unsubstituted alkynyl. In some embodiments, Z is unsubstituted C₁-C₁₂ alkynyl. In some embodiments, Z is unsubstituted C₁-C₆ alkynyl. In some embodiments, Z is unsubstituted C₁-C₃ alkynyl.

In some embodiments, Z is substituted or unsubstituted heteroaliphatic. In some embodiments, Z is substituted or unsubstituted heteroalkyl. In some embodiments, Z is substituted or unsubstituted C₁-C₁₂ heteroalkyl. In some embodiments, Z is substituted or unsubstituted C₁-C₆ heteroalkyl. In some embodiments, Z is substituted or unsubstituted C₁-C₃ heteroalkyl. In some embodiments, Z is substituted heteroalkyl. In some embodiments, Z is substituted C₁-C₁₂ heteroalkyl. In some embodiments, Z is substituted C₁-C₆ heteroalkyl. In some embodiments, Z is substituted C₁-C₃ heteroalkyl. In some embodiments, Z is unsubstituted heteroalkyl. In some embodiments, Z is unsubstituted C₁-C₁₂ heteroalkyl. In some embodiments, Z is unsubstituted C₁-C₆ heteroalkyl. In some embodiments, Z is unsubstituted C₁-C₃ heteroalkyl.

In some embodiments, Z is substituted or unsubstituted carbocyclyl. In some embodiments, Z is substituted or unsubstituted C₃-C₁₀ carbocyclyl. In some embodiments, Z is substituted or unsubstituted C₃-C₆ carbocyclyl. In some embodiments, Z is substituted carbocyclyl. In some embodiments, Z is substituted C₃-C₁₀ carbocyclyl. In some embodiments, Z is substituted C₃-C₆ carbocyclyl. In some embodiments, Z is unsubstituted carbocyclyl. In some embodiments, Z is unsubstituted C₃-C₁₀ carbocyclyl. In some embodiments, Z is unsubstituted C₃-C₆ carbocyclyl.

In some embodiments, Z is substituted or unsubstituted heterocyclyl. In some embodiments, Z is substituted or unsubstituted 3-10 membered heterocyclyl. In some embodiments, Z is substituted or unsubstituted 3-6 membered heterocyclyl. In some embodiments, Z is substituted or unsubstituted heterocyclyl containing 1 ring N atom. In some embodiments, Z is substituted or unsubstituted 3-10 membered heterocyclyl containing 1 ring N atom. In some embodiments, Z is substituted or unsubstituted 3-6 membered heterocyclyl containing 1 ring N atom.

In some embodiments, Z is substituted heterocyclyl. In some embodiments, Z is substituted 3-10 membered heterocyclyl. In some embodiments, Z is substituted 3-6 membered heterocyclyl. In some embodiments, Z is substituted heterocyclyl containing 1 ring N atom. In some embodiments, Z is substituted 3-10 membered heterocyclyl containing 1 ring N atom. In some embodiments, Z is substituted 3-6 membered heterocyclyl containing 1 ring N atom.

In some embodiments, Z is heterocyclyl substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂₀R^{A}, —OSi(R^{A})(OR^{A})₂, —OSi(OR^{A})₃, and/or —B(OR^{A})₂; wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring. In some embodiments, Z is heterocyclyl substituted with ═O. In some embodiments, Z is 3-10 membered heterocyclyl substituted with ═O. In some embodiments, Z is 3-6 membered heterocyclyl substituted with ═O. In some embodiments, Z is heterocyclyl containing 1 ring N atom, wherein the heterocyclyl is substituted with ═O. In some embodiments, Z is 3-10 membered heterocyclyl containing 1 ring N atom, wherein the heterocyclyl is substituted with ═O. In some embodiments, Z is 3-6 membered heterocyclyl containing 1 ring N atom, wherein the heterocyclyl is substituted with ═O. In some embodiments, Z is

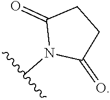 [SMILES: *N1C(=O)CCC1=O]

In some embodiments, X² is —O—, and Z is

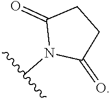 [SMILES: *N1C(=O)CCC1=O]

In some embodiments, X²—Z is

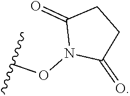 [SMILES: *ON1C(=O)CCC1=O]

In some embodiments, the compound of Formula (I) is of Formula (I-h):

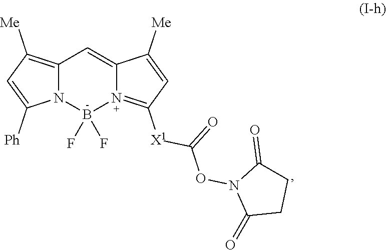

or a salt thereof.

In some embodiments, the compound of Formula (I) is of Formula (I-i):

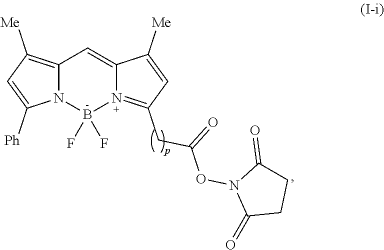

or a salt thereof, wherein p is 1, 2, 3, 4, 5, or 6. In some embodiments of Formula (I-i), p is 1. In some embodiments of Formula (I-i), p is 2. In some embodiments of Formula (I-i), p is 3. In some embodiments of Formula (I-i), p is 4. In some embodiments of Formula (I-i), p is 5. In some embodiments of Formula (I-i), p is 6.

In some embodiments, the compound of Formula (I) is of formula:

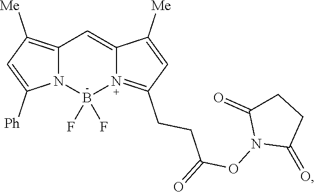

or a salt thereof.

In some embodiments, Z is unsubstituted heterocyclyl. In some embodiments, Z is unsubstituted 3-10 membered heterocyclyl. In some embodiments, Z is unsubstituted 3-6 membered heterocyclyl. In some embodiments, Z is unsubstituted heterocyclyl containing 1 ring N atom. In some embodiments, Z is unsubstituted 3-10 membered heterocyclyl containing 1 ring N atom. In some embodiments, Z is unsubstituted 3-6 membered heterocyclyl containing 1 ring N atom.

In some embodiments, Z is substituted or unsubstituted aryl. In some embodiments, Z is substituted or unsubstituted phenyl. In some embodiments, Z is substituted aryl. In some embodiments, Z is substituted phenyl. In some embodiments, Z is unsubstituted aryl. In some embodiments, Z is unsubstituted phenyl.

In some embodiments, Z is substituted or unsubstituted heteroaryl. In some embodiments, Z is substituted or unsubstituted 5-10 membered heteroaryl. In some embodiments, Z is substituted or unsubstituted 5-6 membered monocyclic heteroaryl. In some embodiments, Z is substituted heteroaryl. In some embodiments, Z is substituted 5-10 membered heteroaryl. In some embodiments, Z is substituted 5-6 membered monocyclic heteroaryl. In some embodiments, Z is unsubstituted heteroaryl. In some embodiments, Z is unsubstituted 5-10 membered heteroaryl. In some embodiments, Z is unsubstituted 5-6 membered monocyclic heteroaryl.

In some embodiments, Z is a polypeptide. In some embodiments, Z is a polypeptide further comprising at least one substituent. In some embodiments, the polypeptide is further substituted. In some embodiments, the polypeptide further comprises at least one substituent. In some embodiments, Z is a polypeptide further comprising a substituent of formula:

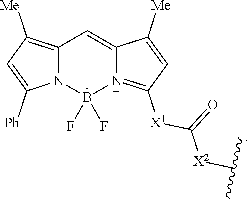

In some embodiments, the polypeptide further comprises a substituent of formula:

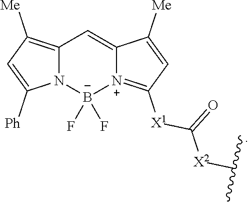

In some embodiments, Z comprises a sequence GGGSGGGSGGGSG (“Linker 1”) (SEQ ID NO: 214); GSAGSAAGSGEF (“Linker 2”) (SEQ ID NO: 215); or GSAGSAAGSGEFGSAGSAAGSGEFGSAGSAAGSGEF (“Linker 3”) (SEQ ID NO: 216). In some embodiments, Z comprises the sequence Linker 1. In some embodiments, Z comprises the sequence Linker 2. In some embodiments, Z comprises the sequence Linker 3. In some embodiments, Z is a polypeptide shown in Table 1, Table 2, or Table 3. In some embodiments, Z is a polypeptide shown in Table 3. In some embodiments, Z is PS610 (Bis-atClpS2-V1, Linker 2). In some embodiments, Z is PS2132.

In some embodiments, Z is a polynucleotide. In some embodiments, Z is a polynucleotide further comprising at least one substituent. In some embodiments, the polynucleotide is further substituted. In some embodiments, the polynucleotide further comprises at least one substituent. In some embodiments, Z is a polynucleotide further comprising a substituent of formula:

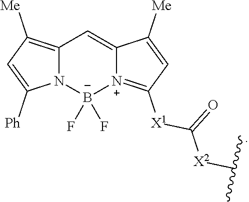

In some embodiments, the polynucleotide further comprises a substituent of formula:

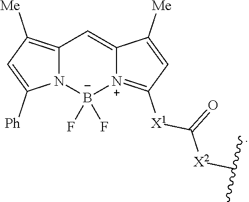

In some embodiments, the compound of Formula (I) is of Formula (I-j):

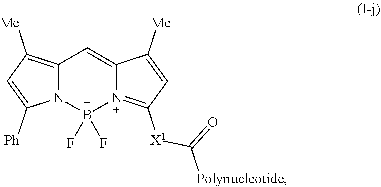

or a salt thereof.

In some embodiments, the compound of Formula (I) is of Formula (I-k):

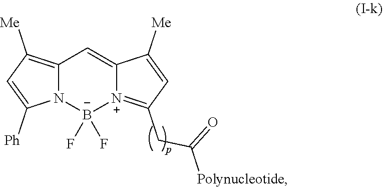

or a salt thereof, wherein p is 1, 2, 3, 4, 5, or 6. In some embodiments of Formula (I-k), p is 1. In some embodiments of Formula (I-k), p is 2. In some embodiments of Formula (I-k), p is 3. In some embodiments of Formula (I-k), p is 4. In some embodiments of Formula (I-k), p is 5. In some embodiments of Formula (I-k), p is 6.

In some embodiments, the compound of Formula (I) is of Formula (I-m):

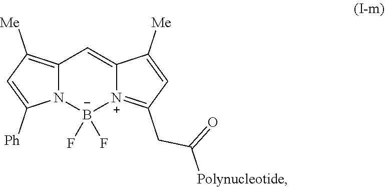

or a salt thereof.

### Amino Acid Recognition Molecules and Compositions

In another aspect, provided herein is an amino acid recognition molecule, or a salt thereof, comprising at least one instance of Formula (II):

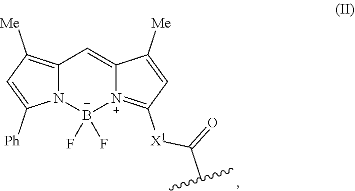

or a salt thereof, wherein:
each instance of X¹ is substituted or unsubstituted C₁-C₆ alkylene.

As generally described herein, each instance of X¹ is substituted or unsubstituted C₁-C₆ alkylene.

In some embodiments, at least one instance of X¹ is substituted C₁-C₆ alkylene, substituted C₁-C₅ alkylene, substituted C₁-C₄ alkylene, substituted C₁-C₃ alkylene, or substituted C₁-C₂ alkylene. In some embodiments, at least one instance of X¹ is substituted C₁-C₆ alkylene. In some embodiments, at least one instance of X¹ is substituted C₁-C₅ alkylene. In some embodiments, at least one instance of X¹ is substituted C₁-C₄ alkylene. In some embodiments, at least one instance of X¹ is substituted C₁-C₃ alkylene. In some embodiments, at least one instance of X¹ is substituted C₁-C₂ alkylene.

In some embodiments, at least one instance of X¹ is C₁-C₆ alkylene, C₁-C₅ alkylene, C₁-C₄ alkylene, C₁-C₃ alkylene, or C₁-C₂ alkylene substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂; wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring.

In some embodiments, at least one instance of X¹ is unsubstituted C₁-C₆ alkylene, unsubstituted C₁-C₅ alkylene, unsubstituted C₁-C₄ alkylene, unsubstituted C₁-C₃ alkylene, or unsubstituted C₁-C₂ alkylene. In some embodiments, at least one instance of X¹ is unsubstituted C₁-C₆ alkylene or unsubstituted C₁-C₃ alkylene. In some embodiments, at least one instance of X¹ is unsubstituted C₁-C₆ alkylene. In some embodiments, at least one instance of X¹ is unsubstituted C₁-C₅ alkylene. In some embodiments, at least one instance of X¹ is unsubstituted C₁-C₄ alkylene. In some embodiments, at least one instance of X¹ is unsubstituted C₁-C₃ alkylene. In some embodiments, at least one instance of X¹ is unsubstituted C₁-C₂ alkylene.

In some embodiments, at least one instance of X¹ is methylene, ethylene, n-propylene, isopropylene, n-butylene, tert-butylene, sec-butylene, isobutylene, n-pentylene, 3-pentanylene, amylene, neopentylene, 3-methyl-2-butanylene, tert-amylene, or n-hexylene.

In some embodiments, at least one instance of X¹ is methylene (—CH₂—), ethylene (—(CH₂)₂—), n-propylene (—(CH₂)₃—), n-butylene (—(CH₂)₄—), n-pentylene (—(CH₂)₅—), or n-hexylene (—(CH₂)₆—). In some embodiments, at least one instance of X¹ is —CH₂—, —(CH₂)₂—, or —(CH₂)₃—. In some embodiments, at least one instance of X¹ is —(CH₂)₂—.

In some embodiments, the amino acid recognition molecule, or salt thereof, comprises at least one instance of Formula (II), wherein the at least one instance of Formula (II) is of Formula (II-a):

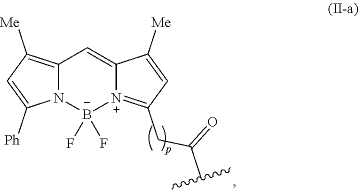

or a salt thereof, wherein each instance of p is independently 1, 2, 3, 4, 5, or 6. In some embodiments of Formula (II-a), at least one instance of p is 1. In some embodiments of Formula (II-a), at least one instance of p is 2. In some embodiments of Formula (II-a), at least one instance of p is 3. In some embodiments of Formula (II-a), at least one instance of p is 4. In some embodiments of Formula (II-a), at least one instance of p is 5. In some embodiments of Formula (II-a), at least one instance of p is 6.

In some embodiments, the amino acid recognition molecule, or salt thereof, comprises at least one instance of Formula (II), wherein the at least one instance of Formula (II) is of formula:

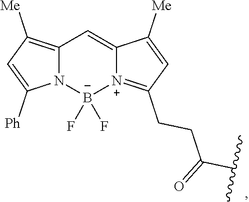 [SMILES: *C(=O)CCC1=[N+]2C(=Cc3c(C)cc(-c4ccccc4)n3[B-]2(F)F)C(C)=C1]

or a salt thereof.

In some embodiments, the amino acid recognition molecule, or salt thereof, is linked to at least one instance of Formula (II), or salt thereof, via a linker. In some embodiments, the linker comprises one or more moieties selected from substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, and polynucleotide. In some embodiments, the linker comprises one or more moieties selected from substituted or unsubstituted alkylene, substituted or unsubstituted alkenylene, or substituted or unsubstituted alkynylene, substituted or unsubstituted heteroalkylene, substituted or unsubstituted carbocyclylene, substituted or unsubstituted heterocyclylene, substituted or unsubstituted arylene, substituted or unsubstituted heteroarylene, and polynucleotide.

In some embodiments, the linker comprises substituted or unsubstituted aliphatic. In some embodiments, the linker comprises substituted or unsubstituted alkylene, substituted or unsubstituted alkenylene, or substituted or unsubstituted alkynylene.

In some embodiments, the linker comprises substituted or unsubstituted alkylene. In some embodiments, the linker comprises substituted or unsubstituted C₁-C₁₂ alkylene. In some embodiments, the linker comprises substituted or unsubstituted C₁-C₆ alkylene. In some embodiments, the linker comprises substituted or unsubstituted C₁-C₃ alkylene. In some embodiments, the linker comprises substituted alkylene. In some embodiments, the linker comprises substituted C₁-C₁₂ alkylene. In some embodiments, the linker comprises substituted C₁-C₆ alkylene. In some embodiments, the linker comprises substituted C₁-C₃ alkylene. In some embodiments, the linker comprises acylene. In some embodiments, the linker comprises unsubstituted alkylene. In some embodiments, the linker comprises unsubstituted C₁-C₁₂ alkylene. In some embodiments, the linker comprises unsubstituted C₁-C₆ alkylene. In some embodiments, the linker comprises unsubstituted C₁-C₃ alkylene.

In some embodiments, the linker comprises substituted or unsubstituted alkenylene. In some embodiments, the linker comprises substituted or unsubstituted C₁-C₁₂ alkenylene. In some embodiments, the linker comprises substituted or unsubstituted C₁-C₆ alkenylene. In some embodiments, the linker comprises substituted or unsubstituted C₁-C₃ alkenylene. In some embodiments, the linker comprises substituted alkenylene. In some embodiments, the linker comprises substituted C₁-C₁₂ alkenylene. In some embodiments, the linker comprises substituted C₁-C₆ alkenylene. In some embodiments, the linker comprises substituted C₁-C₃ alkenylene. In some embodiments, the linker comprises unsubstituted alkenylene. In some embodiments, the linker comprises unsubstituted C₁-C₁₂ alkenylene. In some embodiments, the linker comprises unsubstituted C₁-C₆ alkenylene. In some embodiments, the linker comprises unsubstituted C₁-C₃ alkenylene.

In some embodiments, the linker comprises substituted or unsubstituted alkynylene. In some embodiments, the linker comprises substituted or unsubstituted C₁-C₁₂ alkynylene. In some embodiments, the linker comprises substituted or unsubstituted C₁-C₆ alkynylene. In some embodiments, the linker comprises substituted or unsubstituted C₁-C₃ alkynylene. In some embodiments, the linker comprises substituted alkynylene. In some embodiments, the linker comprises substituted C₁-C₁₂ alkynylene. In some embodiments, the linker comprises substituted C₁-C₆ alkynylene. In some embodiments, the linker comprises substituted C₁-C₃ alkynylene. In some embodiments, the linker comprises unsubstituted alkynylene. In some embodiments, the linker comprises unsubstituted C₁-C₁₂ alkynylene. In some embodiments, the linker comprises unsubstituted C₁-C₆ alkynylene. In some embodiments, the linker comprises unsubstituted C₁-C₃ alkynylene.

In some embodiments, the linker comprises substituted or unsubstituted heteroaliphatic. In some embodiments, the linker comprises substituted or unsubstituted heteroalkylene. In some embodiments, the linker comprises substituted or unsubstituted C₁-C₁₂ heteroalkylene. In some embodiments, the linker comprises substituted or unsubstituted C₁-C₆ heteroalkylene. In some embodiments, the linker comprises substituted or unsubstituted C₁-C₃ heteroalkylene. In some embodiments, the linker comprises substituted heteroalkylene. In some embodiments, the linker comprises substituted C₁-C₁₂ heteroalkylene. In some embodiments, the linker comprises substituted C₁-C₆ heteroalkylene. In some embodiments, the linker comprises substituted C₁-C₃ heteroalkylene. In some embodiments, the linker comprises unsubstituted heteroalkylene. In some embodiments, the linker comprises unsubstituted C₁-C₁₂ heteroalkylene. In some embodiments, the linker comprises unsubstituted C₁-C₆ heteroalkylene. In some embodiments, the linker comprises unsubstituted C₁-C₃ heteroalkylene.

In some embodiments, the linker comprises substituted or unsubstituted carbocyclylene. In some embodiments, the linker comprises substituted or unsubstituted C₃-C₁₀ carbocyclylene. In some embodiments, the linker comprises substituted or unsubstituted C₃-C₆ carbocyclylene. In some embodiments, the linker comprises substituted carbocyclylene. In some embodiments, the linker comprises substituted C₃-C₁₀ carbocyclylene. In some embodiments, the linker comprises substituted C₃-C₆ carbocyclylene. In some embodiments, the linker comprises unsubstituted carbocyclylene. In some embodiments, the linker comprises unsubstituted C₃-C₁₀ carbocyclylene. In some embodiments, the linker comprises unsubstituted C₃-C₆ carbocyclylene.

In some embodiments, the linker comprises substituted or unsubstituted heterocyclylene. In some embodiments, the linker comprises substituted or unsubstituted 3-10 membered heterocyclylene. In some embodiments, the linker comprises substituted or unsubstituted 3-6 membered heterocyclylene. In some embodiments, the linker comprises substituted heterocyclylene. In some embodiments, the linker comprises substituted 3-10 membered heterocyclylene. In some embodiments, the linker comprises substituted 3-6 membered heterocyclylene. In some embodiments, the linker comprises unsubstituted heterocyclylene. In some embodiments, the linker comprises unsubstituted 3-10 membered heterocyclylene. In some embodiments, the linker comprises unsubstituted 3-6 membered heterocyclylene.

In some embodiments, the linker comprises substituted or unsubstituted arylene. In some embodiments, the linker comprises substituted or unsubstituted phenylene. In some embodiments, the linker comprises substituted arylene. In some embodiments, the linker comprises substituted phenylene. In some embodiments, the linker comprises unsubstituted arylene. In some embodiments, the linker comprises unsubstituted phenylene.

In some embodiments, the linker comprises substituted or unsubstituted heteroarylene. In some embodiments, the linker comprises substituted or unsubstituted 5-10 membered heteroarylene. In some embodiments, the linker comprises substituted or unsubstituted 5-6 membered monocyclic heteroarylene. In some embodiments, the linker comprises substituted heteroarylene. In some embodiments, the linker comprises substituted 5-10 membered heteroarylene. In some embodiments, the linker comprises substituted 5-6 membered monocyclic heteroarylene. In some embodiments, the linker comprises unsubstituted heteroarylene. In some embodiments, the linker comprises unsubstituted 5-10 membered heteroarylene. In some embodiments, the linker comprises unsubstituted 5-6 membered monocyclic heteroarylene.

In some embodiments, the linker comprises a polynucleotide. In some embodiments, the linker comprises a polynucleotide further comprising at least one substituent. In some embodiments, the polynucleotide is further substituted. In some embodiments, the polynucleotide further comprises at least one substituent.

In some embodiments, the amino acid recognition molecule, or salt thereof, comprises at least 1, 2, 3, 4, or 5 instances of Formula (II), or a salt thereof. In some embodiments, the amino acid recognition molecule, or salt thereof, comprises at least 1 instance of Formula (II), or a salt thereof. In some embodiments, the amino acid recognition molecule, or salt thereof, comprises at least 2 instances of Formula (II), or a salt thereof. In some embodiments, the amino acid recognition molecule, or salt thereof, comprises at least 3 instances of Formula (II), or a salt thereof. In some embodiments, the amino acid recognition molecule, or salt thereof, comprises at least 4 instances of Formula (II), or a salt thereof. In some embodiments, the amino acid recognition molecule, or salt thereof, comprises at least 5 instances of Formula (II), or a salt thereof. In some embodiments, the amino acid recognition molecule, or salt thereof, comprises 1 instance of Formula (II), or a salt thereof. In some embodiments, the amino acid recognition molecule, or salt thereof, comprises 2 instances of Formula (II), or a salt thereof. In some embodiments, the amino acid recognition molecule, or salt thereof, comprises 3 instances of Formula (II), or a salt thereof. In some embodiments, the amino acid recognition molecule, or salt thereof, comprises 4 instances of Formula (II), or a salt thereof. In some embodiments, the amino acid recognition molecule, or salt thereof, comprises 5 instances of Formula (II), or a salt thereof.

In some embodiments, at least one instance of Formula (II), or a salt thereof, is thermally stable. In some embodiments, thermal stability comprises at least one instance of Formula (II), or a salt thereof, maintaining at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% of its maximum fluorescence when the amino acid recognition molecule, or salt thereof, is maintained at a temperature of about 15° C. to about 65° C. for a time of at least about 5 minutes. In some embodiments, thermal stability comprises at least one instance of Formula (II), or a salt thereof, maintaining at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% of its maximum fluorescence when the amino acid recognition molecule, or salt thereof, is maintained at a temperature of about 15° C. to about 65° C. for a time of at least about 5 minutes.

In some embodiments, thermal stability comprises at least one instance of Formula (II), or a salt thereof, maintaining at least about 50% of its maximum fluorescence when the amino acid recognition molecule, or salt thereof, is maintained at a temperature of about 15° C. to about 65° C. for a time of at least about 5 minutes. In some embodiments, thermal stability comprises at least one instance of Formula (II), or a salt thereof, maintaining at least about 60% of its maximum fluorescence when the amino acid recognition molecule, or salt thereof, is maintained at a temperature of about 15° C. to about 65° C. for a time of at least about 5 minutes. In some embodiments, thermal stability comprises at least one instance of Formula (II), or a salt thereof, maintaining at least about 70% of its maximum fluorescence when the amino acid recognition molecule, or salt thereof, is maintained at a temperature of about 15° C. to about 65° C. for a time of at least about 5 minutes. In some embodiments, thermal stability comprises at least one instance of Formula (II), or a salt thereof, maintaining at least about 80% of its maximum fluorescence when the amino acid recognition molecule, or salt thereof, is maintained at a temperature of about 15° C. to about 65° C. for a time of at least about 5 minutes. In some embodiments, thermal stability comprises at least one instance of Formula (II), or a salt thereof, maintaining at least about 90% of its maximum fluorescence when the amino acid recognition molecule, or salt thereof, is maintained at a temperature of about 15° C. to about 65° C. for a time of at least about 5 minutes. In some embodiments, thermal stability comprises at least one instance of Formula (II), or a salt thereof, maintaining at least about 100% of its maximum fluorescence when the amino acid recognition molecule, or salt thereof, is maintained at a temperature of about 15° C. to about 65° C. for a time of at least about 5 minutes.

In some embodiments, at least one instance of Formula (II), or a salt thereof, maintains at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% of its maximum fluorescence when the amino acid recognition molecule, or salt thereof, is maintained at a temperature of about 15° C. to about 65° C. for a time of at least about 5 minutes. In some embodiments, at least one instance of Formula (II), or a salt thereof, maintains at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% of its maximum fluorescence when the amino acid recognition molecule, or salt thereof, is maintained at a temperature of about 15° C. to about 65° C. for a time of at least about 5 minutes.

In some embodiments, at least one instance of Formula (II), or a salt thereof, maintains at least about 50% of its maximum fluorescence when the amino acid recognition molecule, or salt thereof, is maintained at a temperature of about 15° C. to about 65° C. for a time of at least about 5 minutes. In some embodiments, at least one instance of Formula (II), or a salt thereof, maintains at least about 60% of its maximum fluorescence when the amino acid recognition molecule, or salt thereof, is maintained at a temperature of about 15° C. to about 65° C. for a time of at least about 5 minutes. In some embodiments, at least one instance of Formula (II), or a salt thereof, maintains at least about 70% of its maximum fluorescence when the amino acid recognition molecule, or salt thereof, is maintained at a temperature of about 15° C. to about 65° C. for a time of at least about 5 minutes. In some embodiments, at least one instance of Formula (II), or a salt thereof, maintains at least about 80% of its maximum fluorescence when the amino acid recognition molecule, or salt thereof, is maintained at a temperature of about 15° C. to about 65° C. for a time of at least about 5 minutes. In some embodiments, at least one instance of Formula (II), or a salt thereof, maintains at least about 90% of its maximum fluorescence when the amino acid recognition molecule, or salt thereof, is maintained at a temperature of about 15° C. to about 65° C. for a time of at least about 5 minutes. In some embodiments, at least one instance of Formula (II), or a salt thereof, maintains at least about 100% of its maximum fluorescence when the amino acid recognition molecule, or salt thereof, is maintained at a temperature of about 15° C. to about 65° C. for a time of at least about 5 minutes.

In some embodiments, at least one instance of Formula (II), or a salt thereof, maintains about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, or about 100% of its maximum fluorescence when the amino acid recognition molecule, or salt thereof, is maintained at a temperature of about 15° C. to about 65° C. for about 5 minutes. In some embodiments, at least one instance of Formula (II), or a salt thereof, maintains about 50%, about 55%, about 60%, about 65%, about 70%, about 75%, about 80%, about 85%, about 90%, about 95%, about 98%, about 99%, or about 100% of its maximum fluorescence when the amino acid recognition molecule, or salt thereof, is maintained at a temperature of about 15° C. to about 65° C. for about 5 minutes.

In some embodiments, at least one instance of Formula (II), or a salt thereof, maintains about 50% of its maximum fluorescence when the amino acid recognition molecule, or salt thereof, is maintained at a temperature of about 15° C. to about 65° C. for about 5 minutes. In some embodiments, at least one instance of Formula (II), or a salt thereof, maintains about 60% of its maximum fluorescence when the amino acid recognition molecule, or salt thereof, is maintained at a temperature of about 15° C. to about 65° C. for about 5 minutes. In some embodiments, at least one instance of Formula (II), or a salt thereof, maintains about 70% of its maximum fluorescence when the amino acid recognition molecule, or salt thereof, is maintained at a temperature of about 15° C. to about 65° C. for about 5 minutes. In some embodiments, at least one instance of Formula (II), or a salt thereof, maintains about 80% of its maximum fluorescence when the amino acid recognition molecule, or salt thereof, is maintained at a temperature of about 15° C. to about 65° C. for about 5 minutes. In some embodiments, at least one instance of Formula (II), or a salt thereof, maintains about 90% of its maximum fluorescence when the amino acid recognition molecule, or salt thereof, is maintained at a temperature of about 15° C. to about 65° C. for about 5 minutes. In some embodiments, at least one instance of Formula (II), or a salt thereof, maintains about 100% of its maximum fluorescence when the amino acid recognition molecule, or salt thereof, is maintained at a temperature of about 15° C. to about 65° C. for about 5 minutes.

In some embodiments, the temperature is between about 15° C. and about 65° C., about 20° C. and about 65° C., about 25° C. and about 65° C., about 30° C. and about 65° C., about 35° C. and about 65° C., about 40° C. and about 65° C., about 45° C. and about 65° C., about 50° C. and about 65° C., about 55° C. and about 65° C., or about 60° C. and about 65° C. In some embodiments, the temperature is about 37° C. In some embodiments, the temperature is about 65° C.

In some embodiments, the time is at least about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 1.5 hours, about 2 hours, about 2.5 hours, about 3 hours, about 3.5 hours, about 4 hours, about 4.5 hours, about 5 hours, about 6 hours, about 7 hours, about 8 hours, about 9 hours, or about 10 hours.

In some embodiments, the time is at least about 5 minutes. In some embodiments, the time is at least about 10 minutes. In some embodiments, the time is at least about 15 minutes. In some embodiments, the time is at least about 20 minutes. In some embodiments, the time is at least about 25 minutes. In some embodiments, the time is at least about 30 minutes. In some embodiments, the time is at least about 35 minutes. In some embodiments, the time is at least about 40 minutes. In some embodiments, the time is at least about 45 minutes. In some embodiments, the time is at least about 50 minutes. In some embodiments, the time is at least about 55 minutes. In some embodiments, the time is at least about 1 hour. In some embodiments, the time is at least about 1.5 hours. In some embodiments, the time is at least about 2 hours. In some embodiments, the time is at least about 2.5 hours. In some embodiments, the time is at least about 3 hours. In some embodiments, the time is at least about 3.5 hours. In some embodiments, the time is at least about 4 hours. In some embodiments, the time is at least about 4.5 hours. In some embodiments, the time is at least about 5 hours. In some embodiments, the time is at least about 6 hours. In some embodiments, the time is at least about 7 hours. In some embodiments, the time is at least about 8 hours. In some embodiments, the time is at least about 9 hours. In some embodiments, the time is at least about 10 hours.

In some embodiments, the compound of Formula (II) is PS2132-bis-BDP3037.

In another aspect, provided herein is a composition comprising an amino acid recognition molecule, or a salt thereof, comprising at least one instance of Formula (II):

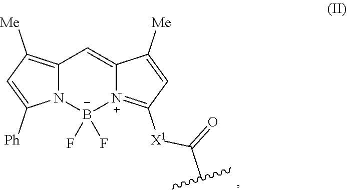

or a salt thereof, wherein:
each instance of X¹ is substituted or unsubstituted C₁-C₆ alkylene.

In some embodiments, the composition further comprises one or more amino acid recognition molecules, or salts thereof, conjugated to a dye, wherein the dye is not a 1,9-dimethyl-3-phenyl BODIPY dye. In some embodiments, the dye is a fluorophore. In some embodiments, the dye comprises an aromatic or heteroaromatic compound. In some embodiment, the dye comprises a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluorescein, rhodamine, xanthene, or other like compound.

In some embodiments, the dye is one or more dyes selected from: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor®405, Alexa Fluor®430, Alexa Fluor®480, Alexa Fluor®488, Alexa Fluor®514, Alexa Fluor®532, Alexa Fluor®546, Alexa Fluor®555, Alexa Fluor®568, Alexa Fluor®594, Alexa Fluor® 610-X, Alexa Fluor®633, Alexa Fluor®647, Alexa Fluor®660, Alexa Fluor®680, Alexa Fluor®700, Alexa Fluor®750, Alexa Fluor®790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R², DyLight® 543Q, DyLight® 550, DyLight® 554-R0, DyLight® 554-R¹, DyLight® 590-R², DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar®570, Quasar®670, Quasar®705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™633, Seta™650, Seta™660, Seta™670, Seta™680, Seta™700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7. In some embodiments, the dye is one or more dyes selected from: Janelia Fluor® 525, Janelia Fluor® 526, Janelia Fluor® 549, Janelia Fluor® 585, Janelia Fluor® 635, Janelia Fluor® 646, Janelia Fluor® 669, JFX549, JFX554, JFX646, JFX650, iFluor® 350, iFluor® 405, iFluor® 530, iFluor® 440, iFluor® 450, iFluor® 460, iFluor® 488, iFluor® 510, iFluor® 514, iFluor® 532, iFluor® 540, iFluor® 546, iFluor® 555, iFluor® 560, iFluor® 568, iFluor® 570, iFluor® 594, iFluor® 597, iFluor® 605, iFluor® 610, iFluor® 620, iFluor® 625, iFluor® 633, iFluor® 647, iFluor® 660, iFluor® 665, iFluor® 670, iFluor® 675, iFluor® 680, iFluor® 690, iFluor® 700, iFluor® 710, iFluor® 720, iFluor® 740, iFluor® 750, iFluor® A7, iFluor® 770, iFluor® 780, iFluor® 790, iFluor® 800, iFluor® 810, iFluor® 820, iFluor® 830, iFluor® 840, and iFluor® 860. In some embodiments, the dye is one or more dyes selected from 4-Cy3B, 4-SGCy3, C2C, 3-AttoRho6G, and R1C1. In some embodiments, the dye is one or more dyes selected from Cy®3, Cy®3B, and ATTO Rho6G.

In some embodiments, the composition further comprises a triplet quencher.

In some embodiments, the triplet quencher is a compound of Formula (V):

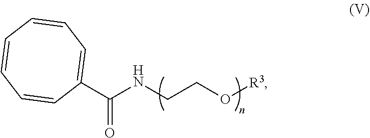

or a salt thereof, wherein:
R³ is substituted or unsubstituted aliphatic; and
n is 1, 2, 3, 4, 5, 6, 7, 8, 9, or 10.

As generally described herein, R³ is substituted or unsubstituted aliphatic. In some embodiments, R³ is substituted or unsubstituted alkyl, substituted or unsubstituted alkenyl, or substituted or unsubstituted alkynyl.

In some embodiments, R³ is substituted or unsubstituted alkyl. In some embodiments, R³ is substituted or unsubstituted C₁-C₁₂ alkyl, substituted or unsubstituted C₁-C₁₁ alkyl, substituted or unsubstituted C₁-C₁₀ alkyl, substituted or unsubstituted C₁-C₉ alkyl, substituted or unsubstituted C₁-C₈ alkyl, substituted or unsubstituted C₁-C₇ alkyl, substituted or unsubstituted C₁-C₆ alkyl, substituted or unsubstituted C₁-C₅ alkyl, substituted or unsubstituted C₁-C₄ alkyl, substituted or unsubstituted C₁-C₃ alkyl, or substituted or unsubstituted C₁-C₂ alkyl. In some embodiments, R³ is substituted or unsubstituted C₁-C₆ alkyl, substituted or unsubstituted C₁-C₅ alkyl, substituted or unsubstituted C₁-C₄ alkyl, substituted or unsubstituted C₁-C₃ alkyl, or substituted or unsubstituted C₁-C₂ alkyl.

In some embodiments, R³ is substituted or unsubstituted C₁-C₁₂ alkyl. In some embodiments, R³ is substituted or unsubstituted C₁-C₁₁ alkyl. In some embodiments, R³ is substituted or unsubstituted C₁-C₁₀ alkyl. In some embodiments, R³ is substituted or unsubstituted C₁-C₉ alkyl. In some embodiments, R³ is substituted or unsubstituted C₁-C₈ alkyl. In some embodiments, R³ is substituted or unsubstituted C₁-C₇ alkyl. In some embodiments, R³ is substituted or unsubstituted C₁-C₆ alkyl. In some embodiments, R³ is substituted or unsubstituted C₁-C₅ alkyl. In some embodiments, R³ is substituted or unsubstituted C₁-C₄ alkyl. In some embodiments, R³ is substituted or unsubstituted C₁-C₃ alkyl. In some embodiments, R³ is substituted or unsubstituted C₁-C₂ alkyl.

In some embodiments, R³ is substituted alkyl. In some embodiments, R³ is substituted C₁-C₁₂ alkyl, substituted C₁-C₁₁ alkyl, substituted C₁-C₁₀ alkyl, substituted C₁-C₉ alkyl, substituted C₁-C₈ alkyl, substituted C₁-C₇ alkyl, substituted C₁-C₆ alkyl, substituted C₁-C₅ alkyl, substituted C₁-C₄ alkyl, substituted C₁-C₃ alkyl, or substituted C₁-C₂ alkyl. In some embodiments, R³ is substituted C₁-C₆ alkyl, substituted C₁-C₅ alkyl, substituted C₁-C₄ alkyl, substituted C₁-C₃ alkyl, or substituted C₁-C₂ alkyl. In some embodiments, R³ is substituted C₁-C₆ alkyl, substituted C₁-C₅ alkyl, substituted C₁-C₄ alkyl, substituted C₁-C₃ alkyl, or substituted C₁-C₂ alkyl.

In some embodiments, R³ is substituted C₁-C₁₂ alkyl. In some embodiments, R³ is substituted C₁-C₁₁ alkyl. In some embodiments, R³ is substituted C₁-C₁₀ alkyl. In some embodiments, R³ is substituted C₁-C₉ alkyl. In some embodiments, R³ is substituted C₁-C₈ alkyl. In some embodiments, R³ is substituted C₁-C₇ alkyl. In some embodiments, R³ is substituted C₁-C₆ alkyl. In some embodiments, R³ is substituted C₁-C₅ alkyl. In some embodiments, R³ is substituted C₁-C₄ alkyl. In some embodiments, R³ is substituted C₁-C₃ alkyl. In some embodiments, R³ is substituted C₁-C₂ alkyl.

In some embodiments, R³ is C₁-C₆ alkyl, C₁-C₅ alkyl, C₁-C₄ alkyl, C₁-C₃ alkyl, or C₁-C₂ alkyl substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, —B(OR^{A})₂, and/or —N(R^{A})₃+, wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring. In some embodiments, R³ is C₁-C₆ alkyl, C₁-C₅ alkyl, C₁-C₄ alkyl, C₁-C₃ alkyl, or C₁-C₂ alkyl substituted with —S(═O)₂OR^{A} and/or —N(R^{A})₃⁺. In some embodiments, R³ is C₁-C₆ alkyl substituted with —S(═O)₂OR^{A} and/or —N(R^{A})₃⁺. In some embodiments, R³ is C₁-C₅ alkyl substituted with —S(═O)₂OR^{A} and/or —N(R^{A})₃⁺. In some embodiments, R³ is C₁-C₄ alkyl substituted with —S(═O)₂OR^{A} and/or —N(R^{A})₃⁺. In some embodiments, R³ is C₁-C₃ alkyl substituted with —S(═O)₂OR^{A} and/or —N(R^{A})₃⁺. In some embodiments, R³ is C₁-C₂ alkyl substituted with —S(═O)₂OR^{A} and/or —N(R^{A})₃⁺.

In some embodiments, R³ is of formula:

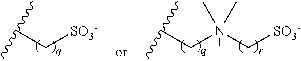

wherein: q is 1, 2, 3, 4, 5, or 6; and r is 1, 2, 3, 4, 5, or 6. In some embodiments, q is 1. In some embodiments, q is 2. In some embodiments, q is 3. In some embodiments, q is 4. In some embodiments, q is 5. In some embodiments, q is 6. In some embodiments, r is 1. In some embodiments, r is 2. In some embodiments, r is 3. In some embodiments, r is 4. In some embodiments, r is 5. In some embodiments, r is 6. In some embodiments, q is 2, and r is 3.

In some embodiments, R³ is

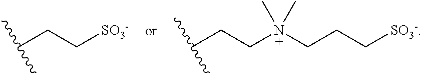

In some embodiments, R³ is unsubstituted alkyl. In some embodiments, R³ is unsubstituted C₁-C₁₂ alkyl, unsubstituted C₁-C₁₁ alkyl, unsubstituted C₁-C₁₀ alkyl, unsubstituted C₁-C₉ alkyl, unsubstituted C₁-C₈ alkyl, unsubstituted C₁-C₇ alkyl, unsubstituted C₁-C₆ alkyl, unsubstituted C₁-C₅ alkyl, unsubstituted C₁-C₄ alkyl, unsubstituted C₁-C₃ alkyl, or unsubstituted C₁-C₂ alkyl. In some embodiments, R³ is unsubstituted C₁-C₆ alkyl, unsubstituted C₁-C₅ alkyl, unsubstituted C₁-C₄ alkyl, unsubstituted C₁-C₃ alkyl, or unsubstituted C₁-C₂ alkyl.

In some embodiments, R³ is unsubstituted C₁-C₁₂ alkyl. In some embodiments, R³ is unsubstituted C₁-C₁₁ alkyl. In some embodiments, R³ is unsubstituted C₁-C₁₀ alkyl. In some embodiments, R³ is unsubstituted C₁-C₉ alkyl. In some embodiments, R³ is unsubstituted C₁-C₈ alkyl. In some embodiments, R³ is unsubstituted C₁-C₇ alkyl. In some embodiments, R³ is unsubstituted C₁-C₆ alkyl. In some embodiments, R³ is unsubstituted C₁-C₅ alkyl. In some embodiments, R³ is unsubstituted C₁-C₄ alkyl. In some embodiments, R³ is unsubstituted C₁-C₃ alkyl. In some embodiments, R³ is unsubstituted C₁-C₂ alkyl.

In some embodiments, R³ is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R³ is methyl, ethyl, n-propyl, n-butyl, n-pentyl, or n-hexyl. In some embodiments, R³ is methyl, ethyl, or n-propyl. In some embodiments, R³ is methyl (—CH₃).

In some embodiments, R³ is substituted or unsubstituted alkenyl. In some embodiments, R³ is substituted or unsubstituted C₁-C₁₂ alkenyl. In some embodiments, R³ is substituted or unsubstituted C₁-C₆ alkenyl. In some embodiments, R³ is substituted or unsubstituted C₁-C₃ alkenyl. In some embodiments, R³ is substituted alkenyl. In some embodiments, R³ is substituted C₁-C₁₂ alkenyl. In some embodiments, R³ is substituted C₁-C₆ alkenyl. In some embodiments, R³ is substituted C₁-C₃ alkenyl. In some embodiments, R³ is unsubstituted alkenyl. In some embodiments, R³ is unsubstituted C₁-C₁₂ alkenyl. In some embodiments, R³ is unsubstituted C₁-C₆ alkenyl. In some embodiments, R³ is unsubstituted C₁-C₃ alkenyl.

In some embodiments, R³ is substituted or unsubstituted alkynyl. In some embodiments, R³ is substituted or unsubstituted C₁-C₁₂ alkynyl. In some embodiments, R³ is substituted or unsubstituted C₁-C₆ alkynyl. In some embodiments, R³ is substituted or unsubstituted C₁-C₃ alkynyl. In some embodiments, R³ is substituted alkynyl. In some embodiments, R³ is substituted C₁-C₁₂ alkynyl. In some embodiments, R³ is substituted C₁-C₆ alkynyl. In some embodiments, R³ is substituted C₁-C₃ alkynyl. In some embodiments, R³ is unsubstituted alkynyl. In some embodiments, R³ is unsubstituted C₁-C₁₂ alkynyl. In some embodiments, R³ is unsubstituted C₁-C₆ alkynyl. In some embodiments, R³ is unsubstituted C₁-C₃ alkynyl.

As generally described herein, n is 1, 2, 3, 4, 5, 6, 7, 8, 9, or 10.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 1, 2, 3, 4, 5, 6, 7, 8, or 9. In some embodiments, n is 1, 2, 3, 4, 5, 6, 7, or 8. In some embodiments, n is 1, 2, 3, 4, 5, 6, or 7. In some embodiments, n is 1, 2, 3, 4, 5, or 6. In some embodiments, n is 1, 2, 3, 4, or 5. In some embodiments, n is 1, 2, 3, or 4. In some embodiments, n is 1, 2, or 3. In some embodiments, n is 1 or 2. In some embodiments, n is 1, 3, or 5.

In some embodiments, R³ is substituted or unsubstituted C₁-C₃ alkyl, and n is 1, 2, 3, 4, or 5. In some embodiments, R³ is substituted or unsubstituted C₁-C₃ alkyl, and n is 1. In some embodiments, R³ is substituted or unsubstituted C₁-C₃ alkyl, and n is 2. In some embodiments, R³ is substituted or unsubstituted C₁-C₃ alkyl, and n is 3. In some embodiments, R³ is substituted or unsubstituted C₁-C₃ alkyl, and n is 4. In some embodiments, R³ is substituted or unsubstituted C₁-C₃ alkyl, and n is 5.

In some embodiments, R³ is substituted C₁-C₃ alkyl, and n is 1, 2, 3, 4, or 5. In some embodiments, R³ is substituted C₁-C₃ alkyl, and n is 1. In some embodiments, R³ is substituted C₁-C₃ alkyl, and n is 2. In some embodiments, R³ is substituted C₁-C₃ alkyl, and n is 3. In some embodiments, R³ is substituted C₁-C₃ alkyl, and n is 4. In some embodiments, R³ is substituted C₁-C₃ alkyl, and n is 5.

In some embodiments, R³ is

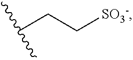

and n is 1, 2, 3, 4, or 5. In some embodiments, R³ is

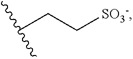

and n is 1. In some embodiments, R³ is

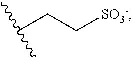

and n is 2. In some embodiments, R³ is

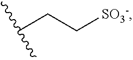

and n is 3. In some embodiments, R³ is

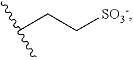

and n is 4. In some embodiments, R³ is

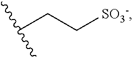

and n is 5.

In some embodiments, R³ is

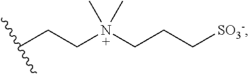

and n is 1, 2, 3, 4, or 5. In some embodiments, R³ is

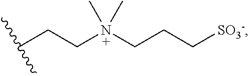

and n is 1. In some embodiments, R³ is

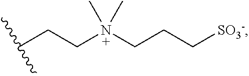

and n is 2. In some embodiments, R³ is

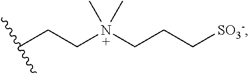

and n is 3. In some embodiments, R³ is

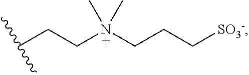

and n is 4. In some embodiments, R³ is

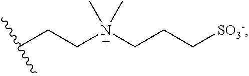

and n is 5.

In some embodiments, R³ is unsubstituted C₁-C₃ alkyl, and n is 1, 2, 3, 4, or 5. In some embodiments, R³ is unsubstituted C₁-C₃ alkyl, and n is 1. In some embodiments, R³ is unsubstituted C₁-C₃ alkyl, and n is 2. In some embodiments, R³ is unsubstituted C₁-C₃ alkyl, and n is 3. In some embodiments, R³ is unsubstituted C₁-C₃ alkyl, and n is 4. In some embodiments, R³ is unsubstituted C₁-C₃ alkyl, and n is 5.

In some embodiments, R³ is —CH₃, and n is 1, 2, 3, 4, or 5. In some embodiments, R³ is —CH₃, and n is 1. In some embodiments, R³ is —CH₃, and n is 2. In some embodiments, R³ is —CH₃, and n is 3. In some embodiments, R³ is —CH₃, and n is 4. In some embodiments, R³ is —CH₃, and n is 5.

In some embodiments, the triplet quencher is a compound of formula:

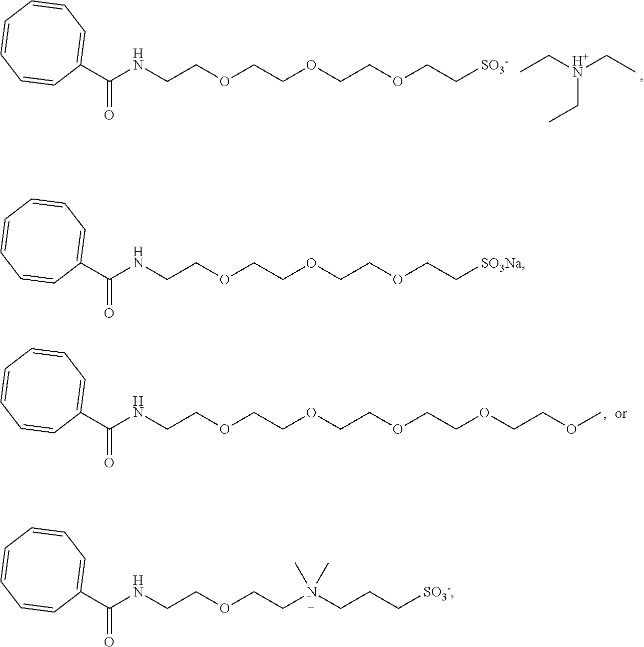

or a salt thereof.

In some embodiments, the amino acid recognition molecule, or salt thereof, has a molecular weight of between about 5 kDa and about 100 kDa, between about 5 kDa and about 95 kDa, between about 5 kDa and about 90 kDa, between about 5 kDa and about 85 kDa, between about 5 kDa and about 80 kDa, between about 5 kDa and about 75 kDa, between about 5 kDa and about 70 kDa, between about 5 kDa and about 65 kDa, between about 5 kDa and about 60 kDa, between about 5 kDa and about 55 kDa, between about 5 kDa and about 50 kDa, between about 5 kDa and about 45 kDa, between about 5 kDa and about 40 kDa, between about 5 kDa and about 35 kDa, between about 5 kDa and about 30 kDa, between about 5 kDa and about 25 kDa, between about 5 kDa and about 20 kDa, between about 5 kDa and about 15 kDa, or between about 5 kDa and about 10 kDa. In some embodiments, the amino acid recognition molecule, or salt thereof, has a molecular weight of between about 5 kDa and about 100 kDa. In some embodiments, the amino acid recognition molecule, or salt thereof, has a molecular weight of, at most, about 100 kDa.

In some embodiments, methods provided herein comprise contacting a polypeptide with an amino acid recognition molecule, which may or may not comprise a label, that selectively binds at least one type of terminal amino acid. As used herein, in some embodiments, a terminal amino acid may refer to an amino-terminal amino acid of a polypeptide or a carboxy-terminal amino acid of a polypeptide. In some embodiments, a labeled recognition molecule selectively binds one type of terminal amino acid over other types of terminal amino acids. In some embodiments, a labeled recognition molecule selectively binds one type of terminal amino acid over an internal amino acid of the same type. In yet other embodiments, a labeled recognition molecule selectively binds one type of amino acid at any position of a polypeptide, e.g., the same type of amino acid as a terminal amino acid and an internal amino acid.

As used herein, in some embodiments, the term “bond” or “bonds” refers to any non-covalent interaction (e.g., a hydrogen bond, a van der Waals interaction, an aromatic interaction, an electrostatic interaction) or covalent interaction between specified binding components or any plurality thereof, and the terms “bind,” “binding,” “bound,” and like terms refer to the formation and/or existence of any such bonds. As an illustrative example, a binding event between an amino acid recognizer and an amino acid may comprise the formation of one or more non-covalent or covalent interactions between the amino acid recognizer and the amino acid.

As used herein, in some embodiments, a type of amino acid refers to one of the twenty naturally occurring amino acids or a subset of types thereof. In some embodiments, a type of amino acid refers to a modified variant of one of the twenty naturally occurring amino acids or a subset of unmodified and/or modified variants thereof. Examples of modified amino acid variants include, without limitation, post-translationally-modified variants (e.g., acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination), chemically modified variants, unnatural amino acids, and proteinogenic amino acids such as selenocysteine and pyrrolysine. In some embodiments, a subset of types of amino acids includes more than one and fewer than twenty amino acids having one or more similar biochemical properties. For example, in some embodiments, a type of amino acid refers to one type selected from amino acids with charged side chains (e.g., positively and/or negatively charged side chains), amino acids with polar side chains (e.g., polar uncharged side chains), amino acids with nonpolar side chains (e.g., nonpolar aliphatic and/or aromatic side chains), and amino acids with hydrophobic side chains.

In some embodiments, methods provided herein comprise contacting a polypeptide with one or more labeled recognition molecules that selectively bind one or more types of terminal amino acids. As an illustrative and non-limiting example, where four labeled recognition molecules are used in a method of the disclosure, any one recognition molecule selectively binds one type of terminal amino acid that is different from another type of amino acid to which any of the other three selectively binds (e.g., a first recognition molecule binds a first type, a second recognition molecule binds a second type, a third recognition molecule binds a third type, and a fourth recognition molecule binds a fourth type of terminal amino acid). For the purposes of this discussion, one or more labeled recognition molecules in the context of a method described herein may be alternatively referred to as a set of labeled recognition molecules.

In some embodiments, a set of labeled recognition molecules comprises at least one and up to six labeled recognition molecules. For example, in some embodiments, a set of labeled recognition molecules comprises one, two, three, four, five, or six labeled recognition molecules. In some embodiments, a set of labeled recognition molecules comprises ten or fewer labeled recognition molecules. In some embodiments, a set of labeled recognition molecules comprises eight or fewer labeled recognition molecules. In some embodiments, a set of labeled recognition molecules comprises six or fewer labeled recognition molecules. In some embodiments, a set of labeled recognition molecules comprises four or fewer labeled recognition molecules. In some embodiments, a set of labeled recognition molecules comprises three or fewer labeled recognition molecules. In some embodiments, a set of labeled recognition molecules comprises two or fewer labeled recognition molecules. In some embodiments, a set of labeled recognition molecules comprises four labeled recognition molecules. In some embodiments, a set of labeled recognition molecules comprises at least two and up to twenty (e.g., at least two and up to ten, at least two and up to eight, at least four and up to twenty, at least four and up to ten) labeled recognition molecules. In some embodiments, a set of labeled recognition molecules comprises more than twenty (e.g., 20 to 25, 20 to 30) recognition molecules. It should be appreciated, however, that any number of recognition molecules may be used in accordance with a method of the disclosure to accommodate a desired use.

In accordance with the disclosure, in some embodiments, one or more types of amino acids are identified by detecting luminescence of a labeled recognition molecule. In some embodiments, a labeled recognition molecule comprises a recognition molecule that selectively binds one type of amino acid and a luminescent label having a luminescence that is associated with the recognition molecule. In this way, the luminescence (e.g., luminescence lifetime, luminescence intensity, and other luminescence properties described elsewhere herein) may be associated with the selective binding of the recognition molecule to identify an amino acid of a polypeptide. In some embodiments, a plurality of types of labeled recognition molecules may be used in a method according to the disclosure, where each type comprises a luminescent label having a luminescence that is uniquely identifiable from among the plurality. In some embodiments, the luminescent label of each type of labeled recognition molecule is uniquely identifiable from among the plurality by luminescence intensity alone. Suitable luminescent labels may include luminescent molecules, such as fluorophore dyes, and are described elsewhere herein.

In some embodiments, an amino acid recognition molecule may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide. In some embodiments, desirable properties include an ability to bind selectively and with low affinity (e.g., with a K_{D} of about 50 nM or higher, for example, between about 50 nM and about 50 μM, between about 100 nM and about 10 μM, between about 500 nM and about 50 μM) to more than one type of amino acid. For example, in some aspects, the disclosure provides methods of sequencing by detecting reversible binding interactions during a polypeptide degradation process. Advantageously, such methods may be performed using a recognition molecule that reversibly binds with low affinity to more than one type of amino acid (e.g., a subset of amino acid types).

As used herein, in some embodiments, the terms “selective” and “specific” (and variations thereof, e.g., selectively, specifically, selectivity, specificity) refer to a preferential binding interaction. For example, in some embodiments, an amino acid recognition molecule that selectively binds one type of amino acid preferentially binds the one type over another type of amino acid. A selective binding interaction will discriminate between one type of amino acid (e.g., one type of terminal amino acid) and other types of amino acids (e.g., other types of terminal amino acids), typically more than about 10- to 100-fold or more (e.g., more than about 1,000- or 10,000-fold). Accordingly, it should be appreciated that a selective binding interaction can refer to any binding interaction that is uniquely identifiable to one type of amino acid over other types of amino acids. For example, in some aspects, the disclosure provides methods of polypeptide sequencing by obtaining data indicative of association of one or more amino acid recognition molecules with a polypeptide molecule. In some embodiments, the data comprises a series of signal pulses corresponding to a series of reversible amino acid recognition molecule binding interactions with an amino acid of the polypeptide molecule, and the data may be used to determine the identity of the amino acid. As such, in some embodiments, a “selective” or “specific” binding interaction refers to a detected binding interaction that discriminates between one type of amino acid and other types of amino acids.

In some embodiments, an amino acid recognition molecule binds one type of amino acid with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other types of amino acids. In some embodiments, an amino acid recognition molecule binds one type of amino acid (e.g., one type of terminal amino acid) with a K_{D} of less than about 100 nM, less than about 50 nM, less than about 25 nM, less than about 10 nM, or less than about 1 nM. In some embodiments, an amino acid recognition molecule binds one type of amino acid with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10⁻⁹ μM and about 50 μM). In some embodiments, an amino acid recognition molecule binds one type of amino acid with a K_{D} of about 50 nM.

In some embodiments, an amino acid recognition molecule binds two or more types of amino acids with a K_{D} of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M). In some embodiments, an amino acid recognition molecule binds two or more types of amino acids with a K_{D} of less than about 100 nM, less than about 50 nM, less than about 25 nM, less than about 10 nM, or less than about 1 nM. In some embodiments, an amino acid recognition molecule binds two or more types of amino acids with a K_{D} of between about 50 nM and about 50 M (e.g., between about 50 nM and about 500 nM, between about 50 nM and about 5 M, between about 500 nM and about 50 M, between about 5 M and about 50 M, or between about 10 M and about 50 M). In some embodiments, an amino acid recognition molecule binds two or more types of amino acids with a K_{D} of about 50 nM.

In some embodiments, an amino acid recognition molecule binds at least one type of amino acid with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some embodiments, the value for K_{D} or k_{off} can be a known literature value, or the value can be determined empirically. In some embodiments, the value for k_{off} can be determined empirically based on signal pulse information obtained in a single-molecule assay as described elsewhere herein. For example, the value for k_{off} can be approximated by the reciprocal of the mean pulse duration. In some embodiments, an amino acid recognition molecule binds two or more types of amino acids with a different K_{D} or k_{off} for each of the two or more types. In some embodiments, a first K_{D} or k_{off} for a first type of amino acid differs from a second K_{D} or k_{off} for a second type of amino acid by at least 10% (e.g., at least 25%, at least 50%, at least 100%, or more). In some embodiments, the first and second values for K_{D} or k_{off} differ by about 10⁻²⁵%, 25-50%, 50-75%, 75-100%, or more than 100%, for example by about 2-fold, 3-fold, 4-fold, 5-fold, or more.

As described herein, an amino acid recognition molecule may comprise any biomolecule capable of selectively or specifically binding one molecule over another molecule (e.g., one type of amino acid over another type of amino acid). In some embodiments, a recognition molecule does not comprise a peptidase or does not comprise peptidase activity. For example, in some embodiments, methods of polypeptide sequencing of the disclosure involve contacting a polypeptide molecule with one or more recognition molecules and a cleaving reagent. In such embodiments, the one or more recognition molecules do not comprise peptidase activity, and removal of one or more amino acids from the polypeptide molecule (e.g., amino acid removal from a terminus of the polypeptide molecule) is performed by the cleaving reagent.

Recognition molecules include, for example, proteins and nucleic acids, which may be synthetic or recombinant. In some embodiments, a recognition molecule may comprise an antibody or an antigen-binding portion of an antibody, an SH2 domain-containing protein or fragment thereof, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some embodiments, a recognition molecule comprises a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, a recognition molecule comprises an N-end rule pathway protein selected from a Gid protein (e.g., Gid4 or Gid10 protein), a UBR-box protein (e.g., UBR1, UBR2) or UBR-box domain-containing protein fragment thereof, a p62 protein or ZZ domain-containing fragment thereof, and a ClpS protein (e.g., CipS1, ClpS2). Accordingly, in some embodiments, a labeled recognition molecule comprises a degradation pathway protein. In some embodiments, a labeled recognition molecule comprises a ClpS protein.

In some embodiments, a recognition molecule comprises a ClpS protein, such as Agrobacterium tumifaciens ClpS1, Agrobacterium tumifaciens ClpS2, Synechococcus elongatus ClpS1, Synechococcus elongatus ClpS2, Thermosynechococcus elongatus ClpS, Escherichia coli ClpS, or Plasmodium falciparum ClpS. In some embodiments, the recognition molecule comprises an L/F transferase, such as Escherichia coli leucyl/phenylalanyl-tRNA-protein transferase. In some embodiments, the recognition molecule comprises a D/E leucyltransferase, such as Vibrio vulnificus Aspartate/glutamate leucyltransferase Bpt. In some embodiments, the recognition molecule comprises a UBR protein or UBR-box domain, such as the UBR protein or UBR-box domain of human UBR1 and UBR2 or Saccharomyces cerevisiae UBR1. In some embodiments, the recognition molecule comprises a p62 protein, such as H. sapiens p62 protein or Rattus norvegicus p62 protein, or truncation variants thereof that minimally include a ZZ domain. In some embodiments, the recognition molecule comprises a Gid4 protein, such as H. sapiens GID4 or Saccharomyces cerevisiae GID4. In some embodiments, the recognition molecule comprises a Gid10 protein, such as Saccharomyces cerevisiae GID10. In some embodiments, the recognition molecule comprises an N-meristoyltransferase, such as Leishmania major N-meristoyltransferase or H. sapiens N-meristoyltransferase NMT1. In some embodiments, the recognition molecule comprises a BIR2 protein, such as Drosophila melanogaster BIR2. In some embodiments, the recognition molecule comprises a tyrosine kinase or SH2 domain of a tyrosine kinase, such as H. sapiens Fyn SH2 domain, H. sapiens Src tyrosine kinase SH2 domain, or variants thereof, such as H. sapiens Fyn SH2 domain triple mutant superbinder. In some embodiments, the recognition molecule comprises an antibody or antibody fragment, such as a single-chain antibody variable fragment (scFv) against phosphotyrosine or another post-translationally modified amino acid variant described herein.

In some embodiments, the amino acid recognition molecule, or salt thereof, comprises a sequence shown in Table 1 or Table 2. In some embodiments, the amino acid recognition molecule comprises a sequence shown in Table 1 or Table 2. Also shown in Table 1 and Table 2 are the amino acid binding preferences of each molecule with respect to amino acid identity at a terminal position of a polypeptide unless otherwise specified in Table 1 and Table 2. It should be appreciated that these sequences and other examples described herein are meant to be non-limiting, and recognition molecules in accordance with the application can comprise any homologs, variants thereof, or fragments thereof minimally containing domains or subdomains responsible for peptide recognition.

TABLE 1
Non-limiting examples of ClpS amino acid recognition proteins.

| | | SEQ | |
|---|---|---|---|
| | Binding | ID | |
| Name | Pref.* | NO: | Sequence |
| PS368 | F, Y, W, L | 1 | MASAPSTTLDKSTQVVKKTYPNYKVIVLNDDLNTFDHVA |
| | | | NCLIKYIPDMTTDRAWELTNQVHYQGQAIVWTGPQEQAE |
| | | | LYHQQLRREGLTMAPLEAA |
| PS369 | F, Y, W, L | 2 | MTSTLRARPARDTDLQHRPYPHYRIIVLDDDVNTFQHVV |
| | | | NCLVTFLPGMTRDQAWAMAQQVDGEGSAVVWTGPQEQAE |
| | | | LYHVQLGNHGLTMAPLEPV |
| PS370 | F, L | 3 | MFNSLGTVLDPKKSKAKYPEARVIVLDDNFNTFQHVANC |
| | | | LLAIIPRMCEQRAWDLTIKVDKAGSAEVWRGNLEQAELY |
| | | | HEQLFSKGLTMAPIEKT |
| PS371 | F, Y, W, L | 4 | MATETIERPRTRDPGSGLGGHWLVIVLNDDHNTFDHVAK |
| | | | TLARVIPGVTVDDGYRFADQIHQRGQAIVWRGPKEPAEH |
| | | | YWEQLQDAGLSMAPLERH |
| PS372 | L, I, V | 5 | MAFPARGKTAPKNEVRRQPPYNVILLNDDDHTYRYVIEM |
| | | | LQKIFGFPPEKGFQIAEEVDRTGRVILLTTSKEHAELKQ |
| | | | DQVHSYGPDPYLGRPCSGSMTCVIEPAV |
| PS373 | | 6 | MNRIKQEAVRTENLLICSESIRRTPGTMSNEESMEDEVV |
| | | | AVAVAEPETQHDERRGTKPKRQPPYHVILWDDTDHSFDY |
| | | | VIMMMKRLFRMPIEKGFQVAKEVDSSGRAICMTTTLELA |
| | | | ELKRDQIHAFGKDELLPRCKGSMSATIEPAEG |
| PS374 | F, Y, W, L | 7 | MRWEDPLAAEPVTPGVAPVVEEETDAAVETPWRVILYDD |
| | | | DIHTFEEVILQLMKATGCTPEQGERHAWTVHTRGKDCVY |
| | | | QGDFFDCFRVQGVLREIQLVTEIEG |
| PS375 | F, L | 8 | MEAEPETKVLASIPGVGTSEPFRVVLENDEEHSFDEVIF |
| | | | QIIKAVRCSRAKAEALTMEVHNSGRSIVYTGPIEQCIRV |
| | | | SAVLEEIELRTEIQS |
| PS376 | F, W, L | 9 | MPTNDLDLLEKQDVKIERPKMYQVVMYNDDFTPFDFVVA |
| | | | VLMQFFNKGMDEATAIMMQVHMQGKGICGVFPKDIAETK |
| | | | ATEVMKWAKVEQHPLRLQVEAQA |
| PS377 | W | 10 | MADISKSRPEIGGPKGPQFGDSDRGGGVAVITKPVTKKK |
| | | | FKRKSQTEYEPYWHVLLHHDNVHTFEYATGAIVKVVRTV |
| | | | SRKTAHRITMQAHVSGVATVTTTWKAQAEEYCKGLQMHG |
| | | | LTSSIAPDSSFTH |
| PS378 | F, Y, W, L | 11 | MXPQEVEEVSFLESKEHEIVLYNDDVNTFDHVIECLVKI |
| | | | CNHNYLQAEQCAYIVHHSGKCGVKTGSLEELIPKCNALL |
| | | | EEGLSAEVI |
| PS379 | | 12 | MSTQEEVLEEVKTTTQKENEIVLYNDDYNTFDHVIETLI |
| | | | YACEHTPVQAEQCAILVHYKGKCTVKTGSFDELKPRCSK |
| | | | LLEEGLSAEIV |
| PS380 | F, W | 13 | MGDIYGESNPEEVSCIDSLSEEGNELILENDNIHTFEYV |
| | | | IDCLVAICSLSYEQASNCAYIVDRKGLCTVKHGSYDELL |
| | | | IMYHALVEKDLKVEIR |
| PS381 | | 14 | MVAFSKKWKKDELDKSTGKQKMLILHNDSVNSFDYVIKT |
| | | | LCEVCDHDTIQAEQCAFLTHFKGQCEIAVGEVADLVPLK |
| | | | NKLLNKNLIVSIH |
| PS382 | F, Y, W, L | 15 | MSDSPVIKEIKKDNIKEADEHEKKEREKETSAWKVILYN |
| | | | DDIHNFTYVTDVIVKVVGQISKAKAHTITVEAHSTGQAL |
| | | | ILSTWKSKAEKYCQELQQNGLTVSIIHESQLKDKQKK |
| PS388 | F, Y, L | 16 | MVTTLSADVYGMATAPTVAPERSNQVVRKTYPNYKVIVL |
| | | | NDDFNTFQHVAECLMKYIPGMSSDRAWDLTNQVHYEGQA |
| | | | IVWVGPQEPAELYHQQLRRAGLTMAPLEAA |
| PS389 | F, Y, L | 17 | MLNSAAFKAASASPVIAPERSGQVTQKPYPTYKVIVLND |
| | | | DFNTFQHVHDCLVKYIPGMTSDRAWQLTHQVHNDGQAIV |
| | | | WVGPQEQAELYHQQLSRAGLTMAPIEAA |
| PS390 | F, Y, L | 18 | MLSIAAVTEAPSKGVQTADPKTVRKPYPNYKVIVLNDDF |
| | | | NTFQHVSSCLLKYIPGMSEARAWELTNQVHFEGLAVVWV |
| | | | GPQEQAELYYAQLKNAGLTMAPPEPA |
| PS391 | F, Y, W, L | 19 | MGQTVEKPRVEGPGTGLGGSWRVIVRNDDHNTFDHVART |
| | | | LARFIPGVSLERGHEIAKVIHTTGRAVVYTGHKEAAEHY |
| | | | WQQLKGAGLTMAPLEQG |
| PS392 | F, Y, W | 20 | MSVEIIEKRSTVRKLAPRYRVLLHNDDENPMEYVVQTLM |
| | | | ATVPSLTQPQAVNVMMEAHINGMGLVIVCALEHAEFYAE |
| | | | TLNNHGLGSSIEPDD |
| PS393 | F, Y, W, L | 21 | MSDEDGEDGDENAVGIATRTRTRTKKPTPYRVLLLNDDY |
| | | | TPMEFVVLVLQRFFRMSIEDATRVMLQVHQKGVGVCGVF |
| | | | TYEVAETKVSQVIDFARQNQHPLQCTLEKA |
| PS394 | F, Y, W, L | 22 | MAERRDTGDDEGTGLGIATKTRSKTKKPTPYRVLMLNDD |
| | | | YTPMEFVVLCLQRFFRMNMEEATRVMLHVHQKGVGVCGV |
| | | | FSYEVAETKVGQVIDFARANQHPLQCTLEKA |
| PS395 | F, Y, W, L | 23 | MTVSQSKTQGAPAAQSATELEYEGLWRVVVLNDPVNLMS |
| | | | YVVLVFKKVFGFDETTARKHMLEVHEQGRSVVWSGMREK |
| | | | AEAYAFTLQQWHLTTVLEQDEVR |
| PS396 | F, W | 24 | MSDNDVALKPKIKSKPKLERPKLYKVILVNDDFTPREFV |
| | | | IAVLKMVFRMSEETGYRVMLTAHRLGTSVVVVCARDIAE |
| | | | TKAKEAVDFGKEAGFPLMFTTEPEE |
| PS397 | F, Y, W | 25 | MSDNEVAPKRKTRVKPKLERPRLYKVILVNDDYTPRDFV |
| | | | VMVLKAIFRMSEEAGYRVMMTAHKLGTSVVVVCARDIAE |
| | | | TKAKEATDLGKEAGFPLMFTTEPEE |
| PS398 | F, W | 26 | MPLKAQNRSIVGRRDEWPPPTTQSSSETKSESKRVSDTG |
| | | | ADTKRKTKTVPKVEKPRLYKVILVNDDYTPREFVLVVLK |
| | | | AVFRMSEDQGYKVMITAHQKGSCVVAVYTRDIAETKAKE |
| | | | AVDLAKEIGFPLMFRTEPEE |
| PS404 | F, Y, W | 27 | MPVSVTAPQTKTKTKPKVERPKLYKVILVNDDFTPREFV |
| | | | VRVLKAEFRMSEDQAAKVMMTAHQRGVCVVAVFTRDVAE |
| | | | TKATRATDAGRAKGYPLLFTTEPEE |
| PS405 | F, Y, W | 28 | MVSIGAATVACAEGRPIFSGYFDWLAAMPETVTVPRTRL |
| | | | RPKTERPKLHKVILVNDDYTPREFVVTVLKGEFHMSEDQ |
| | | | AQRVMITAHRRGVCVVAVFTKDVAETKATRASDAGRAKG |
| | | | YPLQFTTEPEE |
| PS406 | F, Y, W | 29 | MPDATTTPRTKTLTRTARPPLHKVILVNDDFTPREFVVR |
| | | | LLKAEFRTTGDEAQRIMITAHMKGSCVVAVFTREIAESK |
| | | | ATRATETARAEGFPLLFTTEPEE |
| PS407 | F, Y, W, L | 30 | MPSNKRQMCLSDIKNSFNESGIVDWHISPRLANEPSEEG |
| | | | DSDLAVQTVPPELKRPPLYAVVLLNDDYTPMEFVIEILQ |
| | | | QYFAMNLDQATQVMLTVHYEGKGVAGVYPRDIAETKANQ |
| | | | VNNYARSQGHPLLCQIEPKD |
| PS408 | F, Y, W, L | 31 | MTDPPSKGREDVDLATRTKPKTQRPPLYKVLLLNDDFTP |
| | | | MEFVVHILERLFGMTHAQAIEIMLTVHRKGVAVVGVFSH |
| | | | EIAETKVAQVMELARRQQHPLQCTMEKE |
| PS409 | F, Y, W, L | 32 | MPARLTDIEGEPNTDPVEDVLLADPELKKPQMYAVVMYN |
| | | | DDYTPMEFVVDVLQNHFKHTLDSAISIMLAIHQQGKGIA |
| | | | GIYPKDIAETKAQTVNRKARQAGYPLLSQIEPQG |
| PS410 | F, W, L | 33 | MGDDDQSSREGEGDVAFQTADPELKRPSLYRVVLLNDDY |
| | | | TPMEFVVHILEQFFAMNREKATQVMLAVHTQGKGVCGVY |
| | | | TKDIAETKAALVNDYSRENQHPLLCEVEELDDESR |
| PS411 | F, Y, W, L | 34 | MTRPDAPEYDDDLAVEPAEPELARPPLYKVVLHNDDFTP |
| | | | MEFVVEVLQEFFNMDSEQAVQVMLAVHTQGKATCGIFTR |
| | | | DIAETKSYQVNEYARECEHPLMCDIEAAD |
| PS412 | F, Y, W, L | 35 | MATKREGSTLLEPTAAKVKPPPLYKVLLLNDDYTPMEFV |
| | | | VLVLKKFFGIDQERATQIMLKVHTEGVGVCGVYPRDIAH |
| | | | TKVEQVVDFARQHQHPLQCTMEES |
| PS413 | F, Y, W, L | 36 | MMKQCGSYFLIKAVQDFKPLSKHRSDTDVITETKIQVKQ |
| | | | PKLYTVIMYNDNYTTMDFVVYVLVEIFQHSIDKAYQLMM |
| | | | QIHESGQAAVALLPYDLAEMKVDEVTALAEQESYPLLTT |
| | | | IEPA |
| PS414 | F, Y, W, L | 37 | MQAAGNEPPDPQNPGDVGNGGDGGNQDGSNTGVVVKTRT |
| | | | RTRKPAMYKVLMLNDDYTPMEFVVHVLERFFQKNREEAT |
| | | | RIMLHVHRRGVGVCGVYTYEVAETKVTQVMDLARQNQHP |
| | | | L?CTIEKE |
| PS415 | F, Y, W | 38 | MALPETRTKIKPDVNIKEPPNYRVIYLNDDKTSMEFVIG |
| | | | SLMQHFSYPQQQAVEKTEEVHEHGSSTVAVLPYEMAEQK |
| | | | GIEVTLDARAEGFPLQVKIEPAER |
| PS416 | F, Y, W, L | 39 | MTSQTDTLVKPNIQPPSLFKVIYINDSVTTMEFVVESLM |
| | | | SVENHSADEATRLTQLVHEEGAAVVAILPYELAEQKGME |
| | | | VTLLARNNGFPLAIRLEPAV |
| PS417 | F, Y, W | 40 | MSNLDTDVLIDEKVKVVTTEPEKYRVILLNDDVTPMDFV |
| | | | INILVSIFKHSTDTAKDLTLKIHKEGSAIVGVYTYEIAE |
| | | | QKGIEATNESRQHGFPLQVKIERENTL |
| PS418 | F, Y, W, L | 41 | MSDHNIDHDTSVAVHLDVVVREPPMYRVVLLNDDFTPME |
| | | | FVVELLMHFFRKTAEQATQIMLNIHHEGVGVCGTYPREI |
| | | | AETKVAQVHQHARTNGHPLKCRMEPS |
| PS419 | F, Y, W, L | 42 | MEKEQSLCKEKTHVELSEPKHYKVVFHNDDFTTMDFVVK |
| | | | VLQLVFFKSQLQAEDLTMKIHLEGSATAGIYSYDIAQSK |
| | | | AQKTTQMAREEGFPLRLTVEPEDN |
| PS420 | F, Y, W, L | 43 | MSDYSNQISQAGSGVAEDASITLPPERKVVFYNDDFTTM |
| | | | EFVVDVLVSIFNKSHSEAEELMQTVHQEGSSVVGVYTYD |
| | | | IAVSRTNLTIQAARKNGFPLRVEVE |
| PS421 | F, Y, W, L | 44 | MTTPNKRPEFEPEIGLEDEVGEPRKYKVLLHNDDYTTMD |
| | | | FVVQVLIEVERKSETEATHIMLTIHEKGVGTCGIYPAEV |
| | | | AETKINEVHTRARREGFPLRASMEEV |
| PS422 | F, Y, W, L | 45 | MTQIKPQTIPDTDVISQTQSDWQMPDLYAVIMHNDDYTT |
| | | | MDFVVFLLNAVEDKPIEQAYQLMMQIHQTGRAVVAILPY |
| | | | EIAEMKVDEATSLAEQEQFPLFISIEQA |
| PS423 | F, Y, W | 46 | MAPTPAGAAVLDKQQQRRHKHASRYRVLLHNDPVNTMEY |
| | | | VVESLRQVVPQLSEQDAIAVMVEAHNTGVGLVIVCDIEP |
| | | | AEFYCEQLKTKGLTSSIEPED |
| PS424 | F, Y, W | 47 | MSVETIEKRSTTRKLAPQYRVLLHNDDYNSMEYVVQVLM |
| | | | TSVPSITQPQAVNIMMEAHNSGLALVITCAQEHAEFYCE |
| | | | TLKGHGLSSTIEPD |
| PS425 | F, Y, W, L | 48 | MTHYFSNILRDQESPKINPKELEQIDVLEEKEHQIILYN |
| | | | DDVNTFEHVIDCLVKICEHNYLQAEQCAYIVHHSGKCSV |
| | | | KTGSLDELVPKCNALLEEGLSAEVV |
| PS426 | F, Y, W, L | 49 | MSIIEKTQENVAILEKVSINHEIILYNDDVNTFDHVIET |
| | | | LIRVCNHEELQAEQCAILVHYTGKCAVKTGSFDELQPLC |
| | | | LALLDAGLSAEIT |
| PS427 | F, W | 50 | MSTKEKVKERVREKEAISFNNEIIVYNDDVNTFDHVIET |
| | | | LIRVCNHTPEQAEQCSLIVHYNGKCTVKTGSMDKLKPQC |
| | | | TQLLEAGLSAEIV |
| PS428 | F | 51 | MSTKEKVKERVREKEAVGENNEIIVYNDDVNTFDHVIDT |
| | | | LMRVCSHTPEQAEQCSLIVHYNGKCTVKTGPMDKLKPQC |
| | | | TQLLEAGLSAEIV |
| PS429 | | 52 | MSVQEEVLEEVKTKERVNKQNQIIVFNDDVNTFDHVIDM |
| | | | LIATCDHDPIQAEQCTMLIHYKGKCEVKTGDYDDLKPRC |
| | | | SKLLDAGISAEIQ |
| PS430 | F, Y, W, L | 53 | MQPFEETYTDVLDEVVDTDVHNLVVENDDVNTFDHVIET |
| | | | LIDVCKHTPEQAEQCTLLIHYKGKCSVKNGSWEELVPMR |
| | | | NEICRRGISAEVLK |
| PS431 | | 54 | MIISSVKSSPSTETLSRTELQLGGVWRVVVLNDPVNLMS |
| | | | YVMMIFKKIFGFNETVARRHMLEVHEKGRSVVWSGLREK |
| | | | AEAYVFTLQQWHLTAVLESDETH |
| PS432 | F, W | 55 | MIGVEARTSSAPELAIETEIRLAGLWHVIVINDPVNLMS |
| | | | YVVMVLRKIFGFDDTKARKHMLEVHENGRSIVWSGEREP |
| | | | AEAYANTLHQWHLSAVLERDETD |
| PS433 | F, Y, W, L | 56 | MMSSLKECSIQALPSLDEKTKTEEDLSVPWKVIVLNDPV |
| | | | NLMSYVVMVFRKVFGYNENKATKHMMEVHQLGKSVLWTG |
| | | | QREEAECYAYQLQRWRLQTILEKDD |
| PS434 | F, Y, W, L | 57 | MSRLPWKQEAKFAATVIIDFPDATLEAPTIEKKEATEQQ |
| | | | IEMPWNVVVHNDPVNLMSYVTMVFQRVFGYPRERAEKHM |
| | | | LEVHHSGRSILWSGLRERAELYVQQLHGYLLLATIEKTV |
| PS435 | F, Y, W, L | 58 | MTLSVALGPDTQESTQTGTAVSTDTLTAPDIPWNLVIWN |
| | | | DPVNLMSYVSYVFQSYFGYSETKANKLMMEVHKKGRSIV |
| | | | AHGSKEQVEQHAVAMHGYGLWATVEKATGGNSGGGKSGG |
| | | | PGKGKGKRG |
| Planctomycetales | I, L, V | 59 | MSEPMTLPAIPQPRLKERTQRQPPYNVILLNDDDHSYEY |
| bacterium | | | VIAMLQVLFGYPREKGYQMAKEVDSTGRVILLTTTREHA |
| (PS545) | | | ELKQEQIHAFGPDPLMARCQGSMTAVIEPAV |
| Planctomycetia | I, L, V | 60 | MSDTITLPGRPEVERDERTRRQPPYNVILHNDDDHTFEY |
| bacterium | | | VIVMLNQLFGYPPEKGYEMAKEVHLNGRVIVLTTSKEHA |
| (PS546) | | | ELKRDQIHAFGPDPFSSKDCKGSMSASIEPAY |
| Gemmataceae | I, L, V | 61 | MGFPTDFRQSIEISTPLGSQQPRESNASSEPALADPVLV |
| bacterium | | | INPRIQPRYHVILLNDDDHTYRYVIEMMLIVEGHPPEKG |
| (PS547) | | | FLIAKEVDKAGRAICLTTSLEHAEFKQEQVHAYGADPYF |
| | | | GPKCKGSMTAVLEPAE |
| Gemmataceae | I, L, V | 62 | MSDTITLPEEKTDVRTKRQPPYHVILLNDDDHTYQYVIY |
| bacterium | | | MLQTLFGHPPETGFKMAQEVDKTGRVIVDTTSLERAELK |
| (PS548) | | | RDQIHAFGPDPYIERCKGSMSAMIEPSE |
| Planctomycetes | I, L, V | 63 | MSESITTLPKKSRRLKEEEEQKTKRQPPYNVILLNDDDH |
| bacterium | | | TFEYVIFMLQKLFGHPPERGMQMAKEVHTTGRVIVMTTA |
| (PS549) | | | LELAELKRDQIHAFGPDPLIDRCKGSMSATIEPAPI |
| Planctomycetes | I, L, V | 64 | MPTFTEPEVVNDTRILPPYHVILLNDDDHTYEYVIHMLQ |
| bacterium | | | TLFGHPQERGFQLAVEVDKKGKAIVFTTSKEHAEFKRDQ |
| (PS550) | | | IHAFGADPLSSKNCKGSMSAVIEPSF |
| Rubrobacter | I, L, V | 65 | MPSAAPAKPKTKRQSRTQGMPPYNVVLLDDDDHTYGYVI |
| indicoceani | | | EMLNKVFGHPPEKGFELATEVDKNGRVIVMTTNLEVAEL |
| (PS551) | | | KRDEVHAFGPDPLMPRSKGSMSAVVERAG |
| Fimbriiglobus | I, L, V | 66 | MSKTSTLPEVESESAQKLKYQPPYHVILLNDDDHSYVYV |
| ruber | | | ITMLKELFGHPEQKGYQLADAVDKQGRAIVFTTTREHAE |
| (PS552) | | | LKQEQIHAYGPDPTIPRCKGAMTAVIEPAE |
| Planctomycetes | I, L, V | 67 | MPASASAVTEPPVSLPEAAAPRPKDRPKRQPRYHVILWN |
| bacterium | | | DDDHTYQYVVAMLRQLFGHPPEKGFTLAKQVDKDGRVVV |
| (PS553) | | | LITTKEHAELKRDQIHAFGADRLLARSKGSMSASIEPEA |
| | | | STG |
| Planctomycetia | I, L, V | 68 | MSDSASATVEVQADPPADATARSQPTPARSTGSKPKRQP |
| bacterium | | | RYHVVLWNDDDHTYEYVIAMLRRLFGIEPEKGFRIAEEV |
| (PS554) | | | DQSGRAVVLTTTREHAELKRDQIHAFGADRLLARSKGSM |
| | | | SASIEPEA |
| Planctomycetes | I, L, V | 69 | MADSAQTGVAEPIQETLRRRKLRDDRRPKRQPPYHVILW |
| bacterium | | | NDNDHTYAYVVVMLMQLFGYPAEKGYQLASEVDTQGRAV |
| RBG_16_64_12 | | | VLTTTKEHAELKRDQIHAYGKDGLIEKCKGSMWATIEPA |
| (PS555) | | | PGE |
| Blastopirellula | I, L, V | 70 | MGDSNTSVAEPGEVTVVTTKPAPKKAKPKRQPKYHVVLW |
| marina | | | NDDDHTYEYVILMMHELFGHPVEKGFQIAKTVDADGRAI |
| (PS556) | | | CLTTTKEHAELKRDQIHAYGKDELIARCRGSMSSTIEPE |
| | | | C |
| Planctomycetia | I, L, V | 71 | MPTAASATESAIEDTPAPARPEVDGRTKPKRQPRYHVVL |
| bacterium | | | WNDDDHTYQYVVVMLQSLFGHPPERGYRLAKEVDTQGRV |
| (PS557) | | | IVLTTTREHAELKRDQIHAFGYDRLLARSKGSMKASIEA |
| | | | EE |
| Planctomycetia | I, L, V | 72 | MTATTADPDRTTAEKTTKKARRSGQPKRQPRYHVILWND |
| bacterium | | | NDHTYQYVVAMLQQLFGHPATTGLKLATEVDRTGRAVIL |
| (PS558) | | | TTTREHAELKRDQIHAFGADRLLARSKGAMSASIEPEAE |
| Planctomycetaceae | I, L, V | 73 | MNQAAISPNPDIKPNPSTHKKRASQRQPRYHVILWNDND |
| bacterium | | | HTYHYVVTMLQKLFGHPPRTGIKMATEVDKKGKVIVLTT |
| (PS559) | | | SREHAELKRDQIHAFGADKLIRRSKGAMAASIEPES |
| Planctomycetes | I, L, V | 74 | MTETITTPAERTQTQAEPRSDRAWLWNVVLLDDDEHTYE |
| bacterium | | | YVIRMLHTLFGMPVERAFRLAEEVDARGRAVVLTTHKEH |
| (PS560) | | | AELKRDQVHAFGKDALIASCAGSMSAVLEPAECGSDDED |
| Roseimaritima sp. | I, L, V | 75 | MAELQTAVVEPTTRPEQDEKQSQSRPKRQPRYNVILWDD |
| JC651 | | | PDHSYDYVIMMLKELFGHPRQRGHQMAEEVDTTGRVICL |
| (PS561) | | | TTTMEHAELKRDQIHAYGSDEGITRCKGSMSASIEPVPE |
| Rubripirellula | I, L, V | 76 | MSDQQSMVAEPEVVVHTQDEKKLEKQNKRKKQPRYNVVL |
| amarantea | | | WDDTDHSYDYVVLMMKQLFHHPIETGFQIAKQVDKGGKA |
| (PS562) | | | ICLTTTMEHAELKRDQIHAFGKDDLIARCTGSMSATIEP |
| | | | VPE |
| Acidobacteria | I, L, V | 77 | MSSRSATAYPEVEDDTSDQLQPLYHVILLNDEDHTYDYV |
| bacterium | | | IEMLQKIFGFPESKAFSHAVEVDTKGTTILLTCDLEQAE |
| (PS563) | | | RKRDLIHSYGPDWRLPRSLGSMAAVVEPAAG |
| Planctomycetes | I, L, V | 78 | MFEEVVSVAVAEPKTKKQSRTKPKRQPPYHVILWDDTDH |
| bacterium Poly21 | | | TFDYVIKMMGELFRMPREKGYQLAKEVDTSGRAICMTTT |
| (PS564) | | | LELAELKRDQIHAFGRDDASAHCKGSMSATIEPAEG |
| Aquisphaera sp. | I, L, V | 79 | MSEFDHEHSGDTSVADPIVTTKTAPKPQKHAENETETRR |
| JC650 | | | QPPYNVIILNDEEHTEDYVIELLCKVFRHSLATAQELTW |
| (PS565) | | | RIHLTGRAVVLTTHKELAELKRDQVLAYGPDPRMSVSKG |
| | | | PLDCFIEPAPGG |
| Planctomycetaceae | I, L, V | 80 | MSSPSSLDDVQVSTSRAKPANETRTRKQPPYAVIVENDD |
| bacterium | | | HHTFLYVIEALMKVCGHAPEKGFVLAQQIHTQGKAMVWS |
| (PS566) | | | GTLELAELKRDQLRGFGPDNYAPRPVTFPLGVTIEPLP |
| Planctomycetaceae | I, L, V | 81 | MADYEDAGEDALEDDFDHGTVTVAPQKPEPKKQSENKRQ |
| bacterium | | | ANRQPRYNVLLWDSEDHTFEYVEKMLRELFGHIKKQCQI |
| (PS567) | | | IAEQVDQEGRAVVLITTLEHAELKRDQIHAYGKDQLEGS |
| | | | KGSMWSTIEAVD |
| Dehalococcoidia | I, L, V | 82 | MTTPSLPTRETEVEERTEVEPERLYHLVLLDDDQHSYQY |
| bacterium | | | VIEMLASIFGYGSEKAWTLARIVDTEGRAILETASHAQC |
| (PS568) | | | ERHQSQIHAYGADSRIPTSVGSMSAVIEEAGTPPQT |
| Planctomycetes | I, L, V | 83 | MYSKNQIKIYCSEDDKGQTATPLLEKKPKFAPLYHVILW |
| bacterium | | | DDNTHTYEYVIKLLMSLFRMTFEKAYQHTLEVDKKGRTI |
| (PS569) | | | CITTHLEKAELKQEQISNFGPDILMQNSKGPMSATIEPA |
| | | | N |
| Leptospira | I, L, V | 84 | MTGAGASQPSILEETEVRPRLSDGPWKVVLWDDDFHTYE |
| congkakensis | | | YVIEMLMDVCQMPWEKAFQHAVEVDTRKKTIVFSGELEH |
| (PS570) | | | AEFVHERILNYGPDPRMGSSKGSMTATLEQ |
| Leptospira meyeri | I, L, V | 85 | MTSSGASQPSILEETERKPRLSDGPWKVVLWDDDFHTYE |
| (PS571) | | | YVIEMLMDVCQMPWEKAFQHAVEVDTRKKTIVFFGELEH |
| | | | AEFVHERILNYGPDPRMGTSKGSMTATLEK |
| Blastopirellula | I, L, V | 86 | MSSEELSLQTRPKRQPPFGVILHNDDLNSFDYVIDSIRK |
| marina | | | VFHYELEKCFQLTLEAHETGRSLLWTGTLEGAELKQELL |
| (PS572) | | | LSCGPDPIMLDKGGLPLKVTLEELPQ |
| Leptospira | I, L, V | 87 | MSQTPVIEETTVKDPVKTGGPWKVVLWDDDEHTYDYVIE |
| fluminis | | | MLMEVCVMTMEQAFHHAVEVDTQKKTVVYSGEFEHAEHI |
| (PS573) | | | QELILEYGPDPRMAVSKGSMSATLEKS |
| Gemmata | I, L, V | 88 | MANATPTPDVVPEEETETRTRRQPPYAVVLHNDDTNTMD |
| obscuriglobus | | | FVVTVLRKVFGYTVEKCVELMLEAHTQGKVAVWIGALEV |
| (PS574) | | | AELKADQIKSFGPDPHVTKNGHPLGVTVEPAA |
| Leptospira kmetyi | I, L, V | 89 | MASTQTPDLNEITEESTKSTGGPWRVVLWDDNEHTYEYV |
| (PS575) | | | IEMLMEICTMTVEKAFLHAVQVDQEKRTVVFSGEFEHAE |
| | | | HVQERILTYGADPRMSNSKGSMSATLEK |
| Leptospira | I, L, V | 90 | MASTQTPDLNEITEESTKSTGGPWRVVLWDDNEHTYEYV |
| interrogans | | | IEMLVEICMMTVEKAFLHAVQVDKEKRTVDFSGELEHAE |
| (PS576) | | | HVQERILNYGADPRMSNSKGSMSATLER |
| Tuwongella | I, L, V | 91 | MSASSSQPGTTTKPDLDIQPRLLPPFHVILENDEFHSME |
| immobilis | | | FVIDTLRKVLGVSIERAYQLMMTAHESGQAIIWTGPKEV |
| (PS577) | | | AELKYEQVIGFHEKRSDGRDLGPLGCRIEPAV |
| Planctomycetes | I, L, V | 92 | MSGTVVESKPRNSTQLAPRWKVIVHDDPVTTFDFVLGVL |
| bacterium | | | RRVFAKPPGEARRITREAHDTGSALVDVLALEQAEFRRD |
| (PS578) | | | QAHSLARAEGFPLTLTLEPAD |
| Agrobacterium | F, W, Y, L | 93 | MIAEPICMQGEGDGEDGGTNRGTSVITRVKPKTKRPNLY |
| tumifaciens ClpS1 | | | RVLLLNDDYTPMEFVIHILERFFQKDREAATRIMLHVHQ |
| (atClpS1) | | | HGVGECGVFTYEVAETKVSQVMDFARQHQHPLQCVMEKK |
| Agrobacterium | F, W, Y | 94 | MSDSPVDLKPKPKVKPKLERPKLYKVMLLNDDYTPREFV |
| tumifaciens ClpS2 | | | TVVLKAVFRMSEDTGRRVMMTAHRFGSAVVVVCERDIAE |
| (atClpS2) | | | TKAKEATDLGKEAGFPLMFTTEPEE |
| atClpS2 | F, W, Y | 95 | MSDSPVDLKPKPKVKPKLERPKLYKVILLNDDYTPMEFV |
| thermostable | | | VEVLKRVFNMSEEQARRVMMTAHKKGKAVVGVCPRDIAE |
| variant | | | TKAKQATDLAREAGFPLMFTTEPEE |
| PS489 | M | 96 | MSDSPVDLKPKPKVKPKLERLKLYKVILLNDDYTTAFFV |
| | | | VKVLKRVFNMSEEQARRVMMTAHKKGKAVVGVCPRDIAE |
| | | | TKAKQATDLAREAGFPLMFTTEPEE |
| PS490 | M | 97 | MSDSPVDLKPKPKVKPKLERLKLYKVILLNDDYTTMRFV |
| | | | VLVLKRVFNMSEEQARRVMMTAHKKGKAVVGVCPRDIAE |
| | | | TKAKQATDLAREAGFPLMFTTEPEE |
| PS218 | F, W, Y, L | 98 | MIAEPICMQGEGDGEDGGTNRGTSVITRVKPKTKRPNLY |
| | | | RVLLLNDDYTPFQFVIHILERFFQKDREAAWRITLHVHQ |
| | | | HGVGECGVFTYEVAETKVSQVMDFARQHQHPLQCVMEKK |
| atClpS2-V1 | F, W, Y | 99 | MSDSPVDLKPKPKVKPKLERPKLYKVMLLNDDYTPMSFV |
| | | | TVVLKAVFRMSEDTGRRVMMTAHRFGSAVVVVCERDIAE |
| | | | TKAKEATDLGKEAGFPLMFTTEPEE |
| atClpS2 C72S | F, W, Y | 100 | MSDSPVDLKPKPKVKPKLERPKLYKVMLLNDDYTPREFV |
| | | | TVVLKAVFRMSEDTGRRVMMTAHRFGSAVVVVSERDIAE |
| | | | TKAKEATDLGKEAGFPLMFTTEPEE |
| atClpS2-V1 + | F, W, Y | 101 | MSDSPVDLKPKPKVKPKLERPKLYKVMLLNDDYTPMSFV |
| C72S | | | TVVLKAVFRMSEDTGRRVMMTAHRFGSAVVVVSERDIAE |
| | | | TKAKEATDLGKEAGFPLMFTTEPEE |
| atClpS2 | F, W, Y | 102 | MSDSPVDLKPKPKVKPKLERPKLYKVILLNDDYTPMEFV |
| thermostable | | | VEVLKRVFNMSEEQARRVMMTAHKKGKAVVGVSPRDIAE |
| variant + C72S | | | TKAKQATDLAREAGFPLMFTTEPEE |
| atClpS1 C7S | F, W, Y, L | 103 | MIAEPISMQGEGDGEDGGTNRGTSVITRVKPKTKRPNLY |
| | | | RVLLLNDDYTPMEFVIHILERFFQKDREAATRIMLHVHQ |
| | | | HGVGECGVFTYEVAETKVSQVMDFARQHQHPLQCVMEKK |
| atClpS1 C7S, | F, W, Y, L | 104 | MIAEPISMQGEGDGEDGGTNRGTSVITRVKPKTKRPNLY |
| C84S, C112S | | | RVLLLNDDYTPMEFVIHILERFFQKDREAATRIMLHVHQ |
| | | | HGVGESGVFTYEVAETKVSQVMDFARQHQHPLQSVMEKK |
| Synechococcus | F, W, Y | 105 | MAVETIQKPETTTKRKIAPRYRVLLHNDDENPMEYVVMV |
| elongatus ClpS1 | | | LMQTVPSLTQPQAVDIMMEAHTNGTGLVITCDIEPAEFY |
| | | | CEQLKSHGLSSSIEPDD |
| Synechococcus | F, W, Y, L | 106 | MSPQPDESVLSILGVPRPCVKKRSRNDAFVLTVLTCSLQ |
| elongatus ClpS2 | | | AIAAPATAPGTTTTRVRQPYPHFRVIVLDDDVNTFQHVA |
| | | | ECLLKYIPGMTGDRAWDLTNQVHYEGAATVWSGPQEQAE |
| | | | LYHEQLRREGLTMAPLEAA |
| Thermosynechococcus | F, W, Y, L | 107 | MPQERQQVTRKHYPNYKVIVLNDDENTFQHVAACLMKYI |
| elongatus ClpS | | | PNMTSDRAWELTNQVHYEGQAIVWVGPQEQAELYHEQLL |
| | | | RAGLTMAPLEPE |
| Escherichia coli | F, W, Y, L | 108 | MGKTNDWLDFDQLAEEKVRDALKPPSMYKVILVNDDYTP |
| ClpS | | | MEFVIDVLQKFFSYDVERATQLMLAVHYQGKAICGVFTA |
| | | | EVAETKVAMVNKYARENEHPLLCTLEKA |
| Escherichia coli | F, W, Y, L | 109 | MGKTNDWLDFDQLAEEKVRDALKPPSMYKVILVNDDYTP |
| ClpS M40A | | | AEFVIDVLQKFFSYDVERATQLMLAVHYQGKAICGVFTA |
| | | | EVAETKVAMVNKYARENEHPLLCTLEKA |
| Plasmodium | F, W, Y, L | 110 | MFKDLKPFFLCIILLLLLIYKCTHSYNIKNKNCPLNEMN |
| falciparum ClpS | | | SCVRINNVNKNTNISFPKELQKRPSLVYSQKNFNLEKIK |
| | | | KLRNVIKEIKKDNIKEADEHEKKEREKETSAWKVILYND |
| | | | DIHNFTYVTDVIVKVVGQISKAKAHTITVEAHSTGQALI |
| | | | LSTWKSKAEKYCQELQQNGLTVSIIHESQLKDKQKK |

*Binding preferences are inferred from published scientific literature and/or further demonstrated by the inventors in single-molecule and/or ensemble experiments, as described herein.
**Binding to phosphotyrosine may occur at a peptide terminus or at an internal position.

TABLE 2
Non-limiting examples of amino acid recognition proteins.

| | | SEQ | |
|---|---|---|---|
| | Binding | ID | |
| Name | Pref.* | NO: | Sequence |
| Escherichia coli | K, R | 111 | MRLVQLSRHSIAFPSPEGALREPNGLLALGGDLSPARLL |
| leucyl/phenylalanyl- | | | MAYQRGIFPWFSPGDPILWWSPDPRAVLWPESLHISRSM |
| tRNA-protein | | | KRFHKRSPYRVTMNYAFGQVIEGCASDREEGTWITRGVV |
| transferase | | | EAYHRLHELGHAHSIEVWREDELVGGMYGVAQGTLFCGE |
| | | | SMFSRMENASKTALLVFCEEFIGHGGKLIDCQVLNDHTA |
| | | | SLGACEIPRRDYLNYLNQMRLGRLPNNFWVPRCLFSPQE |
| | | | LE |
| Vibrio vulnificus | D, E | 112 | MSSDIHQIKIGLTDNHPCSYLPERKERVAVALEADMHTA |
| Aspartate/glutamate | | | DNYEVLLANGFRRSGNTIYKPHCDSCHSCQPIRISVPDI |
| leucyltransferase | | | ELSRSQKRLLAKARSLSWSMKRNMDENWEDLYSRYIVAR |
| Bpt | | | HRNGTMYPPKKDDFAHFSRNQWLTTQFLHIYEGQRLIAV |
| | | | AVTDIMDHCASAFYTFFEPEHELSLGTLAVLFQLEFCQE |
| | | | EKKQWLYLGYQIDECPAMNYKVRFHRHQKLVNQRWQ |
| H. sapiens GID4 | P | 113 | MSGSKFRGHQKSKGNSYDVEVVLQHVDTGNSYLCGYLKI |
| | | | KGLTEEYPTLTTFFEGEIISKKHPFLTRKWDADEDVDRK |
| | | | HWGKFLAFYQYAKSFNSDDFDYEELKNGDYVFMRWKEQF |
| | | | LVPDHTIKDISGASFAGFYYICFQKSAASIEGYYYHRSS |
| | | | EWYQSLNLTHV |
| Saccharomyces | P | 114 | MINNPKVDSVAEKPKAVTSKQSEQAASPEPTPAPPVSRN |
| cerevisiae GID4 | | | QYPITFNLTSTAPFHLHDRHRYLQEQDLYKCASRDSLSS |
| | | | LQQLAHTPNGSTRKKYIVEDQSPYSSENPVIVTSSYNHT |
| | | | VCTNYLRPRMQFTGYQISGYKRYQVTVNLKTVDLPKKDC |
| | | | TSLSPHLSGFLSIRGLTNQHPEISTYFEAYAVNHKELGE |
| | | | LSSSWKDEPVLNEFKATDQTDLEHWINFPSFRQLFLMSQ |
| | | | KNGLNSTDDNGTTNAAKKLPPQQLPTTPSADAGNISRIF |
| | | | SQEKQFDNYLNERFIFMKWKEKFLVPDALLMEGVDGASY |
| | | | DGFYYIVHDQVTGNIQGFYYHQDAEKFQQLELVPSLKNK |
| | | | VESSDCSFEFA |
| Single-chain antibody | phospho- | 115 | MMEVQLQQSGPELVKPGASVMISCRTSAYTFTENTVHWV |
| variable fragment | Y | | KQSHGESLEWIGGINPYYGGSIFSPKFKGKATLTVDKSS |
| (scFv) against | | | STAYMELRSLTSEDSAVYYCARRAGAYYFDYWGQGTTLT |
| phosphotyrosine** | | | VSSGGGSGGGSGGGSENVLTQSPAIMSASPGEKVTMTCR |
| | | | ASSSVSSSYLHWYRQKSGASPKLWIYSTSNLASGVPARF |
| | | | SGSGSGTSYSLTISSVEAEDAATYYCQQYSGYRTFGGGT |
| | | | KLEIKR |
| H. sapiens Fyn SH2 | phospho- | 116 | MGAMDSIQAEEWYFGKLGRKDAERQLLSFGNPRGTFLIR |
| domain** | Y | | ESETTKGAYSLSIRDWDDMKGDHVKHYKIRKLDNGGYYI |
| | | | TTRAQFETLQQLVQHYSERAAGLSSRLVVPSHK |
| H. sapiens Fyn SH2 | phospho- | 117 | MGAMDSIQAEEWYFGKLGRKDAERQLLSFGNPRGTFLIR |
| domain triple mutant | Y | | ESETVKGAYALSIRDWDDMKGDHVKHYLIRKLDNGGYYI |
| superbinder** | | | TTRAQFETLQQLVQHYSERAAGLSSRLVVPSHK |
| H. sapiens Src | phospho- | 118 | MGAMDSIQAEEWYFGKITRRESERLLLNAENPRGTFLVR |
| tyrosine kinase SH2 | Y | | ESETTKGAYSLSVSDFDNAKGLNVKHYKIRKLDSGGFYI |
| domain** | | | TSRTQFNSLQQLVAYYSKHADGLCHRLTTVCPTSK |
| H. sapiens Src | phospho- | 119 | MGAMDSIQAEEWYFGKITRRESERLLLNAENPRGTFLVR |
| tyrosine kinase SH2 | Y | | ESEVTKGAYALSVSDFDNAKGLNVKHYLIRKLDSGGFYI |
| domain triple | | | TSRTQFNSLQQLVAYYSKHADGLCHRLTTVCPTSK |
| mutant** | | | |
| H. sapiens p62 | K, R, H, | 120 | MASLTVKAYLLGKEDAAREIRRFSFCCSPEPEAEAEAAA |
| fragment 1-310 | W, F, Y | | GPGPCERLLSRVAALFPALRPGGFQAHYRDEDGDLVAFS |
| | | | SDEELTMAMSYVKDDIFRIYIKEKKECRRDHRPPCAQEA |
| | | | PRNMVHPNVICDGCNGPVVGTRYKCSVCPDYDLCSVCEG |
| | | | KGLHRGHTKLAFPSPFGHLSEGFSHSRWLRKVKHGHFGW |
| | | | PGWEMGPPGNWSPRPPRAGEARPGPTAESASGPSEDPSV |
| | | | NFLKNVGESVAAALSPLGIEVDIDVEHGGKRSRLTPVSP |
| | | | ESSSTEEKSSSQPSSCCSDPSKPGGNVEGATQSLAEQ |
| H. sapiens p62 | K, R, H, | 121 | MASLTVKAYLLGKEDAAREIRRFSFCCSPEPEAEAEAAA |
| fragment 1-180 | W, F, Y | | GPGPCERLLSRVAALFPALRPGGFQAHYRDEDGDLVAFS |
| | | | SDEELTMAMSYVKDDIFRIYIKEKKECRRDHRPPCAQEA |
| | | | PRNMVHPNVICDGCNGPVVGTRYKCSVCPDYDLCSVCEG |
| | | | KGLHRGHTKLAFPSPFGHLSEGFSHSRWLRKVKHGHFGW |
| | | | PGWEMGPPGNWSPRPPRAGEARPGPTAESASGPSEDPSV |
| | | | NFLKNVGESVAAALSPLGIEVDIDVEHGGKRSRLTPVSP |
| | | | ESSSTEEKSSSQPSSCCSDPSKPGGNVEGATQSLAEQ |
| H. sapiens p62 | K, R, H, | 122 | MASLTVKAYLLGKEDAAREIRRFSFCCSPEPEAEAEAAA |
| fragment 126-180 | W, F, Y | | GPGPCERLLSRVAALFPALRPGGFQAHYRDEDGDLVAFS |
| | | | SDEELTMAMSYVKDDIFRIYIKEKKECRRDHRPPCAQEA |
| | | | PRNMVHPNVICDGCNGPVVGTRYKCSVCPDYDLCSVCEG |
| | | | KGLHRGHTKLAFPSPFGHLSEGFSHSRWLRKVKHGHFGW |
| | | | PGWEMGPPGNWSPRPPRAGEARPGPTAESASGPSEDPSV |
| | | | NFLKNVGESVAAALSPLGIEVDIDVEHGGKRSRLTPVSP |
| | | | ESSSTEEKSSSQPSSCCSDPSKPGGNVEGATQSLAEQ |
| H. sapiens p62 | K, R, H, | 123 | MASLTVKAYLLGKEDAAREIRRFSFCCSPEPEAEAEAAA |
| protein | W, F, Y | | GPGPCERLLSRVAALFPALRPGGFQAHYRDEDGDLVAFS |
| | | | SDEELTMAMSYVKDDIFRIYIKEKKECRRDHRPPCAQEA |
| | | | PRNMVHPNVICDGCNGPVVGTRYKCSVCPDYDLCSVCEG |
| | | | KGLHRGHTKLAFPSPFGHLSEGFSHSRWLRKVKHGHFGW |
| | | | PGWEMGPPGNWSPRPPRAGEARPGPTAESASGPSEDPSV |
| | | | NFLKNVGESVAAALSPLGIEVDIDVEHGGKRSRLTPVSP |
| | | | ESSSTEEKSSSQPSSCCSDPSKPGGNVEGATQSLAEQMR |
| | | | KIALESEGRPEEQMESDNCSGGDDDWTHLSSKEVDPSTG |
| | | | ELQSLQMPESEGPSSLDPSQEGPTGLKEAALYPHLPPEA |
| | | | DPRLIESLSQMLSMGFSDEGGWLTRLLQTKNYDIGAALD |
| | | | TIQYSKHPPPL |
| Rattus norvegicus | K, R, H, | 124 | MASLTVKAYLLGKEEAAREIRRFSFCFSPEPEAEAAAGP |
| p62 protein | W, F, Y | | GPCERLLSRVAVLFPALRPGGFQAHYRDEDGDLVAFSSD |
| | | | EELTMAMSYVKDDIFRIYIKEKKECRREHRPPCAQEARS |
| | | | MVHPNVICDGCNGPVVGTRYKCSVCPDYDLCSVCEGKGL |
| | | | HREHSKLIFPNPFGHLSDSFSHSRWLRKLKHGHFGWPGW |
| | | | EMGPPGNWSPRPPRAGDGRPCPTAESASAPSEDPNVNFL |
| | | | KNVGESVAAALSPLGIEVDIDVEHGGKRSRLTPTSAESS |
| | | | STGTEDKSGTQPSSCSSEVSKPDGAGEGPAQSLTEQMKK |
| | | | IALESVGQPEELMESDNCSGGDDDWTHLSSKEVDPSTGE |
| | | | LQSLQMPESEGPSSLDPSQEGPTGLKEAALYPHLPPEAD |
| | | | PRLIESLSQMLSMGFSDEGGWLTRLLQTKNYDIGAALDT |
| | | | IQYSKHPPPL |
| Saccharomyces | P, M, V | 125 | MTSLNIMGRKFILERAKRNDNIEEIYTSAYVSLPSSTDT |
| cerevisiae GID10 | | | RLPHFKAKEEDCDVYEEGTNLVGKNAKYTYRSLGRHLDF |
| | | | LRPGLRFGGSQSSKYTYYTVEVKIDTVNLPLYKDSRSLD |
| | | | PHVTGTFTIKNLTPVLDKVVTLFEGYVINYNQFPLCSLH |
| | | | WPAEETLDPYMAQRESDCSHWKRFGHFGSDNWSLTERNE |
| | | | GQYNHESAEFMNQRYIYLKWKERFLLDDEEQENQMLDDN |
| | | | HHLEGASFEGFYYVCLDQLTGSVEGYYYHPACELFQKLE |
| | | | LVPTNCDALNTYSSGFEIA |
| Leishmania major N- | G | 126 | MSRNPSNSDAAHAFWSTQPVPQTEDETEKIVFAGPMDEP |
| meristoyltransferase | | | KTVADIPEEPYPIASTFEWWTPNMEAADDIHAIYELLRD |
| | | | NYVEDDDSMFRFNYSEEFLQWALCPPNYIPDWHVAVRRK |
| | | | ADKKLLAFIAGVPVTLRMGTPKYMKVKAQEKGEGEEAAK |
| | | | YDEPRHICEINFLCVHKQLREKRLAPILIKEATRRVNRT |
| | | | NVWQAVYTAGVLLPTPYASGQYFHRSLNPEKLVEIRFSG |
| | | | IPAQYQKFQNPMAMLKRNYQLPSAPKNSGLREMKPSDVP |
| | | | QVRRILMNYLDSFDVGPVFSDAEISHYLLPRDGVVFTYV |
| | | | VENDKKVTDFFSFYRIPSTVIGNSNYNLLNAAYVHYYAA |
| | | | TSIPLHQLILDLLIVAHSRGFDVCNMVEILDNRSFVEQL |
| | | | KFGAGDGHLRYYFYNWAYPKIKPSQVALVML |
| H. sapiens N- | G | 127 | MADESETAVKPPAPPLPQMMEGNGNGHEHCSDCENEEDN |
| meristoyltransferase | | | SYNRGGLSPANDTGAKKKKKKQKKKKEKGSETDSAQDQP |
| NMT1 | | | VKMNSLPAERIQEIQKAIELFSVGQGPAKTMEEASKRSY |
| | | | QFWDTQPVPKLGEVVNTHGPVEPDKDNIRQEPYTLPQGF |
| | | | TWDALDLGDRGVLKELYTLLNENYVEDDDNMFRFDYSPE |
| | | | FLLWALRPPGWLPQWHCGVRVVSSRKLVGFISAIPANIH |
| | | | IYDTEKKMVEINFLCVHKKLRSKRVAPVLIREITRRVHL |
| | | | EGIFQAVYTAGVVLPKPVGTCRYWHRSLNPRKLIEVKFS |
| | | | HLSRNMTMQRTMKLYRLPETPKTAGLRPMETKDIPVVHQ |
| | | | LLTRYLKQFHLTPVMSQEEVEHWFYPQENIIDTFVVENA |
| | | | NGEVTDFLSFYTLPSTIMNHPTHKSLKAAYSFYNVHTQT |
| | | | PLLDLMSDALVLAKMKGFDVFNALDLMENKTFLEKLKFG |
| | | | IGDGNLQYYLYNWKCPSMGAEKVGLVLQ |
| Drosophila | A | 128 | MGDVQPETCRPSAASGNYFPQYPEYAIETARLRTFEAWP |
| melanogaster BIR2 | | | RNLKQKPHQLAEAGFFYTGVGDRVRCFSCGGGLMDWNDN |
| | | | DEPWEQHALWLSQCRFVKLMKGQLYIDTVAAKPVLAEEK |
| | | | EESTSIGGDT |
| Amanita thiersii | K, R, H | 129 | MICGQIIGKGESCFRCRDCGLDESCVMCSQCFHATDHIN |
| Skay4041 | | | HNVSFFVSQQPGGCCDCGDEEAWKKPMNCPYHPP |
| UBR-box domain | | | |
| (PS501) | | | |
| Helobdella robusta | K, R, H | 130 | MVCLKVFKLGEPTYSCRSVTCGMDPTCVLCVDCFQNSSH |
| UBR-box domain | | | KLHKYKMSTSGGGGYCDCGDLEAWKADPLCDLHKL |
| (PS502) | | | |
| Hydra vulgaris | K, R, H | 131 | MFCGRLFKVGDPTYTCKDCAADPTCVFCHDCFHQSVHTK |
| UBR-box domain | | | HKYKLFASQGRGGYCDCGDKEAWTNDPACNKHKE |
| (PS503) | | | |
| Galleria mellonella | K, R, H | 132 | MLCGKVFKQGEPAYSCRECGMDNTCVLCVECFKVSPHRH |
| UBR-box domain | | | HKYKMGQSGGGGCCDCGDTEAWKRDPFCERHAK |
| (PS504) | | | |
| Brachionus plicatilis | K, R, H | 133 | MVCGRVFKSGEPSYFCRECGTDPTCVLCSICFRHSKHRY |
| UBR-box domain | | | HKYVMMTSGGGGYCDCGDPEAWKSDPCCELHMP |
| (PS505) | | | |
| Capitella teleta | K, R, H | 134 | MLCGKVFKMGELTYSCRDCGTDPTCVLCMDCFQHSAHKK |
| UBR-box domain | | | HRYKMAASGGGGYCDCGDREAWKAEPFCDVHKR |
| (PS506) | | | |
| Sparassis crispa | K, R, H | 135 | MPCGHIFKKGESCFRCKDCALDDSCVLCSKCFEATDHAN |
| UBR-box domain | | | HNVSFFIAQQSGGCCDCGDIEAWLVPIDCPFHPV |
| (PS507) | | | |
| Anabarilius graham | K, R, H | 136 | MLCGRVFKEGETVYSCRDCAIDPTCVLCIECFQKSVHKS |
| UBR-box domain | | | HRYKMHASAGGGFCDCGDLEAWKTGPCCSQHDP |
| (PS508) | | | |
| Lottia gigantean | K, R, H | 137 | MICGHGFKTGEPTYSCRDCATDPTCVLCISCFQKSPHRE |
| UBR-box domain | | | HRYKMSASGGGGYCDCGDPEAWKIEPFCEQHKP |
| (PS509) | | | |
| Camponotus | K, R, H | 138 | MICGRMFKMGEPTYSCRQCGMDSTCVLCVDCFKQSAHRN |
| floridanus | | | HKYKMGTSSGGGCCDCGDTEAWKNEPFCKIHLA |
| UBR-box domain | | | |
| (PS510) | | | |
| Habropoda laboriosa | K, R, H | 139 | MICGKVFKMGEATYSCKECGVDPTCVLCADCFKQSAHRH |
| UBR-box domain | | | HKYRMGTSSGGGFCDCGDIEAWKKEPFCNTHLA |
| (PS511) | | | |
| Mastacembelus | K, R, H | 140 | MLCGRVFKEGETVYSCRDCAIDPTCVLCMDCFQDSVHKS |
| armatus | | | HRYKMHASAGGGFCDCGDVEAWKIGPYCSKHDP |
| UBR-box domain | | | |
| (PS512) | | | |
| Pyrenophora | K, R, H | 141 | MPCGHIFKNGEATYRCKTCTADDTCVLCARCFDASDHEG |
| Vseminiperda CCB06 | | | HQVFVSVSPGNSGCCDCGDDEAWVRPVHCNIHSA |
| UBR-box domain | | | |
| (PS513) | | | |
| Tribolium castaneum | K, R, H | 142 | MVCGRVFKLGEPTYSCRECGMDNTCVLCVNCFKNSEHRF |
| UBR-box domain | | | HKYKMGTSQGGGCCDCGDVEAWKKAPFCDVHIA |
| (PS514) | | | |
| Wasmannia | K, R, H | 143 | MICGKMFKIGEPTYSCRECGMDSTCVLCVDCFKQSAHRN |
| auropunctata | | | HKYKMGTSSGGGCCDCGDTEAWKKEPFCKTHVV |
| UBR-box domain | | | |
| (PS515) | | | |
| Crassostrea gigas | K, R, H | 144 | MLCGKVFKTGEPTYSCRDCANDPTCVLCIDCFQNGAHKN |
| UBR-box domain | | | HRYKMNTSGGGGYCDCGDQEAWTSHPFCNLHSP |
| (PS516) | | | |
| Harpegnathos | K, R, H | 145 | MMCGRVFKMGEPTYSCRECGVDSTCVLCVGCFQQSAHRD |
| saltator | | | HKYKMGTSGGGGCCDCGDTEAWKRDPFCEIHMV |
| UBR-box domain | | | |
| (PS517) | | | |
| Nilaparvata lugens | K, R, H | 146 | MVCGRVFKMGEPSYHCRECGMDATCVLCVDCFKKSSHRN |
| UBR-box domain | | | HKYKMGTSIGGGCCDCGDVEAWKTEPYCEVHIA |
| (PS518) | | | |
| Manduca sexta | K, R, H | 147 | MLCGRVFKQGEPAYSCRECGMDNTCVLCVECFKVSAHRH |
| UBR-box domain | | | HKYKMGQSGGGGCCDCGDTEAWKRDPFCELHAA |
| (PS519) | | | |
| Monopterus albus | K, R, H | 148 | MLCGRVFKEGETVYSCRDCAIDPTCVLCMDCFQDSVHKS |
| UBR-box domain | | | HRYKMHASSGGGFCDCGDVEAWKIGPCCSKHDP |
| (PS520) | | | |
| Lingula anatine | K, R, H | 149 | MLCGRVFRSGEPTYSCRDCAVDPTCVLCIDCFNNGAHRK |
| UBR-box domain | | | HKYRMSTSSGGGYCDCGDKEAWKTDPLCEIHRK |
| (PS521) | | | |
| Vombatus ursinus | K, R, H | 150 | MLCGKVFKSGETTYSCRDCAIDPTCVLCMNCFQSSVHKN |
| UBR-box domain | | | HRYKMHTSTGGGFCDCGDTEAWKTGPFCTIHEP |
| (PS522) | | | |
| Saccharomycesaceae | K, R, H | 151 | MAKSHRHTGRNCGRAFQPGEPLYRCQECAYDDTCVLCIS |
| sp. Ashbya aceri | | | CFNPDDHVNHHVSTHICNELHDGICDCGDAEAWNVPLHC |
| UBR-box domain | | | KAEED |
| (PS523) | | | |
| Drosophila ficusphila | K, R, H | 152 | MVCGKVFKNGEPTYSCRECGVDPTCVLCVNCFKRSAHRF |
| UBR-box domain | | | HKYKMSTSGGGGCCDCGDDEAWKKDHYCQLHLA |
| (PS524) | | | |
| Mus musculus | K, R, H | 153 | MLCGKVFKSGETTYSCRDCAIDPTCVLCMDCFQSSVHKN |
| UBR-box domain | | | HRYKMHTSTGGGFCDCGDTEAWKTGPFCVDHEP |
| (PS525) | | | |
| Maylandia zebra | K, R, H | 154 | MLCGRVFKEGETVYSCRDCAIDPTCVLCMDCFQDSVHKS |
| UBR-box domain | | | HRYKMHASSGGGFCDCGDVEAWKIGPYCSKHDP |
| (PS526) | | | |
| Mizuhopecten | K, R, H | 155 | MLCGKVFKYGEPTYSCRDCANDPTCVLCIDCFQKSAHKK |
| yessoensis | | | HRYKMSTSGGGGYCDCGDSEAWKTAPFCSNHKA |
| UBR-box domain | | | |
| (PS527) | | | |
| Kluyveromyces lactis | K, R, H | 156 | MHSKFNHAGRICGAKFRVGEPIYRCKECSFDDTCVLCVN |
| UBR-box domain | | | CFNPKDHVGHHVYTSICTEFNNGICDCGDKEAWNHELNC |
| (PS528) | | | KGAED |
| Chelonia mydas | K, R, H | 157 | MLCGKVFKGGETTYSCRDCAIDPTCVLCMDCFQNSIHKN |
| UBR-box domain | | | HRYKMHTSTGGGFCDCGDTEAWKTGPLCANHEP |
| (PS529) | | | |
| Acropora millepora | K, R, H | 158 | MLCGKVFKVGEPTYSCRDCGYDNTCVLCINCFQKSIHKN |
| UBR-box domain | | | HHYKMNTSGGGGVCDCGDVEAWKEGEACEIHQQ |
| (PS530) | | | |
| Musca domestica | K, R, H | 159 | MVCGKVFKIGEPTYSCRECGMDQTCVLCVNCFKQSAHRY |
| UBR-box domain | | | HKYKMSTSGGGGCCDCGDEEAWKKDHYCEEHLR |
| (PS531) | | | |
| Schizosaccharomyces | K, R, H | 160 | MSCGRIFKKGEVFYRCKTCSVDSNSALCVKCFRATDHHG |
| Vcryophilus OY26 | | | HETSFTISAGSGGCCDCGNSAAWIRDMPCKIHNR |
| UBR-box domain | | | |
| (PS532) | | | |
| Contarinia nasturtii | K, R, H | 161 | MVCGRVFKMNEPFYSCRECGMDPTCVLCVNCFKQSAHRH |
| UBR-box domain | | | HKYKMGTSAGGGCCDCGDNEAWKQDHYCDEHTK |
| (PS533) | | | |
| Schizosaccharomyces | K, R, H | 162 | MKCGHIFRKGEVFYRCKTCSVDSNSALCVKCFRATSHKD |
| pombe | | | HETSFTVSAGSGGCCDCGNAAAWIGDVSCKIHSH |
| UBR-box domain | | | |
| (PS534) | | | |
| Mus musculus | K, R, H | 163 | MLCGRVFKVGEPTYSCRDCAVDPTCVLCMECFLGSIHRD |
| UBR-box domain | | | HRYRMTTSGGGGFCDCGDTEAWKEGPYCQKHKL |
| (PS535) | | | |
| Aphis gossypii | K, R, H | 164 | MVCGRVFKMGEPTYNCRECGMDSTCVLCVDCFKRSPHKN |
| UBR-box domain | | | HKYKMGTSYGGGCCDCGDVEAWKHDPYCQTHKL |
| (PS536) | | | |
| Aedes aegypti | K, R, H | 165 | MVCGRVFKIGEPTYSCRECSMDPTCVLCSSCFKKSSHRL |
| UBR-box domain | | | HKYKMSTSGGGGCCDCGDHEAWKRDPSCEEHAV |
| (PS537) | | | |
| Saccharomyces | K, R, H | 166 | MGDVHKHTGRNCGRKFKIGEPLYRCHECGCDDTCVLCIH |
| cerevisiae | | | CFNPKDHVNHHVCTDICTEFTSGICDCGDEEAWNSPLHC |
| UBR-box domain | | | KAEEQ |
| (PS538) | | | |
| Saccharomyces | K, R, H | 167 | MGSVHKHTGRNCGRKFKIGEPLYRCHECGCDDTCVLCIH |
| cerevisiae | | | CFNPKDHVNHHVCTDICTEFTSGICDCGDEEAWNSPLHC |
| UBR1 D3S variant | | | KAEEQ |
| (PS25) | | | |
| Kazachstania | K, R, H | 168 | MQTSFTHKGRNCGRKFKVGEPLYRCHECGFDDTCVLCIH |
| africana CBS 2517 | | | CFNPADHENHHIYTDICNDFTSGICDCGDTEAWNGDLHC |
| UBR-box domain | | | KAEEI |
| (PS539) | | | |
| Clathrospora elynae | K, R, H | 169 | MPCGHIFKNGEATYRCKTCTADDTCVLCARCFDASDHEG |
| UBR-box domain | | | HQVFVSVSPGNSGCCDCGDDEAWVRPVHCNMHSA |
| (PS540) | | | |
| Aspergillus neoniger | K, R, H | 170 | MRCGHIFRAGEATYRCITCAADDTCVLCSRCFDASDHTG |
| CBS 115656 | | | HQYQISLSSGNCGCCDCGDEEAWRLPLFCAIHTD |
| UBR-box domain | | | |
| (PS541) | | | |
| Trichuris suis | K, R, H | 171 | MRCNHVFANGEATYSCRGCAADPTCVLCASCFELSAHKE |
| UBR-box domain | | | HKYMITTSSGTGYCDCGDPEAWKADPFCQQHQP |
| (PS542) | | | |
| Trichinella spiralis | K, R, H | 172 | MKCNRQLICGEPTYCCLDCACDQTCIFCHACFQSSEHKN |
| UBR-box domain | | | HRYSMSTSEGSGTCDCGDKEAWKSNYYCLNHKP |
| (PS543) | | | |
| Homo sapiens | K, R, H | 173 | MGPLGSLCGRVFKSGETTYSCRDCAIDPTCVLCMDCFQD |
| UBR1 | | | SVHKNHRYKMHTSTGGGFCDCGDTEAWKTGPFCVNHEP |
| (PS544) | | | |
| Homo sapiens | K, R, H | 174 | MGPLGSLCGRVFKVGEPTYSCRDCAVDPTCVLCMECFLG |
| UBR2 | | | SIHRDHRYRMTTSGGGGFCDCGDTEAWKEGPYCQKHE |
| Kluyveromyces | K, R, H | 175 | MVNEHRGSQCSKQCHGTETVYYCFDCTKNPLYEICEECE |
| marxianus | | | DETQHMGHRYTSRVVTRPEGKVCHCGDISGYNNPEKAFQ |
| UBR2 (PS615) | | | CKI |
| Kluyveromyces lactis | K, R, H | 176 | MHNDHRGSQCSKQCHGTETVYYCFDCTKNPLYEICEDCF |
| UBR2 | | | DESQHIGHRYTSRVVTRPEGKVCHCGDISSYNDPKKAFQ |
| (PS616) | | | CRI |
| Eremothecium | K, R, H | 177 | MPKEHRGTSCNKHCQPTETVYYCFDCTKNPLYEICEECF |
| sinecaudum | | | DADKHLGHRWTSKVVSRPEGKICHCGDPSGLTDPENGYE |
| UBR2 (PS617) | | | CKN |
| Zygosaccharomyces | K, R, H | 178 | MNASHKGAMCSKQCYPTETVFYCFTCTTNPLYEICESCF |
| bailii | | | DEEKHRGHLYTAKVVVRPEGRVCHCGDPFVFKEPRFAFL |
| UBR2 (PS618) | | | CKN |
| Vanderwaltozyma | K, R, H | 179 | MENLHIGSCCNRQCYPTQTVYYCLICTINPLYEICELCF |
| polyspora UBR2 | | | DEDKHVGHTYISKSVIRPEGKVCHCGNPNVFKKPEFAFN |
| (PS619) | | | CKN |
| Saccharomyces | K, R, H | 180 | MGNMHIGTACTRLCFPSETIYYCFTCSINPLYEICELCF |
| cerevisiae | | | DKEKHVNHSYVAKVVMRPEGRICHCGDPFAFNDPSDAFK |
| UBR2 (PS620) | | | CKN |
| Kluyveromyces | K, R, H | 181 | MHSKFSHAGRICGAKFKVGEPIYRCKECSFDDTCVLCVN |
| marxianus | | | CFNPKDHTGHHVYTTICTEFNNGICDCGDKEAWNHTLFC |
| UBR1 (PS621) | | | KAEEG |
| Kluyveromyces | K, R, H | 182 | MHSRFNHAGRICASKFKVGEPIYRCKECSFDDTCVICVN |
| dobzhanskii | | | CFNPKDHVGHHVYTSICSEFNNGICDCGDTEAWNHDMHC |
| UBR1 (PS622) | | | KADEN |
| Kazachstania | K, R, H | 183 | MSKQFRHKGRNCGRKFRLGEPLYRCQECGYDDTCVLCIN |
| naganishii | | | CFNPKDHEGHHIYTDICNDFTSGICDCGDEEAWLSPLHC |
| UBR1 (PS623) | | | KAEED |
| Eremothecium | K, R, H | 184 | MPKNHNHKGRNCGRSFQPGEPLYRCQECAYDDTCVLCIR |
| sinecaudum | | | CFNPLDHVNHHVSTHICSEFNDGICDCGDVEAWNVELNC |
| UBR1 (PS624) | | | KAEED |
| Saccharomyces | K, R, H | 185 | MGDVHKHTGRNCGRKFKIGEPLYRCHECGCDDTCVLCIH |
| eubayanus | | | CFNPKDHINHHVCTDICSEFTSGICDCGDEEAWNSSLHC |
| UBR1 (PS625) | | | KAEEQ |
| Zygosaccharomyces | K, R, H | 186 | MYHVYKHSGRNCGRKFKVGEPIYRCHECGYDETCVLCIH |
| parabailii | | | CFNPKDHDSHHVYIDICSEFSTGICDCGDTEAFVNPLHC |
| UBR1 (PS626) | | | KAEED |
| Zygosaccharomyces | K, R, H | 187 | MPKYHQHSGRYCGRKFKVGEPIYRCHECGFDETCVICIH |
| mellis | | | CFNAKDHETHHVSVSICSEYSTGICDCGDTEAFVNPLHC |
| UBR1 (PS627) | | | RAEEV |
| Candida albicans | K, R, H | 188 | MSHRAYHKNSPCGRIFRKGEPIHRCLTCGFDDTCALCSH |
| UBR1 | | | CFQPEYHEGHKVHIGICQRENGGVCDCGDPEAWTQELFC |
| (PS628) | | | PYAVD |
| Pichia pastoris | K, R, H | 189 | MCPNYKHHGRPCARQFKQGEPIYRCYECGFDETCVMCMH |
| UBR1 | | | CFNREQHRDHEVSISIASSSNDGICDCGDPQAWNIELHC |
| (PS629) | | | QSELD |

*Binding preferences are inferred from published scientific literature and/or further demonstrated by the inventors in single-molecule and/or ensemble experiments, as described herein.
**Binding to phosphotyrosine may occur at a peptide terminus or at an internal position.

In some embodiments, an amino acid recognition molecule comprises a single polypeptide having tandem copies of two or more amino acid binding proteins (e.g., two or more binders). As used herein, in some embodiments, a tandem arrangement or orientation of elements in a molecule refers to an end-to-end joining of each element to the next element in a linear fashion such that the elements are fused in series. For example, in some embodiments, a polypeptide having tandem copies of two binders refers to a fusion polypeptide in which the C-terminus of one binder is fused to the N-terminus of the other binder. Similarly, a polypeptide having tandem copies of two or more binders refers to a fusion polypeptide in which the C-terminus of a first binder is fused to the N-terminus of a second binder, the C-terminus of the second binder is fused to the N-terminus of a third binder, and so forth. Such fusion polypeptides can comprise multiple copies of the same binder or multiple copies of different binders. In some embodiments, a fusion polypeptide of the application has at least two and up to ten binders (e.g., at least 2 binders and up to eight, six, five, four, or three binders). In some embodiments, a fusion polypeptide of the application has five or fewer binders (e.g., two, three, four, or five binders).

In some embodiments, a fusion polypeptide is provided by expression of a single coding sequence containing segments encoding monomeric binder subunits separated by segments encoding flexible linkers, where expression of the single coding sequence produces a single full-length polypeptide having two or more independent binding sites. In some embodiments, one or more of the monomeric subunits (e.g., binders) are ClpS proteins. In some embodiments, ClpS subunits may be identical or non-identical. Where non-identical, ClpS subunits may be distinct variants of the same parent ClpS protein, or they may be derived from different parent ClpS proteins. In some embodiments, a fusion polypeptide comprises one or more ClpS monomers and one or more non-ClpS monomers. In some embodiments, the monomeric subunits comprise non-ClpS monomers. In some embodiments, the monomeric subunits comprise one or more degradation pathway proteins. For example, in some embodiments, the monomeric subunits comprise one or more of a Gid protein, a UBR-box protein or UBR-box domain-containing protein fragment thereof, a p62 protein or ZZ domain-containing fragment thereof, and a ClpS protein (e.g., ClpS1, ClpS2).

In some embodiments, at least one binder of a fusion polypeptide has an amino acid sequence selected from Table 1 or Table 2 (or having an amino acid sequence that has at least 50%, at least 60%, at least 70%, at least 80%, 80-90%, 90-95%, 95-99%, or higher, amino acid sequence identity to an amino acid sequence selected from Table 1 or Table 2). In some embodiments, each binder of a fusion polypeptide has an amino acid sequence that is at least 80% (e.g., 80-90%, 90-95%, 95-99%, or higher) identical to an amino acid sequence selected from Table 1 or Table 2 (or having an amino acid sequence that has at least 50%, at least 60%, at least 70%, at least 80%, 80-90%, 90-95%, 95-99%, or higher, amino acid sequence identity to an amino acid sequence selected from Table 1 or Table 2). In some embodiments, a binder of a fusion polypeptide is modified and includes one or more amino acid deletions, additions, or mutations relative to a sequence set forth in Table 1 or Table 2. In some embodiments, a binder of a fusion polypeptide includes a deletion, addition, or mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids (which may or may not be consecutive amino acids) relative to a sequence set forth in Table 1 or Table 2.

In some embodiments, binders of a fusion polypeptide recognize the same set of one or more amino acids. In some embodiments, binders of a fusion polypeptide recognize a distinct set of one or more amino acids. In some embodiments, binders of a fusion polypeptide recognize an overlapping set of amino acids. In some embodiments, where the binders of a fusion polypeptide recognize the same amino acid, they may recognize the amino acid with the same characteristic pulsing pattern or with different characteristic pulsing patterns.

In some embodiments, binders of a fusion polypeptide are joined end-to-end, either by a covalent bond or a linker that covalently joins the C-terminus of one binder to the N-terminus of another binder. In the context of fusion polypeptides of the application, a linker refers to one or more amino acids within a fusion polypeptide that joins two binders and that does not form part of the polypeptide sequence corresponding to either of the two binders. In some embodiments, a linker comprises at least two amino acids (e.g., at least 2, 3, 4, 5, 6, 8, 10, 15, 25, 50, 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 5 and about 50, between about 2 and about 20, between about 5 and about 20, or between about 2 and about 30, amino acids).

In some embodiments, the amino acid recognition molecule comprises a sequence GGGSGGGSGGGSG (“Linker 1”) (SEQ ID NO: 214); GSAGSAAGSGEF (“Linker 2”) (SEQ ID NO: 215); or GSAGSAAGSGEFGSAGSAAGSGEFGSAGSAAGSGEF (“Linker 3”) (SEQ ID NO: 216). In some embodiments, the amino acid recognition molecule comprises the sequence Linker 1. In some embodiments, the amino acid recognition molecule comprises the sequence Linker 2. In some embodiments, the amino acid recognition molecule, or salt thereof, comprises the sequence Linker 3. In some embodiments, the amino acid recognition molecule, or salt thereof, comprises a sequence shown in Table 1, Table 2, or Table 3. In some embodiments, the amino acid recognition molecule, or salt thereof, comprises a sequence shown in Table 3. In some embodiments, the amino acid recognition molecule, or salt thereof, comprises PS610 (Bis-atClpS2-V1, Linker 2).

In some embodiments, the amino acid recognition molecule comprises a sequence that is at least 40%, at least 50%, at least 60%, at least 70%, at least 80%, at least 85%, at least 90%, at least 95%, at least 98%, or 100% identical to PS2132 (SEQ ID NO: 213). In some embodiments, the amino acid recognition molecule comprises a sequence that is between about 40% and about 100% (e.g., 80-98%, 80-95%, 80-90%, 85-95%, 90-98%, 50-100%, 60-100%, 70-100%, 80-100%, 90-100%, or 95-100%) identical to PS2132 (SEQ ID NO: 213).

TABLE 3
Non-limiting examples of multivalent binders.

| | SEQ | |
|---|---|---|
| | ID | |
| Name | NO: | Sequence |
| PS609 | 190 | MSDSPVDLKPKPKVKPKLERPKLYKVMLLNDDYTPMSFVTVVLKAVFRMSEDTGRRV |
| (Bis- | | MMTAHRFGSAVVVVCERDIAETKAKEATDLGKEAGFPLMFTTEPEEGGGSGGGSGGG |
| atClpS2- | | SGMSDSPVDLKPKPKVKPKLERPKLYKVMLLNDDYTPMSFVTVVLKAVFRMSEDTGR |
| V1, | | RVMMTAHRFGSAVVVVCERDIAETKAKEATDLGKEAGFPLMFTTEPEEGHHHHHHHH |
| Linker 1) | | HHGGGSGGGSGGGSGLNDFFEAQKIEWHEGGGSGGGSGGGSGLNDFFEAQKIEWHE |
| PS610 | 191 | MSDSPVDLKPKPKVKPKLERPKLYKVMLLNDDYTPMSFVTVVLKAVFRMSEDTGRRV |
| (Bis- | | MMTAHRFGSAVVVVCERDIAETKAKEATDLGKEAGFPLMFTTEPEEGSAGSAAGSGE |
| atClpS2- | | FMSDSPVDLKPKPKVKPKLERPKLYKVMLLNDDYTPMSFVTVVLKAVFRMSEDTGRR |
| V1, | | VMMTAHRFGSAVVVVCERDIAETKAKEATDLGKEAGFPLMFTTEPEEGHHHHHHHHH |
| Linker 2) | | HGGGSGGGSGGGSGLNDFFEAQKIEWHEGGGSGGGSGGGSGLNDFFEAQKIEWHE |
| PS611 | 192 | MSDSPVDLKPKPKVKPKLERPKLYKVMLLNDDYTPMSFVTVVLKAVERMSEDTGRRV |
| (Bis- | | MMTAHRFGSAVVVVCERDIAETKAKEATDLGKEAGFPLMFTTEPEEGSAGSAAGSGE |
| atClpS2- | | FGSAGSAAGSGEFGSAGSAAGSGEFMSDSPVDLKPKPKVKPKLERPKLYKVMLLNDD |
| V1, | | YTPMSFVTVVLKAVFRMSEDTGRRVMMTAHRFGSAVVVVCERDIAETKAKEATDLGK |
| Linker 3) | | EAGFPLMFTTEPEEGHHHHHHHHHHGGGSGGGSGGGSGLNDFFEAQKIEWHEGGGSG |
| | | GGSGGGSGLNDFFEAQKIEWHE |
| PS612 | 193 | MSDSPVDLKPKPKVKPKLERPKLYKVMLLNDDYTPMSFVTVVLKAVFRMSEDTGRRV |
| (atClpS2- | | MMTAHRFGSAVVVVCERDIAETKAKEATDLGKEAGFPLMFTTEPEEGSAGSAAGSGE |
| V1 + | | FMAFPARGKTAPKNEVRRQPPYNVILLNDDDHTYRYVIEMLQKIFGFPPEKGFQIAE |
| PS372, | | EVDRTGRVILLTTSKEHAELKQDQVHSYGPDPYLGRPCSGSMTCVIEPAVGGSHHHH |
| Linker 2) | | HHHHHHGGGSGGGSGGGSGLNDFFEAQKIEWHEGGGSGGGSGGGSGLNDFFEAQKIE |
| | | WHE |
| PS613 | 19 | MAFPARGKTAPKNEVRRQPPYNVILLNDDDHTYRYVIEMLQKIFGFPPEKGFQIAEE |
| (Bis- | | VDRTGRVILLTTSKEHAELKQDQVHSYGPDPYLGRPCSGSMTCVIEPAVGSAGSAAG |
| PS372, | | SGEFMSDSPVDLKPKPKVKPKLERPKLYKVMLLNDDYTPMSFVTVVLKAVERMSEDT |
| Linker 2) | | GRRVMMTAHRFGSAVVVVCERDIAETKAKEATDLGKEAGFPLMFTTEPEEGHHHHHH |
| | | HHHHGGGSGGGSGGGSGLNDFFEAQKIEWHEGGGSGGGSGGGSGLNDFFEAQKIEWH |
| | | E |
| PS614 | 195 | MAFPARGKTAPKNEVRRQPPYNVILLNDDDHTYRYVIEMLQKIFGFPPEKGFQIAEE |
| (PS372 + | | VDRTGRVILLTTSKEHAELKQDQVHSYGPDPYLGRPCSGSMTCVIEPAVGSAGSAAG |
| atClpS2- | | SGEFMAFPARGKTAPKNEVRRQPPYNVILLNDDDHTYRYVIEMLQKIFGFPPEKGFQ |
| V1, | | IAEEVDRTGRVILLTTSKEHAELKQDQVHSYGPDPYLGRPCSGSMTCVIEPAVGGSH |
| Linker 2) | | HHHHHHHHHGGGSGGGSGGGSGLNDFFEAQKIEWHEGGGSGGGSGGGSGLNDFFEAQ |
| | | KIEWHE |
| PS637 | 196 | MPTAASATESAIEDTPAPARPEVDGRTKPKRQPRYHVVLWNDDDHTYQYVVVMLQSL |
| (Bis- | | FGHPPERGYRLAKEVDTQGRVIVLITTREHAELKRDQIHAFGYDRLLARSKGSMKAS |
| PS557, | | IEAEEGGGSGGGSGGGSGMPTAASATESAIEDTPAPARPEVDGRTKPKRQPRYHVVL |
| Linker 1) | | WNDDDHTYQYVVVMLQSLFGHPPERGYRLAKEVDTQGRVIVLTTTREHAELKRDQIH |
| | | AFGYDRLLARSKGSMKASIEAEEGGSHHHHHHHHHHGGGSGGGSGGGSGLNDFFEAQ |
| | | KIEWHEGGGSGGGSGGGSGLNDFFEAQKIEWHE |
| PS638 | 197 | MPTAASATESAIEDTPAPARPEVDGRTKPKRQPRYHVVLWNDDDHTYQYVVVMLQSL |
| (Bis- | | FGHPPERGYRLAKEVDTQGRVIVLITTREHAELKRDQIHAFGYDRLLARSKGSMKAS |
| PS557, | | IEAEEGSAGSAAGSGEFMPTAASATESAIEDTPAPARPEVDGRTKPKRQPRYHVVLW |
| Linker 2) | | NDDDHTYQYVVVMLQSLFGHPPERGYRLAKEVDTQGRVIVLITTREHAELKRDQIHA |
| | | FGYDRLLARSKGSMKASIEAEEGGSHHHHHHHHHHGGGSGGGSGGGSGLNDFFEAQK |
| | | IEWHEGGGSGGGSGGGSGLNDFFEAQKIEWHE |
| PS639 | 198 | MPTAASATESAIEDTPAPARPEVDGRTKPKRQPRYHVVLWNDDDHTYQYVVVMLQSL |
| (Bis- | | FGHPPERGYRLAKEVDTQGRVIVLTTTREHAELKRDQIHAFGYDRLLARSKGSMKAS |
| PS557, | | IEAEEGSAGSAAGSGEFGSAGSAAGSGEFGSAGSAAGSGEFMPTAASATESAIEDTP |
| Linker 3) | | APARPEVDGRTKPKRQPRYHVVLWNDDDHTYQYVVVMLQSLFGHPPERGYRLAKEVD |
| | | TQGRVIVLTTTREHAELKRDQIHAFGYDRLLARSKGSMKASIEAEEGGSHHHHHHHH |
| | | HHGGGSGGGSGGGSGLNDFFEAQKIEWHEGGGSGGGSGGGSGLNDFFEAQKIEWHE |
| PS640 | 199 | MSDSPVDLKPKPKVKPKLERPKLYKVMLLNDDYTPMSFVTVVLKAVFRMSEDTGRRV |
| (atClpS2- | | MMTAHRFGSAVVVVCERDIAETKAKEATDLGKEAGFPLMFTTEPEEGSAGSAAGSGE |
| V1+ | | FMPTAASATESAIEDTPAPARPEVDGRTKPKRQPRYHVVLWNDDDHTYQYVVVMLQS |
| PS557, | | LFGHPPERGYRLAKEVDTQGRVIVLITTREHAELKRDQIHAFGYDRLLARSKGSMKA |
| Linker 2) | | SIEAEEGGSHHHHHHHHHHGGGSGGGSGGGSGLNDFFEAQKIEWHEGGGSGGGSGGG |
| | | SGLNDFFEAQKIEWHE |
| PS641 | 200 | MPTAASATESAIEDTPAPARPEVDGRTKPKRQPRYHVVLWNDDDHTYQYVVVMLQSL |
| (PS557 + | | FGHPPERGYRLAKEVDTQGRVIVLTTTREHAELKRDQIHAFGYDRLLARSKGSMKAS |
| atClpS2- | | IEAEEGSAGSAAGSGEFMSDSPVDLKPKPKVKPKLERPKLYKVMLLNDDYTPMSFVT |
| V1, | | VVLKAVFRMSEDTGRRVMMTAHRFGSAVVVVCERDIAETKAKEATDLGKEAGFPLMF |
| Linker 2) | | TTEPEEGGSHHHHHHHHHHGGGSGGGSGGGSGLNDFFEAQKIEWHEGGGSGGGSGGG |
| | | SGLNDFFEAQKIEWHE |
| PS651 | 201 | MSDSPVDLKPKPKVKPKLERPKLYKVMLLNDDYTPMSFVTVVLKAVFRMSEDTGRRV |
| (3xatClpS | | MMTAHRFGSAVVVVCERDIAETKAKEATDLGKEAGFPLMFTTEPEEGSAGSAAGSGE |
| 2-V1, | | FMSDSPVDLKPKPKVKPKLERPKLYKVMLLNDDYTPMSFVTVVLKAVFRMSEDTGRR |
| Linker 2) | | VMMTAHRFGSAVVVVCERDIAETKAKEATDLGKEAGFPLMFTTEPEEGSAGSAAGSG |
| | | EFMSDSPVDLKPKPKVKPKLERPKLYKVMLLNDDYTPMSFVTVVLKAVFRMSEDTGR |
| | | RVMMTAHRFGSAVVVVCERDIAETKAKEATDLGKEAGFPLMFTTEPEEGHHHHHHHH |
| | | HHGGGSGGGSGGGSGLNDFFEAQKIEWHEGGGSGGGSGGGSGLNDFFEAQKIEWHE |
| PS652 | 202 | MSDSPVDLKPKPKVKPKLERPKLYKVMLLNDDYTPMSFVTVVLKAVFRMSEDTGRRV |
| (4xatClpS | | MMTAHRFGSAVVVVCERDIAETKAKEATDLGKEAGFPLMFTTEPEEGSAGSAAGSGE |
| 2-V1, | | FMSDSPVDLKPKPKVKPKLERPKLYKVMLLNDDYTPMSFVTVVLKAVFRMSEDTGRR |
| Linker 2) | | VMMTAHRFGSAVVVVCERDIAETKAKEATDLGKEAGFPLMFTTEPEEGSAGSAAGSG |
| | | EFMSDSPVDLKPKPKVKPKLERPKLYKVMLLNDDYTPMSFVTVVLKAVFRMSEDTGR |
| | | RVMMTAHRFGSAVVVVCERDIAETKAKEATDLGKEAGFPLMFTTEPEEGSAGSAAGS |
| | | GEFMSDSPVDLKPKPKVKPKLERPKLYKVMLLNDDYTPMSFVTVVLKAVFRMSEDTG |
| | | RRVMMTAHRFGSAVVVVCERDIAETKAKEATDLGKEAGFPLMFTTEPEEGHHHHHHH |
| | | HHHGGGSGGGSGGGSGLNDFFEAQKIEWHEGGGSGGGSGGGSGLNDFFEAQKIEWHE |
| PS653 | 203 | MAFPARGKTAPKNEVRRQPPYNVILLNDDDHTYRYVIEMLQKIFGFPPEKGFQIAEE |
| (3xPS372, | | VDRTGRVILLTTSKEHAELKQDQVHSYGPDPYLGRPCSGSMTCVIEPAVGSAGSAAG |
| Linker 2) | | SGEFMAFPARGKTAPKNEVRRQPPYNVILLNDDDHTYRYVIEMLQKIFGFPPEKGFQ |
| | | IAEEVDRIGRVILLTTSKEHAELKQDQVHSYGPDPYLGRPCSGSMTCVIEPAVGSAG |
| | | SAAGSGEFMAFPARGKTAPKNEVRRQPPYNVILLNDDDHTYRYVIEMLQKIFGFPPE |
| | | KGFQIAEEVDRTGRVILLTTSKEHAELKQDQVHSYGPDPYLGRPCSGSMTCVIEPAV |
| | | GGSHHHHHHHHHHGGGSGGGSGGGSGLNDFFEAQKIEWHEGGGSGGGSGGGSGLNDF |
| | | FEAQKIEWHE |
| PS654 | 204 | MAFPARGKTAPKNEVRRQPPYNVILLNDDDHTYRYVIEMLQKIFGFPPEKGFQIAEE |
| (4×PS372, | | VDRTGRVILLTTSKEHAELKQDQVHSYGPDPYLGRPCSGSMTCVIEPAVGSAGSAAG |
| Linker 2) | | SGEFMAFPARGKTAPKNEVRRQPPYNVILLNDDDHTYRYVIEMLQKIFGFPPEKGFQ |
| | | IAEEVDRTGRVILLTTSKEHAELKQDQVHSYGPDPYLGRPCSGSMTCVIEPAVGSAG |
| | | SAAGSGEFMAFPARGKTAPKNEVRRQPPYNVILLNDDDHTYRYVIEMLQKIFGFPPE |
| | | KGFQIAEEVDRTGRVILLTTSKEHAELKQDQVHSYGPDPYLGRPCSGSMTCVIEPAV |
| | | GSAGSAAGSGEFMAFPARGKTAPKNEVRRQPPYNVILLNDDDHTYRYVIEMLQKIFG |
| | | FPPEKGFQIAEEVDRTGRVILLTTSKEHAELKQDQVHSYGPDPYLGRPCSGSMTCVI |
| | | EPAVGGSHHHHHHHHHHGGGSGGGSGGGSGLNDFFEAQKIEWHEGGGSGGGSGGGSG |
| | | LNDFFEAQKIEWHE |
| PS655 | 205 | MPTAASATESAIEDTPAPARPEVDGRTKPKRQPRYHVVLWNDDDHTYQYVVVMLQSL |
| (3×PS557, | | FGHPPERGYRLAKEVDTQGRVIVLITTREHAELKRDQIHAFGYDRLLARSKGSMKAS |
| Linker 2) | | IEAEEGSAGSAAGSGEFMPTAASATESAIEDTPAPARPEVDGRTKPKRQPRYHVVLW |
| | | NDDDHTYQYVVVMLQSLFGHPPERGYRLAKEVDTQGRVIVLTTTREHAELKRDQIHA |
| | | FGYDRLLARSKGSMKASIEAEEGSAGSAAGSGEFMPTAASATESAIEDTPAPARPEV |
| | | DGRTKPKRQPRYHVVLWNDDDHTYQYVVVMLQSLFGHPPERGYRLAKEVDTQGRVIV |
| | | LTTTREHAELKRDQIHAFGYDRLLARSKGSMKASIEAEEGGSHHHHHHHHHHGGGSG |
| | | GGSGGGSGLNDFFEAQKIEWHEGGGSGGGSGGGSGLNDFFEAQKIEWHE |
| PS656 | 206 | MPTAASATESAIEDTPAPARPEVDGRTKPKRQPRYHVVLWNDDDHTYQYVVVMLQSL |
| (4×PS557, | | FGHPPERGYRLAKEVDTQGRVIVLITTREHAELKRDQIHAFGYDRLLARSKGSMKAS |
| Linker 2) | | IEAEEGSAGSAAGSGEFMPTAASATESAIEDTPAPARPEVDGRTKPKRQPRYHVVLW |
| | | NDDDHTYQYVVVMLQSLFGHPPERGYRLAKEVDTQGRVIVLITTREHAELKRDQIHA |
| | | FGYDRLLARSKGSMKASIEAEEGSAGSAAGSGEFMPTAASATESAIEDTPAPARPEV |
| | | DGRTKPKRQPRYHVVLWNDDDHTYQYVVVMLQSLFGHPPERGYRLAKEVDTQGRVIV |
| | | LTTTREHAELKRDQIHAFGYDRLLARSKGSMKASIEAEEGSAGSAAGSGEFMPTAAS |
| | | ATESAIEDTPAPARPEVDGRTKPKRQPRYHVVLWNDDDHTYQYVVVMLQSLFGHPPE |
| | | RGYRLAKEVDTQGRVIVLITTREHAELKRDQIHAFGYDRLLARSKGSMKASIEAEEG |
| | | GSHHHHHHHHHHGGGSGGGSGGGSGLNDFFEAQKIEWHEGGGSGGGSGGGSGLNDFF |
| | | EAQKIEWHE |
| PS690 | 207 | MHSKFSHAGRICGAKFKVGEPIYRCKECSFDDTCVLCVNCFNPKDHTGHHVYTTICT |
| (Bis- | | EFNNGICDCGDKEAWNHTLFCKAEEGGGGSGGGSGGGSGMHSKFSHAGRICGAKFKV |
| PS621, | | GEPIYRCKECSFDDTCVLCVNCFNPKDHTGHHVYTTICTEFNNGICDCGDKEAWNHT |
| Linker 1) | | LFCKAEEGGGSHHHHHHHHHHGGGSGGGSGGGSGLNDFFEAQKIEWHEGGGSGGGSG |
| | | GGSGLNDFFEAQKIEWHE |
| PS691 | 208 | MHSKFSHAGRICGAKFKVGEPIYRCKECSFDDTCVLCVNCFNPKDHTGHHVYTTICT |
| (Bis- | | EFNNGICDCGDKEAWNHTLFCKAEEGGSAGSAAGSGEFMHSKFSHAGRICGAKFKVG |
| PS621, | | EPIYRCKECSFDDTCVLCVNCFNPKDHTGHHVYTTICTEFNNGICDCGDKEAWNHTL |
| Linker 2) | | FCKAEEGGGSHHHHHHHHHHGGGSGGGSGGGSGLNDFFEAQKIEWHEGGGSGGGSGG |
| | | GSGLNDFFEAQKIEWHE |
| PS692 | 209 | MHSKFSHAGRICGAKFKVGEPIYRCKECSFDDTCVLCVNCFNPKDHTGHHVYTTICT |
| (Bis- | | EFNNGICDCGDKEAWNHTLFCKAEEGGSAGSAAGSGEFGSAGSAAGSGEFGSAGSAA |
| PS621, | | GSGEFMHSKFSHAGRICGAKFKVGEPIYRCKECSFDDTCVLCVNCFNPKDHTGHHVY |
| Linker 3) | | TTICTEFNNGICDCGDKEAWNHTLFCKAEEGGGSHHHHHHHHHHGGGSGGGSGGGSG |
| | | LNDFFEAQKIEWHEGGGSGGGSGGGSGLNDFFEAQKIEWHE |
| PS693 | 210 | MHSKFNHAGRICGAKFRVGEPIYRCKECSFDDTCVLCVNCFNPKDHVGHHVYTSICT |
| (Bis- | | EFNNGICDCGDKEAWNHELNCKGAEDGGGSGGGSGGGSGMHSKFNHAGRICGAKFRV |
| PS528, | | GEPIYRCKECSFDDTCVLCVNCFNPKDHVGHHVYTSICTEFNNGICDCGDKEAWNHE |
| Linker 1) | | LNCKGAEDGGSHHHHHHHHHHGGGSGGGSGGGSGLNDFFEAQKIEWHEGGGSGGGSG |
| | | GGSGLNDFFEAQKIEWHE |
| PS694 | 211 | MHSKFNHAGRICGAKFRVGEPIYRCKECSFDDTCVLCVNCFNPKDHVGHHVYTSICT |
| (Bis- | | EFNNGICDCGDKEAWNHELNCKGAEDGSAGSAAGSGEFMHSKFNHAGRICGAKFRVG |
| PS528, | | EPIYRCKECSFDDTCVLCVNCFNPKDHVGHHVYTSICTEFNNGICDCGDKEAWNHEL |
| Linker 2) | | NCKGAEDGGSHHHHHHHHHHGGGSGGGSGGGSGLNDFFEAQKIEWHEGGGSGGGSGG |
| | | GSGLNDFFEAQKIEWHE |
| PS695 | 212 | MHSKFNHAGRICGAKFRVGEPIYRCKECSFDDTCVLCVNCFNPKDHVGHHVYTSICT |
| (Bis- | | EFNNGICDCGDKEAWNHELNCKGAEDGSAGSAAGSGEFGSAGSAAGSGEFGSAGSAA |
| PS528, | | GSGEFMHSKFNHAGRICGAKFRVGEPIYRCKECSFDDTCVLCVNCFNPKDHVGHHVY |
| Linker 3) | | TSICTEFNNGICDCGDKEAWNHELNCKGAEDGGSHHHHHHHHHHGGGSGGGSGGGSG |
| | | LNDFFEAQKIEWHEGGGSGGGSGGGSGLNDFFEAQKIEWHE |
| PS2132 | 213 | MNGLSAQHERIAPARHECVYTECYSEENVWKLCEHIKTSKRCPLGDVYAVFISNERK |
| | | MVPIWKQKSGRGEEPVIWDYKVILLHDTHKEQTFIYDLDTTLPFPCPFDTYVKEAFK |
| | | SDNYIRPAFWRKLRVVPADVFLQNFASDRSHMKDASGGWRMPPPPYPCIETAESRMN |
| | | LDDFISMNPSVGWGHVYTLEEFVQHFGKT |

In some embodiments, a recognition molecule of the disclosure is an amino acid binding protein which can be used with other types of amino acid binding molecules, such as a peptidase and/or a nucleic acid aptamer, in a sequencing method. A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, a labeled recognition molecule comprises a peptidase that has been modified to inactivate exopeptidase or endopeptidase activity. In this way, the labeled recognition molecule selectively binds without also cleaving the amino acid from a polypeptide. In yet other embodiments, a peptidase that has not been modified to inactivate exopeptidase or endopeptidase activity may be used with an amino acid binding protein of the disclosure. For example, in some embodiments, a labeled recognition molecule comprises a labeled exopeptidase.

In some embodiments, an amino acid recognition molecule comprises one or more labels. In some embodiments, the one or more labels comprise a luminescent label (e.g., a dye) or a conductivity label as described elsewhere herein. In some embodiments, the one or more labels comprise one or more polyol moieties (e.g., one or more moieties selected from dextran, polyvinylpyrrolidone, polyethylene glycol, polypropylene glycol, polyoxyethylene glycol, and polyvinyl alcohol). For example, in some embodiments, an amino acid recognition molecule is PEGylated. In some embodiments, polyol modification (e.g., PEGylation) can limit the extent of non-specific sticking to a substrate (e.g., sequencing chip) surface. In some embodiments, polyol modification can limit the extent of aggregation or interaction between an amino acid recognition molecule with other recognition molecules, with a cleaving reagent, or with other species present in a sequencing reaction mixture. PEGylation can be performed by incubating a recognition molecule (e.g., an amino acid binding protein, such as a ClpS protein) with mPEG4-NHS ester, which labels primary amines such as surface-exposed lysine side chains. Other types of PEG and other methods of polyol modification are known in the art.

In some embodiments, the one or more labels comprise a tag sequence. For example, in some embodiments, an amino acid recognition molecule comprises a tag sequence that provides one or more functions other than amino acid binding. In some embodiments, a tag sequence comprises at least one biotin ligase recognition sequence that permits biotinylation of the recognition molecule (e.g., incorporation of one or more biotin molecules, including biotin and bis-biotin moieties). In some embodiments, the tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. Additional examples of functional sequences in a tag sequence include purification tags, cleavage sites, and other moieties useful for purification and/or modification of recognition molecules.

Examples of amino acid recognition molecules (e.g., amino acid binding proteins) for use in accordance with the disclosure are described more fully in PCT International Application No. PCT/US2019/061831, filed Nov. 15, 2019, and PCT International Application No. PCT/US2021/033493, filed May 20, 2021, the relevant contents of which are incorporated by reference in their entireties.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position). Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

Some aspects are directed to a set of luminescent labels comprising a plurality of luminescent labels (e.g., comprising a plurality of dyes). In some embodiments, each luminescent label of the set of luminescent labels has a distinct value for one or more luminescent characteristics. In some cases, a set of luminescent labels may advantageously be used to label a set of reaction components (e.g., amino acid recognition molecules) to ensure that each type of reaction component can be identified during protein sequencing and/or nucleic acid sequencing. In some embodiments, the set of luminescent labels may comprise one or more luminescently labeled oligonucleotide structures as described herein. In some embodiments, the set of luminescent labels may comprise one or more fluorophores known in the art (e.g., Cy®3, Cy®3B, ATTO Rho6G).

Non-limiting examples of luminescent characteristics include luminescent lifetime, luminescent intensity, bin ratio, and luminescent wavelength. In certain embodiments, each luminescent label has a value for a luminescent characteristic that differs from the value for the luminescent characteristic of each other luminescent label of the set of luminescent labels. In certain embodiments, a minimum percentage difference between luminescent characteristic values for any two luminescent labels of a set of luminescent labels is at least 1%, at least 5%, at least 10%, at least 20%, at least 30%, at least 50%, at least 100%, at least 150%, at least 200%, or at least 500%. In certain embodiments, a minimum percentage difference between luminescent characteristic values for any two luminescent labels of a set of luminescent labels is in a range from 1-5%, 1-10%, 1-20%, 1-30%, 1-50%, 1-100%, 1-150%, 1-200%, 1-500%, 5-10%, 5-20%, 5-30%, 5-50%, 5- 100%, 5-150%, 5-200%, 5-500%, 10-20%, 10-30%, 10-50%, 10-100%, 10-150%, 10-200%, 10-500%, 20-50%, 20-100%, 20-150%, 20-200%, 20-500%, 50-100%, 50-150%, 50-200%, 50-500%, 100-200%, 100-500%, or 200-500%.

A set of luminescent labels may have any suitable number of luminescent labels. In certain embodiments, the set of luminescent labels comprises two or more luminescent labels, three or more luminescent labels four or more luminescent labels, four or more luminescent labels, five or more luminescent labels, six or more luminescent labels, seven or more luminescent labels, eight or more luminescent labels, nine or more luminescent labels, or ten or more luminescent labels. In some embodiments, the set of luminescent labels comprises two, three, four, five, six, seven, eight, nine, or ten luminescent labels, or more.

In some embodiments, the luminescent characteristic comprises a bin ratio. In certain cases, bin ratio may be a measurement of luminescent lifetime. In some cases, the bin ratio of a luminescent label may be obtained using an integrated device described herein. In some embodiments, the bin ratio of a luminescent label may refer to a ratio of photoelectrons collected during a first time period (bin 0) to photoelectrons collected during a second time period (bin 1). In certain embodiments, the first time period may start a relatively long time after an excitation pulse (e.g., 3 ns after an excitation pulse). In certain embodiments, the second time period may start a relatively short time after an excitation pulse (e.g., 1 ns after an excitation pulse). In some cases, a relatively low bin ratio may indicate that a dye has a relatively short luminescent lifetime. In some cases, a relatively high bin ratio may indicate that a dye has a relatively long luminescent lifetime.

In some embodiments, each luminescent label of a set of luminescent labels may have a distinct bin ratio value. In certain embodiments, a minimum difference between bin ratio values of a set of luminescent labels is at least 0.05, at least 0.1, at least 0.2, at least 0.3, at least 0.4, at least 0.5, at least 0.6, at least 0.7, at least 0.8, at least 0.9, or at least 1.0. In certain embodiments, a minimum difference between bin ratio values of a set of luminescent labels is in a range from 0.05 to 0.2, 0.05 to 0.3, 0.05 to 0.4, 0.05 to 0.5, 0.05 to 0.6, 0.05 to 0.7, 0.05 to 0.8, 0.05 to 0.9, 0.05 to 1.0, 0.1 to 0.2, 0.1 to 0.3, 0.1 to 0.4, 0.1 to 0.5, 0.1 to 0.6, 0.1 to 0.7, 0.1 to 0.8, 0.1 to 0.9, 0.1 to 1.0, 0.2 to 0.5, 0.2 to 0.6, 0.2 to 0.7, 0.2 to 0.8, 0.2 to 0.9, 0.2 to 1.0, 0.5 to 1.0, 0.6 to 1.0, 0.7 to 1.0, 0.8 to 1.0, or 0.9 to 1.0. In certain embodiments, a minimum percentage difference between bin ratio values of a set of luminescent labels is at least 1%, at least 5%, at least 10%, at least 20%, at least 30%, at least 50%, at least 100%, at least 150%, at least 200%, or at least 500%. In certain embodiments, a minimum percentage difference between bin ratio values of a set of luminescent labels is in a range from 1-5%, 1-10%, 1-20%, 1-30%, 1-50%, 1-100%, 1-150%, 1-200%, 1-500%, 5-10%, 5-20%, 5-30%, 5-50%, 5-100%, 5-150%, 5-200%, 5-500%, 10⁻²⁰%, 10⁻³⁰%, 10⁻⁵⁰%, 10⁻¹⁰⁰%, 10⁻¹⁵⁰%, 10⁻²⁰⁰%, 10-500%, 20-50%, 20-100%, 20-150%, 20-200%, 20-500%, 50-100%, 50-150%, 50-200%, 50-500%, 100-200%, 100-500%, or 200-500%.

In some embodiments, each luminescent label of a set of luminescent labels has a unique combination of two or more different luminescence characteristics. In some embodiments, a system comprises a first luminescent label having a first ordered pair of characteristics comprising a first value of a first characteristic and a first value of a second characteristic. In some embodiments, a system comprises a second luminescent label having a second ordered pair of characteristics comprising a second value of the first characteristic and a second value of the second characteristic. In some embodiments, a system comprises a third luminescent label having a third ordered pair of characteristics comprising a third value of the first characteristic and a third value of the second characteristic. In certain embodiments, the first ordered pair, the second ordered pair, and the third ordered pair differ from one another in at least one of the respective values of the first and/or second characteristics. In certain embodiments, the first ordered pair, the second ordered pair, and the third ordered pair are separated by a certain minimum distance.

In some embodiments, a method comprises providing a first luminescent label having a first ordered pair of characteristics comprising a first value of a first characteristic and a first value of a second characteristic. In some embodiments, the method comprises providing a second luminescent label having a second ordered pair of characteristics comprising a second value of the first characteristic and a second value of the second characteristic. In some embodiments, the method comprises providing a third luminescent label comprising a luminescently labeled oligonucleotide structure comprising a first single-stranded oligonucleotide comprising one or more first fluorophores and a first complementary single-stranded oligonucleotide comprising one or more second fluorophores, wherein the third luminescent label has a third ordered pair of characteristics comprising a third value of the first characteristic and a third value of the second characteristic. In some embodiments, the method comprises modifying the numbers and/or identities of the one or more first fluorophores and/or the one or more second fluorophores such that the first ordered pair, the second ordered pair, and the third ordered pair differ from one another in at least one of the respective values of the first and/or second characteristics.

In some instances, a set of luminescent labels comprises a plurality of luminescent labels, where each luminescent label occupies of a distinct spatial region (e.g., a different location) of a two-dimensional plot of two luminescence characteristics. In certain instances, the two-dimensional plot is a plot of intensity vs. bin ratio. In some embodiments, an ordered pair of characteristics associated with a luminescent label represents a centroid of a cluster of points associated with the luminescent label on a two-dimensional plot of two luminescence characteristics.

In some embodiments, a set of luminescent labels comprises one or more, two or more, three or more, four or more, or five or more of a first luminescent label comprising R1C1, a second luminescent label comprising C2C, a third luminescent label comprising SG4Cy3, a fourth luminescent label comprising one or more copies of ATRho6G, and a fifth luminescent label comprising one or more copies of Cy3B. In some embodiments, a set of luminescent labels comprises one or more, two or more, three or more, four or more, five or more, or six or more of a first luminescent label comprising R1C1, a second luminescent label comprising C2C, a third luminescent label comprising SG4Cy3, a fourth luminescent label comprising one or more copies of ATRho6G, a fifth luminescent label comprising one or more copies of Cy3B, and a sixth luminescent label comprising one or more copies of PS610-tris-BDP3037. In some embodiments, a set of luminescent labels comprises one or more, two or more, three or more, four or more, five or more, or six or more of a first luminescent label comprising R1C1, a second luminescent label comprising C2C, a third luminescent label comprising SG4Cy3, a fourth luminescent label comprising one or more copies of ATRho6G, a fifth luminescent label comprising one or more copies of Cy3B, and a sixth luminescent label comprising one or more copies of PS610-bis-BDP3037.

### Methods of Sequencing a Polypeptide

In another aspect, provided herein is a method of sequencing a polypeptide, the method comprising:
(i) directing a series of pulses of one or more excitation energies towards a composition comprising a polypeptide, and an amino acid recognition molecule, or a salt thereof, wherein the amino acid recognition molecule, or salt thereof, comprises at least one instance of Formula (II):

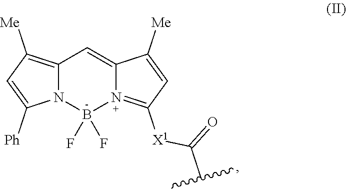

or a salt thereof, wherein:
each instance of X¹ is substituted or unsubstituted C₁-C₆ alkylene;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

As generally described herein, each instance of X¹ is substituted or unsubstituted C₁-C₆ alkylene.

In some embodiments, at least one instance of X¹ is substituted C₁-C₆ alkylene, substituted C₁-C₅ alkylene, substituted C₁-C₄ alkylene, substituted C₁-C₃ alkylene, or substituted C₁-C₂ alkylene. In some embodiments, at least one instance of X¹ is substituted C₁-C₆ alkylene. In some embodiments, at least one instance of X¹ is substituted C₁-C₅ alkylene. In some embodiments, at least one instance of X¹ is substituted C₁-C₄ alkylene. In some embodiments, at least one instance of X¹ is substituted C₁-C₃ alkylene. In some embodiments, at least one instance of X¹ is substituted C₁-C₂ alkylene.

In some embodiments, at least one instance of X¹ is C₁-C₆ alkylene, C₁-C₅ alkylene, C₁-C₄ alkylene, C₁-C₃ alkylene, or C₁-C₂ alkylene substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂; wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring.

In some embodiments, at least one instance of X¹ is unsubstituted C₁-C₆ alkylene, unsubstituted C₁-C₅ alkylene, unsubstituted C₁-C₄ alkylene, unsubstituted C₁-C₃ alkylene, or unsubstituted C₁-C₂ alkylene. In some embodiments, at least one instance of X¹ is unsubstituted C₁-C₆ alkylene or unsubstituted C₁-C₃ alkylene. In some embodiments, at least one instance of X¹ is unsubstituted C₁-C₆ alkylene. In some embodiments, at least one instance of X¹ is unsubstituted C₁-C₅ alkylene. In some embodiments, at least one instance of X¹ is unsubstituted C₁-C₄ alkylene. In some embodiments, at least one instance of X¹ is unsubstituted C₁-C₃ alkylene. In some embodiments, at least one instance of X¹ is unsubstituted C₁-C₂ alkylene.

In some embodiments, at least one instance of X¹ is methylene, ethylene, n-propylene, isopropylene, n-butylene, tert-butylene, sec-butylene, isobutylene, n-pentylene, 3-pentanylene, amylene, neopentylene, 3-methyl-2-butanylene, tert-amylene, or n-hexylene.

In some embodiments, at least one instance of X¹ is methylene (—CH₂—), ethylene (—(CH₂)₂—), n-propylene (—(CH₂)₃—), n-butylene (—(CH₂)₄—), n-pentylene (—(CH₂)₅—), or n-hexylene (—(CH₂)₆—). In some embodiments, at least one instance of X¹ is —CH₂—, —(CH₂)₂—, or —(CH₂)₃—. In some embodiments, at least one instance of X¹ is —(CH₂)₂—.

In some embodiments, the amino acid recognition molecule, or salt thereof, comprises at least one instance of Formula (II), wherein the at least one instance of Formula (II) is of Formula (II-a):

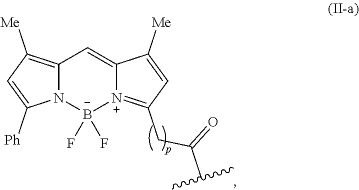

or a salt thereof, wherein each instance of p is independently 1, 2, 3, 4, 5, or 6. In some embodiments of Formula (II-a), at least one instance of p is 1. In some embodiments of Formula (II-a), at least one instance of p is 2. In some embodiments of Formula (II-a), at least one instance of p is 3. In some embodiments of Formula (II-a), at least one instance of p is 4. In some embodiments of Formula (II-a), at least one instance of p is 5. In some embodiments of Formula (II-a), at least one instance of p is 6.

In some embodiments, the amino acid recognition molecule, or salt thereof, comprises at least one instance of Formula (II), wherein the at least one instance of Formula (II) is of formula:

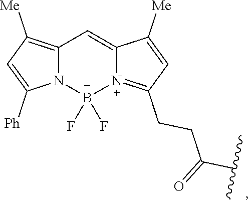 [SMILES: *C(=O)CCC1=[N+]2C(=Cc3c(C)cc(-c4ccccc4)n3[B-]2(F)F)C(C)=C1]

or a salt thereof.

In some embodiments, the amino acid recognition molecule, or salt thereof, is linked to at least one instance of Formula (II), or salt thereof, via a linker. In some embodiments, the linker comprises one or more moieties selected from substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, and polynucleotide. In some embodiments, the linker comprises one or more moieties selected from substituted or unsubstituted alkylene, substituted or unsubstituted alkenylene, or substituted or unsubstituted alkynylene, substituted or unsubstituted heteroalkylene, substituted or unsubstituted carbocyclylene, substituted or unsubstituted heterocyclylene, substituted or unsubstituted arylene, substituted or unsubstituted heteroarylene, and polynucleotide.

In some embodiments, the linker comprises substituted or unsubstituted aliphatic. In some embodiments, the linker comprises substituted or unsubstituted alkylene, substituted or unsubstituted alkenylene, or substituted or unsubstituted alkynylene.

In some embodiments, the linker comprises substituted or unsubstituted alkylene. In some embodiments, the linker comprises substituted or unsubstituted C₁-C₁₂ alkylene. In some embodiments, the linker comprises substituted or unsubstituted C₁-C₆ alkylene. In some embodiments, the linker comprises substituted or unsubstituted C₁-C₃ alkylene. In some embodiments, the linker comprises substituted alkylene. In some embodiments, the linker comprises substituted C₁-C₁₂ alkylene. In some embodiments, the linker comprises substituted C₁-C₆ alkylene. In some embodiments, the linker comprises substituted C₁-C₃ alkylene. In some embodiments, the linker comprises acylene. In some embodiments, the linker comprises unsubstituted alkylene. In some embodiments, the linker comprises unsubstituted C₁-C₁₂ alkylene. In some embodiments, the linker comprises unsubstituted C₁-C₆ alkylene. In some embodiments, the linker comprises unsubstituted C₁-C₃ alkylene.

In some embodiments, the linker comprises substituted or unsubstituted alkenylene. In some embodiments, the linker comprises substituted or unsubstituted C₁-C₁₂ alkenylene. In some embodiments, the linker comprises substituted or unsubstituted C₁-C₆ alkenylene. In some embodiments, the linker comprises substituted or unsubstituted C₁-C₃ alkenylene. In some embodiments, the linker comprises substituted alkenylene. In some embodiments, the linker comprises substituted C₁-C₁₂ alkenylene. In some embodiments, the linker comprises substituted C₁-C₆ alkenylene. In some embodiments, the linker comprises substituted C₁-C₃ alkenylene. In some embodiments, the linker comprises unsubstituted alkenylene. In some embodiments, the linker comprises unsubstituted C₁-C₁₂ alkenylene. In some embodiments, the linker comprises unsubstituted C₁-C₆ alkenylene. In some embodiments, the linker comprises unsubstituted C₁-C₃ alkenylene.

In some embodiments, the linker comprises substituted or unsubstituted alkynylene. In some embodiments, the linker comprises substituted or unsubstituted C₁-C₁₂ alkynylene. In some embodiments, the linker comprises substituted or unsubstituted C₁-C₆ alkynylene. In some embodiments, the linker comprises substituted or unsubstituted C₁-C₃ alkynylene. In some embodiments, the linker comprises substituted alkynylene. In some embodiments, the linker comprises substituted C₁-C₁₂ alkynylene. In some embodiments, the linker comprises substituted C₁-C₆ alkynylene. In some embodiments, the linker comprises substituted C₁-C₃ alkynylene. In some embodiments, the linker comprises unsubstituted alkynylene. In some embodiments, the linker comprises unsubstituted C₁-C₁₂ alkynylene. In some embodiments, the linker comprises unsubstituted C₁-C₆ alkynylene. In some embodiments, the linker comprises unsubstituted C₁-C₃ alkynylene.

In some embodiments, the linker comprises substituted or unsubstituted heteroaliphatic. In some embodiments, the linker comprises substituted or unsubstituted heteroalkylene. In some embodiments, the linker comprises substituted or unsubstituted C₁-C₁₂ heteroalkylene. In some embodiments, the linker comprises substituted or unsubstituted C₁-C₆ heteroalkylene. In some embodiments, the linker comprises substituted or unsubstituted C₁-C₃ heteroalkylene. In some embodiments, the linker comprises substituted heteroalkylene. In some embodiments, the linker comprises substituted C₁-C₁₂ heteroalkylene. In some embodiments, the linker comprises substituted C₁-C₆ heteroalkylene. In some embodiments, the linker comprises substituted C₁-C₃ heteroalkylene. In some embodiments, the linker comprises unsubstituted heteroalkylene. In some embodiments, the linker comprises unsubstituted C₁-C₁₂ heteroalkylene. In some embodiments, the linker comprises unsubstituted C₁-C₆ heteroalkylene. In some embodiments, the linker comprises unsubstituted C₁-C₃ heteroalkylene.

In some embodiments, the linker comprises substituted or unsubstituted carbocyclylene. In some embodiments, the linker comprises substituted or unsubstituted C₃-C₁₀ carbocyclylene. In some embodiments, the linker comprises substituted or unsubstituted C₃-C₆ carbocyclylene. In some embodiments, the linker comprises substituted carbocyclylene. In some embodiments, the linker comprises substituted C₃-C₁₀ carbocyclylene. In some embodiments, the linker comprises substituted C₃-C₆ carbocyclylene. In some embodiments, the linker comprises unsubstituted carbocyclylene. In some embodiments, the linker comprises unsubstituted C₃-C₁₀ carbocyclylene. In some embodiments, the linker comprises unsubstituted C₃-C₆ carbocyclylene.

In some embodiments, the linker comprises substituted or unsubstituted heterocyclylene. In some embodiments, the linker comprises substituted or unsubstituted 3-10 membered heterocyclylene. In some embodiments, the linker comprises substituted or unsubstituted 3-6 membered heterocyclylene. In some embodiments, the linker comprises substituted heterocyclylene. In some embodiments, the linker comprises substituted 3-10 membered heterocyclylene. In some embodiments, the linker comprises substituted 3-6 membered heterocyclylene. In some embodiments, the linker comprises unsubstituted heterocyclylene. In some embodiments, the linker comprises unsubstituted 3-10 membered heterocyclylene. In some embodiments, the linker comprises unsubstituted 3-6 membered heterocyclylene.

In some embodiments, the linker comprises substituted or unsubstituted arylene. In some embodiments, the linker comprises substituted or unsubstituted phenylene. In some embodiments, the linker comprises substituted arylene. In some embodiments, the linker comprises substituted phenylene. In some embodiments, the linker comprises unsubstituted arylene. In some embodiments, the linker comprises unsubstituted phenylene.

In some embodiments, the linker comprises substituted or unsubstituted heteroarylene. In some embodiments, the linker comprises substituted or unsubstituted 5-10 membered heteroarylene. In some embodiments, the linker comprises substituted or unsubstituted 5-6 membered monocyclic heteroarylene. In some embodiments, the linker comprises substituted heteroarylene. In some embodiments, the linker comprises substituted 5-10 membered heteroarylene. In some embodiments, the linker comprises substituted 5-6 membered monocyclic heteroarylene. In some embodiments, the linker comprises unsubstituted heteroarylene. In some embodiments, the linker comprises unsubstituted 5-10 membered heteroarylene. In some embodiments, the linker comprises unsubstituted 5-6 membered monocyclic heteroarylene.

In some embodiments, the linker comprises a polynucleotide. In some embodiments, the linker comprises a polynucleotide further comprising at least one substituent. In some embodiments, the polynucleotide is further substituted. In some embodiments, the polynucleotide further comprises at least one substituent.

In some embodiments, the amino acid recognition molecule, or salt thereof, comprises at least 1, 2, 3, 4, or 5 instances of Formula (II), or a salt thereof. In some embodiments, the amino acid recognition molecule, or salt thereof, comprises at least 1 instance of Formula (II), or a salt thereof. In some embodiments, the amino acid recognition molecule, or salt thereof, comprises at least 2 instances of Formula (II), or a salt thereof. In some embodiments, the amino acid recognition molecule, or salt thereof, comprises at least 3 instances of Formula (II), or a salt thereof. In some embodiments, the amino acid recognition molecule, or salt thereof, comprises at least 4 instances of Formula (II), or a salt thereof. In some embodiments, the amino acid recognition molecule, or salt thereof, comprises at least 5 instances of Formula (II), or a salt thereof. In some embodiments, the amino acid recognition molecule, or salt thereof, comprises 1 instance of Formula (II), or a salt thereof. In some embodiments, the amino acid recognition molecule, or salt thereof, comprises 2 instances of Formula (II), or a salt thereof. In some embodiments, the amino acid recognition molecule, or salt thereof, comprises 3 instances of Formula (II), or a salt thereof. In some embodiments, the amino acid recognition molecule, or salt thereof, comprises 4 instances of Formula (II), or a salt thereof. In some embodiments, the amino acid recognition molecule, or salt thereof, comprises 5 instances of Formula (II), or a salt thereof.

In another aspect, provided herein is a method of sequencing a polypeptide, the method comprising:
(i) directing a series of pulses of one or more excitation energies towards a composition comprising a polypeptide, and an amino acid recognition molecule, or a salt thereof, wherein the amino acid recognition molecule, or salt thereof, comprises at least one instance of Formula (IV):

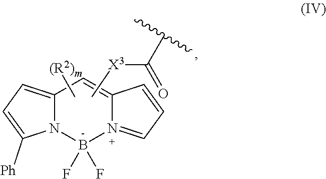

or a salt thereof, wherein:
each instance of R² is substituted or unsubstituted C₁-C₆ alkyl;
each instance of X³ is substituted or unsubstituted C₁-C₆ alkylene;
each instance of m is 1, 2, 3, or 5; and
each instance of [symbol] is a bond to the amino acid recognition molecule, or salt thereof;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

As generally described herein, each instance of R² is substituted or unsubstituted C₁-C₆ alkyl.

In some embodiments, at least one instance of R² is substituted or unsubstituted C₁-C₆ alkyl, substituted or unsubstituted C₁-C₅ alkyl, substituted or unsubstituted C₁-C₄ alkyl, substituted or unsubstituted C₁-C₃ alkyl, or substituted or unsubstituted C₁-C₂ alkyl. In some embodiments, at least one instance of R² is substituted or unsubstituted C₁-C₆ alkyl. In some embodiments, at least one instance of R² is substituted or unsubstituted C₁-C₅ alkyl. In some embodiments, at least one instance of R² is substituted or unsubstituted C₁-C₄ alkyl. In some embodiments, at least one instance of R² is substituted or unsubstituted C₁-C₃ alkyl. In some embodiments, at least one instance of R² is substituted or unsubstituted C₁-C₂ alkyl.

In some embodiments, at least one instance of R² is substituted C₁-C₆ alkyl, substituted C₁-C₅ alkyl, substituted C₁-C₄ alkyl, substituted C₁-C₃ alkyl, or substituted C₁-C₂ alkyl. In some embodiments, at least one instance of R² is substituted C₁-C₆ alkyl. In some embodiments, at least one instance of R² is substituted C₁-C₅ alkyl. In some embodiments, at least one instance of R² is substituted C₁-C₄ alkyl. In some embodiments, at least one instance of R² is substituted C₁-C₃ alkyl. In some embodiments, at least one instance of R² is substituted C₁-C₂ alkyl.

In some embodiments, at least one instance of R² is C₁-C₆ alkyl, C₁-C₅ alkyl, C₁-C₄ alkyl, C₁-C₃ alkyl, or C₁-C₂ alkyl substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂, wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring.

In some embodiments, at least one instance of R² is unsubstituted C₁-C₆ alkyl, unsubstituted C₁-C₅ alkyl, unsubstituted C₁-C₄ alkyl, unsubstituted C₁-C₃ alkyl, or unsubstituted C₁-C₂ alkyl. In some embodiments, at least one instance of R² is unsubstituted C₁-C₆ alkyl. In some embodiments, at least one instance of R² is unsubstituted C₁-C₅ alkyl. In some embodiments, at least one instance of R² is unsubstituted C₁-C₄ alkyl. In some embodiments, at least one instance of R² is unsubstituted C₁-C₃ alkyl. In some embodiments, at least one instance of R² is unsubstituted C₁-C₂ alkyl.

In some embodiments, at least one instance of R² is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, at least one instance of R² is methyl, ethyl, n-propyl, n-butyl, n-pentyl, or n-hexyl. In some embodiments, at least one instance of R² is methyl, ethyl, or n-propyl. In some embodiments, at least one instance of R² is methyl (—CH₃).

As generally described herein, each instance of X³ is substituted or unsubstituted C₁-C₆ alkylene.

In some embodiments, at least one instance of X³ is substituted C₁-C₆ alkylene, substituted C₁-C₅ alkylene, substituted C₁-C₄ alkylene, substituted C₁-C₃ alkylene, or substituted C₁-C₂ alkylene. In some embodiments, at least one instance of X³ is substituted C₁-C₆ alkylene. In some embodiments, at least one instance of X³ is substituted C₁-C₅ alkylene. In some embodiments, at least one instance of X³ is substituted C₁-C₄ alkylene. In some embodiments, at least one instance of X³ is substituted C₁-C₃ alkylene. In some embodiments, at least one instance of X³ is substituted C₁-C₂ alkylene.

In some embodiments, at least one instance of X³ is C₁-C₆ alkylene, C₁-C₅ alkylene, C₁-C₄ alkylene, C₁-C₃ alkylene, or C₁-C₂ alkylene substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂; wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring.

In some embodiments, at least one instance of X³ is unsubstituted C₁-C₆ alkylene, unsubstituted C₁-C₅ alkylene, unsubstituted C₁-C₄ alkylene, unsubstituted C₁-C₃ alkylene, or unsubstituted C₁-C₂ alkylene. In some embodiments, at least one instance of X³ is unsubstituted C₁-C₆ alkylene or unsubstituted C₁-C₃ alkylene. In some embodiments, at least one instance of X³ is unsubstituted C₁-C₆ alkylene. In some embodiments, at least one instance of X³ is unsubstituted C₁-C₅ alkylene. In some embodiments, at least one instance of X³ is unsubstituted C₁-C₄ alkylene. In some embodiments, at least one instance of X³ is unsubstituted C₁-C₃ alkylene. In some embodiments, at least one instance of X³ is unsubstituted C₁-C₂ alkylene.

In some embodiments, at least one instance of X³ is methylene, ethylene, n-propylene, isopropylene, n-butylene, tert-butylene, sec-butylene, isobutylene, n-pentylene, 3-pentanylene, amylene, neopentylene, 3-methyl-2-butanylene, tert-amylene, or n-hexylene.

In some embodiments, at least one instance of X³ is methylene (—CH₂—), ethylene (—(CH₂)₂—), n-propylene (—(CH₂)₃—), n-butylene (—(CH₂)₄—), n-pentylene (—(CH₂)₅—), or n-hexylene (—(CH₂)₆—). In some embodiments, at least one instance of X³ is —CH₂—, —(CH₂)₂—, or —(CH₂)₃—. In some embodiments, at least one instance of X³ is —(CH₂)₂—.

As generally described herein, each instance of m is 1, 2, 3, 4, or 5.

In some embodiments, at least one instance of m is 1, 2, 3, 4, or 5. In some embodiments, at least one instance of m is 1, 2, 3, or 4. In some embodiments, at least one instance of m is 1, 2, or 3. In some embodiments, at least one instance of m is 1 or 2. In some embodiments, at least one instance of m is 1. In some embodiments, at least one instance of m is 2. In some embodiments, at least one instance of m is 3. In some embodiments, at least one instance of m is 4. In some embodiments, at least one instance of m is 5.

As generally described herein, each instance of [symbol] is a bond to the amino acid recognition molecule, or salt thereof.

In some embodiments, at least one instance of Formula (IV) is of Formula (IV-a):

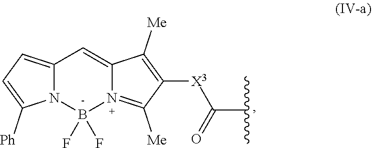

or a salt thereof.

In some embodiments, at least one instance of Formula (IV) is of Formula (IV-b):

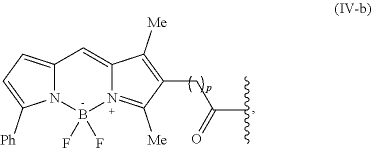

or a salt thereof, wherein p is 1, 2, 3, 4, 5, or 6. In some embodiments of Formula (IV-b), p is 1. In some embodiments of Formula (IV-b), p is 2. In some embodiments of Formula (IV-b), p is 3. In some embodiments of Formula (IV-b), p is 4. In some embodiments of Formula (IV-b), p is 5. In some embodiments of Formula (IV-b), p is 6.

In some embodiments, at least one instance of Formula (IV) is of formula:

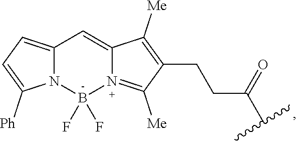

or a salt thereof.

In some embodiments, the amino acid recognition molecule, or salt thereof, is linked to at least one instance of Formula (IV), or salt thereof, via a linker. In some embodiments, the linker comprises one or more moieties selected from substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, and polynucleotide. In some embodiments, the linker comprises one or more moieties selected from substituted or unsubstituted alkylene, substituted or unsubstituted alkenylene, or substituted or unsubstituted alkynylene, substituted or unsubstituted heteroalkylene, substituted or unsubstituted carbocyclylene, substituted or unsubstituted heterocyclylene, substituted or unsubstituted arylene, substituted or unsubstituted heteroarylene, and polynucleotide.

In some embodiments, the linker comprises substituted or unsubstituted aliphatic. In some embodiments, the linker comprises substituted or unsubstituted alkylene, substituted or unsubstituted alkenylene, or substituted or unsubstituted alkynylene.

In some embodiments, the linker comprises substituted or unsubstituted alkylene. In some embodiments, the linker comprises substituted or unsubstituted C₁-C₁₂ alkylene. In some embodiments, the linker comprises substituted or unsubstituted C₁-C₆ alkylene. In some embodiments, the linker comprises substituted or unsubstituted C₁-C₃ alkylene. In some embodiments, the linker comprises substituted alkylene. In some embodiments, the linker comprises substituted C₁-C₁₂ alkylene. In some embodiments, the linker comprises substituted C₁-C₆ alkylene. In some embodiments, the linker comprises substituted C₁-C₃ alkylene. In some embodiments, the linker comprises acylene. In some embodiments, the linker comprises unsubstituted alkylene. In some embodiments, the linker comprises unsubstituted C₁-C₁₂ alkylene. In some embodiments, the linker comprises unsubstituted C₁-C₆ alkylene. In some embodiments, the linker comprises unsubstituted C₁-C₃ alkylene.

In some embodiments, the linker comprises substituted or unsubstituted alkenylene. In some embodiments, the linker comprises substituted or unsubstituted C₁-C₁₂ alkenylene. In some embodiments, the linker comprises substituted or unsubstituted C₁-C₆ alkenylene. In some embodiments, the linker comprises substituted or unsubstituted C₁-C₃ alkenylene. In some embodiments, the linker comprises substituted alkenylene. In some embodiments, the linker comprises substituted C₁-C₁₂ alkenylene. In some embodiments, the linker comprises substituted C₁-C₆ alkenylene. In some embodiments, the linker comprises substituted C₁-C₃ alkenylene. In some embodiments, the linker comprises unsubstituted alkenylene. In some embodiments, the linker comprises unsubstituted C₁-C₁₂ alkenylene. In some embodiments, the linker comprises unsubstituted C₁-C₆ alkenylene. In some embodiments, the linker comprises unsubstituted C₁-C₃ alkenylene.

In some embodiments, the linker comprises substituted or unsubstituted alkynylene. In some embodiments, the linker comprises substituted or unsubstituted C₁-C₁₂ alkynylene. In some embodiments, the linker comprises substituted or unsubstituted C₁-C₆ alkynylene. In some embodiments, the linker comprises substituted or unsubstituted C₁-C₃ alkynylene. In some embodiments, the linker comprises substituted alkynylene. In some embodiments, the linker comprises substituted C₁-C₁₂ alkynylene. In some embodiments, the linker comprises substituted C₁-C₆ alkynylene. In some embodiments, the linker comprises substituted C₁-C₃ alkynylene. In some embodiments, the linker comprises unsubstituted alkynylene. In some embodiments, the linker comprises unsubstituted C₁-C₁₂ alkynylene. In some embodiments, the linker comprises unsubstituted C₁-C₆ alkynylene. In some embodiments, the linker comprises unsubstituted C₁-C₃ alkynylene.

In some embodiments, the linker comprises substituted or unsubstituted heteroaliphatic. In some embodiments, the linker comprises substituted or unsubstituted heteroalkylene. In some embodiments, the linker comprises substituted or unsubstituted C₁-C₁₂ heteroalkylene. In some embodiments, the linker comprises substituted or unsubstituted C₁-C₆ heteroalkylene. In some embodiments, the linker comprises substituted or unsubstituted C₁-C₃ heteroalkylene. In some embodiments, the linker comprises substituted heteroalkylene. In some embodiments, the linker comprises substituted C₁-C₁₂ heteroalkylene. In some embodiments, the linker comprises substituted C₁-C₆ heteroalkylene. In some embodiments, the linker comprises substituted C₁-C₃ heteroalkylene. In some embodiments, the linker comprises unsubstituted heteroalkylene. In some embodiments, the linker comprises unsubstituted C₁-C₁₂ heteroalkylene. In some embodiments, the linker comprises unsubstituted C₁-C₆ heteroalkylene. In some embodiments, the linker comprises unsubstituted C₁-C₃ heteroalkylene.

In some embodiments, the linker comprises substituted or unsubstituted carbocyclylene. In some embodiments, the linker comprises substituted or unsubstituted C₃-C₁₀ carbocyclylene. In some embodiments, the linker comprises substituted or unsubstituted C₃-C₆ carbocyclylene. In some embodiments, the linker comprises substituted carbocyclylene. In some embodiments, the linker comprises substituted C₃-C₁₀ carbocyclylene. In some embodiments, the linker comprises substituted C₃-C₆ carbocyclylene. In some embodiments, the linker comprises unsubstituted carbocyclylene. In some embodiments, the linker comprises unsubstituted C₃-C₁₀ carbocyclylene. In some embodiments, the linker comprises unsubstituted C₃-C₆ carbocyclylene.

In some embodiments, the linker comprises substituted or unsubstituted heterocyclylene. In some embodiments, the linker comprises substituted or unsubstituted 3-10 membered heterocyclylene. In some embodiments, the linker comprises substituted or unsubstituted 3-6 membered heterocyclylene. In some embodiments, the linker comprises substituted heterocyclylene. In some embodiments, the linker comprises substituted 3-10 membered heterocyclylene. In some embodiments, the linker comprises substituted 3-6 membered heterocyclylene. In some embodiments, the linker comprises unsubstituted heterocyclylene. In some embodiments, the linker comprises unsubstituted 3-10 membered heterocyclylene. In some embodiments, the linker comprises unsubstituted 3-6 membered heterocyclylene.

In some embodiments, the linker comprises substituted or unsubstituted arylene. In some embodiments, the linker comprises substituted or unsubstituted phenylene. In some embodiments, the linker comprises substituted arylene. In some embodiments, the linker comprises substituted phenylene. In some embodiments, the linker comprises unsubstituted arylene. In some embodiments, the linker comprises unsubstituted phenylene.

In some embodiments, the linker comprises substituted or unsubstituted heteroarylene. In some embodiments, the linker comprises substituted or unsubstituted 5-10 membered heteroarylene. In some embodiments, the linker comprises substituted or unsubstituted 5-6 membered monocyclic heteroarylene. In some embodiments, the linker comprises substituted heteroarylene. In some embodiments, the linker comprises substituted 5-10 membered heteroarylene. In some embodiments, the linker comprises substituted 5-6 membered monocyclic heteroarylene. In some embodiments, the linker comprises unsubstituted heteroarylene. In some embodiments, the linker comprises unsubstituted 5-10 membered heteroarylene. In some embodiments, the linker comprises unsubstituted 5-6 membered monocyclic heteroarylene.

In some embodiments, the linker comprises a polynucleotide. In some embodiments, the linker comprises a polynucleotide further comprising at least one substituent. In some embodiments, the polynucleotide is further substituted. In some embodiments, the polynucleotide further comprises at least one substituent.

In some embodiments, the amino acid recognition molecule, or salt thereof, comprises at least 1, 2, 3, 4, or 5 instances of Formula (IV), or a salt thereof. In some embodiments, the amino acid recognition molecule, or salt thereof, comprises at least 1 instance of Formula (IV), or a salt thereof. In some embodiments, the amino acid recognition molecule, or salt thereof, comprises at least 2 instances of Formula (IV), or a salt thereof. In some embodiments, the amino acid recognition molecule, or salt thereof, comprises at least 3 instances of Formula (IV), or a salt thereof. In some embodiments, the amino acid recognition molecule, or salt thereof, comprises at least 4 instances of Formula (IV), or a salt thereof. In some embodiments, the amino acid recognition molecule, or salt thereof, comprises at least 5 instances of Formula (IV), or a salt thereof. In some embodiments, the amino acid recognition molecule, or salt thereof, comprises 1 instance of Formula (IV), or a salt thereof. In some embodiments, the amino acid recognition molecule, or salt thereof, comprises 2 instances of Formula (IV), or a salt thereof. In some embodiments, the amino acid recognition molecule, or salt thereof, comprises 3 instances of Formula (IV), or a salt thereof. In some embodiments, the amino acid recognition molecule, or salt thereof, comprises 4 instances of Formula (IV), or a salt thereof. In some embodiments, the amino acid recognition molecule, or salt thereof, comprises 5 instances of Formula (IV), or a salt thereof.

In some embodiments, the composition further comprises one or more amino acid recognition molecules, or salts thereof, conjugated to a dye, wherein the dye is not a 1,9-dimethyl-3-phenyl BODIPY dye. In some embodiments, the dye is a fluorophore. In some embodiments, the dye comprises an aromatic or heteroaromatic compound. In some embodiment, the dye comprises a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluorescein, rhodamine, xanthene, or other like compound.

In some embodiments, the dye is one or more dyes selected from: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor®405, Alexa Fluor®430, Alexa Fluor®480, Alexa Fluor®488, Alexa Fluor®514, Alexa Fluor®532, Alexa Fluor®546, Alexa Fluor®555, Alexa Fluor®568, Alexa Fluor®594, Alexa Fluor® 610-X, Alexa Fluor®633, Alexa Fluor®647, Alexa Fluor®660, Alexa Fluor®680, Alexa Fluor®700, Alexa Fluor®750, Alexa Fluor®790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar®570, Quasar®670, Quasar®705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™633, Seta™650, Seta™660, Seta™670, Seta™680, Seta™700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7. In some embodiments, the dye is one or more dyes selected from 4-Cy3B, 4-SGCy3, C2C, 3-AttoRho6G, and R1C1. In some embodiments, the dye is one or more dyes selected from Cy®3, Cy®3B, and ATTO Rho6G.

In some embodiments, the composition further comprises a triplet quencher.

In some embodiments, the triplet quencher is a compound of Formula (V):

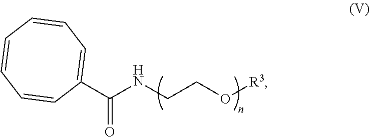

or a salt thereof, wherein:
R³ is substituted or unsubstituted aliphatic; and
n is 1, 2, 3, 4, 5, 6, 7, 8, 9, or 10.

As generally described herein, R³ is substituted or unsubstituted aliphatic. In some embodiments, R³ is substituted or unsubstituted alkyl, substituted or unsubstituted alkenyl, or substituted or unsubstituted alkynyl.

In some embodiments, R³ is substituted or unsubstituted alkyl. In some embodiments, R³ is substituted or unsubstituted C₁-C₁₂ alkyl, substituted or unsubstituted C₁-C₁₁ alkyl, substituted or unsubstituted C₁-C₁₀ alkyl, substituted or unsubstituted C₁-C₉ alkyl, substituted or unsubstituted C₁-C₈ alkyl, substituted or unsubstituted C₁-C₇ alkyl, substituted or unsubstituted C₁-C₆ alkyl, substituted or unsubstituted C₁-C₅ alkyl, substituted or unsubstituted C₁-C₄ alkyl, substituted or unsubstituted C₁-C₃ alkyl, or substituted or unsubstituted C₁-C₂ alkyl. In some embodiments, R³ is substituted or unsubstituted C₁-C₆ alkyl, substituted or unsubstituted C₁-C₅ alkyl, substituted or unsubstituted C₁-C₄ alkyl, substituted or unsubstituted C₁-C₃ alkyl, or substituted or unsubstituted C₁-C₂ alkyl.

In some embodiments, R³ is substituted or unsubstituted C₁-C₁₂ alkyl. In some embodiments, R³ is substituted or unsubstituted C₁-C₁₁ alkyl. In some embodiments, R³ is substituted or unsubstituted C₁-C₁₀ alkyl. In some embodiments, R³ is substituted or unsubstituted C₁-C₉ alkyl. In some embodiments, R³ is substituted or unsubstituted C₁-C₈ alkyl. In some embodiments, R³ is substituted or unsubstituted C₁-C₇ alkyl. In some embodiments, R³ is substituted or unsubstituted C₁-C₆ alkyl. In some embodiments, R³ is substituted or unsubstituted C₁-C₅ alkyl. In some embodiments, R³ is substituted or unsubstituted C₁-C₄ alkyl. In some embodiments, R³ is substituted or unsubstituted C₁-C₃ alkyl. In some embodiments, R³ is substituted or unsubstituted C₁-C₂ alkyl.

In some embodiments, R³ is substituted alkyl. In some embodiments, R³ is substituted C₁-C₁₂ alkyl, substituted C₁-C₁₁ alkyl, substituted C₁-C₁₀ alkyl, substituted C₁-C₉ alkyl, substituted C₁-C₈ alkyl, substituted C₁-C₇ alkyl, substituted C₁-C₆ alkyl, substituted C₁-C₅ alkyl, substituted C₁-C₄ alkyl, substituted C₁-C₃ alkyl, or substituted C₁-C₂ alkyl. In some embodiments, R³ is substituted C₁-C₆ alkyl, substituted C₁-C₅ alkyl, substituted C₁-C₄ alkyl, substituted C₁-C₃ alkyl, or substituted C₁-C₂ alkyl. In some embodiments, R³ is substituted C₁-C₆ alkyl, substituted C₁-C₅ alkyl, substituted C₁-C₄ alkyl, substituted C₁-C₃ alkyl, or substituted C₁-C₂ alkyl.

In some embodiments, R³ is substituted C₁-C₁₂ alkyl. In some embodiments, R³ is substituted C₁-C₁₁ alkyl. In some embodiments, R³ is substituted C₁-C₁₀ alkyl. In some embodiments, R³ is substituted C₁-C₉ alkyl. In some embodiments, R³ is substituted C₁-C₈ alkyl. In some embodiments, R³ is substituted C₁-C₇ alkyl. In some embodiments, R³ is substituted C₁-C₆ alkyl. In some embodiments, R³ is substituted C₁-C₅ alkyl. In some embodiments, R³ is substituted C₁-C₄ alkyl. In some embodiments, R³ is substituted C₁-C₃ alkyl. In some embodiments, R³ is substituted C₁-C₂ alkyl.

In some embodiments, R³ is C₁-C₆ alkyl, C₁-C₅ alkyl, C₁-C₄ alkyl, C₁-C₃ alkyl, or C₁-C₂ alkyl substituted with halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, ═O, ═S, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, —B(OR^{A})₂, and/or —N(R^{A})₃+, wherein each instance of R^{A} is independently hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom(s) to form an substituted or unsubstituted heterocyclic ring or substituted or unsubstituted heteroaryl ring. In some embodiments, R³ is C₁-C₆ alkyl, C₁-C₅ alkyl, C₁-C₄ alkyl, C₁-C₃ alkyl, or C₁-C₂ alkyl substituted with —S(═O)₂OR^{A} and/or —N(R^{A})₃+. In some embodiments, R³ is C₁-C₆ alkyl substituted with —S(═O)₂OR^{A} and/or —N(R^{A})₃⁺. In some embodiments, R³ is C₁-C₅ alkyl substituted with —S(═O)₂OR^{A} and/or —N(R^{A})₃⁺. In some embodiments, R³ is C₁-C₄ alkyl substituted with —S(═O)₂OR^{A} and/or —N(R^{A})₃⁺. In some embodiments, R³ is C₁-C₃ alkyl substituted with —S(═O)₂OR^{A} and/or —N(R^{A})₃⁺. In some embodiments, R³ is C₁-C₂ alkyl substituted with —S(═O)₂OR^{A} and/or —N(R^{A})₃⁺.

In some embodiments, R³ is of formula:

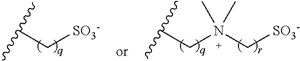

wherein: q is 1, 2, 3, 4, 5, or 6; and r is 1, 2, 3, 4, 5, or 6. In some embodiments, q is 1. In some embodiments, q is 2. In some embodiments, q is 3. In some embodiments, q is 4. In some embodiments, q is 5. In some embodiments, q is 6. In some embodiments, r is 1. In some embodiments, r is 2. In some embodiments, r is 3. In some embodiments, r is 4. In some embodiments, r is 5. In some embodiments, r is 6. In some embodiments, q is 2, and r is 3.

In some embodiments, R³ is

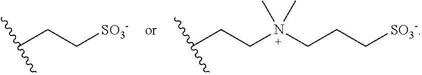

In some embodiments, R³ is unsubstituted alkyl. In some embodiments, R³ is unsubstituted C₁-C₁₂ alkyl, unsubstituted C₁-C₁₁ alkyl, unsubstituted C₁-C₁₀ alkyl, unsubstituted C₁-C₉ alkyl, unsubstituted C₁-C₈ alkyl, unsubstituted C₁-C₇ alkyl, unsubstituted C₁-C₆ alkyl, unsubstituted C₁-C₅ alkyl, unsubstituted C₁-C₄ alkyl, unsubstituted C₁-C₃ alkyl, or unsubstituted C₁-C₂ alkyl. In some embodiments, R³ is unsubstituted C₁-C₆ alkyl, unsubstituted C₁-C₅ alkyl, unsubstituted C₁-C₄ alkyl, unsubstituted C₁-C₃ alkyl, or unsubstituted C₁-C₂ alkyl.

In some embodiments, R³ is unsubstituted C₁-C₁₂ alkyl. In some embodiments, R³ is unsubstituted C₁-C₁₁ alkyl. In some embodiments, R³ is unsubstituted C₁-C₁₀ alkyl. In some embodiments, R³ is unsubstituted C₁-C₉ alkyl. In some embodiments, R³ is unsubstituted C₁-C₈ alkyl. In some embodiments, R³ is unsubstituted C₁-C₇ alkyl. In some embodiments, R³ is unsubstituted C₁-C₆ alkyl. In some embodiments, R³ is unsubstituted C₁-C₅ alkyl. In some embodiments, R³ is unsubstituted C₁-C₄ alkyl. In some embodiments, R³ is unsubstituted C₁-C₃ alkyl. In some embodiments, R³ is unsubstituted C₁-C₂ alkyl.

In some embodiments, R³ is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R³ is methyl, ethyl, n-propyl, n-butyl, n-pentyl, or n-hexyl. In some embodiments, R³ is methyl, ethyl, or n-propyl. In some embodiments, R³ is methyl (—CH₃).

In some embodiments, R³ is substituted or unsubstituted alkenyl. In some embodiments, R³ is substituted or unsubstituted C₁-C₁₂ alkenyl. In some embodiments, R³ is substituted or unsubstituted C₁-C₆ alkenyl. In some embodiments, R³ is substituted or unsubstituted C₁-C₃ alkenyl. In some embodiments, R³ is substituted alkenyl. In some embodiments, R³ is substituted C₁-C₁₂ alkenyl. In some embodiments, R³ is substituted C₁-C₆ alkenyl. In some embodiments, R³ is substituted C₁-C₃ alkenyl. In some embodiments, R³ is unsubstituted alkenyl. In some embodiments, R³ is unsubstituted C₁-C₁₂ alkenyl. In some embodiments, R³ is unsubstituted C₁-C₆ alkenyl. In some embodiments, R³ is unsubstituted C₁-C₃ alkenyl.

In some embodiments, R³ is substituted or unsubstituted alkynyl. In some embodiments, R³ is substituted or unsubstituted C₁-C₁₂ alkynyl. In some embodiments, R³ is substituted or unsubstituted C₁-C₆ alkynyl. In some embodiments, R³ is substituted or unsubstituted C₁-C₃ alkynyl. In some embodiments, R³ is substituted alkynyl. In some embodiments, R³ is substituted C₁-C₁₂ alkynyl. In some embodiments, R³ is substituted C₁-C₆ alkynyl. In some embodiments, R³ is substituted C₁-C₃ alkynyl. In some embodiments, R³ is unsubstituted alkynyl. In some embodiments, R³ is unsubstituted C₁-C₁₂ alkynyl. In some embodiments, R³ is unsubstituted C₁-C₆ alkynyl. In some embodiments, R³ is unsubstituted C₁-C₃ alkynyl.

As generally described herein, n is 1, 2, 3, 4, 5, 6, 7, 8, 9, or 10.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 1, 2, 3, 4, 5, 6, 7, 8, or 9. In some embodiments, n is 1, 2, 3, 4, 5, 6, 7, or 8. In some embodiments, n is 1, 2, 3, 4, 5, 6, or 7. In some embodiments, n is 1, 2, 3, 4, 5, or 6. In some embodiments, n is 1, 2, 3, 4, or 5. In some embodiments, n is 1, 2, 3, or 4. In some embodiments, n is 1, 2, or 3. In some embodiments, n is 1 or 2. In some embodiments, n is 1, 3, or 5.

In some embodiments, R³ is substituted or unsubstituted C₁-C₃ alkyl, and n is 1, 2, 3, 4, or 5. In some embodiments, R³ is substituted or unsubstituted C₁-C₃ alkyl, and n is 1. In some embodiments, R³ is substituted or unsubstituted C₁-C₃ alkyl, and n is 2. In some embodiments, R³ is substituted or unsubstituted C₁-C₃ alkyl, and n is 3. In some embodiments, R³ is substituted or unsubstituted C₁-C₃ alkyl, and n is 4. In some embodiments, R³ is substituted or unsubstituted C₁-C₃ alkyl, and n is 5.

In some embodiments, R³ is substituted C₁-C₃ alkyl, and n is 1, 2, 3, 4, or 5. In some embodiments, R³ is substituted C₁-C₃ alkyl, and n is 1. In some embodiments, R³ is substituted C₁-C₃ alkyl, and n is 2. In some embodiments, R³ is substituted C₁-C₃ alkyl, and n is 3. In some embodiments, R³ is substituted C₁-C₃ alkyl, and n is 4. In some embodiments, R³ is substituted C₁-C₃ alkyl, and n is 5.

In some embodiments, R³ is

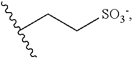

and n is 1, 2, 3, 4, or 5. In some embodiments, R³ is

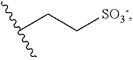

and n is 1. In some embodiments, R³ is

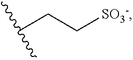

and n is 2. In some embodiments, R³ is

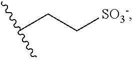

and n is 3. In some embodiments, R³ is

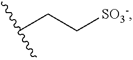

and n is 4. In some embodiments, R³ is

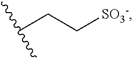

and n is 5.

In some embodiments, R³ is

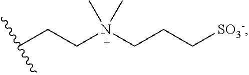

and n is 1, 2, 3, 4, or 5. In some embodiments, R³ is

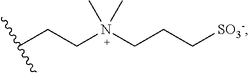

and n is 1. In some embodiments, R³ is

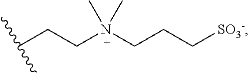

and n is 2. In some embodiments, R³ is

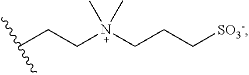

and n is 3. In some embodiments, R³ is

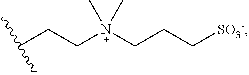

and n is 4. In some embodiments, R³ is

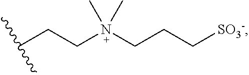

and n is 5.

In some embodiments, R³ is unsubstituted C₁-C₃ alkyl, and n is 1, 2, 3, 4, or 5. In some embodiments, R³ is unsubstituted C₁-C₃ alkyl, and n is 1. In some embodiments, R³ is unsubstituted C₁-C₃ alkyl, and n is 2. In some embodiments, R³ is unsubstituted C₁-C₃ alkyl, and n is 3. In some embodiments, R³ is unsubstituted C₁-C₃ alkyl, and n is 4. In some embodiments, R³ is unsubstituted C₁-C₃ alkyl, and n is 5.

In some embodiments, R³ is —CH₃, and n is 1, 2, 3, 4, or 5. In some embodiments, R³ is —CH₃, and n is 1. In some embodiments, R³ is —CH₃, and n is 2. In some embodiments, R³ is —CH₃, and n is 3. In some embodiments, R³ is —CH₃, and n is 4. In some embodiments, R³ is —CH₃, and n is 5.

In some embodiments, the triplet quencher is a compound of formula:

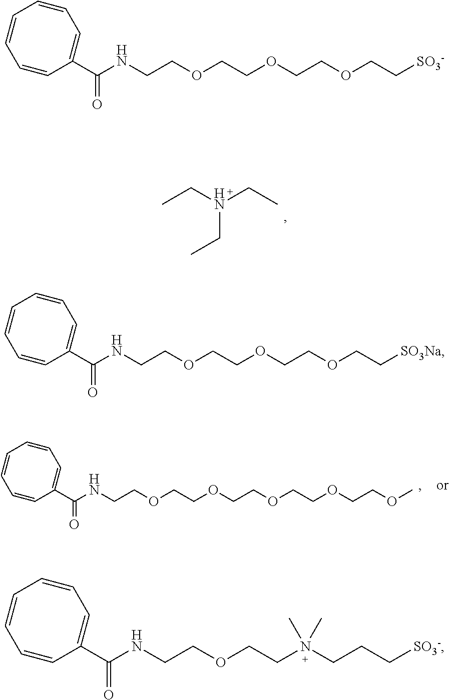

or a salt thereof.

As described herein, in some aspects, the disclosure provides compositions and methods for polypeptide sequencing. In an exemplary dynamic peptide sequencing reaction, individual on-off binding events give rise to signal pulses of a signal output. A polypeptide sample may be fragmented into peptides, which are immobilized in sample wells of an array, where the immobilized peptides are exposed to one or more amino acid recognition molecules (also referred to as recognizers) and one or more cleaving reagents (e.g., aminopeptidases). An amino acid recognition molecule reversibly binds a terminal end of the peptide, and a detectable signal is produced while the recognition molecule is bound to the peptide. As the on-off binding of recognition molecules generally occurs at a faster rate than amino acid cleavage, the binding events preceding amino acid cleavage give rise to a series of signal pulses that can be used to determine at least one chemical characteristic of the peptide (and/or an originating polypeptide). In certain embodiments, determining at least one chemical characteristic of the peptide comprises detecting the presence or absence of a target residue. In certain embodiments, determining at least one chemical characteristic of the peptide comprises determining the location of a target residue in the peptide (and/or an originating polypeptide). In certain embodiments, determining at least one chemical characteristic of the peptide comprises determining if one or more amino acids comprise a post-translational modification. In certain embodiments, determining at least one chemical characteristic of the peptide comprises determining an identity of one or more amino acids of the peptide.

Methods, reagents, and compositions for performing dynamic sequencing are described more fully in PCT International Application No. PCT/US2019/061831, filed Nov. 15, 2019, PCT International Application No. PCT/US2021/033493, filed May 20, 2021, PCT International Application No. PCT/US2023/077470, filed Oct. 20, 2023, and PCT International Application No. PCT/US2023/077481, filed Oct. 20, 2023, each of which is incorporated herein by reference in its entirety.

Accordingly, in some embodiments, polypeptide sequencing is performed by detecting a series of signal pulses indicative of association of one or more amino acid recognition molecules with successive amino acids exposed at the terminus of a polypeptide in an ongoing degradation reaction. The series of signal pulses can be analyzed to determine characteristic patterns in the series of signal pulses, and the time course of characteristic patterns can be used to determine an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10⁻²⁵%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognition molecule to cleaving reagent, ratio of one recognition molecule to another, ratio of one cleaving reagent to another), number of different reagent types (e.g., the number of different types of recognition molecules and/or cleaving reagents, the number of recognition molecule types relative to the number of cleaving reagent types), cleavage activity (e.g., peptidase activity), binding properties (e.g., kinetic and/or thermodynamic binding parameters for recognition molecule binding), reagent modification (e.g., polyol and other protein modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, a polypeptide sequencing reaction in accordance with the disclosure is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. In some embodiments, a polypeptide sequencing reaction is performed in a reaction mixture at a pH of between about 6.5 and about 9.0. In some embodiments, a polypeptide sequencing reaction is performed in a reaction mixture at a pH of between about 7.0 and about 8.5 (e.g., between about 7.0 and about 8.0, between about 7.5 and about 8.5, between about 7.5 and about 8.0, or between about 8.0 and about 8.5).

In some embodiments, a polypeptide sequencing reaction is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10⁻²⁵⁰ mM, 10⁻¹⁰⁰ mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino)propanesulfonic acid).

In some embodiments, a polypeptide sequencing reaction is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in a reaction mixture include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, a polypeptide sequencing reaction is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

In some embodiments, polypeptide sequencing in accordance with the disclosure may be carried out by contacting a polypeptide with a sequencing reaction mixture comprising one or more amino acid recognition molecules and/or one or more cleaving reagents (e.g., peptidases). In some embodiments, a sequencing reaction mixture comprises an amino acid recognition molecule at a concentration of between about 10 nM and about 10 μM. In some embodiments, a sequencing reaction mixture comprises a cleaving reagent at a concentration of between about 500 nM and about 500 μM.

In some embodiments, a sequencing reaction mixture comprises an amino acid recognition molecule at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a sequencing reaction mixture comprises an amino acid recognition molecule at a concentration of about 100 nM, about 250 nM, about 500 nM, about 750 nM, or about 1 μM.

In some embodiments, a sequencing reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a sequencing reaction mixture comprises a cleaving reagent at a concentration of about 1 μM, about 5 μM, about 10 μM, about 30 μM, about 50 μM, about 70 μM, or about 100 μM.

In some embodiments, a sequencing reaction mixture comprises an amino acid recognition molecule at a concentration of between about 10 nM and about 10 μM, and a cleaving reagent at a concentration of between about 500 nM and about 500 μM. In some embodiments, a sequencing reaction mixture comprises an amino acid recognition molecule at a concentration of between about 100 nM and about 1 μM, and a cleaving reagent at a concentration of between about 1 μM and about 100 μM. In some embodiments, a sequencing reaction mixture comprises an amino acid recognition molecule at a concentration of between about 250 nM and about 1 μM, and a cleaving reagent at a concentration of between about 10 μM and about 100 μM. In some embodiments, a sequencing reaction mixture comprises an amino acid recognition molecule at a concentration of about 500 nM, and a cleaving reagent at a concentration of between about 25 μM and about 75 μM. In some embodiments, the concentration of an amino acid recognition molecule and/or the concentration of a cleaving reagent in a reaction mixture is as described elsewhere herein.

In some embodiments, a sequencing reaction mixture comprises an amino acid recognition molecule and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a sequencing reaction mixture comprises an amino acid recognition molecule and a cleaving reagent in a molar ratio of between about 10:1 and about 200:1. In some embodiments, a sequencing reaction mixture comprises an amino acid recognition molecule and a cleaving reagent in a molar ratio of between about 50:1 and about 150:1. In some embodiments, the molar ratio of an amino acid recognition molecule to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognition molecule to a cleaving reagent in a reaction mixture is between about 1:100 and about 1:1 or between about 1:1 and about 10:1. In some embodiments, the molar ratio of an amino acid recognition molecule to a cleaving reagent in a reaction mixture is as described elsewhere herein.

In some embodiments, a sequencing reaction mixture comprises one or more amino acid recognition molecules and one or more cleaving reagents. In some embodiments, a sequencing reaction mixture comprises at least three amino acid recognition molecules and at least one cleaving reagent. In some embodiments, the sequencing reaction mixture comprises two or more cleaving reagents. In some embodiments, the sequencing reaction mixture comprises at least one and up to ten cleaving reagents (e.g., 1-3 cleaving reagents, 2-10 cleaving reagents, 1-5 cleaving reagents, 3-10 cleaving reagents). In some embodiments, the sequencing reaction mixture comprises at least three and up to thirty amino acid recognition molecules (e.g., between 3 and 25, between 3 and 20, between 3 and 10, between 3 and 5, between 5 and 30, between 5 and 20, between 5 and 10, or between 10 and 20, amino acid recognition molecules).

In some embodiments, a sequencing reaction mixture comprises more than one amino acid recognition molecule and/or more than one cleaving reagent. In some embodiments, a sequencing reaction mixture described as comprising more than one amino acid recognition molecule (or cleaving reagent) refers to the mixture as having more than one type of amino acid recognition molecule (or cleaving reagent). For example, in some embodiments, a sequencing reaction mixture comprises two or more amino acid binding proteins. In some embodiments, the two or more amino acid binding proteins refer to two or more types of amino acid binding proteins. In some embodiments, one type of amino acid binding protein has an amino acid sequence that is different from another type of amino acid binding protein in the reaction mixture. In some embodiments, one type of amino acid binding protein has a label that is different from a label of another type of amino acid binding protein in the reaction mixture. In some embodiments, one type of amino acid binding protein associates with (e.g., binds to) an amino acid that is different from an amino acid with which another type of amino acid binding protein in the reaction mixture associates. In some embodiments, one type of amino acid binding protein associates with (e.g., binds to) a subset of amino acids that is different from a subset of amino acids with which another type of amino acid binding protein in the reaction mixture associates.

As described herein, one or more characteristics of the series of signal pulses may be determined, including signal pulse intensity, fluorescence lifetime, wavelength, signal pulse duration, interpulse duration, and/or cleavage rate, among others.

In some embodiments, the characteristic of the series of signal pulses may comprise intensity (e.g., average intensity of the series of signal pulses). Intensity may be determined based on an amount of charge carriers detected in the photodetection region which receives the emission light from the fluorescent labels.

In some embodiments, the characteristic of the series of signal pulses may comprise pulse wavelength (e.g., average pulse wavelength of the series of signal pulses). In particular, emission light from a particular fluorescent label may have a characteristic wavelength such that analyzing wavelength information of emission light may facilitate identification of one or more chemical characteristics of the sample. Wavelength of the emission light may be determined in any suitable manner, for example, using one or more optical filters and/or photodetection regions disposed at different depths.

In some embodiments, the characteristic of the series of signal pulses may comprise fluorescence lifetime (e.g., average fluorescence lifetime of the series of signal pulses). In particular, fluorescent labels, when excited by incident excitation light, fluoresce with a characteristic lifetime (e.g., a characteristic emission decay time period), such that analyzing the lifetime information of emission light may facilitate identification of one or more chemical characteristics of the sample to which the fluorescent dye is attached. Fluorescence lifetime, also referred to herein as simply “lifetime”, is a measure of the time which a fluorescent dye spends in the excited state before returning to a ground state and emitting a photon. In some embodiments, fluorescence lifetime information and/or other timing characteristics described herein may be obtained through techniques for time binning charge carriers generated by photons incident on a photodetection region (e.g., a photodiode).

In some embodiments, the characteristic of the series of signal pulses may comprise pulse duration (e.g., average pulse duration), also referred to herein as pulse width. Pulse duration refers to the interval of time measured across a pulse, in some embodiments, at the full width half maximum of a pulse. As described herein, dye-labeled amino acid recognizers periodically bind and unbind to the polypeptide (e.g., to the amino acid). When bound, the dye-labeled amino acid recognizers may become excited and emit emission light. The average duration of respective signal pulses emitted by the dye-labeled amino acid recognizers comprise the pulse duration of the fluorescent label.

In some embodiments, the characteristic of the series of signal pulses may comprise interpulse duration (e.g., average interpulse duration). Interpulse duration, also referred to herein as interpulse width, refers to the interval of time between adjacent pulses. As described herein, dye-labeled amino acid recognizers periodically bind and unbind to the polypeptide (e.g., to the amino acid). When bound, the dye-labeled amino acid recognizers may become excited and emit emission light. The average durations between signal pulses emitted by the fluorescent label comprise the interpulse duration of the fluorescent label.

In some embodiments, the characteristic of the series of signal pulses may comprise a cleavage rate/time (e.g., an average cleavage rate/time). For example, a terminal amino acid of the polypeptide may be cleaved from the polypeptide fragment disposed in the reaction chamber. In some embodiments, cleaving the terminal amino acid is performed by introducing a solution comprising aminopeptidases into the chamber. In some embodiments, the aminopeptidases may be included in the same solution as the sample chain of amino acids and/or amino acid recognizers. A cleavage rate or cleavage time may comprise a duration between cleavage events. Cleavage events may be determined based on distinguishing respective series of signal pulses between each other. For example, a first series of signal pulses may be indicative of a series of binding events between a first set of one or more amino acid recognizers and an amino acid, such as the terminal amino acid. A second series of signal pulses may be indicative of a series of binding events between a second set of one or more amino acid recognizers and a subsequent amino acid (e.g., an amino acid which becomes the terminal amino acid after the initial terminal amino acid is cleaved). The respective series of signal pulses may have different characteristics, as described herein, which may allow the respective series of signal pulses to be distinguished from each other. Each series of signal pulses may be referred to herein as a recognition segment. Each recognition segment therefore comprises a plurality of on-off binding events between a set of one or more amino acid recognizers and a respective amino acid. The cleavage time may comprise a duration of each recognition segment. In some embodiments, the at least one characteristic comprises a duration of time between recognition segments (e.g., an average intersegment duration).

As described herein, a method may comprise determining at least one chemical characteristic of a polypeptide. In some embodiments, determining at least one chemical characteristic comprises determining the type of amino acid that is present in the polypeptide, including at a terminal end of the polypeptide, and/or the types of amino acids that are present at one or more other positions in the polypeptide, such as downstream, proximate, or contiguous to the amino acid. In some embodiments, determining the type of amino acid comprises determining the amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

As used herein, in some embodiments, “identifying,” “determining the identity,” “determining the type,” and like terms, in reference to an amino acid, include determination of an express identity of an amino acid as well as determination of a probability of an express identity of an amino acid. For example, in some embodiments, an amino acid is identified by determining a probability (e.g., from 0% to 100%) that the amino acid is of a specific type, or by determining a probability for each of a plurality of specific types. Accordingly, in some embodiments, the terms “amino acid sequence,” “polypeptide sequence,” and “protein sequence” as used herein may refer to the polypeptide or protein material itself and is not restricted to the specific sequence information (e.g., the succession of letters representing the order of amino acids from one terminus to another terminus) that biochemically characterizes a specific polypeptide or protein.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid—whether the terminal amino acid or one or more of the amino acids downstream of the terminal amino acid—comprises a post-translational modification. As described herein, the post-translational modification may be to the terminal amino acid or may additionally or alternatively be to one or more other amino acids of the polypeptide. The post-translational modification may affect the series of signals emitted by a dye-labeled amino acid recognizer bound to the peptide (e.g., to the terminal amino acid and, in some embodiments, to one or more amino acids downstream of the terminal amino acid). In some embodiments, the series of signals emitted by the dye-labeled amino acid may be impacted by the post-translational modification even if the post-translational modification is to an amino acid which does not bind to the dye-labeled amino acid recognizer. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), carbonylation (e.g., carbonylated lysine, carbonylated proline, carbonylated arginine, carbonylated threonine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation (e.g., sulfated tyrosine), glycation (e.g., glycated lysine), sumoylation (e.g., sumoylated lysine), and ubiquitination (e.g., ubiquitinated lysine).

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine). In some embodiments, determining at least one chemical characteristic of a polypeptide comprises determining a location of a phosphorylated serine in the polypeptide.

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine. As described herein, the at least one characteristic of the signal pulses may be used to determine at least one chemical characteristic of at least two amino acids. Accordingly, signal pulses from a dye-labeled first type of amino acid recognizer that binds to an amino acid, such as the terminal amino acid or an internal amino acid, may be used to determine one or more chemical characteristics of multiple amino acids. The inventors have recognized that such techniques are advantageous. For example, such techniques may allow for determining chemical characteristics of amino acids which are unrecognized. Such amino acids may be unrecognizable by any amino acid recognizers present in a reaction chamber, in some instances. Such techniques may also save time and/or require less signal collection. Accordingly, obtaining information regarding multiple amino acids based on fewer series of signal pulses and/or using fewer recognizers is advantageous.

In some embodiments, determining at least one chemical characteristic of a polypeptide comprises determining that at least one amino acid is bound (e.g., via a covalent or non-covalent interaction) to a binding component. Non-limiting examples of suitable binding components include a nucleic acid (e.g., DNA, RNA), a linker, and an antibody. In some instances, one or more amino acids of a polypeptide may be bound to a nucleic acid via one or more non-covalent interactions. In some instances, one or more amino acids of a polypeptide may be bound to a linker via one or more covalent interactions.

In some embodiments, one or more characteristics of a first series of pulse signals indicative of a first series of binding events between one or more amino acid recognizers and a first amino acid of a polypeptide (e.g., a terminal amino acid, an internal amino acid) may be impacted by one or more chemical characteristics of the polypeptide. In certain instances, one or more modifications of one or more amino acids (e.g., post-translational modifications, presence of binding components) may promote a covalent or non-covalent interaction between one or more amino acid recognizers and the first amino acid (e.g., through electrostatic attraction, pi stacking, hydrogen bond formation, etc.), thereby increasing pulse duration. In certain instances, one or more modifications of one or more amino acids (e.g., post-translational modifications, presence of binding components) may discourage a covalent or non-covalent interaction between one or more amino acid recognizers and the first amino acid (e.g., through electrostatic repulsion, steric hindrance, etc.), thereby decreasing pulse duration.

Compositions and methods for characterizing a polypeptide and analyzing data obtained therefrom are described more fully in PCT International Application No. PCT/US2019/061831, filed Nov. 15, 2019, and PCT International Application No. PCT/US2021/033493, filed May 20, 2021, each of which is incorporated by reference in its entirety. Examples of luminescent labels, linkers, and other reagents for use in accordance with the disclosure are described more fully in PCT International Application No. PCT/US2023/077470, filed Oct. 20, 2023, and PCT International Application No. PCT/US2023/077477, filed Oct. 20, 2023, each of which is incorporated by reference in its entirety.

### Systems

In another aspect, provided herein is a system for performing a method of sequencing a polypeptide. Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample well may have a suitable size and shape such that at least a portion of the sample well receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light sources external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample well, which may range in number between approximately 10,000 pixels to 1,000,000 pixels according to some embodiments, multiple samples can be analyzed in parallel.

The integrated device may include an optical system for receiving excitation light and directing the excitation light among the reaction chamber array. The optical system may include one or more grating couplers configured to couple excitation light to other optical components of the integrated device and direct the excitation light to the other optical components. For example, the optical system may include optical components that direct the excitation light from the grating coupler(s) towards the reaction chamber array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a reaction chamber and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding reaction chamber. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a reaction chamber and its respective photodetector(s) may be aligned along a common axis. In this manner, the photodetector(s) may overlap with the reaction chamber within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the reaction chambers are carried out by exciting some or all of the samples within the chambers using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each reaction chamber to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Design and Synthesis

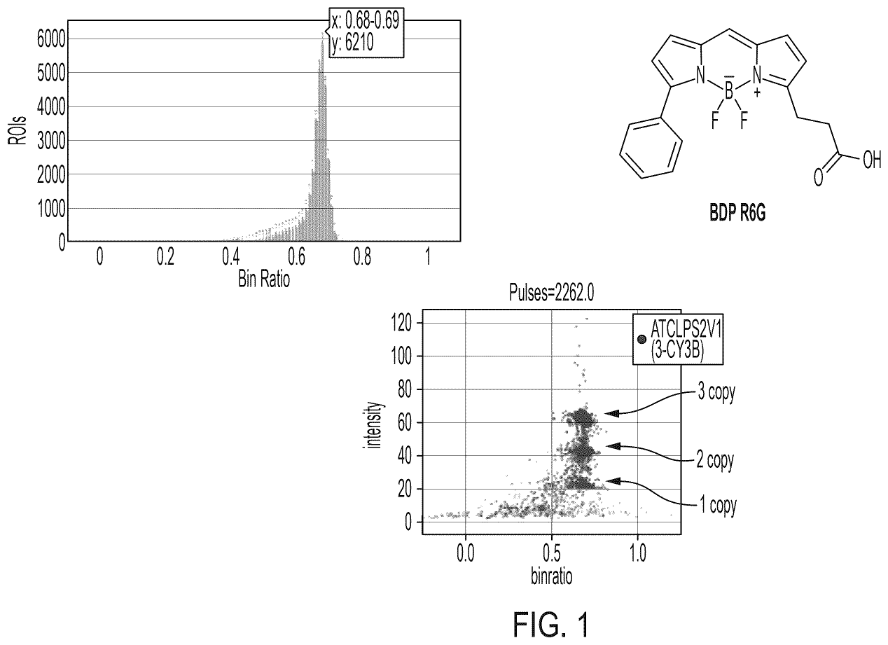

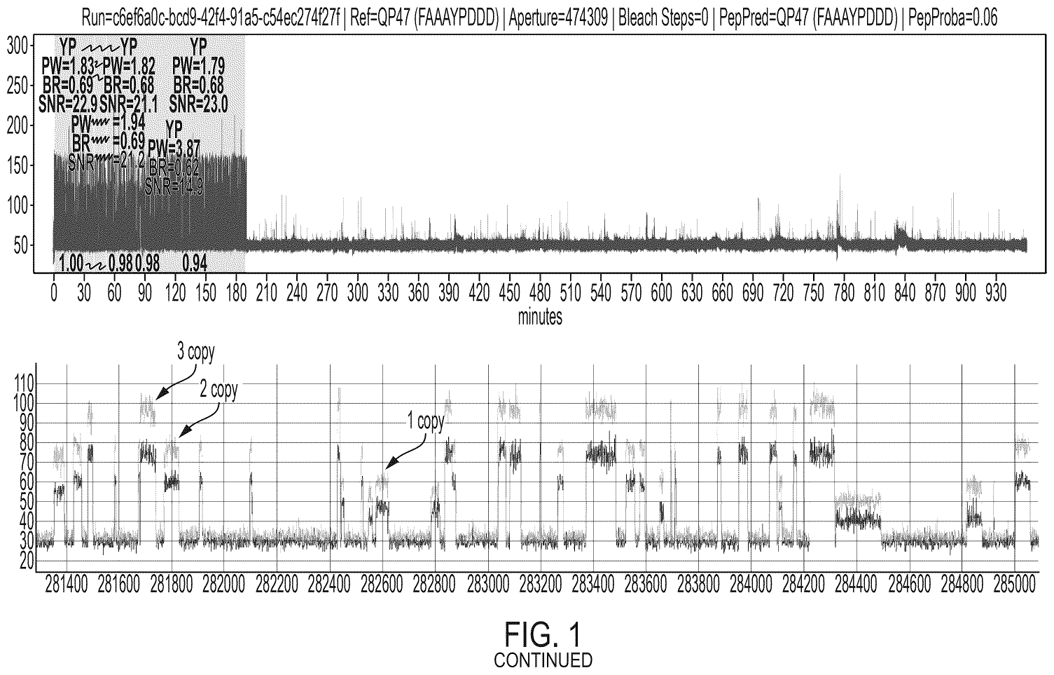

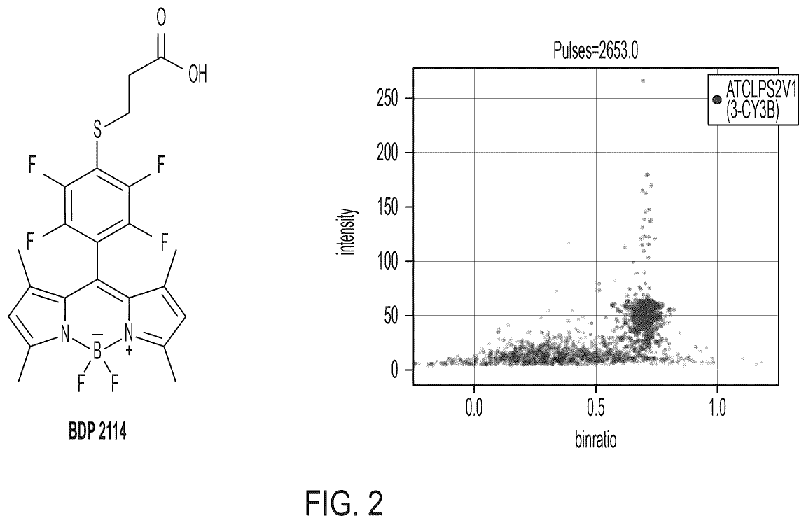

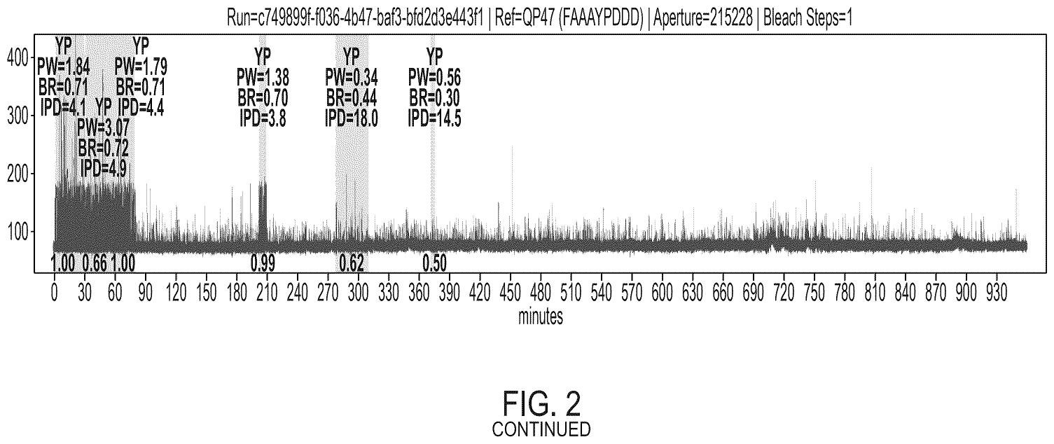

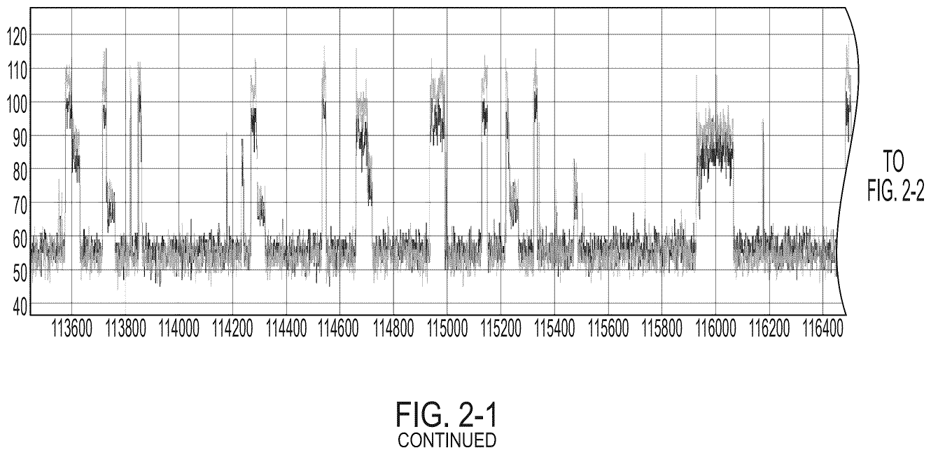

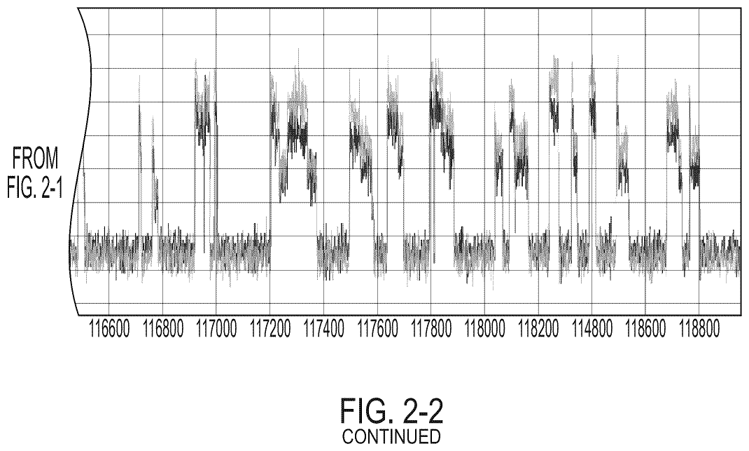

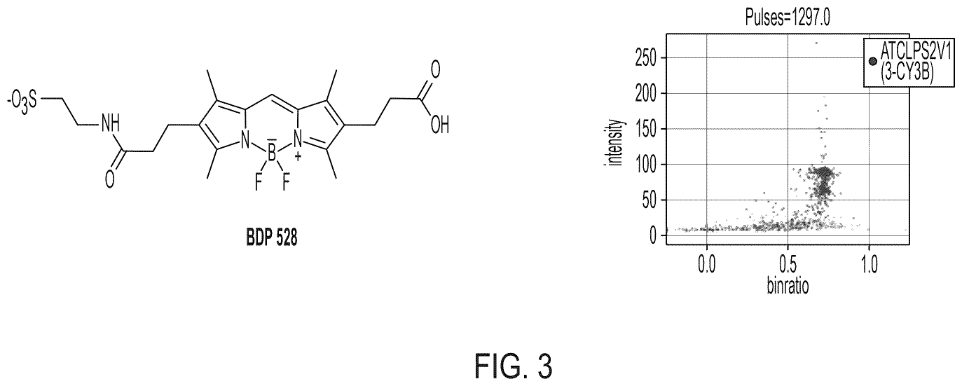

Several BODIPY dyes were evaluated for use in methods of polypeptide sequencing. BODIPY R6G (BDP R6G) was identified as having good bin ratio separation from AttoRho6G and good photostability, but it was found to be thermally unstable and degraded in solution over time (FIG. 1). Dyes such as BDP2114 appear to undergo photoblinking, in which the dye periodically goes into a temporary, non-emissive dark state, leading to intensity fluctuations within a pulse and producing diffuse clustering in bin ratio/intensity plots (FIG. 2). Dyes such as BDP528 show photobleaching, which occurs when laser excitation leads to irreversible destruction of a dye, resulting in prominent subclusters in bin ratio/intensity plots (FIG. 3).

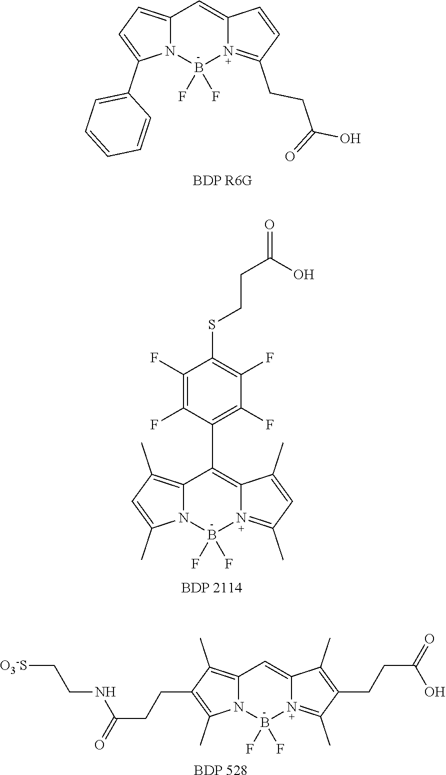

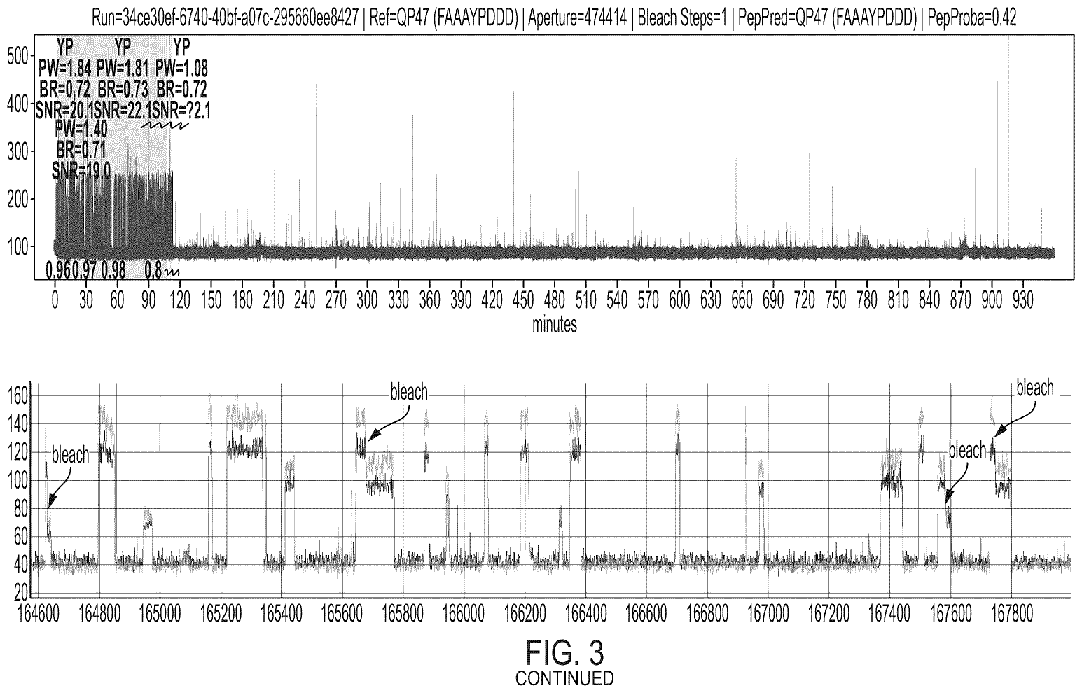

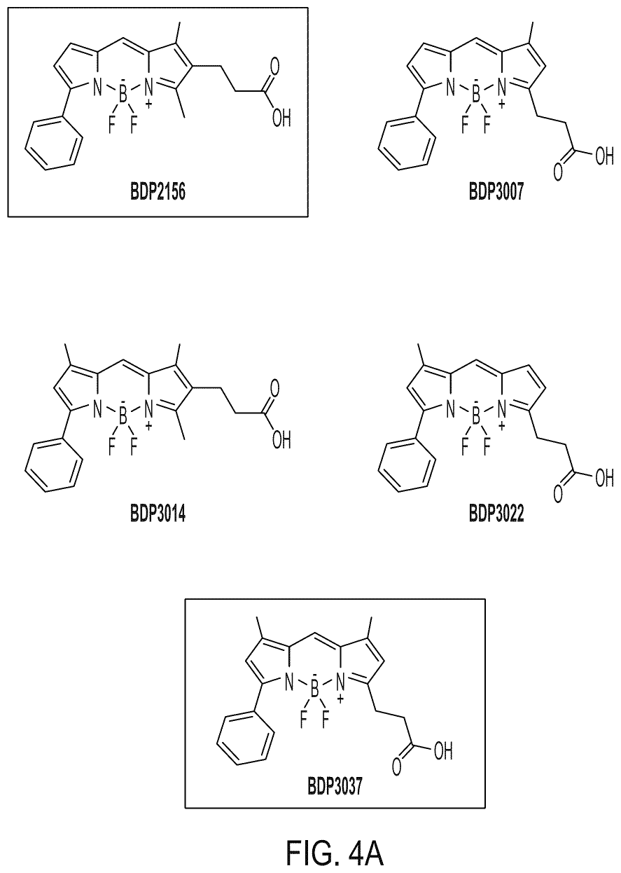

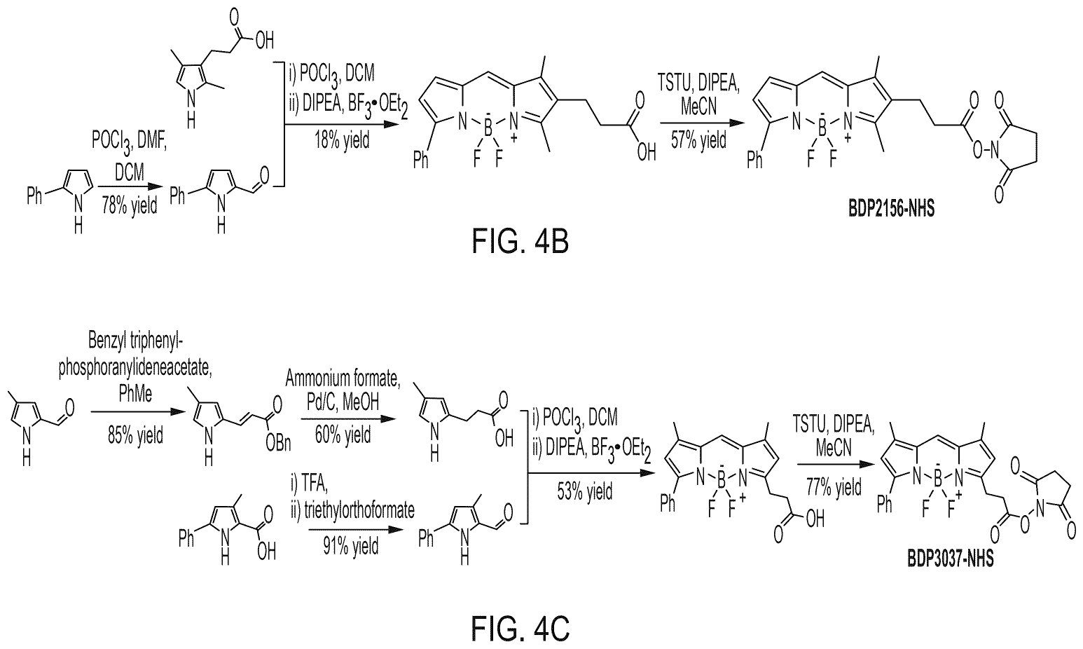

It was hypothesized that poor thermal stability of BDP R6G was from loss of boron due to low electron density in the ring system. A series of five novel dyes with higher electron density through alkylation was synthesized (FIGS. 4A-4C).

### Example 2: Thermal Stability, Recognition Runs, and Dynamic Sequencing

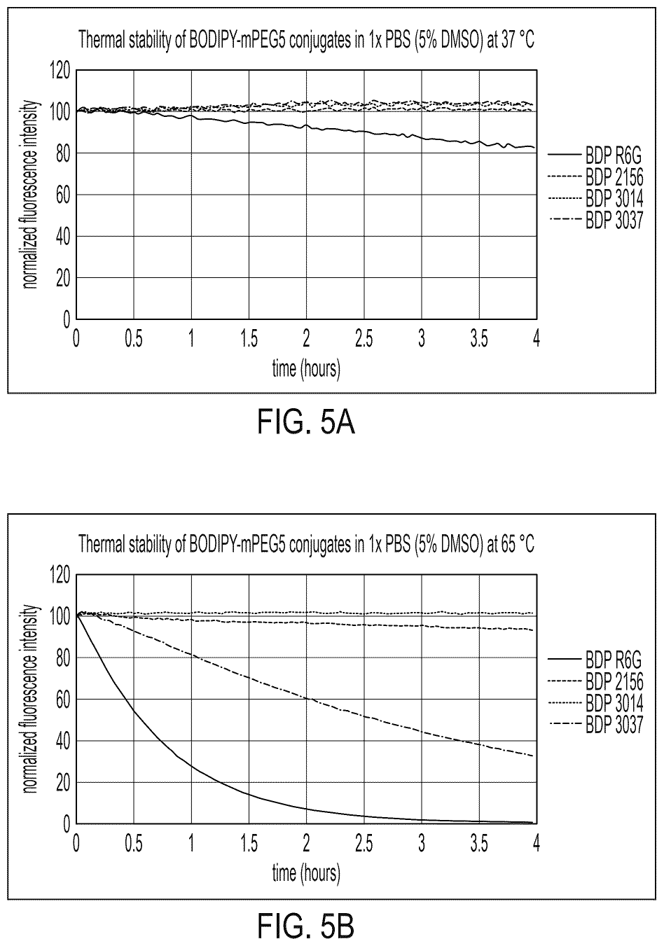

The novel dyes were evaluated on-chip and with mass spectrometry, and all were found to be more thermally stable than BDP R6G (FIGS. 5A-5B). The thermal stability of BDP R6G and three novel dyes was evaluated by first synthesizing a water-soluble mPEG5 conjugate of each dye. The mPEG5 conjugates were heated at 37° C. in 1×PBS (+5% DMSO), and fluorescence intensity was measured over 4 hours (FIG. 5A). BDP R6G showed nearly 20% loss of fluorescence over 4 hours, indicating dye degradation, while the novel dyes showed no discernable loss of fluorescence.

In another experiment, the mPEG5 conjugates were heated at 65° C. in 1×PBS (+5% DMSO), and fluorescence intensity was measured over 4 hours (FIG. 5B). BDP R6G is 50% bleached in roughly 30 minutes at 65° C. BDP2156 and BDP3014 show little loss of fluorescence at 65° C. BDP3037 has a half-life of over 2.5 hours at 65° C., indicating that it will exhibit minimal degradation when heated to 65° C. for 5 minutes during DNA hybridization for binder constructs.

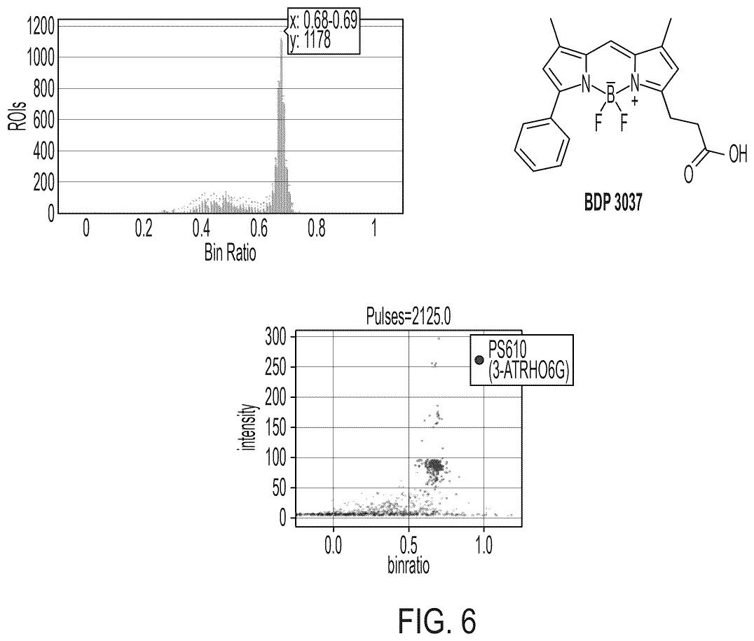

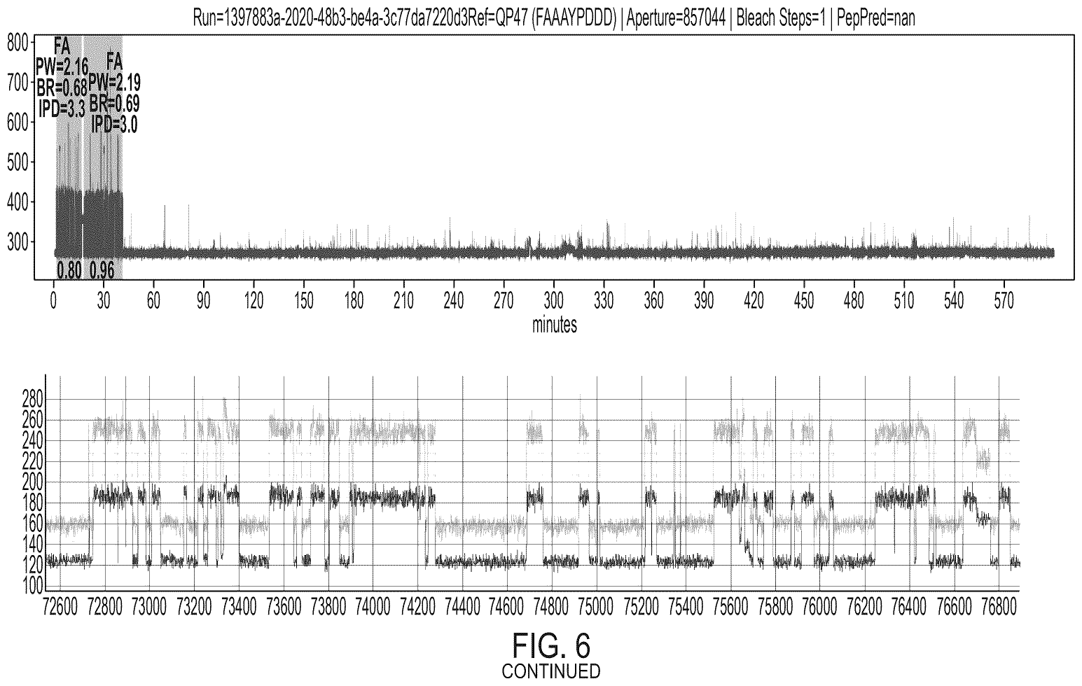

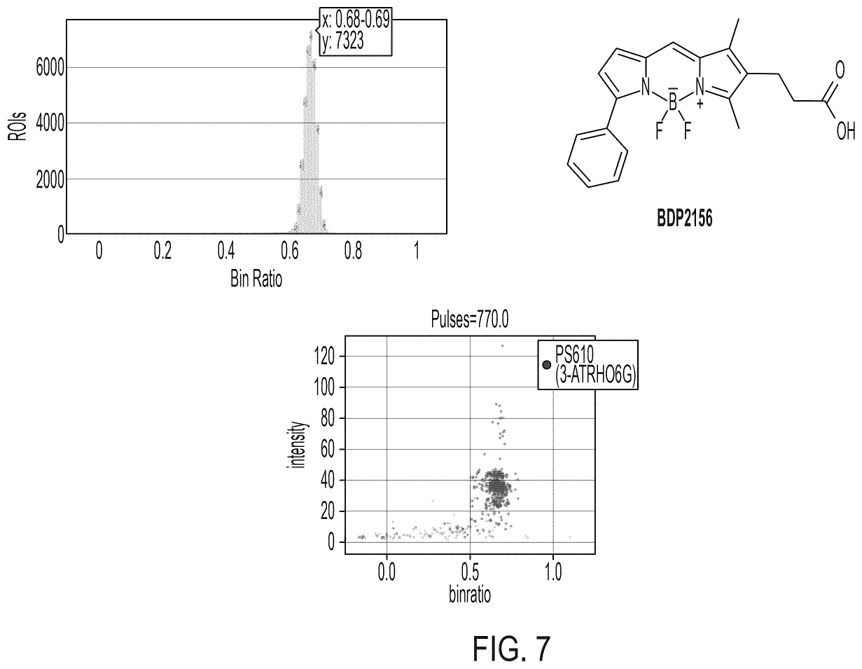

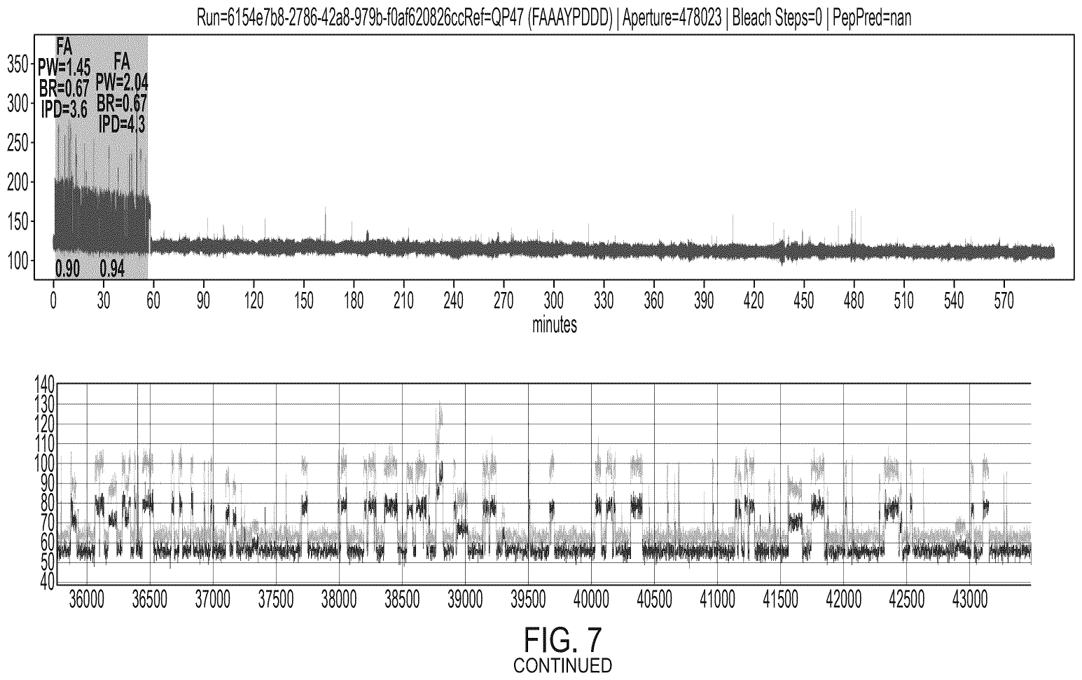

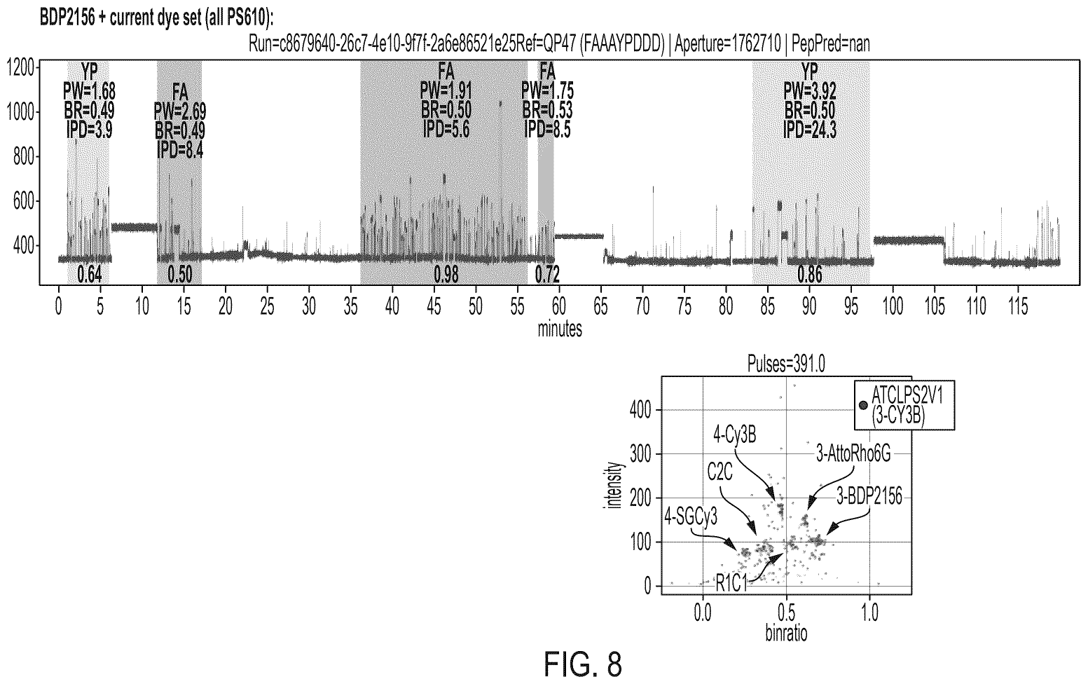

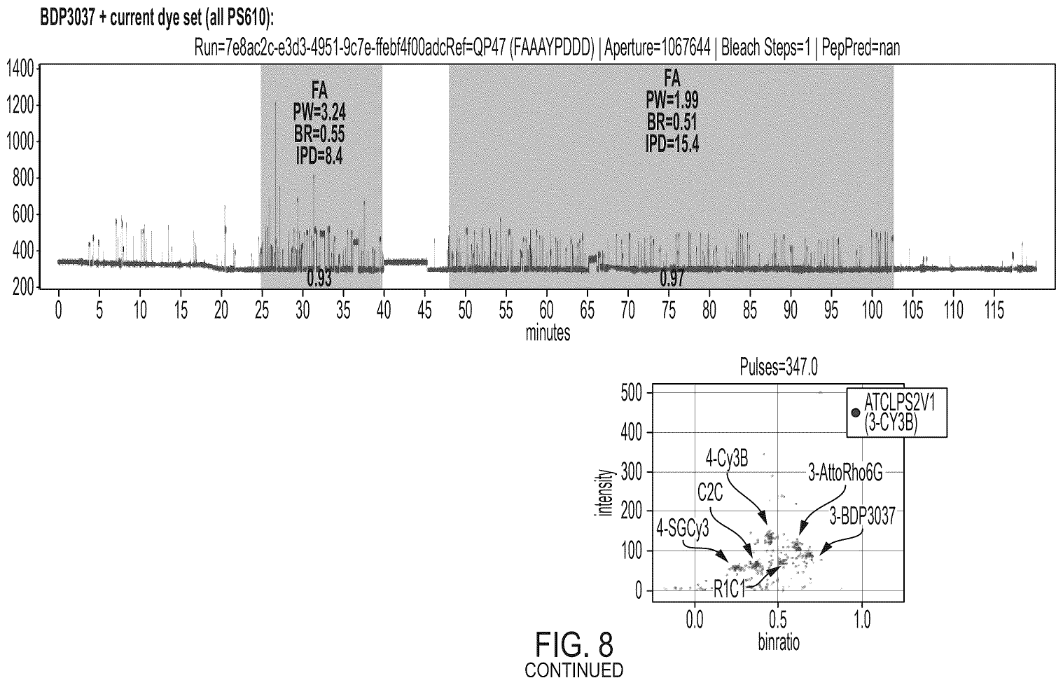

By analyzing the on-chip data for photochemical properties, BDP2156 and BDP3037 were identified as top-performing dyes (FIGS. 6-8). In the intensity versus bin ratio plots of FIG. 8, it is seen that BDP2156 and BDP3037 clusters are clearly distinguishable from the other dye clusters, thus providing two new dye channels that are distinguishable by lifetime and intensity measurements. As such, the new dyes are important tools for use in preparing and analyzing mixtures of multiple labelled amino acid recognizers, for example, as a label to distinguish one recognizer in the mixture from the other labeled recognizers in the mixture.

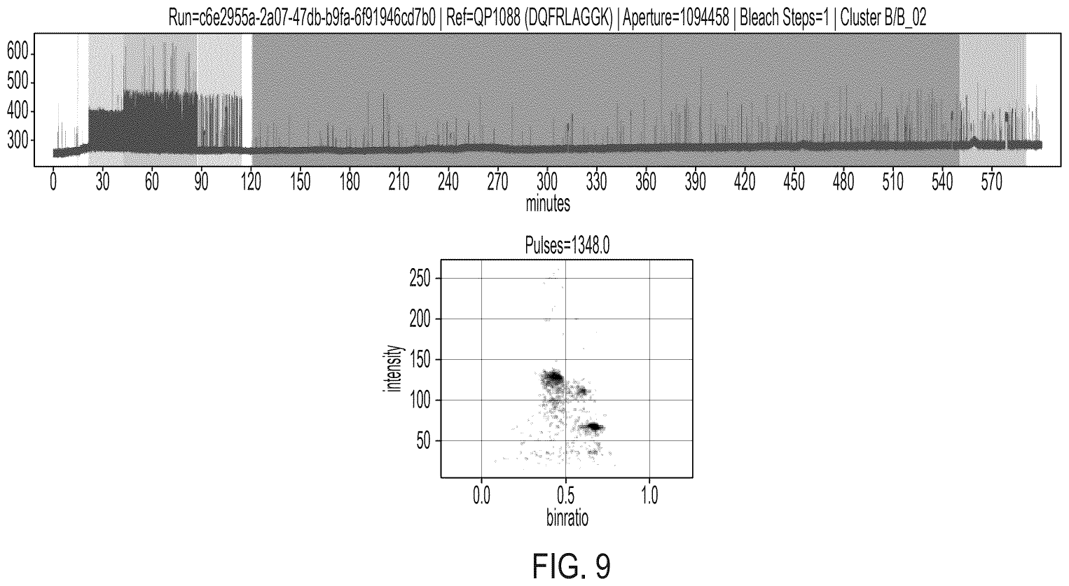

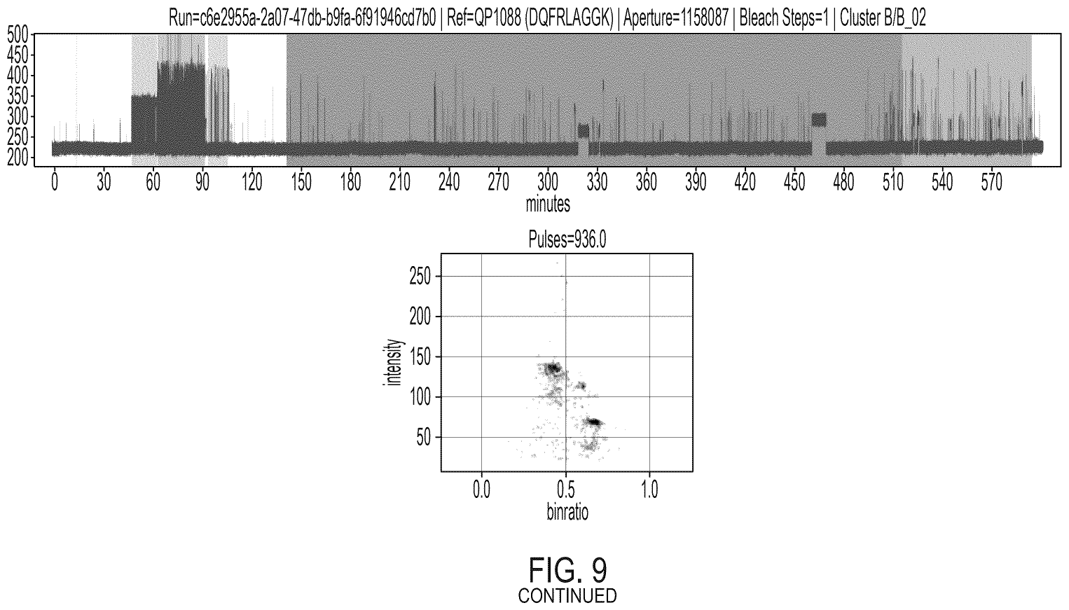

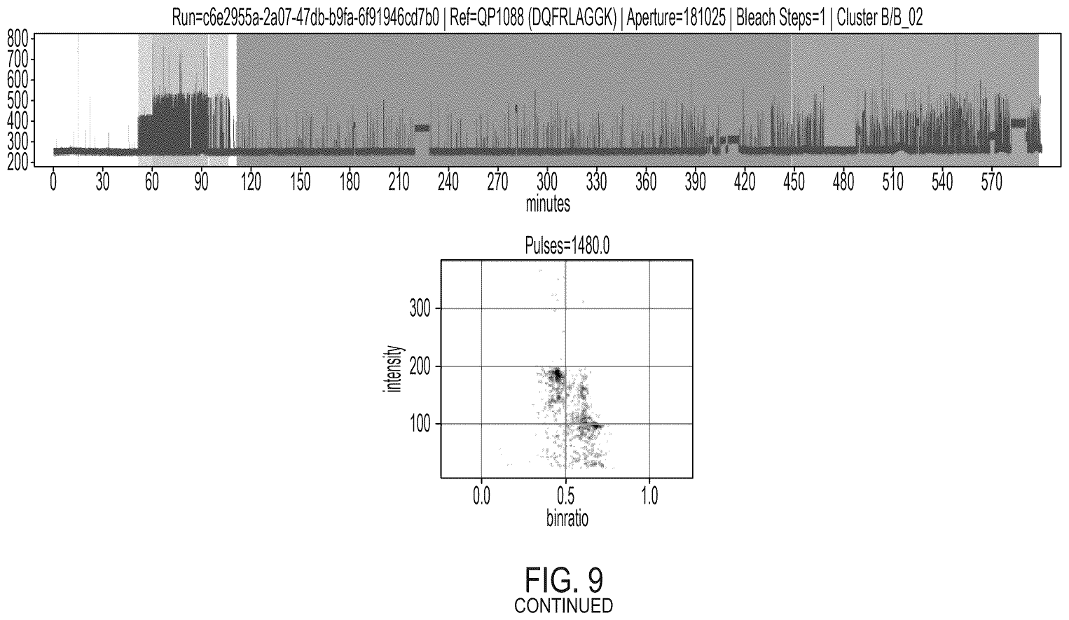

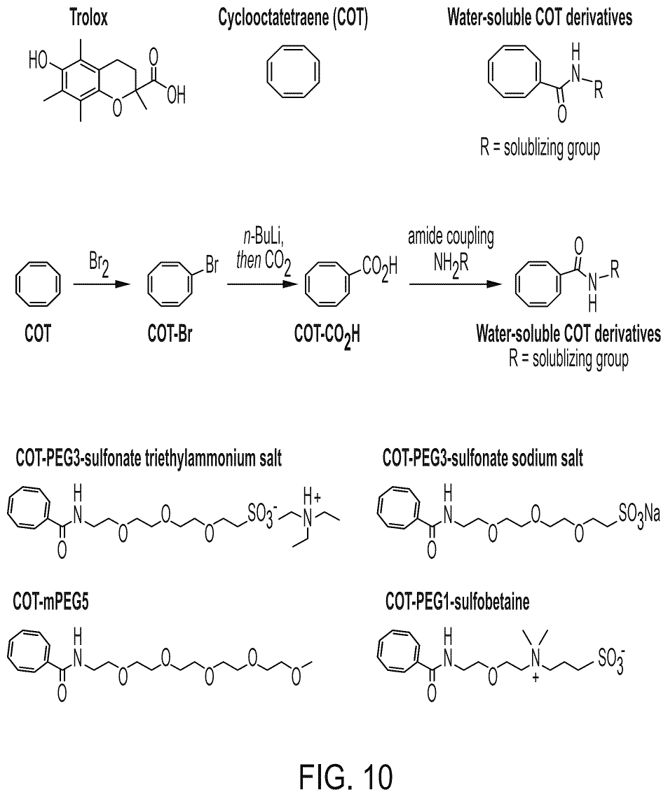

The longer lifetimes of both dyes make them distinguishable over other dyes (e.g., AttoRho6G) using pulsing data, while still being thermally stable and resistant to photobleaching. Both have a bin ratio around 0.68-0.69, and BDP3037 is brighter. Bis-BDP3037 is sufficiently bright for sequencing and is differentiable in intensity from tris-AttoRho6G (FIG. 9). BDP3037 additionally demonstrates the least photobleaching of tested BODIPY dyes (FIG. 10)

### Example 3: Triplet State Quencher for Dyes of the Present Disclosure

Cyclooctatetraene (COT)-based triplet state quenchers (TSQs) were used in place of or in addition to the currently used Trolox TSQ. TSQs can enhance photophysical properties of dyes in single-molecule studies, and the optimal TSQ can depend on the class of dye used. Trolox and COT quench triplet states by different mechanisms. In runs with only Trolox, BODIPY dyes showed decreased intensity and bin ratio, poor photostability, and diffuse clustering. Addition of unsubstituted COT to sequencing buffer was found to improve the signals of these dyes, but unsubstituted COT is not fully water-soluble, and its concentration drops over time.

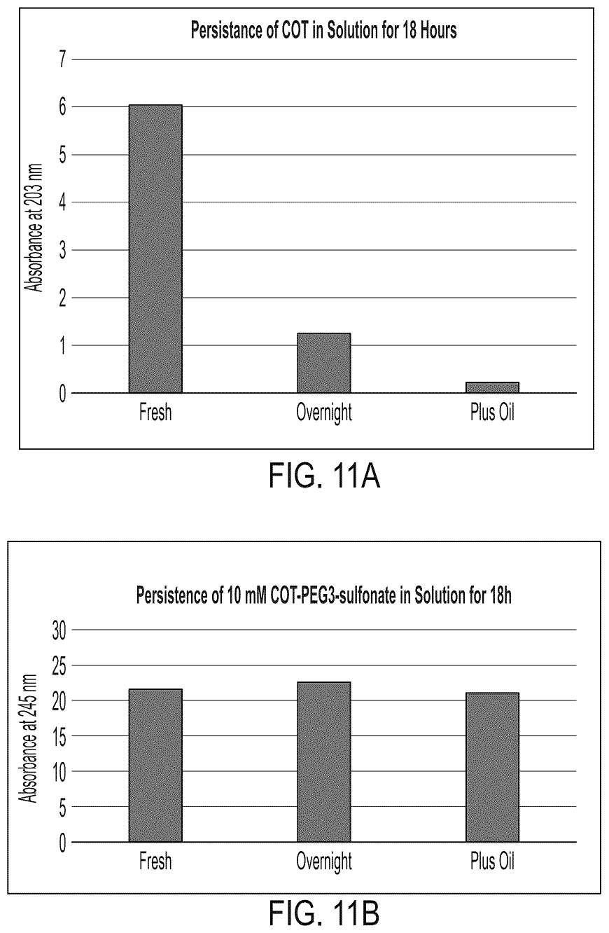

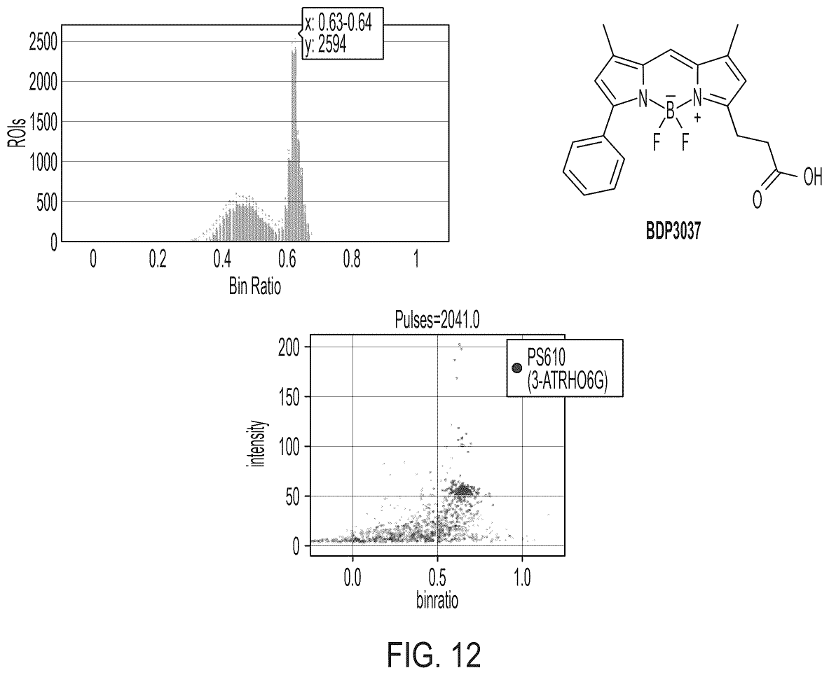

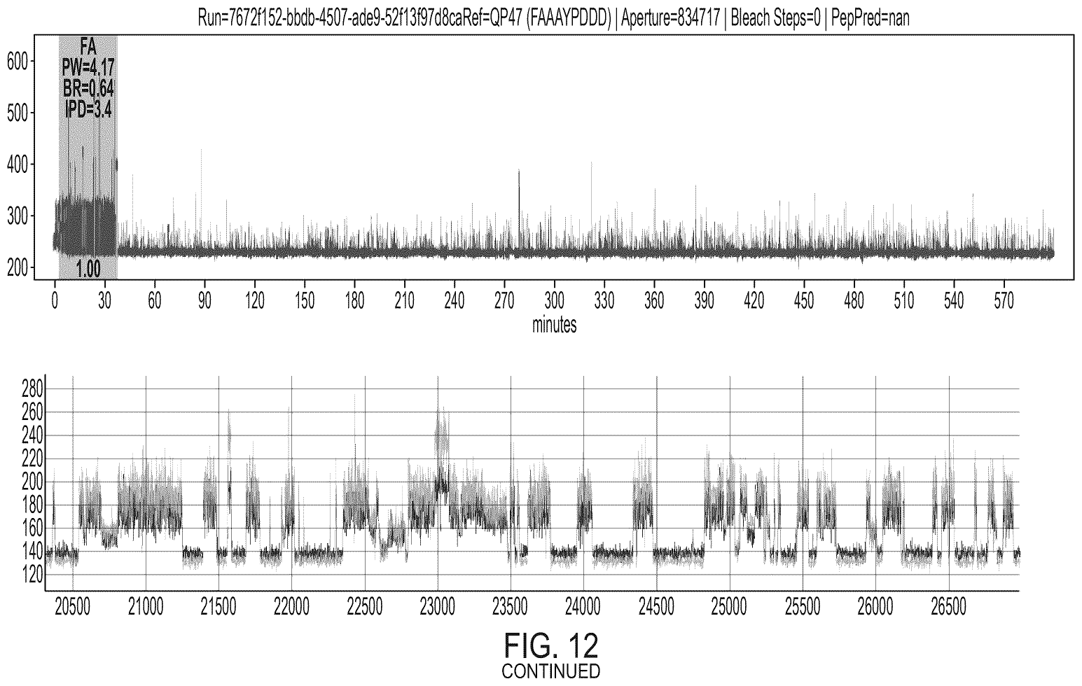

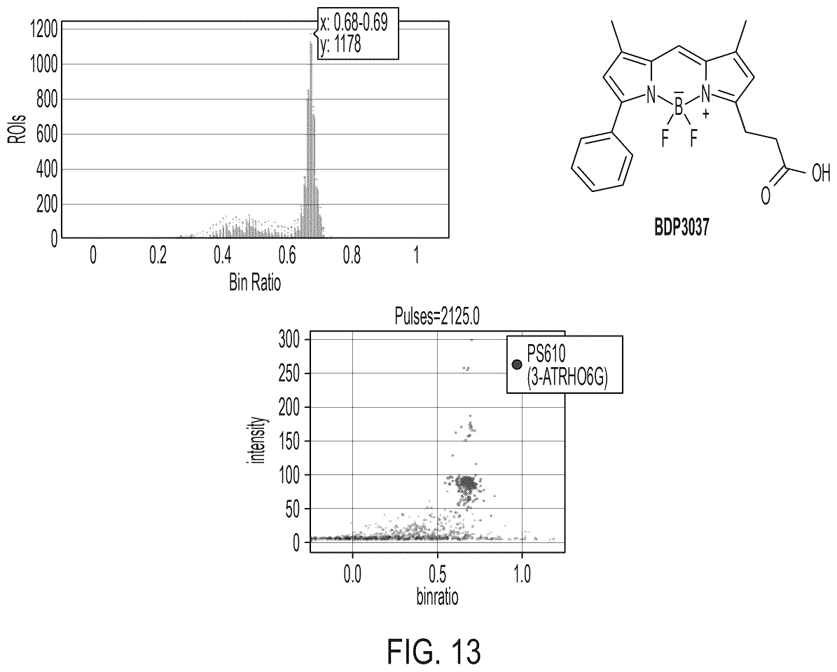

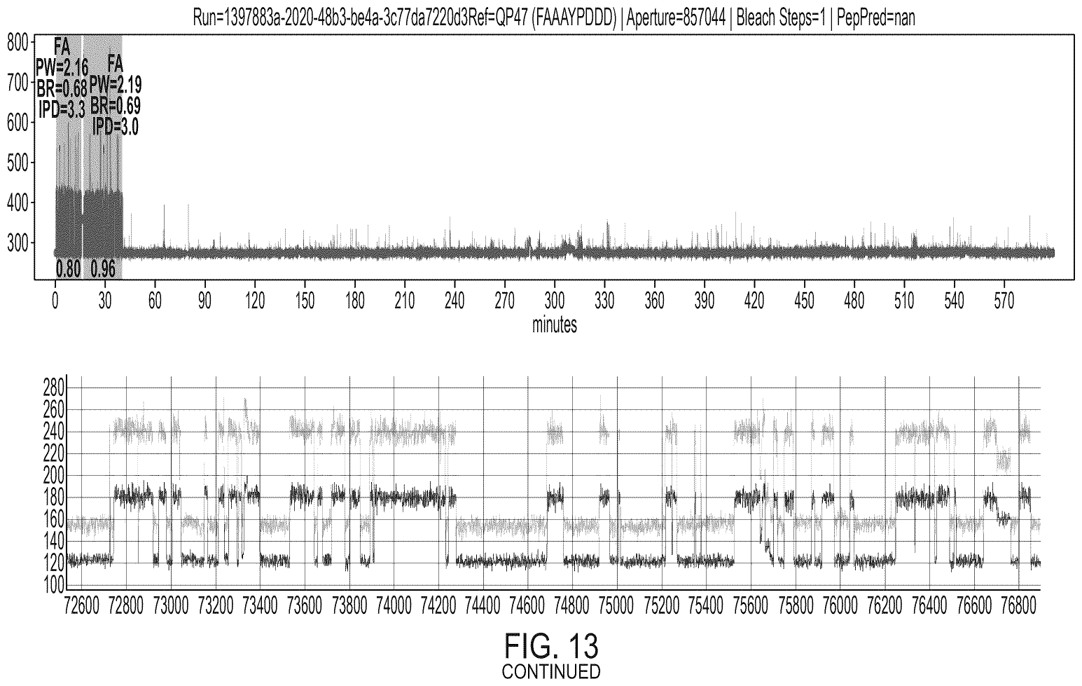

Water-soluble COT conjugates were made to improve solution stability of these TSQs (FIG. 11). The derivatives were made via various amide coupling reactions between COT-CO₂H and the appropriate amine. COT-CO₂H can be purchased or made in two steps from the parent COT compound. COT-PEG3-sulfonate (triethylammonium salt) was more stable in solution that the parent COT compound, even in the presence of mineral oil (FIGS. 12A-12B). When used to replace Trolox, COT-PEG3-sulfonate (triethylammonium salt) provided clean pulses and improved intensity and bin ratio with BDP3037 (FIGS. 13-14).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### EMBODIMENTS

### Embodiments of the Present Disclosure Include:

Embodiment 1. A compound of Formula (I):

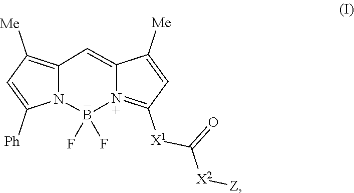

or a salt thereof, wherein:
X¹ is substituted or unsubstituted C₁-C₆ alkylene;
X² is a bond, —O—, or —N(R′)—;
R¹ is hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, or substituted or unsubstituted heteroaryl; and
Z is hydrogen, halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a polypeptide, or a polynucleotide.

Embodiment 2. The compound of embodiment 1, or salt thereof, wherein X¹ is unsubstituted C₁-C₆ alkylene.

Embodiment 3. The compound of any one of embodiments 1 and 2, or salt thereof, wherein X¹ is unsubstituted C₁-C₃ alkylene.

Embodiment 4. The compound of any one of embodiments 1-3, or salt thereof, wherein X¹ is ethylene.

Embodiment 5. The compound of any one of embodiments 1-4, or salt thereof, wherein X² is a bond.

Embodiment 6. The compound of any one of embodiments 1-4, or salt thereof, wherein X² is —O—.

Embodiment 7. The compound of any one of embodiments 1-4, or salt thereof, wherein X² is —N(R′)—.

Embodiment 8. The compound of any one of embodiments 1-4 and 7, or salt thereof, wherein R¹ is hydrogen.

Embodiment 9. The compound of any one of embodiments 1-4 and 7, or salt thereof, wherein R¹ is substituted or unsubstituted alkyl.

Embodiment 10. The compound of any one of embodiments 1-4, 7, and 9, or salt thereof, wherein R¹ is acyl.

Embodiment 11. The compound of any one of embodiments 1-10, or salt thereof, wherein Z is hydrogen.

Embodiment 12. The compound of any one of embodiments 1-4, 6, and 11, or salt thereof, wherein X²—Z is —OH.

Embodiment 13. The compound of embodiment 12, wherein the compound is of formula:

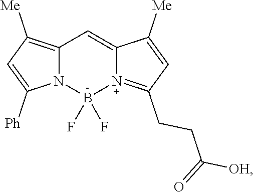

or a salt thereof.

Embodiment 14. The compound of any one of embodiments 1-10, or salt thereof, wherein Z is substituted heterocyclyl.

Embodiment 15. The compound of any one of embodiments 1-10 and 14, or salt thereof, wherein Z is

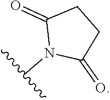 [SMILES: *N1C(=O)CCC1=O]

Embodiment 16. The compound of any one of embodiments 1-4, 6, 14, and 15, or salt thereof, wherein X²—Z is

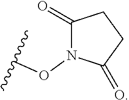 [SMILES: *ON1C(=O)CCC1=O]

Embodiment 17. The compound of any one of embodiments 1-4, 6, and 14-16, wherein the

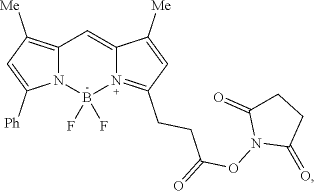

or a salt thereof.

Embodiment 18. The compound of any one of embodiments 1-10, or salt thereof, wherein Z is a polypeptide.

Embodiment 19. The compound of any one of embodiments 1-10, or salt thereof, wherein Z is a polynucleotide.

Embodiment 20. The compound of embodiment 19, or salt thereof, of the formula:

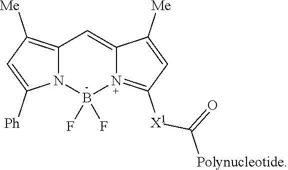

Embodiment 21. The compound of embodiment 20, or salt thereof, of the formula:

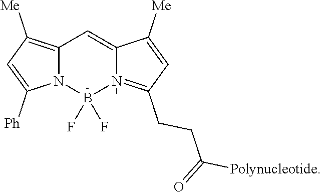

Embodiment 22. An amino acid recognition molecule, or a salt thereof, comprising at least one instance of Formula (II):

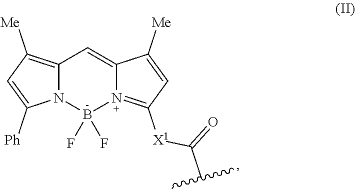

or a salt thereof, wherein:
each instance of X¹ is substituted or unsubstituted C₁-C₆ alkylene.

Embodiment 23. The amino acid recognition molecule of embodiment 22, or salt thereof, wherein the amino acid recognition molecule, or salt thereof, is linked to at least one instance of Formula (II) via a linker.

Embodiment 24. The amino acid recognition molecule of embodiment 23, or salt thereof, wherein the linker comprises one or more moieties selected from substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, and polynucleotide.

Embodiment 25. The amino acid recognition molecule of any one of embodiments 23-24, or salt thereof, wherein the linker comprises a polynucleotide.

Embodiment 26. The amino acid recognition molecule of any one of embodiments 22-25, or salt thereof, wherein at least one instance of X¹ is unsubstituted C₁-C₆ alkylene.

Embodiment 27. The amino acid recognition molecule of any one of embodiments 22-26, or salt thereof, wherein at least one instance of X¹ is unsubstituted C₁-C₃ alkylene.

Embodiment 28. The amino acid recognition molecule of any one of embodiments 22-27, or salt thereof, wherein at least one instance of X¹ is ethylene.

Embodiment 29. The amino acid recognition molecule of any one of embodiments 22-28, or salt thereof, wherein at least one instance of Formula (II) is of formula:

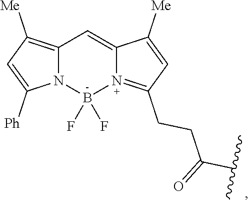

or a salt thereof.

Embodiment 30. The amino acid recognition molecule of any one of embodiments 22-29, or salt thereof, wherein the amino acid recognition molecule, or salt thereof, selectively binds a type of amino acid.

Embodiment 31. The amino acid recognition molecule of embodiment 30, or salt thereof, wherein the amino acid recognition molecule, or salt thereof, has a sequence selected from: Table 1, Table 2, and Table 3.

Embodiment 32. The amino acid recognition molecule of any one of embodiments 22-31, or salt thereof, wherein the amino acid recognition molecule, or salt thereof, has a molecular weight of, at most, about 100 kDa.

Embodiment 33. The amino acid recognition molecule of any one of embodiments 22-32, or salt thereof, wherein the amino acid recognition molecule, or salt thereof, has a molecular weight of between about 5 kDa and about 100 kDa.

Embodiment 34. The amino acid recognition molecule of any one of embodiments 22-33, or salt thereof, wherein the amino acid recognition molecule, or salt thereof, comprises at least 1, 2, 3, 4, or 5 instances of Formula (II), or a salt thereof.

Embodiment 35. The amino acid recognition molecule of any one of embodiments 22-34, or salt thereof, wherein the amino acid recognition molecule, or salt thereof, comprises at least 2 instances of Formula (II), or a salt thereof.

Embodiment 36. The amino acid recognition molecule of any one of embodiments 22-35, or salt thereof, wherein the amino acid recognition molecule, or salt thereof, comprises at least 3 instances of Formula (II), or a salt thereof.

Embodiment 37. The amino acid recognition molecule of any one of embodiments 22-36, or salt thereof, wherein at least one instance of Formula (II), or a salt thereof, is thermally stable.

Embodiment 38. The amino acid recognition molecule of embodiment 37, or salt thereof, wherein thermal stability comprises at least one instance of Formula (II), or a salt thereof, maintaining at least about 50% of its maximum fluorescence when the amino acid recognition molecule, or salt thereof, is maintained at a temperature of about 15° C. to about 65° C. for a time of at least about 5 minutes.

Embodiment 39. The amino acid recognition molecule of embodiment 38, or salt thereof, wherein at least one instance of Formula (II), or a salt thereof, maintains at least about 80% of its maximum fluorescence when the amino acid recognition molecule, or salt thereof, is maintained at a temperature of about 15° C. to about 65° C. for a time of at least about 5 minutes.

Embodiment 40. The amino acid recognition molecule of any one of embodiments 38 and 39, or salt thereof, wherein at least one instance of Formula (II), or a salt thereof, maintains at least about 90% of its maximum fluorescence when the amino acid recognition molecule, or salt thereof, is maintained at a temperature of about 15° C. to about 65° C. for a time of at least about 5 minutes.

Embodiment 41. The amino acid recognition molecule of any one of embodiments 38-40, or salt thereof, wherein the temperature is between about 35° C. and about 65° C.

Embodiment 42. The amino acid recognition molecule of any one of embodiments 38-41, or salt thereof, wherein at least one instance of Formula (II), or a salt thereof, maintains at least about 50% of its maximum fluorescence when the amino acid recognition molecule, or salt thereof, is maintained at a temperature of about 15° C. to about 65° C. for a time of at least about 4 hours.

Embodiment 43. A composition comprising the amino acid recognition molecule of any one of embodiments 22-42, or a salt thereof.

Embodiment 44. The composition of embodiment 43, further comprising one or more amino acid recognition molecules, or salts thereof, conjugated to a dye, wherein the dye is not a 1,9-dimethyl-3-phenyl BODIPY dye.

Embodiment 45. The composition of embodiment 44, wherein the dye is one or more dyes selected from 4-Cy3B, 4-SGCy3, C2C, 3-AttoRho6G, and R1C1.

Embodiment 46. The composition of any one of embodiments 43-45, further comprising a triplet quencher.

Embodiment 47. The composition of embodiment 46, wherein the triplet quencher is a compound of Formula (V):

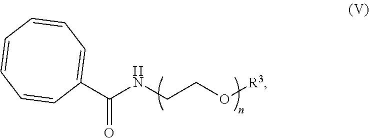

or a salt thereof, wherein:
R³ is substituted or unsubstituted aliphatic; and
n is 1,2, 3, 4, 5, 6, 7, 8, 9, or 10.

Embodiment 48. The composition of embodiment 47, wherein R³ is substituted C₁₋₆ alkyl.

Embodiment 49. The composition of any one of embodiments 47 and 48, wherein R³ is

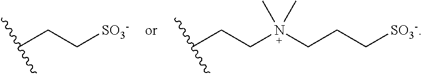

Embodiment 50. The composition of embodiment 47, wherein R³ is unsubstituted C₁₋₆ alkyl.

Embodiment 51. The composition of any one of embodiments 47 and 50, wherein R³ is —CH₃.

Embodiment 52. The composition of any one of embodiments 47-51, wherein n is 1, 2, 3, 4, or 5.

Embodiment 53. The composition of any one of embodiments 46-52, wherein the triplet quencher is a compound of formula:

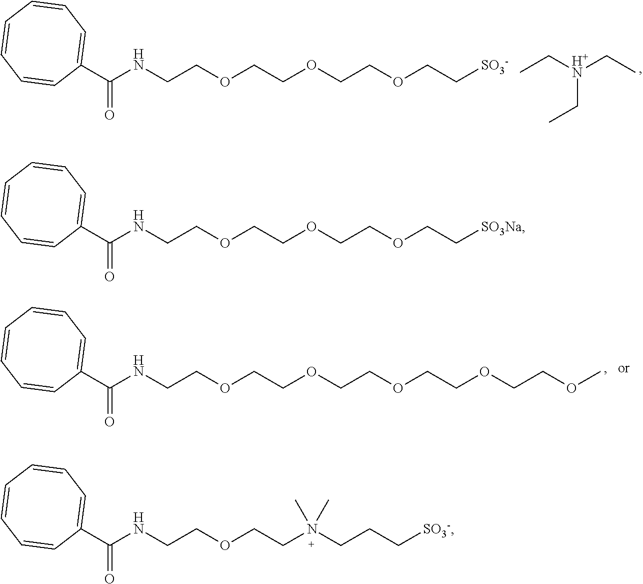

or a salt thereof.

Embodiment 54. A method of sequencing a polypeptide, the method comprising:
(i) directing a series of pulses of one or more excitation energies towards a composition comprising a polypeptide, and an amino acid recognition molecule of any one of embodiments 22-42, or salt thereof;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

Embodiment 55. The method of embodiment 54, wherein the composition further comprises one or more amino acid recognition molecules, or salts thereof, conjugated to a dye, wherein the dye is not a 1,9-dimethyl-3-phenyl BODIPY dye.

Embodiment 56. The method of embodiment 55, wherein the dye is one or more dyes selected from 4-Cy3B, 4-SGCy3, C2C, 3-AttoRho6G, and R1C1.

Embodiment 57. A method of sequencing a polypeptide, the method comprising:
(i) directing a series of pulses of one or more excitation energies towards a composition comprising the polypeptide, and an amino acid recognition molecule, or a salt thereof, wherein the amino acid recognition molecule or salt thereof comprises at least one instance of Formula (IV):

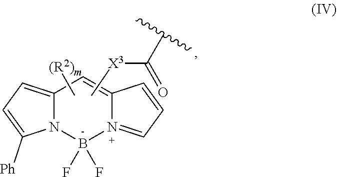

or a salt thereof, wherein:
each instance of R² is substituted or unsubstituted C₁-C₆ alkyl;
each instance of X³ is substituted or unsubstituted C₁-C₆ alkylene;
each instance of m is 1, 2, 3, 4, or 5; and
each instance of [symbol] is a bond to the amino acid recognition molecule, or salt thereof;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

Embodiment 58. The method of embodiment 57, wherein at least one instance of R² is unsubstituted C₁-C₆ alkyl.

Embodiment 59. The method of any one of embodiments 57 and 58, wherein at least one instance of R² is unsubstituted C₁-C₃ alkyl.

Embodiment 60. The method of any one of embodiments 57-59, wherein at least one instance of R² is methyl.

Embodiment 61. The method of any one of embodiments 57-60, wherein at least one instance of m is 1, 2, or 3.

Embodiment 62. The method of any one of embodiments 57-61, wherein at least one instance of Formula (IV) is of Formula (IV-a):

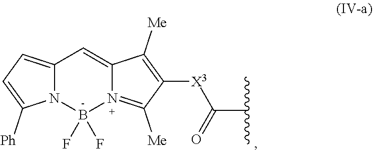

or a salt thereof.

Embodiment 63. The method of any one of embodiments 57-62, wherein at least one instance of X³ is unsubstituted C₁-C₆ alkylene.

Embodiment 64. The method of any one of embodiments 57-63, wherein at least one instance of X³ is unsubstituted C₁-C₃ alkylene.

Embodiment 65. The method of any one of embodiments 57-64, wherein at least one instance of X³ is ethylene.

Embodiment 66. The method of any one of embodiments 57-65, wherein at least one instance of Formula (IV) is of formula:

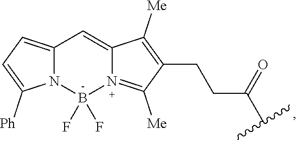

or a salt thereof.

Embodiment 67. The method of any one of embodiments 54-66, wherein the composition further comprises a triplet quencher.

Embodiment 68. The method of embodiment 67, wherein the triplet quencher is a compound of Formula (V):

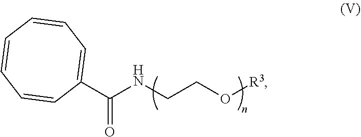

or a salt thereof, wherein:
R³ is substituted or unsubstituted C₁₋₆ alkyl; and
n is 1,2, 3, 4, 5, 6, 7, 8, 9, or 10.

Embodiment 69. The method of embodiment 68, wherein R³ is substituted C₁₋₆ alkyl.

Embodiment 70. The method of any one of embodiments 68 and 69, wherein R³ is

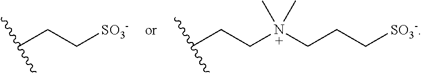

Embodiment 71. The method of embodiment 68, wherein R³ is unsubstituted C₁₋₆ alkyl.

Embodiment 72. The method of any one of embodiments 68 and 71, wherein R³ is —CH₃.

Embodiment 73. The method of any one of embodiments 68-72, wherein n is 1, 2, 3, 4, or 5.

Embodiment 74. The method of any one of embodiments 67-73, wherein the triplet quencher is a compound of formula:

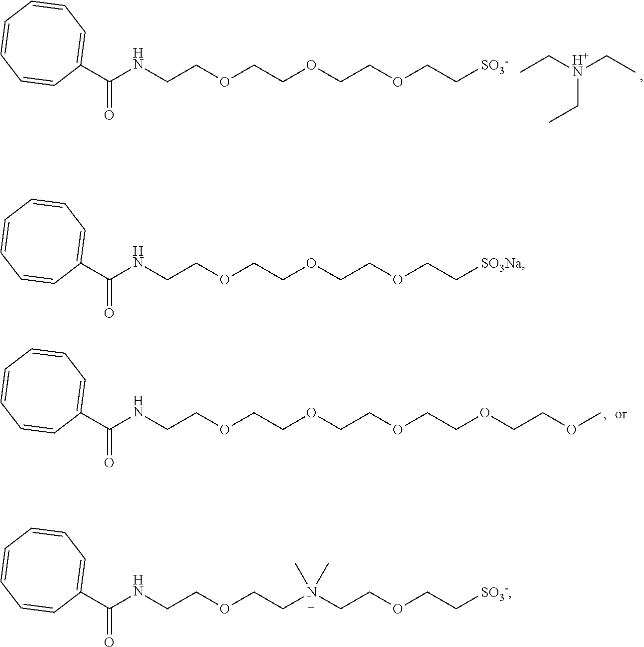

or a salt thereof.

Embodiment 75. A system for performing the method of any one of embodiments 54-74.

1. A compound of Formula (I): 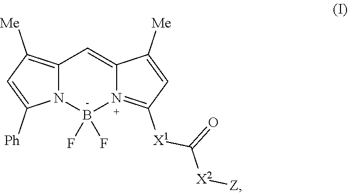
or a salt thereof, wherein:
X¹ is substituted or unsubstituted C₁-C₆ alkylene;
X² is a bond, —O—, or —N(R¹)—;
R¹ is hydrogen, substituted or unsubstituted aliphatic, substituted or unsubstituted heteroaliphatic, substituted or unsubstituted carbocyclyl, substituted or unsubstituted heterocyclyl, substituted or unsubstituted aryl, or substituted or unsubstituted heteroaryl; and
Z is hydrogen, halogen, substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, a polypeptide, or a polynucleotide moiety.

2. The compound of claim 1, or salt thereof, wherein X¹ is unsubstituted C₁-C₆ alkylene.

3. (canceled)

4. The compound of claim 1, or salt thereof, wherein X¹ is ethylene.

5. The compound of claim 1, or salt thereof, wherein X² is a bond or —O—.

6-10. (canceled)

11. The compound of claim 1, or salt thereof, wherein Z is hydrogen or substituted heterocyclyl.

12. The compound of claim 11, or salt thereof, wherein X²—Z is —OH or

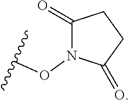 [SMILES: *ON1C(=O)CCC1=O]

13. The compound of claim 12, wherein the compound is of formula: 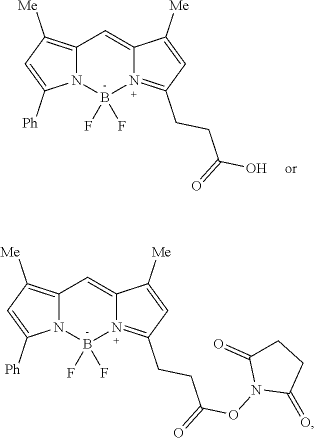
or a salt thereof.

14-17. (canceled)

18. The compound of claim 1, or salt thereof, wherein Z is a polypeptide or a polynucleotide.

19-20. (canceled)

21. The compound of claim 18, or salt thereof, of the formula:

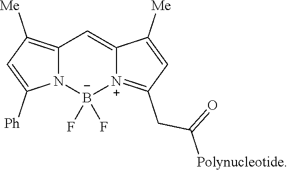

22. An amino acid recognition molecule, or a salt thereof, comprising at least one instance of Formula (II): 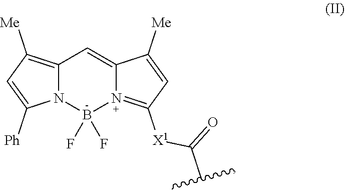
or a salt thereof, wherein:
each instance of X¹ is substituted or unsubstituted C₁-C₆ alkylene.

23. The amino acid recognition molecule of claim 22, or salt thereof, wherein the amino acid recognition molecule, or salt thereof, is linked to at least one instance of Formula (II) via a linker, wherein the linker comprises one or more moieties selected from substituted or unsubstituted aliphatic, substituted or unsubstituted heteroaliphatic, substituted or unsubstituted carbocyclyl, substituted or unsubstituted heterocyclyl, substituted or unsubstituted aryl, substituted or unsubstituted heteroaryl, and polynucleotide.

24-28. (canceled)

29. The amino acid recognition molecule of claim 22, or salt thereof, wherein at least one instance of Formula (II) is of formula: 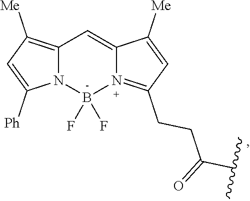
or a salt thereof.

30. (canceled)

31. The amino acid recognition molecule of claim 22, or salt thereof, wherein the amino acid recognition molecule, or salt thereof, has a sequence selected from: Table 1, Table 2, and Table 3.

32-33. (canceled)

34. The amino acid recognition molecule of claim 22, or salt thereof, wherein the amino acid recognition molecule, or salt thereof, comprises at least 1, 2, 3, 4, or 5 instances of Formula (II), or a salt thereof.

35-42. (canceled)

43. A composition comprising the amino acid recognition molecule of claim 22, or a salt thereof.

44. The composition of claim 43, further comprising;
one or more amino acid recognition molecules, or salts thereof, conjugated to a dye, wherein the dye is not a 1,9-dimethyl-3-phenyl BODIPY dye; or
a triplet quencher of Formula (V): 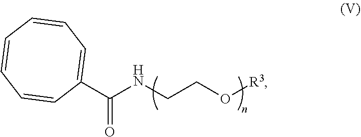
or a salt thereof, wherein:
R³ is substituted or unsubstituted aliphatic; and
n is 1,2, 3, 4, 5, 6, 7, 8, 9, or 10.

45-52. (canceled)

53. The composition of claim 44, wherein the triplet quencher is a compound of formula: 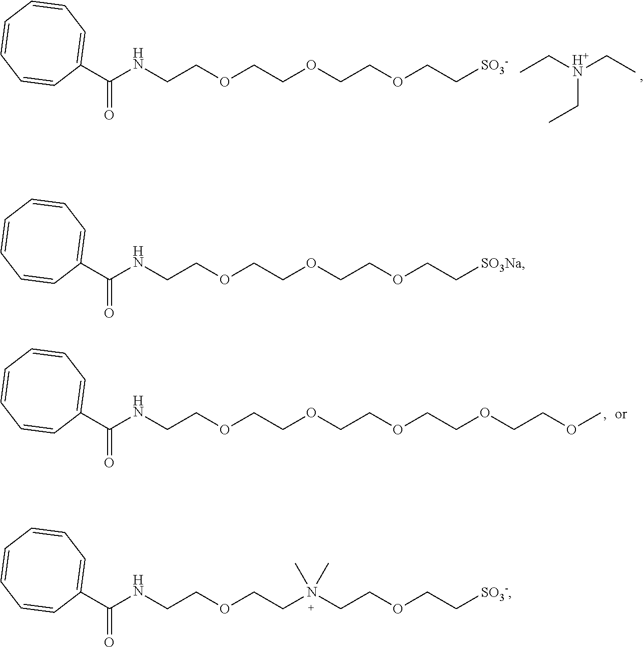
or a salt thereof.

54. A method of sequencing a polypeptide, the method comprising:
(i) directing a series of pulses of one or more excitation energies towards a composition comprising a polypeptide, and the amino acid recognition molecule of claim 22, or salt thereof;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

55-56. (canceled)

57. A method of sequencing a polypeptide, the method comprising:
(i) directing a series of pulses of one or more excitation energies towards a composition comprising the polypeptide, and an amino acid recognition molecule, or a salt thereof, wherein the amino acid recognition molecule or salt thereof comprises at least one instance of Formula (IV): 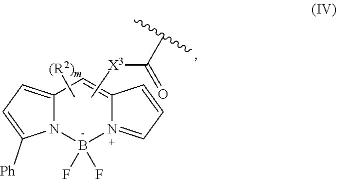
or a salt thereof, wherein:
each instance of R² is substituted or unsubstituted C₁-C₆ alkyl;
each instance of X³ is substituted or unsubstituted C₁-C₆ alkylene;
each instance of m is 1, 2, 3, 4, or 5; and
each instance of [symbol] is a bond to the amino acid recognition molecule, or salt thereof;
(ii) detecting a plurality of emitted photons from the amino acid recognition molecule, or salt thereof; and
(iii) identifying the sequence of incorporated amino acid residues in the polypeptide by determining at least one of luminescence intensity and luminescence lifetime based on the emitted photons.

58-74. (canceled)

75. A system for performing the method of claim 54.


SEQUENCE LISTING (216 sequences)

SEQ ID NO: 1 - protein, 97 aa, synthetic construct
MASAPSTTLDKSTQVVKKTYPNYKVIVLNDDLNTFDHVANCLIKYIPDMTTDRAWELTNQVHYQGQAIVWTGPQEQAELYHQQLRREGLTMAPLEAA

SEQ ID NO: 2 - protein, 97 aa, synthetic construct
MTSTLRARPARDTDLQHRPYPHYRIIVLDDDVNTFQHVVNCLVTFLPGMTRDQAWAMAQQVDGEGSAVVWTGPQEQAELYHVQLGNHGLTMAPLEPV

SEQ ID NO: 3 - protein, 95 aa, synthetic construct
MFNSLGTVLDPKKSKAKYPEARVIVLDDNFNTFQHVANCLLAIIPRMCEQRAWDLTIKVDKAGSAEVWRGNLEQAELYHEQLFSKGLTMAPIEKT

SEQ ID NO: 4 - protein, 96 aa, synthetic construct
MATETIERPRTRDPGSGLGGHWLVIVLNDDHNTFDHVAKTLARVIPGVTVDDGYRFADQIHQRGQAIVWRGPKEPAEHYWEQLQDAGLSMAPLERH

SEQ ID NO: 5 - protein, 106 aa, synthetic construct
MAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAV

SEQ ID NO: 6 - protein, 149 aa, synthetic construct
MNRIKQEAVRTENLLICSESIRRTPGTMSNEESMFDEVVAVAVAEPETQHDERRGTKPKRQPPYHVILWDDTDHSFDYVIMMMKRLFRMPIEKGFQVAKEVDSSGRAICMTTTLELAELKRDQIHAFGKDELLPRCKGSMSATIEPAEG

SEQ ID NO: 7 - protein, 103 aa, synthetic construct
MRWEDPLAAEPVTPGVAPVVEEETDAAVETPWRVILYDDDIHTFEEVILQLMKATGCTPEQGERHAWTVHTRGKDCVYQGDFFDCFRVQGVLREIQLVTEIEG

SEQ ID NO: 8 - protein, 93 aa, synthetic construct
MEAEPETKVLASIPGVGTSEPFRVVLFNDEEHSFDEVIFQIIKAVRCSRAKAEALTMEVHNSGRSIVYTGPIEQCIRVSAVLEEIELRTEIQS

SEQ ID NO: 9 - protein, 101 aa, synthetic construct
MPTNDLDLLEKQDVKIERPKMYQVVMYNDDFTPFDFVVAVLMQFFNKGMDEATAIMMQVHMQGKGICGVFPKDIAETKATEVMKWAKVEQHPLRLQVEAQA

SEQ ID NO: 10 - protein, 130 aa, synthetic construct
MADISKSRPEIGGPKGPQFGDSDRGGGVAVITKPVTKKKFKRKSQTEYEPYWHVLLHHDNVHTFEYATGAIVKVVRTVSRKTAHRITMQAHVSGVATVTTTWKAQAEEYCKGLQMHGLTSSIAPDSSFTH

SEQ ID NO: 11 - protein, 87 aa, synthetic construct
MXPQEVEEVSFLESKEHEIVLYNDDVNTFDHVIECLVKICNHNYLQAEQCAYIVHHSGKCGVKTGSLEELIPKCNALLEEGLSAEVI

SEQ ID NO: 12 - protein, 89 aa, synthetic construct
MSTQEEVLEEVKTTTQKENEIVLYNDDYNTFDHVIETLIYACEHTPVQAEQCAILVHYKGKCTVKTGSFDELKPRCSKLLEEGLSAEIV

SEQ ID NO: 13 - protein, 94 aa, synthetic construct
MGDIYGESNPEEVSCIDSLSEEGNELILFNDNIHTFEYVIDCLVAICSLSYEQASNCAYIVDRKGLCTVKHGSYDELLIMYHALVEKDLKVEIR

SEQ ID NO: 14 - protein, 91 aa, synthetic construct
MVAFSKKWKKDELDKSTGKQKMLILHNDSVNSFDYVIKTLCEVCDHDTIQAEQCAFLTHFKGQCEIAVGEVADLVPLKNKLLNKNLIVSIH

SEQ ID NO: 15 - protein, 115 aa, synthetic construct
MSDSPVIKEIKKDNIKEADEHEKKEREKETSAWKVILYNDDIHNFTYVTDVIVKVVGQISKAKAHTITVEAHSTGQALILSTWKSKAEKYCQELQQNGLTVSIIHESQLKDKQKK

SEQ ID NO: 16 - protein, 108 aa, synthetic construct
MVTTLSADVYGMATAPTVAPERSNQVVRKTYPNYKVIVLNDDFNTFQHVAECLMKYIPGMSSDRAWDLTNQVHYEGQAIVWVGPQEPAELYHQQLRRAGLTMAPLEAA

SEQ ID NO: 17 - protein, 106 aa, synthetic construct
MLNSAAFKAASASPVIAPERSGQVTQKPYPTYKVIVLNDDFNTFQHVHDCLVKYIPGMTSDRAWQLTHQVHNDGQAIVWVGPQEQAELYHQQLSRAGLTMAPIEAA

SEQ ID NO: 18 - protein, 104 aa, synthetic construct
MLSIAAVTEAPSKGVQTADPKTVRKPYPNYKVIVLNDDFNTFQHVSSCLLKYIPGMSEARAWELTNQVHFEGLAVVWVGPQEQAELYYAQLKNAGLTMAPPEPA

SEQ ID NO: 19 - protein, 95 aa, synthetic construct
MGQTVEKPRVEGPGTGLGGSWRVIVRNDDHNTFDHVARTLARFIPGVSLERGHEIAKVIHTTGRAVVYTGHKEAAEHYWQQLKGAGLTMAPLEQG

SEQ ID NO: 20 - protein, 93 aa, synthetic construct
MSVEIIEKRSTVRKLAPRYRVLLHNDDFNPMEYVVQTLMATVPSLTQPQAVNVMMEAHTNGMGLVIVCALEHAEFYAETLNNHGLGSSIEPDD

SEQ ID NO: 21 - protein, 108 aa, synthetic construct
MSDEDGEDGDENAVGIATRTRTRTKKPTPYRVLLLNDDYTPMEFVVLVLQRFFRMSIEDATRVMLQVHQKGVGVCGVFTYEVAETKVSQVIDFARQNQHPLQCTLEKA

SEQ ID NO: 22 - protein, 109 aa, synthetic construct
MAERRDTGDDEGTGLGIATKTRSKTKKPTPYRVLMLNDDYTPMEFVVLCLQRFFRMNMEEATRVMLHVHQKGVGVCGVFSYEVAETKVGQVIDFARANQHPLQCTLEKA

SEQ ID NO: 23 - protein, 101 aa, synthetic construct
MTVSQSKTQGAPAAQSATELEYEGLWRVVVLNDPVNLMSYVVLVFKKVFGFDETTARKHMLEVHEQGRSVVWSGMREKAEAYAFTLQQWHLTTVLEQDEVR

SEQ ID NO: 24 - protein, 103 aa, synthetic construct
MSDNDVALKPKIKSKPKLERPKLYKVILVNDDFTPREFVIAVLKMVFRMSEETGYRVMLTAHRLGTSVVVVCARDIAETKAKEAVDFGKEAGFPLMFTTEPEE

SEQ ID NO: 25 - protein, 103 aa, synthetic construct
MSDNEVAPKRKTRVKPKLERPRLYKVILVNDDYTPRDFVVMVLKAIFRMSEEAGYRVMMTAHKLGTSVVVVCARDIAETKAKEATDLGKEAGFPLMFTTEPEE

SEQ ID NO: 26 - protein, 137 aa, synthetic construct
MPLKAQNRSIVGRRDEWPPPTTQSSSETKSESKRVSDTGADTKRKTKTVPKVEKPRLYKVILVNDDYTPREFVLVVLKAVFRMSEDQGYKVMITAHQKGSCVVAVYTRDIAETKAKEAVDLAKEIGFPLMFRTEPEE

SEQ ID NO: 27 - protein, 103 aa, synthetic construct
MPVSVTAPQTKTKTKPKVERPKLYKVILVNDDFTPREFVVRVLKAEFRMSEDQAAKVMMTAHQRGVCVVAVFTRDVAETKATRATDAGRAKGYPLLFTTEPEE

SEQ ID NO: 28 - protein, 128 aa, synthetic construct
MVSIGAATVACAEGRPIFSGYFDWLAAMPETVTVPRTRLRPKTERPKLHKVILVNDDYTPREFVVTVLKGEFHMSEDQAQRVMITAHRRGVCVVAVFTKDVAETKATRASDAGRAKGYPLQFTTEPEE

SEQ ID NO: 29 - protein, 101 aa, synthetic construct
MPDATTTPRTKTLTRTARPPLHKVILVNDDFTPREFVVRLLKAEFRTTGDEAQRIMITAHMKGSCVVAVFTREIAESKATRATETARAEGFPLLFTTEPEE

SEQ ID NO: 30 - protein, 137 aa, synthetic construct
MPSNKRQMCLSDIKNSFNESGIVDWHISPRLANEPSEEGDSDLAVQTVPPELKRPPLYAVVLLNDDYTPMEFVIEILQQYFAMNLDQATQVMLTVHYEGKGVAGVYPRDIAETKANQVNNYARSQGHPLLCQIEPKD

SEQ ID NO: 31 - protein, 106 aa, synthetic construct
MTDPPSKGREDVDLATRTKPKTQRPPLYKVLLLNDDFTPMEFVVHILERLFGMTHAQAIEIMLTVHRKGVAVVGVFSHEIAETKVAQVMELARRQQHPLQCTMEKE

SEQ ID NO: 32 - protein, 112 aa, synthetic construct
MPARLTDIEGEPNTDPVEDVLLADPELKKPQMYAVVMYNDDYTPMEFVVDVLQNHFKHTLDSAISIMLAIHQQGKGIAGIYPKDIAETKAQTVNRKARQAGYPLLSQIEPQG

SEQ ID NO: 33 - protein, 113 aa, synthetic construct
MGDDDQSSREGEGDVAFQTADPELKRPSLYRVVLLNDDYTPMEFVVHILEQFFAMNREKATQVMLAVHTQGKGVCGVYTKDIAETKAALVNDYSRENQHPLLCEVEELDDESR

SEQ ID NO: 34 - protein, 107 aa, synthetic construct
MTRPDAPEYDDDLAVEPAEPELARPPLYKVVLHNDDFTPMEFVVEVLQEFFNMDSEQAVQVMLAVHTQGKATCGIFTRDIAETKSYQVNEYARECEHPLMCDIEAAD

SEQ ID NO: 35 - protein, 102 aa, synthetic construct
MATKREGSTLLEPTAAKVKPPPLYKVLLLNDDYTPMEFVVLVLKKFFGIDQERATQIMLKVHTEGVGVCGVYPRDIAHTKVEQVVDFARQHQHPLQCTMEES

SEQ ID NO: 36 - protein, 121 aa, synthetic construct
MMKQCGSYFLIKAVQDFKPLSKHRSDTDVITETKIQVKQPKLYTVIMYNDNYTTMDFVVYVLVEIFQHSIDKAYQLMMQIHESGQAAVALLPYDLAEMKVDEVTALAEQESYPLLTTIEPA

SEQ ID NO: 37 - protein, 125 aa, synthetic construct
MQAAGNEPPDPQNPGDVGNGGDGGNQDGSNTGVVVKTRTRTRKPAMYKVLMLNDDYTPMEFVVHVLERFFQKNREEATRIMLHVHRRGVGVCGVYTYEVAETKVTQVMDLARQNQHPLQCTIEKE

SEQ ID NO: 38 - protein, 102 aa, synthetic construct
MALPETRTKIKPDVNIKEPPNYRVIYLNDDKTSMEFVIGSLMQHFSYPQQQAVEKTEEVHEHGSSTVAVLPYEMAEQKGIEVTLDARAEGFPLQVKIEPAER

SEQ ID NO: 39 - protein, 98 aa, synthetic construct
MTSQTDTLVKPNIQPPSLFKVIYINDSVTTMEFVVESLMSVFNHSADEATRLTQLVHEEGAAVVAILPYELAEQKGMEVTLLARNNGFPLAIRLEPAV

SEQ ID NO: 40 - protein, 105 aa, synthetic construct
MSNLDTDVLIDEKVKVVTTEPEKYRVILLNDDVTPMDFVINILVSIFKHSTDTAKDLTLKIHKEGSAIVGVYTYEIAEQKGIEATNESRQHGFPLQVKIERENTL

SEQ ID NO: 41 - protein, 104 aa, synthetic construct
MSDHNIDHDTSVAVHLDVVVREPPMYRVVLLNDDFTPMEFVVELLMHFFRKTAEQATQIMLNIHHEGVGVCGTYPREIAETKVAQVHQHARTNGHPLKCRMEPS

SEQ ID NO: 42 - protein, 102 aa, synthetic construct
MEKEQSLCKEKTHVELSEPKHYKVVFHNDDFTTMDFVVKVLQLVFFKSQLQAEDLTMKIHLEGSATAGIYSYDIAQSKAQKTTQMAREEGFPLRLTVEPEDN

SEQ ID NO: 43 - protein, 103 aa, synthetic construct
MSDYSNQISQAGSGVAEDASITLPPERKVVFYNDDFTTMEFVVDVLVSIFNKSHSEAEELMQTVHQEGSSVVGVYTYDIAVSRTNLTIQAARKNGFPLRVEVE

SEQ ID NO: 44 - protein, 104 aa, synthetic construct
MTTPNKRPEFEPEIGLEDEVGEPRKYKVLLHNDDYTTMDFVVQVLIEVFRKSETEATHIMLTIHEKGVGTCGIYPAEVAETKINEVHTRARREGFPLRASMEEV

SEQ ID NO: 45 - protein, 106 aa, synthetic construct
MTQIKPQTIPDTDVISQTQSDWQMPDLYAVIMHNDDYTTMDFVVFLLNAVFDKPIEQAYQLMMQIHQTGRAVVAILPYEIAEMKVDEATSLAEQEQFPLFISIEQA

SEQ ID NO: 46 - protein, 99 aa, synthetic construct
MAPTPAGAAVLDKQQQRRHKHASRYRVLLHNDPVNTMEYVVESLRQVVPQLSEQDAIAVMVEAHNTGVGLVIVCDIEPAEFYCEQLKTKGLTSSIEPED

SEQ ID NO: 47 - protein, 92 aa, synthetic construct
MSVETIEKRSTTRKLAPQYRVLLHNDDYNSMEYVVQVLMTSVPSITQPQAVNIMMEAHNSGLALVITCAQEHAEFYCETLKGHGLSSTIEPD

SEQ ID NO: 48 - protein, 103 aa, synthetic construct
MTHYFSNILRDQESPKINPKELEQIDVLEEKEHQIILYNDDVNTFEHVIDCLVKICEHNYLQAEQCAYIVHHSGKCSVKTGSLDELVPKCNALLEEGLSAEVV

SEQ ID NO: 49 - protein, 91 aa, synthetic construct
MSIIEKTQENVAILEKVSINHEIILYNDDVNTFDHVIETLIRVCNHEELQAEQCAILVHYTGKCAVKTGSFDELQPLCLALLDAGLSAEIT

SEQ ID NO: 50 - protein, 91 aa, synthetic construct
MSTKEKVKERVREKEAISFNNEIIVYNDDVNTFDHVIETLIRVCNHTPEQAEQCSLIVHYNGKCTVKTGSMDKLKPQCTQLLEAGLSAEIV

SEQ ID NO: 51 - protein, 91 aa, synthetic construct
MSTKEKVKERVREKEAVGFNNEIIVYNDDVNTFDHVIDTLMRVCSHTPEQAEQCSLIVHYNGKCTVKTGPMDKLKPQCTQLLEAGLSAEIV

SEQ ID NO: 52 - protein, 91 aa, synthetic construct
MSVQEEVLEEVKTKERVNKQNQIIVFNDDVNTFDHVIDMLIATCDHDPIQAEQCTMLIHYKGKCEVKTGDYDDLKPRCSKLLDAGISAEIQ

SEQ ID NO: 53 - protein, 92 aa, synthetic construct
MQPFEETYTDVLDEVVDTDVHNLVVFNDDVNTFDHVIETLIDVCKHTPEQAEQCTLLIHYKGKCSVKNGSWEELVPMRNEICRRGISAEVLK

SEQ ID NO: 54 - protein, 101 aa, synthetic construct
MIISSVKSSPSTETLSRTELQLGGVWRVVVLNDPVNLMSYVMMIFKKIFGFNETVARRHMLEVHEKGRSVVWSGLREKAEAYVFTLQQWHLTAVLESDETH

SEQ ID NO: 55 - protein, 101 aa, synthetic construct
MIGVEARTSSAPELAIETEIRLAGLWHVIVINDPVNLMSYVVMVLRKIFGFDDTKARKHMLEVHENGRSIVWSGEREPAEAYANTLHQWHLSAVLERDETD

SEQ ID NO: 56 - protein, 103 aa, synthetic construct
MMSSLKECSIQALPSLDEKTKTEEDLSVPWKVIVLNDPVNLMSYVVMVFRKVFGYNENKATKHMMEVHQLGKSVLWTGQREEAECYAYQLQRWRLQTILEKDD

SEQ ID NO: 57 - protein, 117 aa, synthetic construct
MSRLPWKQEAKFAATVIIDFPDATLEAPTIEKKEATEQQIEMPWNVVVHNDPVNLMSYVTMVFQRVFGYPRERAEKHMLEVHHSGRSILWSGLRERAELYVQQLHGYLLLATIEKTV

SEQ ID NO: 58 - protein, 126 aa, synthetic construct
MTLSVALGPDTQESTQTGTAVSTDTLTAPDIPWNLVIWNDPVNLMSYVSYVFQSYFGYSETKANKLMMEVHKKGRSIVAHGSKEQVEQHAVAMHGYGLWATVEKATGGNSGGGKSGGPGKGKGKRG

SEQ ID NO: 59 - protein, 109 aa, unidentified
  source 1..109: note = Planctomycetales bacterium
MSEPMTLPAIPQPRLKERTQRQPPYNVILLNDDDHSYEYVIAMLQVLFGYPREKGYQMAKEVDSTGRVILLTTTREHAELKQEQIHAFGPDPLMARCQGSMTAVIEPAV

SEQ ID NO: 60 - protein, 110 aa, unidentified
  source 1..110: note = Planctomycetia bacterium
MSDTITLPGRPEVERDERTRRQPPYNVILHNDDDHTFEYVIVMLNQLFGYPPEKGYEMAKEVHLNGRVIVLTTSKEHAELKRDQIHAFGPDPFSSKDCKGSMSASIEPAY

SEQ ID NO: 61 - protein, 133 aa, unidentified
  source 1..133: note = Gemmataceae bacterium
MGFPTDFRQSIEISTPLGSQQPRFSNASSEPALADPVLVINPRIQPRYHVILLNDDDHTYRYVIEMMLIVFGHPPEKGFLIAKEVDKAGRAICLTTSLEHAEFKQEQVHAYGADPYFGPKCKGSMTAVLEPAE

SEQ ID NO: 62 - protein, 106 aa, unidentified
  source 1..106: note = Gemmataceae bacterium
MSDTITLPEEKTDVRTKRQPPYHVILLNDDDHTYQYVIYMLQTLFGHPPETGFKMAQEVDKTGRVIVDTTSLERAELKRDQIHAFGPDPYIERCKGSMSAMIEPSE

SEQ ID NO: 63 - protein, 114 aa, unidentified
  source 1..114: note = Planctomycetes bacterium
MSESITTLPKKSRRLKEEEEQKTKRQPPYNVILLNDDDHTFEYVIFMLQKLFGHPPERGMQMAKEVHTTGRVIVMTTALELAELKRDQIHAFGPDPLIDRCKGSMSATIEPAPI

SEQ ID NO: 64 - protein, 104 aa, unidentified
  source 1..104: note = Planctomycetes bacterium
MPTFTEPEVVNDTRILPPYHVILLNDDDHTYEYVIHMLQTLFGHPQERGFQLAVEVDKKGKAIVFTTSKEHAEFKRDQIHAFGADPLSSKNCKGSMSAVIEPSF

SEQ ID NO: 65 - protein, 107 aa, unidentified
  source 1..107: note = Rubrobacter indicoceani
MPSAAPAKPKTKRQSRTQGMPPYNVVLLDDDDHTYGYVIEMLNKVFGHPPEKGFELATEVDKNGRVIVMTTNLEVAELKRDEVHAFGPDPLMPRSKGSMSAVVERAG

SEQ ID NO: 66 - protein, 108 aa, unidentified
  source 1..108: note = Fimbriiglobus ruber
MSKTSTLPEVESESAQKLKYQPPYHVILLNDDDHSYVYVITMLKELFGHPEQKGYQLADAVDKQGRAIVFTTTREHAELKQEQIHAYGPDPTIPRCKGAMTAVIEPAE

SEQ ID NO: 67 - protein, 120 aa, unidentified
  source 1..120: note = Planctomycetes bacterium
MPASASAVTEPPVSLPEAAAPRPKDRPKRQPRYHVILWNDDDHTYQYVVAMLRQLFGHPPEKGFTLAKQVDKDGRVVVLTTTKEHAELKRDQIHAFGADRLLARSKGSMSASIEPEASTG

SEQ ID NO: 68 - protein, 125 aa, unidentified
  source 1..125: note = Planctomycetia bacterium
MSDSASATVEVQADPPADATARSQPTPARSTGSKPKRQPRYHVVLWNDDDHTYEYVIAMLRRLFGIEPEKGFRIAEEVDQSGRAVVLTTTREHAELKRDQIHAFGADRLLARSKGSMSASIEPEA

SEQ ID NO: 69 - protein, 120 aa, unidentified
  source 1..120: note = Planctomycetes bacterium
MADSAQTGVAEPIQETLRRRKLRDDRRPKRQPPYHVILWNDNDHTYAYVVVMLMQLFGYPAEKGYQLASEVDTQGRAVVLTTTKEHAELKRDQIHAYGKDGLIEKCKGSMWATIEPAPGE

SEQ ID NO: 70 - protein, 118 aa, Blastopirellula marina
MGDSNTSVAEPGEVTVVTTKPAPKKAKPKRQPKYHVVLWNDDDHTYEYVILMMHELFGHPVEKGFQIAKTVDADGRAICLTTTKEHAELKRDQIHAYGKDELIARCRGSMSSTIEPEC

SEQ ID NO: 71 - protein, 119 aa, unidentified
  source 1..119: note = Planctomycetia bacterium
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEE

SEQ ID NO: 72 - protein, 117 aa, unidentified
  source 1..117: note = Planctomycetia bacterium
MTATTADPDRTTAEKTTKKARRSGQPKRQPRYHVILWNDNDHTYQYVVAMLQQLFGHPATTGLKLATEVDRTGRAVILTTTREHAELKRDQIHAFGADRLLARSKGAMSASIEPEAE

SEQ ID NO: 73 - protein, 114 aa, unidentified
  source 1..114: note = Planctomycetaceae bacterium
MNQAAISPNPDIKPNPSTHKKRASQRQPRYHVILWNDNDHTYHYVVTMLQKLFGHPPRTGIKMATEVDKKGKVIVLTTSREHAELKRDQIHAFGADKLIRRSKGAMAASIEPES

SEQ ID NO: 74 - protein, 117 aa, unidentified
  source 1..117: note = Planctomycetes bacterium
MTETITTPAERTQTQAEPRSDRAWLWNVVLLDDDEHTYEYVIRMLHTLFGMPVERAFRLAEEVDARGRAVVLTTHKEHAELKRDQVHAFGKDALIASCAGSMSAVLEPAECGSDDED

SEQ ID NO: 75 - protein, 117 aa, unidentified
  source 1..117: note = Roseimaritima sp.
MAELQTAVVEPTTRPEQDEKQSQSRPKRQPRYNVILWDDPDHSYDYVIMMLKELFGHPRQRGHQMAEEVDTTGRVICLTTTMEHAELKRDQIHAYGSDEGITRCKGSMSASIEPVPE

SEQ ID NO: 76 - protein, 120 aa, unidentified
  source 1..120: note = Rubripirellula amarantea
MSDQQSMVAEPEVVVHTQDEKKLEKQNKRKKQPRYNVVLWDDTDHSYDYVVLMMKQLFHHPIETGFQIAKQVDKGGKAICLTTTMEHAELKRDQIHAFGKDDLIARCTGSMSATIEPVPE

SEQ ID NO: 77 - protein, 109 aa, unidentified
  source 1..109: note = Acidobacteria bacterium
MSSRSATAYPEVEDDTSDQLQPLYHVILLNDEDHTYDYVIEMLQKIFGFPESKAFSHAVEVDTKGTTILLTCDLEQAERKRDLIHSYGPDWRLPRSLGSMAAVVEPAAG

SEQ ID NO: 78 - protein, 114 aa, unidentified
  source 1..114: note = Planctomycetes bacterium
MFEEVVSVAVAEPKTKKQSRTKPKRQPPYHVILWDDTDHTFDYVIKMMGELFRMPREKGYQLAKEVDTSGRAICMTTTLELAELKRDQIHAFGRDDASAHCKGSMSATIEPAEG

SEQ ID NO: 79 - protein, 129 aa, Aquisphaera sp.
MSEFDHEHSGDTSVADPIVTTKTAPKPQKHAENETETRRQPPYNVIILNDEEHTFDYVIELLCKVFRHSLATAQELTWRIHLTGRAVVLTTHKELAELKRDQVLAYGPDPRMSVSKGPLDCFIEPAPGG

SEQ ID NO: 80 - protein, 116 aa, unidentified
  source 1..116: note = Planctomycetaceae bacterium
MSSPSSLDDVQVSTSRAKPANETRTRKQPPYAVIVENDDHHTFLYVIEALMKVCGHAPEKGFVLAQQIHTQGKAMVWSGTLELAELKRDQLRGFGPDNYAPRPVTFPLGVTIEPLP

SEQ ID NO: 81 - protein, 129 aa, unidentified
  source 1..129: note = Planctomycetaceae bacterium
MADYEDAGEDALEDDFDHGTVTVAPQKPEPKKQSENKRQANRQPRYNVLLWDSEDHTFEYVEKMLRELFGHIKKQCQIIAEQVDQEGRAVVLTTTLEHAELKRDQIHAYGKDQLEGSKGSMWSTIEAVD

SEQ ID NO: 82 - protein, 114 aa, unidentified
  source 1..114: note = Dehalococcoidia bacterium
MTTPSLPTRETEVEERTEVEPERLYHLVLLDDDQHSYQYVIEMLASIFGYGSEKAWTLARIVDTEGRAILETASHAQCERHQSQIHAYGADSRIPTSVGSMSAVIEEAGTPPQT

SEQ ID NO: 83 - protein, 118 aa, unidentified
  source 1..118: note = Planctomycetes bacterium
MYSKNQIKIYCSEDDKGQTATPLLEKKPKFAPLYHVILWDDNTHTYEYVIKLLMSLFRMTFEKAYQHTLEVDKKGRTICITTHLEKAELKQEQISNFGPDILMQNSKGPMSATIEPAN

SEQ ID NO: 84 - protein, 108 aa, unidentified
  source 1..108: note = Leptospira congkakensis
MTGAGASQPSILEETEVRPRLSDGPWKVVLWDDDFHTYEYVIEMLMDVCQMPWEKAFQHAVEVDTRKKTIVFSGELEHAEFVHERILNYGPDPRMGSSKGSMTATLEQ

SEQ ID NO: 85 - protein, 108 aa, Leptospira meyeri
MTSSGASQPSILEETERKPRLSDGPWKVVLWDDDFHTYEYVIEMLMDVCQMPWEKAFQHAVEVDTRKKTIVFFGELEHAEFVHERILNYGPDPRMGTSKGSMTATLEK

SEQ ID NO: 86 - protein, 104 aa, Blastopirellula marina
MSSEELSLQTRPKRQPPFGVILHNDDLNSFDYVIDSIRKVFHYELEKCFQLTLEAHETGRSLLWTGTLEGAELKQELLLSCGPDPIMLDKGGLPLKVTLEELPQ

SEQ ID NO: 87 - protein, 105 aa, unidentified
  source 1..105: note = Leptospira fluminis
MSQTPVIEETTVKDPVKTGGPWKVVLWDDDEHTYDYVIEMLMEVCVMTMEQAFHHAVEVDTQKKTVVYSGEFEHAEHIQELILEYGPDPRMAVSKGSMSATLEKS

SEQ ID NO: 88 - protein, 110 aa, Gemmata obscuriglobus
MANATPTPDVVPEEETETRTRRQPPYAVVLHNDDTNTMDFVVTVLRKVFGYTVEKCVELMLEAHTQGKVAVWIGALEVAELKADQIKSFGPDPHVTKNGHPLGVTVEPAA

SEQ ID NO: 89 - protein, 106 aa, Leptospira kmetyi
MASTQTPDLNEITEESTKSTGGPWRVVLWDDNEHTYEYVIEMLMEICTMTVEKAFLHAVQVDQEKRTVVFSGEFEHAEHVQERILTYGADPRMSNSKGSMSATLEK

SEQ ID NO: 90 - protein, 106 aa, Leptospira interrogans
MASTQTPDLNEITEESTKSTGGPWRVVLWDDNEHTYEYVIEMLVEICMMTVEKAFLHAVQVDKEKRTVDFSGELEHAEHVQERILNYGADPRMSNSKGSMSATLER

SEQ ID NO: 91 - protein, 110 aa, unidentified
  source 1..110: note = Tuwongella immobilis
MSASSSQPGTTTKPDLDIQPRLLPPFHVILENDEFHSMEFVIDTLRKVLGVSIERAYQLMMTAHESGQAIIWTGPKEVAELKYEQVIGFHEKRSDGRDLGPLGCRIEPAV

SEQ ID NO: 92 - protein, 99 aa, unidentified
  source 1..99: note = Planctomycetes bacterium
MSGTVVESKPRNSTQLAPRWKVIVHDDPVTTFDFVLGVLRRVFAKPPGEARRITREAHDTGSALVDVLALEQAEFRRDQAHSLARAEGFPLTLTLEPAD

SEQ ID NO: 93 - protein, 117 aa, unidentified
  source 1..117: note = Agrobacterium tumifaciens
MIAEPICMQGEGDGEDGGTNRGTSVITRVKPKTKRPNLYRVLLLNDDYTPMEFVIHILERFFQKDREAATRIMLHVHQHGVGECGVFTYEVAETKVSQVMDFARQHQHPLQCVMEKK

SEQ ID NO: 94 - protein, 103 aa, unidentified
  source 1..103: note = Agrobacterium tumifaciens
MSDSPVDLKPKPKVKPKLERPKLYKVMLLNDDYTPREFVTVVLKAVFRMSEDTGRRVMMTAHRFGSAVVVVCERDIAETKAKEATDLGKEAGFPLMFTTEPEE

SEQ ID NO: 95 - protein, 103 aa, synthetic construct
MSDSPVDLKPKPKVKPKLERPKLYKVILLNDDYTPMEFVVEVLKRVFNMSEEQARRVMMTAHKKGKAVVGVCPRDIAETKAKQATDLAREAGFPLMFTTEPEE

SEQ ID NO: 96 - protein, 103 aa, synthetic construct
MSDSPVDLKPKPKVKPKLERLKLYKVILLNDDYTTAFFVVKVLKRVFNMSEEQARRVMMTAHKKGKAVVGVCPRDIAETKAKQATDLAREAGFPLMFTTEPEE

SEQ ID NO: 97 - protein, 103 aa, synthetic construct
MSDSPVDLKPKPKVKPKLERLKLYKVILLNDDYTTMRFVVLVLKRVFNMSEEQARRVMMTAHKKGKAVVGVCPRDIAETKAKQATDLAREAGFPLMFTTEPEE

SEQ ID NO: 98 - protein, 117 aa, synthetic construct
MIAEPICMQGEGDGEDGGTNRGTSVITRVKPKTKRPNLYRVLLLNDDYTPFQFVIHILERFFQKDREAAWRITLHVHQHGVGECGVFTYEVAETKVSQVMDFARQHQHPLQCVMEKK

SEQ ID NO: 99 - protein, 103 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEE

SEQ ID NO: 100 - protein, 103 aa, synthetic construct
MSDSPVDLKPKPKVKPKLERPKLYKVMLLNDDYTPREFVTVVLKAVFRMSEDTGRRVMMTAHRFGSAVVVVSERDIAETKAKEATDLGKEAGFPLMFTTEPEE

SEQ ID NO: 101 - protein, 103 aa, synthetic construct
MSDSPVDLKPKPKVKPKLERPKLYKVMLLNDDYTPMSFVTVVLKAVFRMSEDTGRRVMMTAHRFGSAVVVVSERDIAETKAKEATDLGKEAGFPLMFTTEPEE

SEQ ID NO: 102 - protein, 103 aa, synthetic construct
MSDSPVDLKPKPKVKPKLERPKLYKVILLNDDYTPMEFVVEVLKRVFNMSEEQARRVMMTAHKKGKAVVGVSPRDIAETKAKQATDLAREAGFPLMFTTEPEE

SEQ ID NO: 103 - protein, 117 aa, synthetic construct
MIAEPISMQGEGDGEDGGTNRGTSVITRVKPKTKRPNLYRVLLLNDDYTPMEFVIHILERFFQKDREAATRIMLHVHQHGVGECGVFTYEVAETKVSQVMDFARQHQHPLQCVMEKK

SEQ ID NO: 104 - protein, 117 aa, synthetic construct
MIAEPISMQGEGDGEDGGTNRGTSVITRVKPKTKRPNLYRVLLLNDDYTPMEFVIHILERFFQKDREAATRIMLHVHQHGVGESGVFTYEVAETKVSQVMDFARQHQHPLQSVMEKK

SEQ ID NO: 105 - protein, 95 aa, Synechococcus elongatus
MAVETIQKPETTTKRKIAPRYRVLLHNDDFNPMEYVVMVLMQTVPSLTQPQAVDIMMEAHTNGTGLVITCDIEPAEFYCEQLKSHGLSSSIEPDD

SEQ ID NO: 106 - protein, 136 aa, Synechococcus elongatus
MSPQPDESVLSILGVPRPCVKKRSRNDAFVLTVLTCSLQAIAAPATAPGTTTTRVRQPYPHFRVIVLDDDVNTFQHVAECLLKYIPGMTGDRAWDLTNQVHYEGAATVWSGPQEQAELYHEQLRREGLTMAPLEAA

SEQ ID NO: 107 - protein, 90 aa, unidentified
  source 1..90: note = Thermosynechococcus elongatus
MPQERQQVTRKHYPNYKVIVLNDDFNTFQHVAACLMKYIPNMTSDRAWELTNQVHYEGQAIVWVGPQEQAELYHEQLLRAGLTMAPLEPE

SEQ ID NO: 108 - protein, 106 aa, Escherichia coli
MGKTNDWLDFDQLAEEKVRDALKPPSMYKVILVNDDYTPMEFVIDVLQKFFSYDVERATQLMLAVHYQGKAICGVFTAEVAETKVAMVNKYARENEHPLLCTLEKA

SEQ ID NO: 109 - protein, 106 aa, Escherichia coli
MGKTNDWLDFDQLAEEKVRDALKPPSMYKVILVNDDYTPAEFVIDVLQKFFSYDVERATQLMLAVHYQGKAICGVFTAEVAETKVAMVNKYARENEHPLLCTLEKA

SEQ ID NO: 110 - protein, 192 aa, unidentified
  source 1..192: note = Plasmodium falciparum
MFKDLKPFFLCIILLLLLIYKCTHSYNIKNKNCPLNFMNSCVRINNVNKNTNISFPKELQKRPSLVYSQKNFNLEKIKKLRNVIKEIKKDNIKEADEHEKKEREKETSAWKVILYNDDIHNFTYVTDVIVKVVGQISKAKAHTITVEAHSTGQALILSTWKSKAEKYCQELQQNGLTVSIIHESQLKDKQKK

SEQ ID NO: 111 - protein, 236 aa, Escherichia coli
MRLVQLSRHSIAFPSPEGALREPNGLLALGGDLSPARLLMAYQRGIFPWFSPGDPILWWSPDPRAVLWPESLHISRSMKRFHKRSPYRVTMNYAFGQVIEGCASDREEGTWITRGVVEAYHRLHELGHAHSIEVWREDELVGGMYGVAQGTLFCGESMFSRMENASKTALLVFCEEFIGHGGKLIDCQVLNDHTASLGACEIPRRDYLNYLNQMRLGRLPNNFWVPRCLFSPQELE

SEQ ID NO: 112 - protein, 231 aa, Vibrio vulnificus
MSSDIHQIKIGLTDNHPCSYLPERKERVAVALEADMHTADNYEVLLANGFRRSGNTIYKPHCDSCHSCQPIRISVPDIELSRSQKRLLAKARSLSWSMKRNMDENWFDLYSRYIVARHRNGTMYPPKKDDFAHFSRNQWLTTQFLHIYEGQRLIAVAVTDIMDHCASAFYTFFEPEHELSLGTLAVLFQLEFCQEEKKQWLYLGYQIDECPAMNYKVRFHRHQKLVNQRWQ

SEQ ID NO: 113 - protein, 167 aa, Homo sapiens
MSGSKFRGHQKSKGNSYDVEVVLQHVDTGNSYLCGYLKIKGLTEEYPTLTTFFEGEIISKKHPFLTRKWDADEDVDRKHWGKFLAFYQYAKSFNSDDFDYEELKNGDYVFMRWKEQFLVPDHTIKDISGASFAGFYYICFQKSAASIEGYYYHRSSEWYQSLNLTHV

SEQ ID NO: 114 - protein, 362 aa, Saccharomyces cerevisiae
MINNPKVDSVAEKPKAVTSKQSEQAASPEPTPAPPVSRNQYPITFNLTSTAPFHLHDRHRYLQEQDLYKCASRDSLSSLQQLAHTPNGSTRKKYIVEDQSPYSSENPVIVTSSYNHTVCTNYLRPRMQFTGYQISGYKRYQVTVNLKTVDLPKKDCTSLSPHLSGFLSIRGLTNQHPEISTYFEAYAVNHKELGFLSSSWKDEPVLNEFKATDQTDLEHWINFPSFRQLFLMSQKNGLNSTDDNGTTNAAKKLPPQQLPTTPSADAGNISRIFSQEKQFDNYLNERFIFMKWKEKFLVPDALLMEGVDGASYDGFYYIVHDQVTGNIQGFYYHQDAEKFQQLELVPSLKNKVESSDCSFEFA

SEQ ID NO: 115 - protein, 240 aa, synthetic construct
MMEVQLQQSGPELVKPGASVMISCRTSAYTFTENTVHWVKQSHGESLEWIGGINPYYGGSIFSPKFKGKATLTVDKSSSTAYMELRSLTSEDSAVYYCARRAGAYYFDYWGQGTTLTVSSGGGSGGGSGGGSENVLTQSPAIMSASPGEKVTMTCRASSSVSSSYLHWYRQKSGASPKLWIYSTSNLASGVPARFSGSGSGTSYSLTISSVEAEDAATYYCQQYSGYRTFGGGTKLEIKR

SEQ ID NO: 116 - protein, 111 aa, Homo sapiens
MGAMDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLSSRLVVPSHK

SEQ ID NO: 117 - protein, 111 aa, Homo sapiens
MGAMDSIQAEEWYFGKLGRKDAERQLLSFGNPRGTFLIRESETVKGAYALSIRDWDDMKGDHVKHYLIRKLDNGGYYITTRAQFETLQQLVQHYSERAAGLSSRLVVPSHK

SEQ ID NO: 118 - protein, 113 aa, Homo sapiens
MGAMDSIQAEEWYFGKITRRESERLLLNAENPRGTFLVRESETTKGAYSLSVSDFDNAKGLNVKHYKIRKLDSGGFYITSRTQFNSLQQLVAYYSKHADGLCHRLTTVCPTSK

SEQ ID NO: 119 - protein, 113 aa, Homo sapiens
MGAMDSIQAEEWYFGKITRRESERLLLNAENPRGTFLVRESEVTKGAYALSVSDFDNAKGLNVKHYLIRKLDSGGFYITSRTQFNSLQQLVAYYSKHADGLCHRLTTVCPTSK

SEQ ID NO: 120 - protein, 310 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQ

SEQ ID NO: 121 - protein, 310 aa, Homo sapiens; identical to SEQ ID NO: 120

SEQ ID NO: 122 - protein, 310 aa, Homo sapiens; identical to SEQ ID NO: 120

SEQ ID NO: 123 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 124 - protein, 439 aa, Rattus norvegicus
MASLTVKAYLLGKEEAAREIRRFSFCFSPEPEAEAAAGPGPCERLLSRVAVLFPALRPGGFQAHYRDEDGDLVAFSSDEELTMAMSYVKDDIFRIYIKEKKECRREHRPPCAQEARSMVHPNVICDGCNGPVVGTRYKCSVCPDYDLCSVCEGKGLHREHSKLIFPNPFGHLSDSFSHSRWLRKLKHGHFGWPGWEMGPPGNWSPRPPRAGDGRPCPTAESASAPSEDPNVNFLKNVGESVAAALSPLGIEVDIDVEHGGKRSRLTPTSAESSSTGTEDKSGTQPSSCSSEVSKPDGAGEGPAQSLTEQMKKIALESVGQPEELMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 125 - protein, 292 aa, Saccharomyces cerevisiae
MTSLNIMGRKFILERAKRNDNIEEIYTSAYVSLPSSTDTRLPHFKAKEEDCDVYEEGTNLVGKNAKYTYRSLGRHLDFLRPGLRFGGSQSSKYTYYTVEVKIDTVNLPLYKDSRSLDPHVTGTFTIKNLTPVLDKVVTLFEGYVINYNQFPLCSLHWPAEETLDPYMAQRESDCSHWKRFGHFGSDNWSLTERNFGQYNHESAEFMNQRYIYLKWKERFLLDDEEQENQMLDDNHHLEGASFEGFYYVCLDQLTGSVEGYYYHPACELFQKLELVPTNCDALNTYSSGFEIA

SEQ ID NO: 126 - protein, 421 aa, unidentified
  source 1..421: note = Leishmania major
MSRNPSNSDAAHAFWSTQPVPQTEDETEKIVFAGPMDEPKTVADIPEEPYPIASTFEWWTPNMEAADDIHAIYELLRDNYVEDDDSMFRFNYSEEFLQWALCPPNYIPDWHVAVRRKADKKLLAFIAGVPVTLRMGTPKYMKVKAQEKGEGEEAAKYDEPRHICEINFLCVHKQLREKRLAPILIKEATRRVNRTNVWQAVYTAGVLLPTPYASGQYFHRSLNPEKLVEIRFSGIPAQYQKFQNPMAMLKRNYQLPSAPKNSGLREMKPSDVPQVRRILMNYLDSFDVGPVFSDAEISHYLLPRDGVVFTYVVENDKKVTDFFSFYRIPSTVIGNSNYNLLNAAYVHYYAATSIPLHQLILDLLIVAHSRGFDVCNMVEILDNRSFVEQLKFGAGDGHLRYYFYNWAYPKIKPSQVALVML

SEQ ID NO: 127 - protein, 496 aa, Homo sapiens
MADESETAVKPPAPPLPQMMEGNGNGHEHCSDCENEEDNSYNRGGLSPANDTGAKKKKKKQKKKKEKGSETDSAQDQPVKMNSLPAERIQEIQKAIELFSVGQGPAKTMEEASKRSYQFWDTQPVPKLGEVVNTHGPVEPDKDNIRQEPYTLPQGFTWDALDLGDRGVLKELYTLLNENYVEDDDNMFRFDYSPEFLLWALRPPGWLPQWHCGVRVVSSRKLVGFISAIPANIHIYDTEKKMVEINFLCVHKKLRSKRVAPVLIREITRRVHLEGIFQAVYTAGVVLPKPVGTCRYWHRSLNPRKLIEVKFSHLSRNMTMQRTMKLYRLPETPKTAGLRPMETKDIPVVHQLLTRYLKQFHLTPVMSQEEVEHWFYPQENIIDTFVVENANGEVTDFLSFYTLPSTIMNHPTHKSLKAAYSFYNVHTQTPLLDLMSDALVLAKMKGFDVFNALDLMENKTFLEKLKFGIGDGNLQYYLYNWKCPSMGAEKVGLVLQ

SEQ ID NO: 128 - protein, 127 aa, Drosophila melanogaster
MGDVQPETCRPSAASGNYFPQYPEYAIETARLRTFEAWPRNLKQKPHQLAEAGFFYTGVGDRVRCFSCGGGLMDWNDNDEPWEQHALWLSQCRFVKLMKGQLYIDTVAAKPVLAEEKEESTSIGGDT

SEQ ID NO: 129 - protein, 73 aa, unidentified
  source 1..73: note = Amanita thiersii
MICGQIIGKGESCFRCRDCGLDESCVMCSQCFHATDHINHNVSFFVSQQPGGCCDCGDEEAWKKPMNCPYHPP

SEQ ID NO: 130 - protein, 74 aa, Helobdella robusta
MVCLKVFKLGEPTYSCRSVTCGMDPTCVLCVDCFQNSSHKLHKYKMSTSGGGGYCDCGDLEAWKADPLCDLHKL

SEQ ID NO: 131 - protein, 73 aa, unidentified
  source 1..73: note = Hydra vulgaris
MFCGRLFKVGDPTYTCKDCAADPTCVFCHDCFHQSVHTKHKYKLFASQGRGGYCDCGDKEAWTNDPACNKHKE

SEQ ID NO: 132 - protein, 72 aa, unidentified
  source 1..72: note = Galleria mellonella
MLCGKVFKQGEPAYSCRECGMDNTCVLCVECFKVSPHRHHKYKMGQSGGGGCCDCGDTEAWKRDPFCERHAK

SEQ ID NO: 133 - protein, 72 aa, Brachionus plicatilis
MVCGRVFKSGEPSYFCRECGTDPTCVLCSICFRHSKHRYHKYVMMTSGGGGYCDCGDPEAWKSDPCCELHMP

SEQ ID NO: 134 - protein, 72 aa, unidentified
  source 1..72: note = Capitella teleta
MLCGKVFKMGELTYSCRDCGTDPTCVLCMDCFQHSAHKKHRYKMAASGGGGYCDCGDREAWKAEPFCDVHKR

SEQ ID NO: 135 - protein, 73 aa, unidentified
  source 1..73: note = Sparassis crispa
MPCGHIFKKGESCFRCKDCALDDSCVLCSKCFEATDHANHNVSFFIAQQSGGCCDCGDIEAWLVPIDCPFHPV

SEQ ID NO: 136 - protein, 72 aa, unidentified
  source 1..72: note = Anabarilius graham
MLCGRVFKEGETVYSCRDCAIDPTCVLCIECFQKSVHKSHRYKMHASAGGGFCDCGDLEAWKTGPCCSQHDP

SEQ ID NO: 137 - protein, 72 aa, unidentified
  source 1..72: note = Lottia gigantean
MICGHGFKTGEPTYSCRDCATDPTCVLCISCFQKSPHREHRYKMSASGGGGYCDCGDPEAWKIEPFCEQHKP

SEQ ID NO: 138 - protein, 72 aa, Camponotus floridanus
MICGRMFKMGEPTYSCRQCGMDSTCVLCVDCFKQSAHRNHKYKMGTSSGGGCCDCGDTEAWKNEPFCKIHLA

SEQ ID NO: 139 - protein, 72 aa, Habropoda laboriosa
MICGKVFKMGEATYSCKECGVDPTCVLCADCFKQSAHRHHKYRMGTSSGGGFCDCGDIEAWKKEPFCNTHLA

SEQ ID NO: 140 - protein, 72 aa, Mastacembelus armatus
MLCGRVFKEGETVYSCRDCAIDPTCVLCMDCFQDSVHKSHRYKMHASAGGGFCDCGDVEAWKIGPYCSKHDP

SEQ ID NO: 141 - protein, 73 aa, unidentified
  source 1..73: note = Pyrenophora seminiperda
MPCGHIFKNGEATYRCKTCTADDTCVLCARCFDASDHEGHQVFVSVSPGNSGCCDCGDDEAWVRPVHCNIHSA

SEQ ID NO: 142 - protein, 72 aa, Tribolium castaneum
MVCGRVFKLGEPTYSCRECGMDNTCVLCVNCFKNSEHRFHKYKMGTSQGGGCCDCGDVEAWKKAPFCDVHIA

SEQ ID NO: 143 - protein, 72 aa, Wasmannia auropunctata
MICGKMFKIGEPTYSCRECGMDSTCVLCVDCFKQSAHRNHKYKMGTSSGGGCCDCGDTEAWKKEPFCKTHVV

SEQ ID NO: 144 - protein, 72 aa, Crassostrea gigas
MLCGKVFKTGEPTYSCRDCANDPTCVLCIDCFQNGAHKNHRYKMNTSGGGGYCDCGDQEAWTSHPFCNLHSP

SEQ ID NO: 145 - protein, 72 aa, Harpegnathos saltator
MMCGRVFKMGEPTYSCRECGVDSTCVLCVGCFQQSAHRDHKYKMGTSGGGGCCDCGDTEAWKRDPFCEIHMV

SEQ ID NO: 146 - protein, 72 aa, Nilaparvata lugens
MVCGRVFKMGEPSYHCRECGMDATCVLCVDCFKKSSHRNHKYKMGTSIGGGCCDCGDVEAWKTEPYCEVHIA

SEQ ID NO: 147 - protein, 72 aa, Manduca sexta
MLCGRVFKQGEPAYSCRECGMDNTCVLCVECFKVSAHRHHKYKMGQSGGGGCCDCGDTEAWKRDPFCELHAA

SEQ ID NO: 148 - protein, 72 aa, Monopterus albus
MLCGRVFKEGETVYSCRDCAIDPTCVLCMDCFQDSVHKSHRYKMHASSGGGFCDCGDVEAWKIGPCCSKHDP

SEQ ID NO: 149 - protein, 72 aa, unidentified
  source 1..72: note = Lingula anatine
MLCGRVFRSGEPTYSCRDCAVDPTCVLCIDCFNNGAHRKHKYRMSTSSGGGYCDCGDKEAWKTDPLCEIHRK

SEQ ID NO: 150 - protein, 72 aa, Vombatus ursinus
MLCGKVFKSGETTYSCRDCAIDPTCVLCMNCFQSSVHKNHRYKMHTSTGGGFCDCGDTEAWKTGPFCTIHEP

SEQ ID NO: 151 - protein, 83 aa, unidentified
  source 1..83: note = Saccharomycetaceae sp.
MAKSHRHTGRNCGRAFQPGEPLYRCQECAYDDTCVLCISCFNPDDHVNHHVSTHICNELHDGICDCGDAEAWNVPLHCKAEED

SEQ ID NO: 152 - protein, 72 aa, unidentified
  source 1..72: note = Drosophila ficusphila
MVCGKVFKNGEPTYSCRECGVDPTCVLCVNCFKRSAHRFHKYKMSTSGGGGCCDCGDDEAWKKDHYCQLHLA

SEQ ID NO: 153 - protein, 72 aa, Mus musculus
MLCGKVFKSGETTYSCRDCAIDPTCVLCMDCFQSSVHKNHRYKMHTSTGGGFCDCGDTEAWKTGPFCVDHEP

SEQ ID NO: 154 - protein, 72 aa, unidentified
  source 1..72: note = Maylandia zebra
MLCGRVFKEGETVYSCRDCAIDPTCVLCMDCFQDSVHKSHRYKMHASSGGGFCDCGDVEAWKIGPYCSKHDP

SEQ ID NO: 155 - protein, 72 aa, unidentified
  source 1..72: note = Mizuhopecten yessoensis
MLCGKVFKYGEPTYSCRDCANDPTCVLCIDCFQKSAHKKHRYKMSTSGGGGYCDCGDSEAWKTAPFCSNHKA

SEQ ID NO: 156 - protein, 83 aa, unidentified
  source 1..83: note = Kluyveromyces lactis
MHSKFNHAGRICGAKFRVGEPIYRCKECSFDDTCVLCVNCFNPKDHVGHHVYTSICTEFNNGICDCGDKEAWNHELNCKGAED

SEQ ID NO: 157 - protein, 72 aa, Chelonia mydas
MLCGKVFKGGETTYSCRDCAIDPTCVLCMDCFQNSIHKNHRYKMHTSTGGGFCDCGDTEAWKTGPLCANHEP

SEQ ID NO: 158 - protein, 72 aa, Acropora millepora
MLCGKVFKVGEPTYSCRDCGYDNTCVLCINCFQKSIHKNHHYKMNTSGGGGVCDCGDVEAWKEGEACEIHQQ

SEQ ID NO: 159 - protein, 72 aa, Musca domestica
MVCGKVFKIGEPTYSCRECGMDQTCVLCVNCFKQSAHRYHKYKMSTSGGGGCCDCGDEEAWKKDHYCEEHLR

SEQ ID NO: 160 - protein, 73 aa, unidentified
  source 1..73: note = Schizosaccharomyces cryophilus
MSCGRIFKKGEVFYRCKTCSVDSNSALCVKCFRATDHHGHETSFTISAGSGGCCDCGNSAAWIRDMPCKIHNR

SEQ ID NO: 161 - protein, 72 aa, unidentified
  source 1..72: note = Contarinia nasturtii
MVCGRVFKMNEPFYSCRECGMDPTCVLCVNCFKQSAHRHHKYKMGTSAGGGCCDCGDNEAWKQDHYCDEHTK

SEQ ID NO: 162 - protein, 73 aa, unidentified
  source 1..73: note = Schizosaccharomyces pombe
MKCGHIFRKGEVFYRCKTCSVDSNSALCVKCFRATSHKDHETSFTVSAGSGGCCDCGNAAAWIGDVSCKIHSH

SEQ ID NO: 163 - protein, 72 aa, Mus musculus
MLCGRVFKVGEPTYSCRDCAVDPTCVLCMECFLGSIHRDHRYRMTTSGGGGFCDCGDTEAWKEGPYCQKHKL

SEQ ID NO: 164 - protein, 72 aa, Aphis gossypii
MVCGRVFKMGEPTYNCRECGMDSTCVLCVDCFKRSPHKNHKYKMGTSYGGGCCDCGDVEAWKHDPYCQTHKL

SEQ ID NO: 165 - protein, 72 aa, Aedes aegypti
MVCGRVFKIGEPTYSCRECSMDPTCVLCSSCFKKSSHRLHKYKMSTSGGGGCCDCGDHEAWKRDPSCEEHAV

SEQ ID NO: 166 - protein, 83 aa, Saccharomyces cerevisiae
MGDVHKHTGRNCGRKFKIGEPLYRCHECGCDDTCVLCIHCFNPKDHVNHHVCTDICTEFTSGICDCGDEEAWNSPLHCKAEEQ

SEQ ID NO: 167 - protein, 83 aa, Saccharomyces cerevisiae
MGSVHKHTGRNCGRKFKIGEPLYRCHECGCDDTCVLCIHCFNPKDHVNHHVCTDICTEFTSGICDCGDEEAWNSPLHCKAEEQ

SEQ ID NO: 168 - protein, 83 aa, unidentified
  source 1..83: note = Kazachstania africana
MQTSFTHKGRNCGRKFKVGEPLYRCHECGFDDTCVLCIHCFNPADHENHHIYTDICNDFTSGICDCGDTEAWNGDLHCKAEEI

SEQ ID NO: 169 - protein, 73 aa, unidentified
  source 1..73: note = Clathrospora elynae
MPCGHIFKNGEATYRCKTCTADDTCVLCARCFDASDHEGHQVFVSVSPGNSGCCDCGDDEAWVRPVHCNMHSA

SEQ ID NO: 170 - protein, 73 aa, unidentified
  source 1..73: note = Aspergillus neoniger
MRCGHIFRAGEATYRCITCAADDTCVLCSRCFDASDHTGHQYQISLSSGNCGCCDCGDEEAWRLPLFCAIHTD

SEQ ID NO: 171 - protein, 72 aa, unidentified
  source 1..72: note = Trichuris suis
MRCNHVFANGEATYSCRGCAADPTCVLCASCFELSAHKEHKYMITTSSGTGYCDCGDPEAWKADPFCQQHQP

SEQ ID NO: 172 - protein, 72 aa, unidentified
  source 1..72: note = Trichinella spiralis
MKCNRQLICGEPTYCCLDCACDQTCIFCHACFQSSEHKNHRYSMSTSEGSGTCDCGDKEAWKSNYYCLNHKP

SEQ ID NO: 173 - protein, 77 aa, Homo sapiens
MGPLGSLCGRVFKSGETTYSCRDCAIDPTCVLCMDCFQDSVHKNHRYKMHTSTGGGFCDCGDTEAWKTGPFCVNHEP

SEQ ID NO: 174 - protein, 76 aa, Homo sapiens
MGPLGSLCGRVFKVGEPTYSCRDCAVDPTCVLCMECFLGSIHRDHRYRMTTSGGGGFCDCGDTEAWKEGPYCQKHE

SEQ ID NO: 175 - protein, 81 aa, unidentified
  source 1..81: note = Kluyveromyces marxianus
MVNEHRGSQCSKQCHGTETVYYCFDCTKNPLYEICEECFDETQHMGHRYTSRVVTRPEGKVCHCGDISGYNNPEKAFQCKI

SEQ ID NO: 176 - protein, 81 aa, unidentified
  source 1..81: note = Kluyveromyces lactis
MHNDHRGSQCSKQCHGTETVYYCFDCTKNPLYEICEDCFDESQHIGHRYTSRVVTRPEGKVCHCGDISSYNDPKKAFQCRI

SEQ ID NO: 177 - protein, 81 aa, unidentified
  source 1..81: note = Eremothecium sinecaudum
MPKEHRGTSCNKHCQPTETVYYCFDCTKNPLYEICEECFDADKHLGHRWTSKVVSRPEGKICHCGDPSGLTDPENGYECKN

SEQ ID NO: 178 - protein, 81 aa, unidentified
  source 1..81: note = Zygosaccharomyces bailii
MNASHKGAMCSKQCYPTETVFYCFTCTTNPLYEICESCFDEEKHRGHLYTAKVVVRPEGRVCHCGDPFVFKEPRFAFLCKN

SEQ ID NO: 179 - protein, 81 aa, unidentified
  source 1..81: note = Vanderwaltozyma polyspora
MENLHIGSCCNRQCYPTQTVYYCLTCTINPLYEICELCFDEDKHVGHTYISKSVIRPEGKVCHCGNPNVFKKPEFAFNCKN

SEQ ID NO: 180 - protein, 81 aa, Saccharomyces cerevisiae
MGNMHIGTACTRLCFPSETIYYCFTCSTNPLYEICELCFDKEKHVNHSYVAKVVMRPEGRICHCGDPFAFNDPSDAFKCKN

SEQ ID NO: 181 - protein, 83 aa, unidentified
  source 1..83: note = Kluyveromyces marxianus
MHSKFSHAGRICGAKFKVGEPIYRCKECSFDDTCVLCVNCFNPKDHTGHHVYTTICTEFNNGICDCGDKEAWNHTLFCKAEEG

SEQ ID NO: 182 - protein, 83 aa, unidentified
  source 1..83: note = Kluyveromyces dobzhanskii
MHSRFNHAGRICASKFKVGEPIYRCKECSFDDTCVICVNCFNPKDHVGHHVYTSICSEFNNGICDCGDTEAWNHDMHCKADEN

SEQ ID NO: 183 - protein, 83 aa, unidentified
  source 1..83: note = Kazachstania naganishii
MSKQFRHKGRNCGRKFRLGEPLYRCQECGYDDTCVLCINCFNPKDHEGHHIYTDICNDFTSGICDCGDEEAWLSPLHCKAEED

SEQ ID NO: 184 - protein, 83 aa, unidentified
  source 1..83: note = Eremothecium sinecaudum
MPKNHNHKGRNCGRSFQPGEPLYRCQECAYDDTCVLCIRCFNPLDHVNHHVSTHICSEFNDGICDCGDVEAWNVELNCKAEED

SEQ ID NO: 185 - protein, 83 aa, unidentified
  source 1..83: note = Saccharomyces eubayanus
MGDVHKHTGRNCGRKFKIGEPLYRCHECGCDDTCVLCIHCFNPKDHINHHVCTDICSEFTSGICDCGDEEAWNSSLHCKAEEQ

SEQ ID NO: 186 - protein, 83 aa, unidentified
  source 1..83: note = Zygosaccharomyces parabailii
MYHVYKHSGRNCGRKFKVGEPIYRCHECGYDETCVLCIHCFNPKDHDSHHVYIDICSEFSTGICDCGDTEAFVNPLHCKAEED

SEQ ID NO: 187 - protein, 83 aa, unidentified
  source 1..83: note = Zygosaccharomyces mellis
MPKYHQHSGRYCGRKFKVGEPIYRCHECGFDETCVICIHCFNAKDHETHHVSVSICSEYSTGICDCGDTEAFVNPLHCRAEEV

SEQ ID NO: 188 - protein, 83 aa, Candida albicans
MSHRAYHKNSPCGRIFRKGEPIHRCLTCGFDDTCALCSHCFQPEYHEGHKVHIGICQRENGGVCDCGDPEAWTQELFCPYAVD

SEQ ID NO: 189 - protein, 83 aa, unidentified
  source 1..83: note = Pichia pastoris
MCPNYKHHGRPCARQFKQGEPIYRCYECGFDETCVMCMHCFNREQHRDHEVSISIASSSNDGICDCGDPQAWNIELHCQSELD

SEQ ID NO: 190 - protein, 284 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGGGSGGGSGGGSGMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHE

SEQ ID NO: 191 - protein, 283 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHE

SEQ ID NO: 192 - protein, 307 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFGSAGSAAGSGEFGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHE

SEQ ID NO: 193 - protein, 288 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGGSHHHHHHHHHHGGGSGGGSGGGSGLNDFFEAQKIEWHEGGGSGGGSGGGSGLNDFFEAQKIEWHE

SEQ ID NO: 194 - protein, 286 aa, synthetic construct
MAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHE

SEQ ID NO: 195 - protein, 291 aa, synthetic construct
MAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGGSHHHHHHHHHHGGGSGGGSGGGSGLNDFFEAQKIEWHEGGGSGGGSGGGSGLNDFFEAQKIEWHE

SEQ ID NO: 196 - protein, 318 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGGSGGGSGGGSGMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGSHHHHHHHHHHGGGSGGGSGGGSGLNDFFEAQKIEWHEGGGSGGGSGGGSGLNDFFEAQKIEWHE

SEQ ID NO: 197 - protein, 317 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGSHHHHHHHHHHGGGSGGGSGGGSGLNDFFEAQKIEWHEGGGSGGGSGGGSGLNDFFEAQKIEWHE

SEQ ID NO: 198 - protein, 341 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFGSAGSAAGSGEFGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGSHHHHHHHHHHGGGSGGGSGGGSGLNDFFEAQKIEWHEGGGSGGGSGGGSGLNDFFEAQKIEWHE

SEQ ID NO: 199 - protein, 301 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGSHHHHHHHHHHGGGSGGGSGGGSGLNDFFEAQKIEWHEGGGSGGGSGGGSGLNDFFEAQKIEWHE

SEQ ID NO: 200 - protein, 301 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGGSHHHHHHHHHHGGGSGGGSGGGSGLNDFFEAQKIEWHEGGGSGGGSGGGSGLNDFFEAQKIEWHE

SEQ ID NO: 201 - protein, 398 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHE

SEQ ID NO: 202 - protein, 513 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHE

SEQ ID NO: 203 - protein, 409 aa, synthetic construct
MAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGGSHHHHHHHHHHGGGSGGGSGGGSGLNDFFEAQKIEWHEGGGSGGGSGGGSGLNDFFEAQKIEWHE

SEQ ID NO: 204 - protein, 527 aa, synthetic construct
MAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGSAGSAAGSGEFMAFPARGKTAPKNEVRRQPPYNVILLNDDDHTYRYVIEMLQKIFGFPPEKGFQIAEEVDRTGRVILLTTSKEHAELKQDQVHSYGPDPYLGRPCSGSMTCVIEPAVGGSHHHHHHHHHHGGGSGGGSGGGSGLNDFFEAQKIEWHEGGGSGGGSGGGSGLNDFFEAQKIEWHE

SEQ ID NO: 205 - protein, 448 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGSHHHHHHHHHHGGGSGGGSGGGSGLNDFFEAQKIEWHEGGGSGGGSGGGSGLNDFFEAQKIEWHE

SEQ ID NO: 206 - protein, 579 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGSAGSAAGSGEFMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEEGGSHHHHHHHHHHGGGSGGGSGGGSGLNDFFEAQKIEWHEGGGSGGGSGGGSGLNDFFEAQKIEWHE

SEQ ID NO: 207 - protein, 246 aa, synthetic construct
MHSKFSHAGRICGAKFKVGEPIYRCKECSFDDTCVLCVNCFNPKDHTGHHVYTTICTEFNNGICDCGDKEAWNHTLFCKAEEGGGGSGGGSGGGSGMHSKFSHAGRICGAKFKVGEPIYRCKECSFDDTCVLCVNCFNPKDHTGHHVYTTICTEFNNGICDCGDKEAWNHTLFCKAEEGGGSHHHHHHHHHHGGGSGGGSGGGSGLNDFFEAQKIEWHEGGGSGGGSGGGSGLNDFFEAQKIEWHE

SEQ ID NO: 208 - protein, 245 aa, synthetic construct
MHSKFSHAGRICGAKFKVGEPIYRCKECSFDDTCVLCVNCFNPKDHTGHHVYTTICTEFNNGICDCGDKEAWNHTLFCKAEEGGSAGSAAGSGEFMHSKFSHAGRICGAKFKVGEPIYRCKECSFDDTCVLCVNCFNPKDHTGHHVYTTICTEFNNGICDCGDKEAWNHTLFCKAEEGGGSHHHHHHHHHHGGGSGGGSGGGSGLNDFFEAQKIEWHEGGGSGGGSGGGSGLNDFFEAQKIEWHE

SEQ ID NO: 209 - protein, 269 aa, synthetic construct
MHSKFSHAGRICGAKFKVGEPIYRCKECSFDDTCVLCVNCFNPKDHTGHHVYTTICTEFNNGICDCGDKEAWNHTLFCKAEEGGSAGSAAGSGEFGSAGSAAGSGEFGSAGSAAGSGEFMHSKFSHAGRICGAKFKVGEPIYRCKECSFDDTCVLCVNCFNPKDHTGHHVYTTICTEFNNGICDCGDKEAWNHTLFCKAEEGGGSHHHHHHHHHHGGGSGGGSGGGSGLNDFFEAQKIEWHEGGGSGGGSGGGSGLNDFFEAQKIEWHE

SEQ ID NO: 210 - protein, 246 aa, synthetic construct
MHSKFNHAGRICGAKFRVGEPIYRCKECSFDDTCVLCVNCFNPKDHVGHHVYTSICTEFNNGICDCGDKEAWNHELNCKGAEDGGGSGGGSGGGSGMHSKFNHAGRICGAKFRVGEPIYRCKECSFDDTCVLCVNCFNPKDHVGHHVYTSICTEFNNGICDCGDKEAWNHELNCKGAEDGGSHHHHHHHHHHGGGSGGGSGGGSGLNDFFEAQKIEWHEGGGSGGGSGGGSGLNDFFEAQKIEWHE

SEQ ID NO: 211 - protein, 245 aa, synthetic construct
MHSKFNHAGRICGAKFRVGEPIYRCKECSFDDTCVLCVNCFNPKDHVGHHVYTSICTEFNNGICDCGDKEAWNHELNCKGAEDGSAGSAAGSGEFMHSKFNHAGRICGAKFRVGEPIYRCKECSFDDTCVLCVNCFNPKDHVGHHVYTSICTEFNNGICDCGDKEAWNHELNCKGAEDGGSHHHHHHHHHHGGGSGGGSGGGSGLNDFFEAQKIEWHEGGGSGGGSGGGSGLNDFFEAQKIEWHE

SEQ ID NO: 212 - protein, 269 aa, synthetic construct
MHSKFNHAGRICGAKFRVGEPIYRCKECSFDDTCVLCVNCFNPKDHVGHHVYTSICTEFNNGICDCGDKEAWNHELNCKGAEDGSAGSAAGSGEFGSAGSAAGSGEFGSAGSAAGSGEFMHSKFNHAGRICGAKFRVGEPIYRCKECSFDDTCVLCVNCFNPKDHVGHHVYTSICTEFNNGICDCGDKEAWNHELNCKGAEDGGSHHHHHHHHHHGGGSGGGSGGGSGLNDFFEAQKIEWHEGGGSGGGSGGGSGLNDFFEAQKIEWHE

SEQ ID NO: 213 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 214 - protein, 13 aa, synthetic construct
GGGSGGGSGGGSG

SEQ ID NO: 215 - protein, 12 aa, synthetic construct
GSAGSAAGSGEF

SEQ ID NO: 216 - protein, 36 aa, synthetic construct
GSAGSAAGSGEFGSAGSAAGSGEFGSAGSAAGSGEF